SEQUENCE LISTING (38 sequences)

SEQ ID NO: 1 - protein, 998 aa, synthetic construct [sequence omitted]

SEQ ID NO: 2 - protein, 976 aa, synthetic construct
  source 1..976: note = N-terminally truncated variant of beta4GalNAcT3 of Homo sapiens (L. tarentolae signal peptide and Flag tag are fused to the putative catalytic domain of human beta4GalNAc-T3 (amino acids 57-999))
MIASSVRHAVILLLVAVAMMAAVIAMDYKDDDDKRYGSWRELAKALASRNIPAVDPHLQFYHPQRLSLEDHDIDQGVSSNSSYLKWNKPVPWLSEFRGRANLHVFEDWCGSSIQQLRRNLHFPLYPHIRTTLRKLAVSPKWTNYGLRIFGYLHPFTDGKIQFAIAADDNAEFWLSLDDQVSGLQLLASVGKTGKEWTAPGEFGKFRSQISKPVSLSASHRYYFEVLHKQNEEGTDHVEVAWRRNDPGAKFTIIDSLSLSLFTNETFLQMDEVGHIPQTAASHVDSSNALPRDEQPPADMLRPDPRDTLYRVPLIPKSHLRHVLPDCPYKPSYLVDGLPLQRYQGLRFVHLSFVYPNDYTRLSHMETHNKCFYQENAYYQDRFSFQEYIKIDQPEKQGLEQPGFEENLLEESQYGEVAEETPASNNQNARMLEGRQTPASTLEQDATDYRLRSLRKLLAQPREGLLAPFSKRNSTASFPGRTSHIPVQQPEKRKQKPSPEPSQDSPHSDKWPPGHPVKNLPQMRGPRPRPAGDSPRKTQWLNQVESYIAEQRRGDRMRPQAPGRGWHGEEEVVAAAGQEGQVEGEEEGEEEEEEEDMSEVFEYVPVFDPVVNWDQTFSARNLDFQALRTDWIDLSCNTSGNLLLPEQEALEVTRVFLKKLNQRSRGRYQLQRIVNVEKRQDQLRGGRYLLELELLEQGQRVVRLSEYVSARGWQGIDPAGGEEVEARNLQGLVWDPHNRRRQVLNTRAQEPKLCWPQGFSWSHRAVVHFVVPVKNQARWVQQFIKDMENLFQVTGDPHFNIVITDYSSEDMDVEMALKRSKLRSYQYVKLSGNFERSAGLQAGIDLVKDPHSIIFLCDLHIHFPAGVIDAIRKHCVEGKMAFAPMVMRLHCGATPQWPEGYWEVNGFGLLGIYKSDLDRIGGMNTKEFRDRWGGEDWELLDRILQAGLDVERLSLRNFFHHFHSKRGMWSRRQMKTL

SEQ ID NO: 3 - protein, 1039 aa, synthetic construct
  source 1..1039: note = 4GalNAcT4 of Homo sapiens
MPRLPVKKIRKQMKLLLLLLLLSCAAWLTYVHLGLVRQGRALRQRLGYGRDGEKLTSETDGRGVHAAPSTQRAEDSSESREEEQAPEGRDLDMLFPGGAGRLPLNFTHQTPPWREEYKGQVNLHVFEDWCGGAVGHLRRNLHFPLFPHTRTTVKKLAVSPKWKNYGLRIFGFIHPARDGDVQFSVASDDNSEFWLSLDESPAAAQLVAFVGKTGSEWTAPGEFTKFSSQVSKPRRLMASRRYYFELLHKQDDRGSDHVEVGWRAFLPGLKFEVISSAHISLYTDESALKMDHVAHVPQSPASHVGGRPPQEETSADMLRPDPRDTFFLTPRMESSSLENVLEPCAYAPTYVVKDFPIARYQGLQFVYLSFVYPNDYTRLTHMETDNKCFYRESPLYLERFGFYKYMKMDKEEGDEDEEDEVQRRAFLFLNPDDFLDDEDEGELLDSLEPTEAAPPRSGPQSPAPAAPAQPGATLAPPTPPRPRDGGTPRHSRALSWAARAARPLPLFLGRAPPPRPAVEQPPPKVYVTRVRPGQRASPRAPAPRAPWPPFPGVFLHPRPLPRVQLRAPPRPPRPHGRRTGGPQATQPRPPARAQATQGGREGQARTLGPAAPTVDSNLSSEARPVTSFLSLSQVSGPQLPGEGEEEEEGEDDGAPGDEAASEDSEEAAGPALGRWREDAIDWQRTFSVGAVDFELLRSDWNDLRCNVSGNLQLPEAEAVDVTAQYMERLNARHGGRFALLRIVNVEKRRDSARGSRFLLELELQERGGGRLRLSEYVFLRLPGARVGDADGESPEPAPAASVRPDGRPELCRPLRLAWRQDVMVHFIVPVKNQARWVAQFLADMAALHARTGDSRFSVVLVDFESEDMDVERALRAARLPRYQYLRRTGNFERSAGLQAGVDAVEDASSIVFLCDLHIHFPPNILDGIRKHCVEGRLAFAPVVMRLSCGSSPRDPHGYWEVNGFGLFGIYKSDFDRVGGMNTEEFRDQWGGEDWELLDRVLQAGLEVERLRLRNFYHHYHSKRGMWSVRSRKGSRTGAS

SEQ ID NO: 4 - protein, 1012 aa, synthetic construct
  source 1..1012: note = N-terminally truncated variant of beta4GalNAcT4 of Homo sapiens L. tarentolae signal peptide and Flag tag are fused to the putative catalytic domain of human beta4GalNAc-T4 (amino acids 62- 1039)
MIASSVRHAVILLLVAVAMMAAVIAMDYKDDDDKRGVHAAPSTQRAEDSSESREEEQAPEGRDLDMLFPGGAGRLPLNFTHQTPPWREEYKGQVNLHVFEDWCGGAVGHLRRNLHFPLFPHTRTTVKKLAVSPKWKNYGLRIFGFIHPARDGDVQFSVASDDNSEFWLSLDESPAAAQLVAFVGKTGSEWTAPGEFTKFSSQVSKPRRLMASRRYYFELLHKQDDRGSDHVEVGWRAFLPGLKFEVISSAHISLYTDESALKMDHVAHVPQSPASHVGGRPPQEETSADMLRPDPRDTFFLTPRMESSSLENVLEPCAYAPTYVVKDFPIARYQGLQFVYLSFVYPNDYTRLTHMETDNKCFYRESPLYLERFGFYKYMKMDKEEGDEDEEDEVQRRAFLFLNPDDFLDDEDEGELLDSLEPTEAAPPRSGPQSPAPAAPAQPGATLAPPTPPRPRDGGTPRHSRALSWAARAARPLPLFLGRAPPPRPAVEQPPPKVYVTRVRPGQRASPRAPAPRAPWPPFPGVFLHPRPLPRVQLRAPPRPPRPHGRRTGGPQATQPRPPARAQATQGGREGQARTLGPAAPTVDSNLSSEARPVTSFLSLSQVSGPQLPGEGEEEEEGEDDGAPGDEAASEDSEEAAGPALGRWREDAIDWQRTFSVGAVDFELLRSDWNDLRCNVSGNLQLPEAEAVDVTAQYMERLNARHGGRFALLRIVNVEKRRDSARGSRFLLELELQERGGGRLRLSEYVFLRLPGARVGDADGESPEPAPAASVRPDGRPELCRPLRLAWRQDVMVHFIVPVKNQARWVAQFLADMAALHARTGDSRFSVVLVDFESEDMDVERALRAARLPRYQYLRRTGNFERSAGLQAGVDAVEDASSIVFLCDLHIHFPPNILDGIRKHCVEGRLAFAPVVMRLSCGSSPRDPHGYWEVNGFGLFGIYKSDFDRVGGMNTEEFRDQWGGEDWELLDRVLQAGLEVERLRLRNFYHHYHSKRGMWSVRSRKGSRTGAS

SEQ ID NO: 5 - protein, 383 aa, synthetic construct
  source 1..383: note = Cebeta4GalNAcT (beta4GalNAcT of Caenorhabditis elegans)
MAFRHLAVARLKSLLVLCAVLLLVHAMIYKIPSLYENLTIGSSTLIADVDAMEAVLGNTASTSDDLLDTWNSTFSPISEVNQTSFMEDIRPILFPDNQTLQFCNQTPPHLVGPIRVFLDEPDFKTLEKIYPDTHAGGHGMPKDCVARHRVAIIVPYRDREAHLRIMLHNLHSLLAKQQLDYAIFIVEQVANQTFNRGKLMNVGYDVASRLYPWQCFIFHDVDLLPEDDRNLYTCPIQPRHMSVAIDKFNYKLPYSAIFGGISALTKDHLKKINGFSNDFWGWGGEDDDLATRTSMAGLKVSRYPTQIARYKMIKHSTEATNPVNKCRYKIMGQTKRRWTRDGLSNLKYKLVNLELKPLYTRAVVDLLEKDCRRELRRDFPTCF

SEQ ID NO: 6 - protein, 393 aa, synthetic construct
  source 1..393: note = N-terminally truncated variant of Cebeta4GalNAcT of Caenorhabditis elegans L. tarentolae signal peptide and His tag are fused to the putative catalytic domain
MIASSVRHAVILLLVAVAMMAAAVIAMGSHHHHHHHHHHKIPSLYENLTIGSSTLIADVDAMEAVLGNTASTSDDLLDTWNSTFSPISEVNQTSFMEDIRPILFPDNQTLQFCNQTPPHLVGPIRVFLDEPDFKTLEKIYPDTHAGGHGMPKDCVARHRVAIIVPYRDREAHLRIMLHNLHSLLAKQQLDYAIFIVEQVANQTFNRGKLMNVGYDVASRLYPWQCFIFHDVDLLPEDDRNLYTCPIQPRHMSVAIDKFNYKLPYSAIFGGISALTKDHLKKINGFSNDFWGWGGEDDDLATRTSMAGLKVSRYPTQIARYKMIKHSTEATNPVNKCRYKIMGQTKRRWTRDGLSNLKYKLVNLELKPLYTRAVVDLLEKDCRRELRRDFPTCF

SEQ ID NO: 7 - protein, 386 aa, synthetic construct
  source 1..386: note = Ptbeta4GalNAcT (beta4GalNAcT of Parasteatoda tepidariorum)
MAQHSYSRITFSRTLICKAVILLVFTVMLLDYSYYLFSDRKPINKIVAPGLAWISHLNQKLSDTKALSNNSFLPLKPHNILLNRSLVSESSNSSKLYSSKVENKNISLNFINSSHLEPSTECPLIPPKLVGRLKVLTESPPFVALEQMFTDLNPGGRFKPNDCLSRSKVALIIPYRDREEHLRIFLHNVHPILQRQQLDYGIYIIEETANMKFNRAMLMNIGYVEASKQYPYDCFIFHDVDLIPEDDRNLYNCPEQPRHMSVAINTMNYRLPYKDIFGGVSALSKKHMKIVNGFSNEFWGWGGEDDDMSNRIKHHGLKISRYSSKIARYTMLKHKKDEPNPDRYNKLYKGKYRYTKDGLSNLKYKRLEIVFKKLYTWVLVEIEPIK

SEQ ID NO: 8 - protein, 388 aa, synthetic construct
  source 1..388: note = N-terminally truncated variant of Ptbeta4GalNAcT of Parasteatoda tepidariorum L. tarentolae signal peptide and His tag are fused to the putative catalytic domain
MIASSVRHAVILLLVAVAMMAAAVIAMGSHHHHHHHHHHSDRKPINKIVAPGLAWISHLNQKLSDTKALSNNSFLPLKPHNILLNRSLVSESSNSSKLYSSKVENKNISLNFINSSHLEPSTECPLIPPKLVGRLKVLTESPPFVALEQMFTDLNPGGRFKPNDCLSRSKVALIIPYRDREEHLRIFLHNVHPILQRQQLDYGIYIIEETANMKFNRAMLMNIGYVEASKQYPYDCFIFHDVDLIPEDDRNLYNCPEQPRHMSVAINTMNYRLPYKDIFGGVSALSKKHMKIVNGFSNEFWGWGGEDDDMSNRIKHHGLKISRYSSKIARYTMLKHKKDEPNPDRYNKLYKGKYRYTKDGLSNLKYKRLEIVFKKLYTWVLVEIEPIK

SEQ ID NO: 9 - protein, 740 aa, synthetic construct
  source 1..740: note = Stbeta4GalNAcT (beta4GalNAcT of Salmo trutta)
MGRVEGKGVLLWDQTVMCSPQYKGQANLHTFEDWCGSSTAQLRMNLHYPLYPHSRTTVKKLAVSPRWMNYGLRIFGYLHPYTDGEFVFAVSSDDNSEFWLSRDNSPLNLQLLAWIGKTGMEWTAPGEFGKYASQTSRPVRLSVQRRYFFEIVHKQNDRGTDHIEVAWQLNQEGLRFTVISSKYISLYSNESALRMSDIIHVPQTAASHTRSPSNQPDNQLSNQPAADMLRVDPRDILYQIPALDSRFLQGVLPDCSYKPSYIIKGFPLLRYQGLQFVHMSYIYPNDYTRLTHMETDNNCFYHDSPLYLDRYGFSRYMRLDEPEGQEGESRGRGRGDTFFFGMDRLVEGVDRGGGGERKGEEVGVSESLWGLEGDSEEVEGLSYDSTPRPVFDPEVNWAQTFQVTPLDLQAMRSDWIDLRCNVSGNLMLRSRDALPVVKAFMDKLNHRHHGRFTLVRVLNVERRVDGAQGSRYFLELELRDVNGQHLRLSHYIYALIRHRLSRPRGLPPLRPAPEMLLCNPVGFHWNPTATVHFIVPVKNQARWVQQLIVDMEGLYKTTGDPNFNLIITDYNSTDMDIETTLKASTLHRYQYLKLSGNFERSAGLQAGIDLITHSIVFLCDLHIHFPPSIIDSVRKHCVEGHMAFAPIVMRLDCGATPREPRGYWEVNGFGLLGIYKSDLDAAGGMNTHDFTDRWGGEDWELLDRILQSGLEVERIYMRNFLHHYHSKRGMWNRRTQRSGT

SEQ ID NO: 10 - protein, 1072 aa, synthetic construct
  source 1..1072: note = Hhbeta4GalNAcT (beta4GalNAcT of Hucho hucho)
MCVFQFGGDASEDSEAWRSSYTPQPWRPEYKGQANLHTFEDWCGSSTAQLRMNLHYPLYPHSRTTVKKLAVSPRWMNYGLRIFGYLHPYTDGEFVFAVSSDDNSEFWLSRDNSPLNLQLLAWIGKTGLEWTAPGEFGKYASQTSRPVRLSVQRRYFFEIVHKQNDRGTDHIEVAWQLNQEGLRFTVISSKYISLYSNESALRMSDITHVPQTAASHTRSPSNQLDNQLSNQPAADMLRDDPRDTLYQIPALDSRFLQGVLPDCSYKPSYIIKGFPLLRYQGLQFVHMSYIYPNDYTRLTHMETDNTCFYHDSPLYLDRYGFSRYMRLDESEGQEGESRVRGRVVGILRRRKAVPEEGLDGEAPPNVGGASKKDSAPPDYGDDYDDYAQRRRRKLFSLPQADKAPTHRNMSDTLTYKKKKRRKRRETNAAAVVVQQAVVQTRATEQDGLEIPPGPGSHTSLQTTPAEQLRLPDRPAEQLRPVKSRAGEQKPEGKPADREDMGPADRKQPRPRERPAEQNSAGLKILPAPQKLDHNLPVEGDGPTVWLPQLQGRHQRVQQLGPRGWGNATPNPLKHLTPRTVSNWSTRVGRGRRLVNRTLELWSEVTKQSTTQLPEVKPPLIKLDHKATVTRKRVQVLETDIILRGQPQIKSRPQPDYDNTLHGDLPPGVVRGMNRLVEGVDRGGGGEREGEEVGVSESLWGLEGDSEEVEGLSYDSTPRPVFDPEVNWAQTFQVTPLDLQAMRSDWIDLRCNVSGNLLLRPRDALPVVKAFMDKLNHRHHGRFTLVRVLNVERRVDGAQGSRYFLELELRDVNGQHLRLSHYIYALIRHRRSRPQGLPPLRPAPEMLLCNPVGFHWNPSATVHFIVPVKNQARWVQQLIVDMEGLYKTTGDPNFNLIITDYNSTDMDIETTLKASTLHRYQYLKLSGNFERSAGLQAGIDLITNEHSIVFLCDLHIYFPPSIIDSVRKHCVEGHMAFAPIVMRLDCGATPREPRGYWEVNGFGLLGIYKSDLDAAGGMNTHDFTDRWGGEDWELLDRILQSGLEVERIYMRNFLHHYHSKRGMWNRRTQRRST

SEQ ID NO: 11 - protein, 25 aa, synthetic construct
  source 1..25: note = SPinv, a modified signal peptide from Leishmania tarentolae invertase
MIASSVRHAVILLLVAVAMMAAVIA

SEQ ID NO: 12 - protein, 26 aa, synthetic construct
  source 1..26: note = SPinv, the native signal peptide from Leishmania tarentolae invertase
MIASSVRHAVILLLVAVAMMAAAVIA

SEQ ID NO: 13 - protein, 445 aa, synthetic construct
  source 1..445: note = MGAT1 (Alpha-1,3-mannosyl-glycoprotein 2-beta-N-acetylglucosaminyltransferase) of Homo sapiens
MLKKQSAGLVLWGAILFVAWNALLLLFFWTRPAPGRPPSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPLTWEGYDPSWN

SEQ ID NO: 14 - protein, 447 aa, synthetic construct
  source 1..447: note = MGAT2 (Alpha-1,6-mannosyl-glycoprotein 2-beta-N-acetylglucosaminyltransferase) of Homo sapiens
MRFRIYKRKVLILTLVVAACGFVLWSSNGRQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 15 - protein, 505 aa, synthetic construct
  source 1..505: note = UAP1 (Isoform AGX1 of UDP-N-acetyl hexosamine pyrophosphorylase of Homo sapiens)
MNINDLKLTLSKAGQEHLLRFWNELEEAQQVELYAELQAMNFEELNFFFQKAIEGFNQSSHQKNVDARMEPVPREVLGSATRDQDQLQAWESEGLFQISQNKVAVLLLAGGQGTRLGVAYPKGMYDVGLPSRKTLFQIQAERILKLQQVAEKYYGNKCIIPWYIMTSGRTMESTKEFFTKHKYFGLKKENVIFFQQGMLPAMSFDGKIILEEKNKVSMAPDGNGGLYRALAAQNIVEDMEQRGIWSIHVYCVDNILVKVADPRFIGFCIQKGADCGAKVVEKTNPTEPVGVVCRVDGVYQVVEYSEISLATAQKRSSDGRLLFNAGNIANHFFTVPFLRDVVNVYEPQLQHHVAQKKIPYVDTQGQLIKPDKPNGIKMEKFVFDIFQFAKKFVVYEVLREDEFSPLKNADSQNGKDNPTTARHALMSLHHCWVLNAGGHFIDENGSRLPAIPRLKDANDVPIQCEISPLISYAGEGLESYVADKEFHAPLIIDENGVHELVKNGI

SEQ ID NO: 16 - protein, 458 aa, synthetic construct
  source 1..458: note = GALK2 (N-acetyl galactosamine kinase of Homo sapiens)
MATESPATRRVQVAEHPRLLKLKEMFNSKFGSIPKFYVRAPGRVNIIGEHIDYCGYSVLPMAVEQDVLIAVEPVKTYALQLANTNPLYPDFSTSANNIQIDKTKPLWHNYFLCGLKGIQEHFGLSNLTGMNCLVDGNIPPSSGLSSSSALVCCAGLVTLTVLGRNLSKVELAEICAKSERYIGTEGGGMDQSISFLAEEGTAKLIEFSPLRATDVKLPSGAVFVIANSCVEMNKAATSHFNIRVMECRLAAKLLAKYKSLQWDKVLRLEEVQAKLGISLEEMLLVTEDALHPEPYNPEEICRCLGISLEELRTQILSPNTQDVLIFKLYQRAKHVYSEAARVLQFKKICEEAPENMVQLLGELMNQSHMSCRDMYECSCPELDQLVDICRKFGAQGSRLTGAGWGGCTVSMVPADKLPSFLANVHKAYYQRSDGSLAPEKQSLFATKPGGGALVLLEA

SEQ ID NO: 17 - protein, 348 aa, synthetic construct
  source 1..348: note = GalE (UDP-glucose 4-epimeraseof Homo sapiens)
MAEKVLVTGGAGYIGSHTVLELLEAGYLPVVIDNFHNAFRGGGSLPESLRRVQELTGRSVEFEEMDILDQGALQRLFKKYSFMAVIHFAGLKAVGESVQKPLDYYRVNLTGTIQLLEIMKAHGVKNLVFSSSATVYGNPQYLPLDEAHPTGGCTNPYGKSKFFIEEMIRDLCQADKTWNAVLLRYFNPTGAHASGCIGEDPQGIPNNLMPYVSQVAIGRREALNVFGNDYDTEDGTGVRDYIHVVDLAKGHIAALRKLKEQCGCRIYNLGTGTGYSVLQMVQAMEKASGKKIPYKVVARREGDVAACYANPSLAQEELGWTAALGLDRMCEDLWRWQKQNPSGFGTQA

SEQ ID NO: 18 - protein, 328 aa, synthetic construct
  source 1..328: note = CjGne (UDP-GlcNAc/Glc 4-epimerase of Campylobacter jejuni)
MKILISGGAGYIGSHTLRQFLKTDHEICVLDNLSKGSKIAIEDLQKTRAFKFFEQDLSDFQGVKALFEREKFDAIVHFAASIEVFESMQNPLKYYMNNTVNTTNLIETCLQTGVNKFIFSSTAATYGEPQTPVVSETSPLAPINPYGRSKLMSEEVLRDASMANPEFKHCILRYFNVAGACMDYTLGQRYPKATLLIKVAAECAAGKRDKLFIFGDDYDTKDGTCIRDFIHVDDISSAHLAALDYLKENESNVFNVGYGHGFSVKEVIEAMKKVSGVDFKVELAPRRAGDPSVLISDASKIRNLTSWQPKYDDLELICKSAFDWEKQC

SEQ ID NO: 19 - protein, 339 aa, synthetic construct
  source 1..339: note = UDP-GalNAc transporter of C. elegans (CeC03H5.2)
MNRANDTSSNLKLISLVVLIVQTTALVLTLRYSQTQKSEGPRYLSSTAVVCAEIIKLITCFFVIYRNNGYRFSGMLNELNREIFASPQTRADSLKVAVPAIMYVIQNNLLFFALKKLDAATYQVTYQLKILTTAIFSVTMLGKSLHRYNWMALILLTAGVALVQYPSGDSTTSKSTAAEHDASDNILGLGAVLAACFSSGFAGVYFEKILKTSKVSLWIRNIQLAFFSVFGALLVCWLYDWQAISDDGFLRGYNGVIWIVVLLQAYGGLVIALVVKYADNILKGFAVSLSIILSSFTSWLVLGDLTITTTFAIGATVVIFATFLYGHEPKSTPAEAHNA

SEQ ID NO: 20 - RNA, 28 nt, synthetic construct
  source 1..28: mol_type = other RNA; note = tracrRNA
agttaaaataaggctagtccgttatcaa

SEQ ID NO: 21 - RNA, 24 nt, synthetic construct
  source 1..24: mol_type = other RNA; note = OGNT1_3prime
gaaatccgatggttttagagctat

SEQ ID NO: 22 - RNA, 24 nt, synthetic construct
  source 1..24: mol_type = other RNA; note = OGNT2_3prime
ggaacggcctggttttagagctat

SEQ ID NO: 23 - RNA, 24 nt, synthetic construct
  source 1..24: mol_type = other RNA; note = OGNT2_5prime
caaccgaatccgttttagagctat

SEQ ID NO: 24 - RNA, 24 nt, synthetic construct
  source 1..24: mol_type = other RNA; note = OGNTL_5prime
cgaactcgcaggttttagagctat

SEQ ID NO: 25 - RNA, 24 nt, synthetic construct
  source 1..24: mol_type = other RNA; note = OGNT1_5prime
cgggtgaggtggttttagagctat

SEQ ID NO: 26 - RNA, 24 nt, synthetic construct
  source 1..24: mol_type = other RNA; note = OGNTL_3prime
gtggcgccaaggttttagagctat

SEQ ID NO: 27 - DNA, 25 bp, synthetic construct
  source 1..25: mol_type = other DNA; note = primer 360
ccatggccagatgccaagcttactc

SEQ ID NO: 28 - DNA, 25 bp, synthetic construct
  source 1..25: mol_type = other DNA; note = primer 363
cggaacacagacctgccatctgtgg

SEQ ID NO: 29 - DNA, 25 bp, synthetic construct
  source 1..25: mol_type = other DNA; note = primer 362
ccacagatggcaggtctgtgttccg

SEQ ID NO: 30 - DNA, 25 bp, synthetic construct
  source 1..25: mol_type = other DNA; note = primer 361
catccacctctgctcgtcgtccttc

SEQ ID NO: 31 - DNA, 25 bp, synthetic construct
  source 1..25: mol_type = other DNA; note = primer 290
cgcacctcggcttcaactgccaatg

SEQ ID NO: 32 - DNA, 25 bp, synthetic construct
  source 1..25: mol_type = other DNA; note = primer 291
aacgtgcctgaagcccgtagctttg

SEQ ID NO: 33 - DNA, 25 bp, synthetic construct
  source 1..25: mol_type = other DNA; note = primer 292
ggcgacggatgatgcccaccatttg

SEQ ID NO: 34 - DNA, 25 bp, synthetic construct
  source 1..25: mol_type = other DNA; note = primer 293
ggaggcaaccaagaaggcgtcagag

SEQ ID NO: 35 - DNA, 35 bp, synthetic construct
  source 1..35: mol_type = other DNA; note = primer 127
gctcagctgcgagagcgtaaacctccgtaacaatg

SEQ ID NO: 36 - DNA, 35 bp, synthetic construct
  source 1..35: mol_type = other DNA; note = primer 128
agctaaactctgcactcatctgcgcttccgcaagg

SEQ ID NO: 37 - DNA, 35 bp, synthetic construct
  source 1..35: mol_type = other DNA; note = primer 161
cgaccgcgatatctgctttccattctgcagttctc

SEQ ID NO: 38 - DNA, 35 bp, synthetic construct
  source 1..35: mol_type = other DNA; note = primer 160
cgcacagggtgacgaggaaagcagtatcgaaacag

(19) United States
(12) Patent Application Publication
(10) Pub. No.: US 2026/0365752 A1
(43) Pub. Date: Nov. 19, 2026

(54) GLYCAN-MEDIATED PROTEIN DEGRADATION

(71) Applicant: GlycoEra AG, Wädenswil (CH)

(72) Inventors: Jonathan Albert Back, Villers-le-Lac (FR); Amirreza Faridmoayer, Oberengstringen (CH); Tanmoy Ganguly, Westford, MA (US); Ganesh Kaundinya, Bedford, MA (US); Manuela Mally, Watt (CH)

(21) Appl. No.: 19/116,229

(22) PCT Filed: Sep. 27, 2023
(86) PCT No.: PCT/EP2023/076745

§ 371 Date: Mar. 27, 2025

Related U.S. Application Data
(60) Provisional application No. 63/410,936, filed on Sep. 28, 2022.
(60) Provisional application No. 63/410,955, filed on Sep. 28, 2022.

(51) Int. Cl. C07K 16/30; C12N 1/10; C12R 1/90

(52) U.S. Cl. CPC C07K 16/30; C12N 1/10; C07K 2317/14; C07K 2317/21; C07K 2317/24; C07K 2317/41; C07K 2317/53; C07K 2317/55; C07K 2317/56; C07K 2317/77; C07K 2319/00; C12R 2001/90

(57) ABSTRACT

The present application relates to glycoengineered bifunctional degraders, populations of glycoengineered bifunctional degraders, Leishmania host cells for producing glycoengineered bifunctional degraders, methods of engineering said Leishmania host cells, methods of culturing said Leishmania host cells, methods of making glycoengineered bifunctional degraders using Leishmania host cells, and methods of using glycoengineered bifunctional degraders. In particular, the glycoengineered bifunctional degraders comprise a biantennary, GalNAc-terminated N-glycan, specifically A2GalNAc2.

### 1. CROSS REFERENCE TO RELATED APPLICATIONS

This application claims the benefit of priority to U.S. Ser. No. 63/410,955, filed Sep. 28, 2022, and U.S. Ser. No. 63/410,936, filed Sep. 28, 2022, each of which is incorporated herein by reference in its entirety.

### 2. SEQUENCE LISTING

This application contains a computer readable Sequence Listing which has been submitted in XML file format with this application, the entire content of which is incorporated by reference herein in its entirety. The Sequence Listing XML file submitted with this application is entitled “14688-005-228_SEQLISTING.xml”, was created on Sep. 22, 2023 and is 52,246 bytes in size.

### 3. INTRODUCTION

The present application relates to glycoengineered bifunctional degraders, populations of glycoengineered bifunctional degraders, Leishmania host cells for producing glycoengineered bifunctional degraders, methods of engineering said Leishmania host cells, methods of culturing said Leishmania host cells, methods of making glycoengineered bifunctional degraders using Leishmania host cells, and methods of using glycoengineered bifunctional degraders. In particular, the glycoengineered bifunctional degraders comprise a biantennary, GalNAc-terminated N-glycan, specifically A2GalNAc2.

### 4. BACKGROUND

A glycoprotein is a glycoconjugate in which a protein carries one or more glycans covalently attached to a polypeptide backbone, usually via N- or O-linkages. An N-glycan (N-linked oligosaccharide, N-[Asn]-linked oligosaccharide) is a sugar chain covalently linked to an asparagine residue of a polypeptide chain, commonly involving a GlcNAc residue in eukaryotes, and the consensus peptide sequence: Asn-X-Ser/Thr (Varki, Ajit (2009): Essentials of glycobiology. 2ed. Cold Spring Harbor, N.Y.: Cold Spring Harbor Laboratory Press).

Protein glycosylation is a ubiquitous post-translational modification found in all domains of life. There is a significant complexity in animal systems and glycan structures have crucial biological and physiological roles, from contributions in protein folding and quality, control to involvement in a large number of biological events, like recognition, stability, action, and turnover of these molecules (Moremen et al. 2012). Therapeutic Glycoproteins like monoclonal antibodies, enzymes, and hormones are the major products of the biotechnology industry (Lagassé, H A Daniel et al. 2017; Dimitrov 2012) and the impact of glycan heterogeneity has more and more been recognized as “critical quality attribute”. Of the many properties determining product quality, glycosylation is regarded as even one of the most important ones: influencing the biological activity, serum half-life and immunogenicity of the protein. Glycans are relevant for increased serum circulation times and many of the biologics approved or under development suffer from an insufficient half-life necessitating frequent applications in order to maintain a therapeutic concentration over an extended period of time. Half-life extension strategies are key to allow the generation of long-lasting therapeutics with improved pharmacokinetics (Kontermann 2016). Glycosylation also appears to improve protein solubility and stability, for example, through a reduced propensity for aggregation and leads to increased circulatory lifetimes due to the prevention of proteolytic degradation. Additionally, N-glycans with different terminating monosaccharides can be recognized by lectins leading to their degradation (Blasko et al., 2013; Varki, 2017). Consequently, monitoring and control of glycosylation is critical in biopharmaceutical manufacturing and a requirement of regulatory agencies (Costa et al. 2014; Eon-Duval et al. 2012; Reusch and Tejada 2015). For these reasons, glycoengineering of expression platforms is increasingly recognized as an important strategy to improve biopharmaceuticals in many aspects (Dicker and Strasser 2015).

Endocytic lectins are involved in receptor-mediated endocytosis by capturing glycosylated proteins via specific glycan structures to mediate degradation (Cummings et al., Cold Spring Harbor Laboratory Press, (2017). Endocytic lectins are ubiquitous in humans and can recognize various glycan structures.

Carbohydrate binding receptors are highly diverse and can be exploited by glycoengineering to develop novel therapeutics with unprecedented effectiveness for different diseases, including but not limited to: inflammatory, blood disorders, autoimmune and cancer. This allows development of novel therapeutics based on the concept of glycan-mediated protein degradation. Leveraging natural protein degradation through the glycosylation of monoclonal antibodies can lead to novel therapeutics. The present invention shows a novel finding of Leishmania host cells are capable of producing polypeptides comprising a biantennary, GalNAc-terminated N-glycan, specifically A2GalNAc2, which mediates protein degradation.

The compositions and methods provided herein address the unmet medical need of patients suffering from various difficult to treat diseases such as cancer, autoimmune and inflammatory diseases, and infectious diseases, treated with glycosylated proteins, such as monoclonal antibodies, and provide related advantages.

### 5. SUMMARY OF THE INVENTION

Provided herein are glycoengineered bifunctional degraders, populations of glycoengineered bifunctional degraders, Leishmania host cells for producing glycoengineered bifunctional degraders, methods of engineering said Leishmania host cells, methods of culturing said Leishmania host cells, methods of making glycoengineered bifunctional degraders using Leishmania host cells, and methods of using glycoengineered bifunctional degraders.

Without being bound by theory, the glycoengineered bifunctional degraders of the present invention, in which peptidyl molecules, such as antibodies and fragments thereof, are modified with one or more A2GalNAc2 glycans, are optimally suited for the bifunctional role of binding to a desired target protein, and engaging an ASGPR receptor via the N-glycan moiety. Without being bound by theory, it is believed that the bifunctional degrader simultaneously binds to the target protein and to an ASGPR receptor(s) present on liver cells, wherein the complex so formed is internalized and targeted for degradation via the lysosomal pathway. The inventors have found that the bifunctional degraders of the present invention, modified with one or more A2GalNAc2 glycans, are unexpectedly efficient for internalization and/or degradation of target proteins, as compared to similar molecules modified with other N-glycans, such as triantennary, GalNAc2-terminated glycans, which have most often been described for use in so-called “Lysosome-Targeting Chimaerae” or “LYTACs.” See, Zhou et al. (2021) ACS Cent. Sci. 7:499-506; Ahn et al. Nat Chem Biol. 2021 September; 17(9):937-946; Zhao et al. (2022) Signal Transduction and Targeted Therapy 7:113; and Zhong et al. (2022) Antibodies 11:5, the disclosure of each of which is hereby incorporated herein in its entirety by reference.

Provided herein is a glycoengineered bifunctional degrader, wherein the bifunctional degrader (i) specifically binds to a target protein and (ii) comprises an N-glycan of the structure:

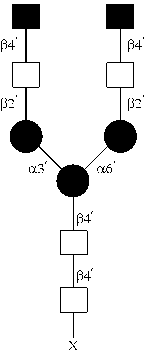

linked to the bifunctional degrader at one or more N-glycosylation sites, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In certain embodiments, the N-glycan is linked to the bifunctional degrader at least one N-glycosylation site. In certain embodiments, the N-glycan is linked to the bifunctional degrader at least two N-glycosylation sites. In certain embodiments, wherein the N-glycan is linked to the bifunctional degrader at one, two, three, or four N-glycosylation sites. In certain embodiments, the N-glycan is linked to the bifunctional degrader at one N-glycosylation site. In certain embodiments, the N-glycan is linked to the bifunctional degrader at two N-glycosylation sites. In certain embodiments, the N-glycan is linked to the bifunctional degrader at three N-glycosylation sites. In certain embodiments, the N-glycan is linked to the bifunctional degrader at four N-glycosylation sites. In certain embodiments, the amino acid residue is Asn. In certain embodiments, the N-glycosylation site comprises a consensus sequence of N-X-S/T or N-X-C, wherein X is any amino acid except proline.

In certain embodiments, the one or more N-glycosylation sites are distal to a target-specific binding location of the bifunctional degrader. In certain embodiments, at least one or at least two N-glycosylation sites are distal to the target-specific binding location. In certain embodiments, all of the N-glycosylation sites are distal to the target-specific binding location. In certain embodiments, the one or more N-glycosylation sites are not present in a wild-type, natural, synthetic, or commercial precursor of the bifunctional degrader. In certain embodiments, the target-specific binding location is a variable region of an antibody or antigen-binding fragment (Fab), or an ectodomain of an Fc-fusion protein.

In certain embodiments, one or more N-glycosylation sites present in a wild-type, natural, synthetic, or commercial precursor of the bifunctional degrader (“natural N-glycosylation sites”) have been deleted, mutated, or functionally inactivated. In certain embodiments, at least one or at least two natural N-glycosylation sites have been deleted, mutated, or functionally inactivated. In certain embodiments, all natural N-glycosylation sites have been deleted, mutated, or functionally inactivated. In certain embodiments, the one or more natural N-glycosylation sites are located at or proximal to the target-specific binding location of the wild-type, natural, synthetic, or commercial precursor of the bifunctional degrader. In certain embodiments, the target-specific binding location is a variable region of an antibody or antigen-binding fragment (Fab), or an ectodomain of an Fc-fusion protein. In certain embodiments, one or more natural N-glycosylation sites have been deleted, mutated, or functionally inactivated and one or more glycoengineered N-glycosylation sites are present in the bifunctional degrader. In certain embodiments, one or more glycoengineered N-glycosylation sites are located distal to a target-specific binding location of the bifunctional degrader.

In certain embodiments, the bifunctional degrader is an antibody or fragment thereof. In certain embodiments, the bifunctional degrader is a Fab fragment of an antibody. In certain embodiments, the glycoengineered bifunctional degrader is an antibody. In certain embodiments, the antibody is a monoclonal or polyclonal antibody. In certain embodiments, the antibody is a recombinant antibody. In certain embodiments, the antibody is humanized, chimeric or fully human. In certain embodiments, the antibody has a glycan to protein ratio of 2 to 1, 4 to 1, 6 to 1, 8 to 1, or 10 to 1. In certain embodiments, the N-glycan is linked to an N-glycosylation site of the light chain of the antibody or fragment thereof. In certain embodiments, the N-glycan is linked to an N-glycosylation site of the heavy chain of the antibody or fragment thereof. In certain embodiments, one or more N-glycosylation sites are located in a constant domain of the antibody or fragment thereof. In certain embodiments, one or more N-glycosylation sites are located in a variable domain of the antibody or fragment thereof. In certain embodiments, one or more N-glycosylation sites are located on the Fab region of the antibody. In certain embodiments, one or more N-glycosylation sites are located on the Fc region of the antibody. In certain embodiments, one or more N-glycosylation sites are located in the hinge region of the antibody. In certain embodiments, at least one of the N-glycosylation sites is not present in a wild-type form of the antibody. In certain embodiments, at least two N-glycosylation sites of the Fab region of the antibody are glycosylated by the N-glycan. In certain embodiments, one N-glycosylation site of the Fab region is located on each of the two heavy chain polypeptides of the antibody, and wherein each of said N-glycosylation sites is glycosylated by the N-glycan. In certain embodiments, at least two N-glycosylation sites of the Fc region of the antibody are glycosylated by the N-glycan. In certain embodiments, the Fab region contains more of the N-glycans than the Fc region. In certain embodiments, the Fab region contains two more of the N-glycans than the Fc region. In certain embodiments, the Fc region contains more of the N-glycans than the Fab region. In certain embodiments, the Fc region contains two or four more of the N-glycans than the Fab region. In certain embodiments, the Fc region and the Fab region contain the same number of the N-glycans. In certain embodiments, the glycoengineered bifunctional degrader binds to an autoantibody and comprises an autoantigen or immunogenic fragment thereof.

In certain embodiments, the glycoengineered bifunctional degrader comprises a moiety that specifically binds to the target protein, and wherein the target protein is associated with a disease. In certain embodiments, the target protein is a cell surface molecule or a non-cell surface molecule. In certain embodiments, the cell surface molecule is a receptor. In certain embodiments, the non-cell surface molecule is an extracellular protein. In certain embodiments, the extracellular protein is an autoantibody, a hormone, a cytokine, a chemokine, a blood protein, or a central nervous system (CNS) protein. In certain embodiments, the target protein associated with a disease is upregulated in the disease compared to a non-disease state. In certain embodiments, the target protein associated with a disease is expressed in the disease compared to a non-disease state. In certain embodiments, the target protein associated with a disease is involved in cancer progression. In certain embodiments, the target protein associated with disease comprises TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, CD38, CD73, or TGF-β, Bombesin R, CAIX, CD13, CD44, v6, Emmprin, Endoglin, EpCAM, EphA2, FAP-α, Folate R, GRP78, IGF-1R, Matriptase, Mesothelin, sMET/HGFR, MT1-MMP, MT6-MMP, PSCA, PSMA, Tn antigen, and uPAR, TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, LILRB1, LILRB2, VEGF-R, CXCR4, CXCL12, CSF-1, CD47, aggregated light chains or aggregated transthyretin. In certain embodiments, the target protein associated with disease is involved in an autoimmune disease, and wherein the target protein is an antibody binding to TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin, or gangliosides GM1, GD3 or GQ1B. In certain embodiments, the disease comprises a cancer. In certain embodiments, the disease comprises an autoimmune disease.

Also provided herein is a composition comprising a population of bifunctional degraders described herein, wherein the population of bifunctional degraders has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s). In certain embodiments, the N-glycan profile is at least 30%, at least 40%, at least 50%, at least 60%, at least 70%, at least 80%, at least 90%, at least 95%, or at least 98% homogenous at one of the N-glycosylation sites. In certain embodiments, the N-glycan profile is at least 30%, at least 40%, at least 50%, at least 60%, at least 70%, at least 80%, at least 90%, at least 95%, or at least 98% homogeneous at two of the N-glycosylation sites. In certain embodiments, the N-glycan profile is at least 50% homogeneous at one or more of the N-glycosylation site(s). In certain embodiments, the N-glycan profile is at least 60% homogeneous at one or more of the N-glycosylation site(s). In certain embodiments, the N-glycan profile is at least 70% homogenous at one or more of the N-glycosylation site(s). In certain embodiments, the N-glycan profile is at least 80% homogeneous at one or more of the N-glycosylation site(s). In certain embodiments, the N-glycan profile is at least 90% homogeneous at one or more of the N-glycosylation site(s). In certain embodiments, the N-glycan profile is at least 95% homogenous at one or more of the N-glycosylation site(s). In certain embodiments, the N-glycan profile is at least 98% homogenous at one or more of the N-glycosylation site(s). In certain embodiments, the homogeneity of the N-glycan profile at one or more of the N-glycosylation sites is determined by N-glycan analysis, glycopeptide analysis or intact protein analysis.

In certain embodiments, the N-glycan profile comprises about 30% to 40%, about 40% to about 50%, about 50% to about 60%, about 60% to about 70%, about 70% to about 80%, about 80% to about 90%, or about 90% to about 100% of an N-glycan of the structure:

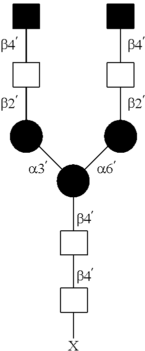

at one or more of the N-glycosylation site(s), wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In certain embodiments, the N-glycan profile comprises about 90% to about 100% of the N-glycan at one N-glycosylation site. In certain embodiments, the N-glycan profile comprises about 95% to about 100% of the N-glycan at one N-glycosylation site. In certain embodiments, the N-glycan profile comprises about 90% to about 100% of the N-glycan at two N-glycosylation sites, respectively. In certain embodiments, the N-glycan profile comprises about 80% to about 90% of the N-glycan at two N-glycosylation sites, collectively. In certain embodiments, the N-glycan profile comprises about 90% to about 100% of the N-glycan at two N-glycosylation sites, collectively. In certain embodiments, the N-glycan profile comprises about 90% to about 100% of the N-glycan at three or more of the N-glycosylation site(s), respectively. In certain embodiments, the N-glycan profile comprises about 70% to about 100% of the N-glycan at three or more of the N-glycosylation site(s), collectively. In certain embodiments, the relative amount of the N-glycan at one or more of the N-glycosylation sites in determined by N-glycan analysis or glycopeptide analysis.

In certain embodiments, the population of bifunctional degraders has an N-glycan profile comprising at least 30%, at least 40%, at least 50%, at least 60%, at least 70%, at least 80%, at least 90%, at least 95%, or at least 98% of an N-glycan of the structure:

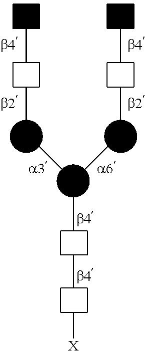

among all glycans in the N-glycan profile, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In certain embodiments, the population of bifunctional degraders has an N-glycan profile comprising at least 30% of the N-glycan among all glycans in the N-glycan profile. In certain embodiments, the population of bifunctional degraders has an N-glycan profile comprising at least 40% of the N-glycan among all glycans in the N-glycan profile. In certain embodiments, the population of bifunctional degraders has an N-glycan profile comprising at least 50% of the N-glycan among all glycans in the N-glycan profile. In certain embodiments, the population of bifunctional degraders has an N-glycan profile comprising at least 60% of the N-glycan among all glycans in the N-glycan profile. In certain embodiments, the population of bifunctional degraders has an N-glycan profile comprising at least 70% of the N-glycan among all glycans in the N-glycan profile. In certain embodiments, the population of bifunctional degraders has an N-glycan profile comprising at least 80% of the N-glycan among all glycans in the N-glycan profile. In certain embodiments, the population of bifunctional degraders has an N-glycan profile comprising at least 90% of the N-glycan among all glycans in the N-glycan profile. In certain embodiments, the population of bifunctional degraders has an N-glycan profile comprising at least 95% of the N-glycan among all glycans in the N-glycan profile. In certain embodiments, the population of bifunctional degraders has an N-glycan profile comprising at least 98% of the N-glycan among all glycans in the N-glycan profile.

In certain embodiments, the population of bifunctional degraders has an N-glycan profile comprising about 30% to about 40%, about 40% to about 50%, about 50% to about 60%, about 60% to about 70%, about 70% to about 80%, about 80% to about 90%, or about 90% to about 100% of the N-glycan among all glycans in the N-glycan profile. In certain embodiments, the population of bifunctional degraders has an N-glycan profile comprising about 30% to about 40% of the N-glycan among all glycans in the N-glycan profile. In certain embodiments, the population of bifunctional degraders has an N-glycan profile comprising about 40% to about 50% of the N-glycan among all glycans in the N-glycan profile. In certain embodiments, the population of bifunctional degraders has an N-glycan profile comprising about 50% to about 60% of the N-glycan among all glycans in the N-glycan profile. In certain embodiments, the population of bifunctional degraders has an N-glycan profile comprising about 60% to about 70% of the N-glycan among all glycans in the N-glycan profile. In certain embodiments, the population of bifunctional degraders has an N-glycan profile comprising about 70% to about 80% of the N-glycan among all glycans in the N-glycan profile. In certain embodiments, the population of bifunctional degraders has an N-glycan profile comprising about 80% to about 90% of the N-glycan among all glycans in the N-glycan profile. In certain embodiments, the population of bifunctional degraders has an N-glycan profile comprising about 90% to about 100% of the N-glycan among all glycans in the N-glycan profile. In certain embodiments, the relative amount of the N-glycan among all glycans in the N-glycan profile is determined by N-glycan analysis, glycopeptide analysis or intact protein analysis.

In certain embodiments, the population has an N-glycan profile that is at least 30%, at least 40%, at least 50%, at least 60%, at least 70%, at least 80%, at least 90%, at least 95%, or at least 98%. In certain embodiments, the population has an N-glycan profile that is at least 30% homogeneous. In certain embodiments, the population has an N-glycan profile that is at least 40% homogeneous. In certain embodiments, the population has an N-glycan profile that is at least 50% homogeneous. In certain embodiments, the population has an N-glycan profile that is at least 60% homogeneous. In certain embodiments, the population has an N-glycan profile that is at least 70% homogeneous. In certain embodiments, the population has an N-glycan profile that is at least 80% homogeneous. In certain embodiments, the population has an N-glycan profile that is at least 90% homogeneous. In certain embodiments, the population has an N-glycan profile that is at least 95% homogeneous. In certain embodiments, the population has an N-glycan profile that is at least 98% homogeneous.

In certain embodiments, the population has an N-glycan profile that is about 30% to about 40%, about 40% to about 50%, about 50% to about 60%, about 60% to about 70%, about 70% to about 80%, about 80% to about 90%, or about 90% to about 100% homogeneous. In certain embodiments, the population has an N-glycan profile that is about 30% to about 40% homogeneous. In certain embodiments, the population has an N-glycan profile that is about 40% to about 50% homogeneous. In certain embodiments, the population has an N-glycan profile that is about 50% to about 60% homogeneous. In certain embodiments, the population has an N-glycan profile that is about 60% to about 70% homogeneous. In certain embodiments, the population has an N-glycan profile that is about 70% to about 80% homogeneous. In certain embodiments, the population has an N-glycan profile that is about 80% to about 90% homogeneous. In certain embodiments, the population has an N-glycan profile that is about 90% to about 100% homogeneous. In certain embodiments, the homogeneity of the N-glycan profile is determined by N-glycan analysis, glycopeptide analysis or intact protein analysis.

In certain embodiments, the bifunctional degrader in said population is expressed from one or more nucleic acid sequences in a Leishmania host cell.

### 5.1 Definitions

As used herein the term “glycan” can refer to an N-glycan. Based on the specific structure, the skilled artisan would know if a specific glycan is an N-linked glycan.

The term “about,” when used in conjunction with a number, refers to any number within ±1, ±5 or ±10% of the referenced number.

As used herein, the term “patient” refers to an animal (e.g., birds, reptiles, and mammals). In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In some embodiments, a subject is a non-human animal. In some embodiments, a subject is a farm animal or pet (e.g., a dog, cat, horse, goat, sheep, pig, donkey, or chicken). In a specific embodiment, a subject is a human. The terms “subject” and “patient” can be used herein interchangeably.

The abbreviations “α[number]”, “α[number], [number]”, “β[number]”, or “β[number], [number]” refer to glycosidic bonds or glycosidic linkages which are covalent bonds that join a carbohydrate residue to another group. An α-glycosidic bond is formed when both carbons have the same stereochemistry, whereas a β-glycosidic bond occurs when the two carbons have different stereochemistry.

As used herein, the term “glycoengineering,” “glycoengineered,” or an equivalent thereof means a process of glycosylating a target protein, or a target protein (e.g., bifunctional degrader) made by such process, wherein the process uses an in vivo host cell system that has one or more enzymes (e.g., pathways) that provides for glycosylation of the target protein. Such a host cell system can be genetically engineered to introduce a glycosylation pathway to selectively glycosylate a target protein with a particular glycan structure. A host cell used to generate a glycoengineered target protein can include, for example, a recombinant nucleic acid encoding a target protein; and a recombinant nucleic acid encoding a heterologous glycosyltransferase. The host cell system used for glycoengineering (e.g., to generate a glycoengineered protein) can introduce N-linked glycosylation. The host cell used for glycoengineering or to generate a glycoengineered target protein can be a mammalian cell, an insect cell, a yeast cell, a bacterial cell, a plant cell, a microalgae, or a protozoa. The protozoa used for glycoengineering can be a species of Leishmania. A glycoengineered bifunctional degrader also includes a bifunctional degrader that has been engineered to be selectively glycosylated at one or more specific sites when generated in the host cell system.

As used herein, the term “glycosite” or “glycosylation site” refers to a site of glycosylation in a protein. Such a glycosite can be naturally present in the amino acid sequence of a protein or recombinantly engineered into the protein by addition or substitution or deletion of amino acids. In a specific embodiment, a glycosite is present in a so-called glycotag that is fused to a bifunctional degrader provided herein. In certain embodiments, a glycotag is fused to a protein to create a bispecific binding protein. As used herein a glycotag refers to a peptide containing consensus N-glycosylation site sequence fused to N- or a C-terminal or both termini of a protein or polypeptide. In some embodiments, the glycotag is fused to the C-terminus of the bifunctional degrader via a peptide linker. In some embodiments, the glycotag is fused to the N-terminus of the bifunctional degrader via a peptide linker. In some embodiments, the peptide linker is a consensus peptide sequence. In some embodiments, the consensus peptide sequence is 1, 2, 3, 4, 5, 6, 7 or more amino acid residues in length. In some embodiments, the bifunctional degrader provided herein contains 1, 2, 3, 4, 5, 6, 7, 8, 9, 10 or more glycotags.

As used herein, a “glycoengineered bifunctional degrader” or “bifunctional degrader” is a polypeptide that mediates the degradation of a target protein by specifically binding to the target protein and engaging with endocytic receptors to activate degradation pathways.

As used herein, a bifunctional degrader provided herein is “glycosylated” by an N-glycan if the N-glycan is linked to the bifunctional degrader at one or more site of the bifunctional degrader. In certain embodiments, the N-glycan is linked to an N-glycosylation site of the bifunctional degrader. As used herein, an N-glycosylation site is “occupied” by an N-glycan if the N-glycan is linked to the N-glycosylation site.

In certain embodiments, N-glycosylation sites that are not present in a wild-type, natural, synthetic, and/or commercial precursor of the bifunctional degrader (i.e. that are engineered into the amino acid sequence of the wild-type, natural, synthetic, and/or commercial precursor of the bifunctional degrader) are referred to herein as “glycoengineered N-glycosylation sites”. In certain embodiments, N-glycosylation sites that are not present in a wild-type precursor of the bifunctional degrader are referred to herein as “glycoengineered N-glycosylation sites”. In certain embodiments, N-glycosylation sites that are not present in a natural precursor of the bifunctional degrader are referred to herein as “glycoengineered N-glycosylation sites”. In this context, the term “natural” encompasses anything made in or derived from a biological system. In certain embodiments, N-glycosylation sites that are not present in a synthetic precursor of the bifunctional degrader are referred to herein as “glycoengineered N-glycosylation sites”. In certain embodiments, N-glycosylation sites that are not present in a commercial precursor of the bifunctional degrader are referred to herein as “glycoengineered N-glycosylation sites”. In certain embodiments, the wild-type, natural, synthetic, and/or commercial precursor of the bifunctional degrader is selected from a wild-type, natural, synthetic, and/or commercial precursor of the degrader described in Section 7.1.

In certain embodiments, the wild-type, natural, synthetic, and/or commercial precursor of the bifunctional degrader is also referred to herein as an “unmuted form of the bifunctional degrader”. In certain embodiments, the wild-type precursor of the bifunctional degrader is referred to herein as an “unmuted form of the bifunctional degrader”. In certain embodiments, the natural precursor of the bifunctional degrader is referred to herein as an “unmuted form of the bifunctional degrader”. In this context, the term “natural” encompasses anything made in or derived from a biological system. In certain embodiments, the synthetic precursor of the bifunctional degrader is referred to herein as an “unmuted form of the bifunctional degrader”. In certain embodiments, the commercial precursor of the bifunctional degrader is referred to herein as an “unmuted form of the bifunctional degrader”. In certain embodiments, the wild-type, natural, synthetic, and/or commercial precursor of the bifunctional degrader is selected from a wild-type, natural, synthetic, and/or commercial precursor of the degrader described in Section 7.1.

In certain embodiments, one or more of the N-glycosylation sites that are present in a wild-type, natural, synthetic, and/or commercial precursor of the bifunctional degrader are referred to herein as “natural N-glycosylation sites”. In certain embodiments, one or more of the N-glycosylation sites that are present in a wild-type precursor of the bifunctional degrader are referred to herein as “natural N-glycosylation sites”. In certain embodiments, one or more of the N-glycosylation sites that are present in a natural precursor (i.e. anything made in or derived from a biological system) of the bifunctional degrader are referred to herein as “natural N-glycosylation sites”. In certain embodiments, one or more of the N-glycosylation sites that are present in a synthetic precursor of the bifunctional degrader are referred to herein as “natural N-glycosylation sites”. In certain embodiments, one or more of the N-glycosylation sites that are present in a commercial precursor of the bifunctional degrader are referred to herein as “natural N-glycosylation sites”. In certain embodiments, the wild-type, natural, synthetic, and/or commercial precursor of the bifunctional degrader is selected from a wild-type, natural, synthetic, and/or commercial precursor of the degrader described in Section 7.1.

As used herein, the terms “distal” and “proximal” refer to the proximity in three-dimensional space of, for example, an N-glycan or N-glycosylation site to a specific region of the bifunctional degrader and, thus, relate to the quaternary structure of the bifunctional degrader as opposed to its primary amino acid sequence.

As used herein, the term “inflammatory disorder” includes disorders, diseases or conditions characterized by inflammation. Examples of inflammatory disorders include allergy, asthma, autoimmune diseases, coeliac disease, glomerulonephritis, hepatitis, inflammatory bowel disease, reperfusion injury and transplant rejection, among others.

As used herein, the term “blood disorder” includes a disorders, diseases or conditions that affect blood. Examples of blood disorders include anemia, bleeding disorders such as hemophilia, blood clots, and blood cancers such as leukemia, lymphoma, and myeloma, among others.

As used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

### 5.2 Conventions and Abbreviations

| Abbreviation | Convention |
|---|---|
| CDS | Coding sequence |
| EPO | Erythropoietin |
| GlcNAc- | N-acetylglucosamine-transferase |
| transferase | |
| IR | Intergenic region |
| GalNAc | N-acetylgalactosamine |
| Fuc | Fucose |
| Glc | Glucose |
| GlcNAc | N-acetylglucosamine |
| HexNAc | N-acetylhexosamines |
| OGNT | UDP-N-acetylglucosamine-peptide |
| | N-acetylglucosaminyltransferase |
| ORF | Open reading frames |
| UTR | Untranslated region |

### 5.3 N-Glycan Nomenclature

| | Short IgG | Symbol |
|---|---|---|
| | nomenclature² | Nomenclature for |
| Oxford/Unifi | or | Glycan |
| nomenclature¹ | alternative name | structure³ |
| M3 | Man3 | 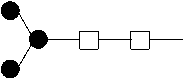 |
| A1 | G0-N | 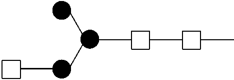 |
| A2 | G0 | 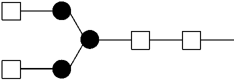 |
| A2G(4)2 | G2 | 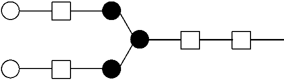 |
| A2G2S2 | G2S2 | 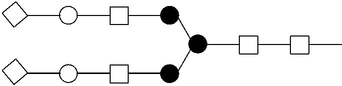 |
| A1[3]GalNAc(4)1 | A1GalNAc1 Or Mono- antennary GalNAc | 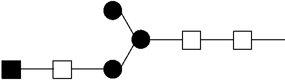 |
| A2[3](2)GalNAc(4)1 or A2[6](2)GalNAc(4)1 | A2GalNAc1 | 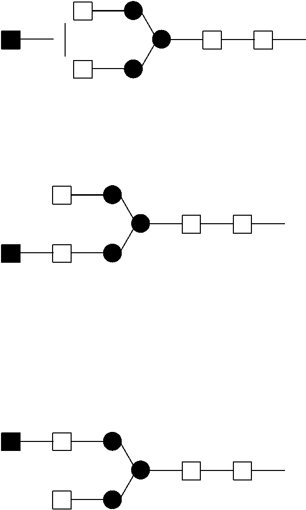 |
| A2GalNAc(4)2 | A2GalNAc2 or Bi-antennary GalNAc or Bi-GalNAc | 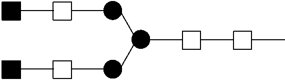 |
| A2G(4)2F(6) | A2F | 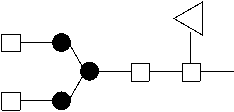 |

¹Mx: number (x) of residues within the oligomannose series; Ax: number (x) of antennae; F: core fucose; Gx: number (x) of galactoses; S: number (x) of sialic acids.
Note:
Linkage information is given in ( ) parentheses if applicable, e.g. A2G1S1(6) − α2-6 linked sialic acid. Brackets [x] before G or GalNAc indicate which arm of the mannosyl core is galactosylated e.g. [3]G1 indicates that the galactose is on the antenna of the α1-3 mannose.
²This typically with IgG associated naming system indicates the presence of core fucose (F), the number of galactoses (G) and the presence of biantennary glycans. It is limited in the number of structures and linkages it can describe but is often used for simplicity.
³black circle represents mannose (Man), white square is N-acetyl glucosamine (GlcNAc), black square is N-acetyl galactosamine (GalNAc), white circle is galactose (Gal), white diamond is sialic acid, N-acetyl neuraminic acid (Neu5Ac) and white triangle is fucose (Fuc).

### 6. BRIEF DESCRIPTION OF THE FIGURES

FIG. 1 shows that an antibody displaying A2GalNAc2 glycan on Fab is highly internalized by HepG2 cells. HepG2 cells were incubated for 4 hours with pHrodo-labeled antibodies. The graph shows the average adjusted MFI of pHrodo of triplicate values ±SEM. Graph shows data from one out of 3 representative experiments.

FIG. 2 shows that internalization of antibodies displaying A2GalNAc2 glycan on Fab by HepG2 cells is mediated by ASGPR. HepG2 cells were incubated for 3 hours with pHrodo-labeled antibodies (3 μg/ml) and indicated inhibitors. The graph shows the average and individual adjusted pHrodo MFI of 2 independent experiments. Black circle: No inhibitor. Black triangle: Fetuin. Open square: Asialofetuin. Black diamond: EGTA. Open circle: Chloroquine. Open triangle: Bafilomycin.

FIG. 3. shows that 50-fold higher concentration of Asialofetuin is required to block 50% of A-84.86-A2GalNAc2 uptake. HepG2 cells were incubated with increasing concentrations (0.002-4 μM) of asialofetuin for 30 min, before incubation with pHrodo-labelled antibodies. The graph shows the data from 1 representative experiment out of 3.

FIGS. 4A-4C show that internalization of A-84.86-A2GalNAc2 is dependent on ASGPR1 and ASGPR2. FIG. 4A shows flow cytometry staining for ASGPR1 and ASGPR2 on HepG2 treated with negative control siRNA or siRNA specific for ASGPR1. The graph shows the normalized expression to the negative control treated cells. ASGPR1 siRNA leads to complete reduction of surface levels of ASGPR1 and also to strong reduction of ASGPR2. ASGPR2 homodimer is not stable on cells. FIG. 4B shows flow cytometry staining for ASGPR1 and ASGPR2 for HepG2 cells treated with negative control siRNA and ASGPR2 siRNA. The graph shows the normalized expression to the negative control treated cells. ASGPR2 siRNA leads to complete reduction of ASGPR2 surface level but not ASGPR1. FIG. 4C shows that uptake of A-84.86-A2GalNAc2 antibody is completely abrogated by ASGPR1 and ASGPR2 siRNA while uptake of H-A2F (not an ASGPR engager) is unchanged. The graph shows the average and individual data from 4 independent experiments (siRNA ASGPR1) or 1 experiment (siRNA ASGPR2). Ns: not statistically significant. ** statistically significant difference (p<0.01) by two-way ANOVA followed by Tukey's multiple comparisons test.

FIG. 5A shows the expression of ASGPR1, by flow cytometry normalized to isotype control staining in HepG2 (Sigma), HepG2 parental (HepG2-wt, Synthego) and HepG2-ASGPR1ko (Synthego). FIG. 5B shows the expression of ASGPR2 normalized to isotype control staining in same cells than in A. FIG. 5C shows the DOL adjusted gMFI of the indicated pHRodo-labeled antibody incubated on same cells than in A & B.

FIG. 6A shows that internalized A-84.86-A2GalNAc2 antibody is colocalized with lysosomal vesicles in HepG2 cells. The graph shows the number of pHrodo spots (DeepRed channel) colocalized with the lysosome ROI and normalized to cell number per well, plotted against time of incubation. Lysosome ROI is defined by lysotracker green signal, as described in Example 5. Each data is an average of 3 wells and the errors bars show the standard deviation. FIG. 6B shows the same readout than in 6A for a competitive inhibition experiment with unlabelled A-84.86-A2GalNAc2 titrated from 100 nM down to 0.160 nM (some curves were removed for clarity) against lnM of pHrodo A-84.86-A2GalNAc2. FIG. 6C shows the same readout than in 6A for a competitive inhibition experiment of asialofetuin, titrated from 100 μM to 0.160 μM, against 1 nM of pHrodo A-84.86-A2GalNAc2.

FIG. 7A shows Western blot images after HepG2 cells were incubated for indicated time with antibodies at 100 ug/ml. Western blot was done using anti-human IgG H+L antibody, detecting both heavy and light chain of adalimumab antibodies. FIG. 7B shows signal quantification from the Western blot images. The graph shows the density of signal corresponding to internalized intact A-84.86-A2GalNAc2 (intact Heavy chain+intact Light chain indicated by the arrows on the right side of the blot at 50 KDa and 25 KDa; black bars) or the degradation fragment (white bars, indicated by the arrows in the blot). Density was normalized to the Beta-actin corresponding signal.

FIGS. 8A-8B show that the antigen is well internalized when complexed with A-84.86-A2GalNAc2 and not with a control antibody. FIG. 8A shows western blot images for anti-lambda light chain blot (HCA202 detection) and Beta-Actin detection. FIG. 8B shows the quantification of the lambda light chain (HCA202) signal, normalized to beta-actin corresponding signal, using iBright system

FIG. 9 shows that the target HCA202 is rapidly internalized by liver cells after GalNAc2 glycosylated antibody injection and that the internalized target is rapidly degraded following internalization. Mice (N=3/group) were injected with HCA202 i.v. followed shortly after by control mAb H-A2F, PBS or A-84.86-A2GalNAc2. Liver of animals were harvested at different timepoint and HCA202 was quantified in liver protein extract by western blot. Panel A shows the western blot anti-Lambda light (HCA202) for 1 representative animal per time point. Panel B shows the normalized Lamdba light chain band intensity of the western blot. Each data point represents mean±SEM of n=3 independent animals.

FIG. 10 shows the amount of antibody depleted from the supernatant after HepG2 cells were plated in a 12-well plate and treated with 5 ng/ml of the indicated antibodies for 24, 48 and 72 hours. The graph shows the average ±SD for the amount of antibody depleted from the supernatant, from 3 independent experiments.

FIG. 11 shows that an A2GalNAc2 glycan displayed by a glycotag on the Fc (C-terminus of HC; 11K2-gt1) drives efficient ASGPR-specific internalization of an antibody. A glycotag located on the LC (LCLgt1) was less efficient than a glycotag on the Fc. The internalization is ASGPR-specific as ASGPRko HepG2 cells did not internalize the variants. A variant displaying glycotags on C-terminus of HC and C-terminus of LC (11K2-LCLgt1.gt1-A2GalNAc2) showed a slightly better internalization than 11K2-gt1-A2GalNac2. Similarly, the variant 11K2-84.gt1-A2GalNAc2 with a glycotag on HC plus a glycosite inserted at position 84 in the variable domain of HC showed a higher internalization than the gt1 variant. The graph shows the average ±SEM of DOL adjusted pHrodo geometric MFI (gMFI) from 4-6 independent experiments, except for 86-A1GalNAc2 variant (N=2). ** statistically significant difference compared to IgG4-PAA-A2F control antibody (p<0.01) by Mann-Whitney test. #indicates that no statistical test was done because only 2 data points were available.

FIG. 12 shows internalization of 11K2 glycovariants quantified by western blot. HepG2 were incubated with antibodies at 0.1 mg/ml. Western blot using anti human IgG H+L & Beta-Actin was performed on cell protein extracts. The graph shows the intensity of total antibody signal (heavy chain+light chain signal+degradation fragment signal) for each antibody, normalized to corresponding Beta-Actin signal.

FIG. 13 shows that glycotag on C-terminus of light chain or heavy chain on a mAb drives efficient clearance in vivo and that A1GalNAc1 glycan is not active. Mice were injected with different glycoengineered variants of 11K2 mAb. The levels of the mAb were measured in serum of the animals by ELISA. IgG4-A2F is the non glycoengineered mAb produced in CHO cells. The graphs show average ±SEM of mAb concentration. The graphs show the data of 2 independent experiments.

FIG. 14 shows that a Fab fragment displaying A2GalNAc2 glycan leads to efficient internalization of its target antigen by HepG2 cells. HepG2-wt and HepG2-ASGPR1ko cells were incubated with 3 μg/ml of pHrodo-BSA-Fentanyl×Fab-Fent complexes for 4 h. The graph shows the average ±SEM of BSA-fentanyl pHrodo geometric MFI (gMFI) in the indicated samples. N=3 **** P<0.0001, two-way ANOVA followed by Tukey's multiple comparisons test. Control Fab-Fent-LCLgt1-A2, displaying a non ASGPR engaging glycan on a glycotag did not lead to significant internalization of BSA-fentanyl antigen. In contrast Fab-Fent-LCLgt1-A2GalNAc2, which displays on its glycotag a single A2GalNAc2 glycan, induced a clear internalization of the antigen. This internalization is ASGPR dependent as it is abrogated in HepG2 knock out for ASGPR. Similarly the variant Fab-Fent-86.LCLgt1-A2GalNAc2 induced an even stronger ASGPR-dependent internalization of the antigen, as compared to the LCLgt1 variant.

FIG. 15 shows that the BSA-Fentanyl antigen, internalized by A2GalNAc2 displaying Fab anti-fentanyl glycovariants is rapidly degraded. HepG2 cells were treated as described in example 9. The Figure shows the western blot image for detection with the anti-fentanyl antibody (upper blot) and for beta-actin (lower blot) at indicated time post wash.

FIG. 16 shows that the MOG-Fc-A2GalNAc2 construct was efficiently internalized by HepG2 cells, to a similar extent than the A-84.86-A2GalNAc2 antibody. The internalization was ASGPR-specific as it was blocked by competition with asialofetuin. The graph shows the average DOL-adjusted pHrodo gMFI and individual data point from 2 independent experiments.

FIG. 17 shows that position the A2GalNAc2 N-Glycan is critical for degrader compounds that are based on the autoantigen to capture autoantibodies. Panel A shows that engagement of the 8-18C5 antibody prevents internalization of MOG-Fc degrader but not of variants containing a glycotag at C-term of the Fc. HepG2 cells were incubated with MOG bifunctional degraders in complex with 8-18C5-pHrodo. The graph shows the pHrodo signal detected in the cells after incubation. Panel B shows the model of ASGPR engagement of endogenous glycan in presence of model autoantibody binding to MOG bifunctional degrader. The model autoantibody is represented by the hashed structure. N-glycans are indicated by the stars. ASGPR cannot engage the endogenous glycan when the autoantibody is bound to bifunctional degrader. However the Fc glycotag (gt1) remains accessible for ASGPR when MOG bifunctional degrader is bound to the autoantibody.

FIG. 18 shows that MOG-Fc-N60Q-gt1-A2GalNAc2 bifunctional degrader efficiently depletes a model autoantibody injected in rat. Rats (N=4/group) were injected i.p with 8-18C5 antibody followed by MOG bifunctional degrader s.c. The levels of total (free and bound) 8-18C5 were quantified in serum. The graph shows the average ±SEM (N=4/group).

FIG. 19 shows that antibodies displaying A2GalNAc2 Glycan structure lead to potent elimination of a target antigen from blood circulation in rat. Rats were injected i.v. with HCA202 (0.5 mg/kg) and with Antibodies i.v. 5 mg/kg. Graph shows average ±SD of HCA202 serum concentration in ng/ml of 3 or 4 animal/group. Black circles show H-A2F (adalimumab, Humira) treated group. Open squares show A-84-A2GalNAc2 treated group. Black triangles show A-84.86-A2GalNAc2 treated group. Black square show A-84.86-A2G2S2 treated group. Open circles show PBS treated group (HCA202 only). Open diamonds and dotted line show A-M3 treated group. For graphical representation, when HCA202 levels were below assay LLOQ (dotted LLOQ/MRD10 line=20 ng/ml), they were set at 19 ng/ml.

FIG. 20 shows that an antibody displaying A2GalNAc2 Glycan structure is distributed to the liver area with a fast kinetic as compared to control antibodies. Mice were injected i.v. with CF750-labeled antibodies at 5 mg/kg and imaged using fluorescence tomography. The graph shows the average fluorescence in pmol ±SD of 3 animals/timepoint in the gated liver region of interest. Open Squares and dotted line show Ptz-A2F treated group. Black Circles show H-A2F treated group. Open diamonds show A-84.86-A2G2S2 treated group. Black triangles show A-84.86-A2G2 treated group.

FIG. 21 shows that antibodies distributed to the liver are degraded. Livers from mice injected intravenously (i.v.) with CF750-labelled antibodies at 5 mg/kg were harvested at indicated time points & protein extracts were obtained from ~100 mg liver homogenized in RIPA buffer (+protease inhibitor). Fluorescence captured in the CF750 channel was quantified using iBright system. The extracts were also blotted for beta-Actin. Blot shows the CF750 fluorescence signal.

FIG. 22 shows the relative densitometric unit of the CF750 signal from the gel in FIG. 21 normalized to Beta-Actin control intensity.

FIG. 23 shows that injection by subcutaneous route (s.c.) prolongs the effect of antigen depletion from blood circulation as compared to intravenous (i.v.) route. Rats were injected i.v with HCA202 (0.5 mg/kg) and 15 min later with PBS i.v. A-84.86-A2GalNAc2 i.v or s.c at 5 mg/kg. Graph shows average ±SD of total (free+bound) HCA202 serum concentration in ng/ml of 3 animal/group. For graphical representation, when HCA202 levels were below assay LLOQ (dotted LLOQ/MRD10 line=20 ng/ml), they were set at 19 ng/ml

FIG. 24 shows that a single s.c injection of A-84.86-A2GalNAc2 route can lead to antigen depletion for up to 48 hours. Rats were injected i.v. with HCA202 (0.5 mg/kg) and 15 min later with PBS s.c. or A-84.86-A2GalNAc2 s.c. at 10 mg/kg. The graph shows average ±SD total (free+bound) HCA202 serum levels. The assay LLOQ is indicated by the dotted line (20 ng/ml).

FIG. 25 shows that a single injection of A-84.86-A2GalNAc2 by s.c route can lead to antigen depletion for up to 96 hours. Rats were injected i.v. with HCA202 at timepoints indicated by the arrows. Fifteen minutes after first HCA202 injection, rats were injected with PBS s.c. or A-84.86-A2GalNAc2 s.c. The graph shows average ±SEM total (free+bound) HCA202 serum levels. N=4 animal/group. The assay LLOQ is indicated by the dotted line.

FIG. 26 shows that when HepG2 knock out for ASGPR1 (HepG2-ASGR1ko) are treated with Ptz-gt1-A2GalNAc2 or Ptz-hgt-A2GalNAc2 variants, no HER2 reduction is observed, as compared to isotype control or Ptz-A2F treated cells. The graph shows Her2 detection in HepG2 parental (-wt) or HepG2 knock out for ASGPR1 (-ASGR1ko) treated with 1 μg/ml of the indicated antibody for 4 hours. Her2 detection is expressed as geometric mean fluorescence intensity (gMFI) normalized to the isotype control treatment

FIG. 27 shows that HER2-Fc/trastuzumab Immune complexes (ICs) form predominantly 600-1300 KDa structures. Panel A shows the overlay of size-exclusion chromatogram of individual injections as well as pre-formed immune complexes of Trastuzumab and HER2-Fc in a molar ratio of 1:1. Sizes of formed ICs was estimated via protein MW standards as well as dynamic light scattering measurements. Panel B shows the structure of the ICs formed and that Ptz-gt1 antibody can bind to the formed ICs.

FIG. 28 shows that large immune complexes can be internalized and degraded by a mAb bifunctional degrader. Panel A shows the anti-human IgG H+L western blot and anti-beta actin western blot of HepG2 cell extracts. The position of heavy (H) and light chains (L) of the different components is indicated in the gel. Trtz is trastuzumab. Ptz-gt1 heavy chain has a higher molecular weight than trastuzumab HC because of the glycotag addition. Panel B shows the quantification graph of total band intensity (whole vertical lane for each condition) of the anti-human IgG H+L western blot using IBright FL1500 analysis tool. The graph shows the adjusted total lane volume (=sum of pixel intensities, including all the bands in a lane) normalized to corresponding beta-actin band intensity.

FIG. 29 shows that HCA202 target depletion by A-gt1-A2GalNAc2 follows a dose response and that depletion requires low degrader (A-gt1) to target (HCA202) ratio. Rats were injected i.v. with HCA202 followed by PBS or A-gt1-A2GalNAc2 i.v. at different doses. The numbers above the graph indicate the molar degrader:target ratio. The graph shows average ±SEM of HCA202 depletion from theoretical C_{zero} from N=4 animal.

FIG. 30 shows that target depletion by Fab-A-FLGT4 follows a dose response and that depletion requires low degrader (Fab-A-FLGT4) to target (HCA202) ratio. Rats (N=4 animals/group) were injected i.v. with HCA202 followed by PBS or Fab-A-FLGT4-A2GalNAc2 i.v. at different doses. The numbers above the graph indicate the molar degrader:target ratio. The graph shows average ±SEM of HCA202 depletion from theoretical C_{zero}.

### 7. DETAILED DESCRIPTION OF THE INVENTION

Described herein are glycoengineered bifunctional degraders and populations comprising the same having improved functionalities as compared to a control antibody. As exemplified herein, the glycoengineered bifunctional degrader is engineered by introduction of glycosylation sites on the glycoengineered bifunctional degrader, resulting in an engineered glycosylation profile that mediates endocytic receptor degradation of the glycoengineered bifunctional degrader and the target to which it binds. By customizing the N-glycosylation, the glycoengineered bifunctional degraders described herein: 1) have homogeneous glycosylation; 2) can degrade large targets such as immune complexes; 3) have a defined ligand-to-antibody ratio; 4) have defined glycosylation sites; 6) can activate more diverse and powerful degradation receptors; and/or 6) can engage in protein degradation in a highly optimized manner. The glycoengineered bifunctional degrader may be employed as novel therapeutics to treat diseases, which include but are not limited to inflammatory disorders, blood disorders, autoimmune disorders, infectious diseases, and cancer.

### 7.1 Glycoengineered Bifunctional Degrader

Provided herein is a glycoengineered bifunctional degrader, wherein the bifunctional degrader (i) specifically binds to a target protein and (ii) comprises an N-glycan of the structure:

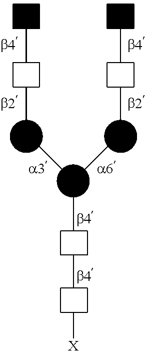

linked to the bifunctional degrader at one or more N-glycosylation sites, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader.

In certain embodiments, the glycoengineered bifunctional degrader specifically binds to one or more target proteins, for example, but not limited to target proteins described in Section 7.7. In certain embodiments, the glycoengineered bifunctional degrader specifically binds to one target protein. In certain embodiments, the glycoengineered bifunctional degrader comprises a moiety that specifically binds to a target protein. In certain embodiments, the moiety comprises a heavy chain variable region and a light chain variable region of an antibody, or a functional fragment thereof. In certain embodiments, the moiety comprises a Fab region of a monoclonal antibody. In certain embodiments, the glycoengineered bifunctional degrader comprises 1, 2, 3, 4, 5, 6, 7, 8, 9, 10 or more moieties that each specifically bind to a target protein so that a single bifunctional degrader can bind to 1, 2, 3, 4, 5, 6, 7, 8, 9, 10 or more target protein molecules. In certain embodiments, each of the moieties specifically binds to the same target protein. In certain embodiments, two, three, four or more of the moieties bind to different target proteins.

In certain embodiments, the bifunctional degrader specifically binds to two different target proteins. In certain embodiments, the bifunctional degrader comprises a first moiety that specifically binds to a first target protein and a second moiety that specifically binds to a second target protein. In certain embodiments, the glycoengineered bifunctional degrader has: (i) 1, 2, 3, 4, 5, 6, 7, 8, 9, 10 or more first moieties that specifically bind to a first target protein so that a single bifunctional degrader can bind to 1, 2, 3, 4, 5, 6, 7, 8, 9, 10 or more of the first target protein molecules; and (ii) 1, 2, 3, 4, 5, 6, 7, 8, 9, 10 or more second moieties that specifically bind to a second target protein so that a single bifunctional degrader can bind to 1, 2, 3, 4, 5, 6, 7, 8, 9, 10 or more of the second target protein molecules.

In certain embodiments, the bifunctional degrader specifically binds to three different target proteins. In certain embodiments, the bifunctional degrader comprises a first moiety that specifically binds to a first target protein, a second moiety that specifically binds to a second target protein, and a third moiety that specifically binds to a third target protein. In certain embodiments, the glycoengineered bifunctional degrader has (i) 1, 2, 3, 4, 5, 6, 7, 8, 9, 10 or more first moieties that specifically bind to a first target protein so that a single bifunctional degrader can bind to 1, 2, 3, 4, 5, 6, 7, 8, 9, 10 or more of the first target protein molecules; and (ii) 1, 2, 3, 4, 5, 6, 7, 8, 9, 10 or more second moieties that specifically bind to a second target protein so that a single bifunctional degrader can bind to 1, 2, 3, 4, 5, 6, 7, 8, 9, 10 or more of the second target protein molecules; and (iii) 1, 2, 3, 4, 5, 6, 7, 8, 9, 10 or more third moieties that specifically bind to a third target protein so that a single bifunctional degrader can bind to 1, 2, 3, 4, 5, 6, 7, 8, 9, 10 or more of the third target protein molecules.

In certain embodiments, the glycoengineered bifunctional degrader described herein activates natural degradation pathways. In certain embodiments, the natural degradation pathways comprise receptor-mediated endocytosis. Without being bound by theory, receptor-mediated endocytosis comprises capture of glycosylated proteins via specific glycan structures (for example, by endocytic lectins) to mediate lysosomal degradation (Cummings et al., Cold Spring Harbor Laboratory Press, (2017). Endocytic lectins are ubiquitous in humans and can recognize various glycan structures. Glycan engagement with endocytic carbohydrate-binding proteins and receptors enables essential biological pathways including, but not limited to, those involved in modulating immune responses, mediating protein clearance, protein turnover, and controlling trafficking of soluble glycoproteins, glycolipids and any natural molecule containing a glycan moiety.

In certain embodiments, activation of the natural degradation pathways by the glycoengineered bifunctional degrader reduces the concentration of a target protein in a subject. In certain embodiments, activation of the natural degradation pathways by the glycoengineered bifunctional degrader reduces the concentration of a target protein in a subject by at least 10%, at least 20%, at least 30%, at least 40%, at least 50%, at least 60%, at least 70%, at least 80%, at least 90%, at least 95%, at least 98%, or about 100% as compared to the concentration in the subject before administration of the glycoengineered bifunctional degrader. In certain embodiments, the glycoengineered bifunctional degrader mediates endocytic receptor degradation of the glycoengineered bifunctional degrader and the target protein to which it binds.

In certain embodiments, the glycoengineered bifunctional degrader comprises an N-glycan of the structure:

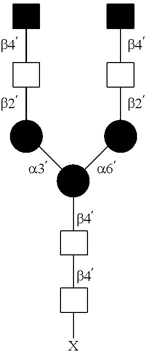

linked to the bifunctional degrader at one or more N-glycosylation sites, wherein the black square represents an N-acetyl galactosamine (GalNAc), the white square represents an N-acetylglucosamine (GlcNAc) residue the black circle represents a mannose (Man) residue, and X represents an amino acid residue of the bifunctional degrader, and wherein the N-glycan specifically binds to one or more endocytic receptors that mediate lysosomal degradation. In certain embodiments, the endocytic receptors are endocytic carbohydrate-binding proteins and/or lectin receptors. In one embodiment, the endocytic carbohydrate-binding protein is ASGPR. In one embodiment, the N-glycan specifically binds to ASGPR.

ASGPR-mediated degradation in the hepatocyte has many applications. ASPGR binding to the N-glycan structure disclosed herein can result in the selective degradation of one or more target protein described in Section 7.7. By way of example, ASGPR-mediated degradation can lead to removal of cytokines, chemokines and hormones. Additionally, ASGPR-mediated degradation can be used for the delivery of the target molecules to the hepatocyte endosome. Thus, ASGPR-mediated degradation is applicable for various liver diseases, while limiting systemic toxicity.

In certain embodiments, the bifunctional degrader comprises 1, 2, 3, 4, 5, 6, 7, 8, 9, 10 or more N-glycosylation sites (or glycosites; such as an N-glycosylation consensus sequence). In certain embodiments, the bifunctional degrader comprises at least 2, at least 3, at least 4, at least 5, at least 6, at least 7, at least 8, at least 9, or at least 10 N-glycosylation sites. In certain embodiments, the bifunctional degrader comprises at least 2 N-glycosylation sites. In certain embodiments, the bifunctional degrader comprises at least 3 N-glycosylation sites. In certain embodiments, the bifunctional degrader comprises at least 4 N-glycosylation sites. In certain embodiments, the bifunctional degrader comprises at least 5 N-glycosylation sites. In certain embodiments, the bifunctional degrader comprises at least 6 N-glycosylation sites. In certain embodiments, the bifunctional degrader comprises at least 7 N-glycosylation sites. In certain embodiments, the bifunctional degrader comprises at least 8 N-glycosylation sites. In certain embodiments, the bifunctional degrader comprises at least 9 N-glycosylation sites. In certain embodiments, the bifunctional degrader comprises at least 10 N-glycosylation sites. In certain embodiments, the bifunctional degrader comprises 2 N-glycosylation sites. In certain embodiments, the bifunctional degrader comprises 3 N-glycosylation sites. In certain embodiments, the bifunctional degrader comprises 4 N-glycosylation sites. In certain embodiments, the bifunctional degrader comprises 5 N-glycosylation sites. In certain embodiments, the bifunctional degrader comprises 6 N-glycosylation sites. In certain embodiments, the bifunctional degrader comprises 7 N-glycosylation sites. In certain embodiments, the bifunctional degrader comprises 8 N-glycosylation sites. In certain embodiments, the bifunctional degrader comprises 9 N-glycosylation sites. In certain embodiments, the bifunctional degrader comprises 10 N-glycosylation sites.

In certain embodiments, one or more of the N-glycosylation sites are engineered into the amino acid sequence of the bifunctional degrader (i.e. one or more of the N-glycosylation sites are not present in a wild-type, natural, synthetic, or commercial precursor of the bifunctional degrader; such sites are also referred to herein as “glycoengineered N-glycosylation sites”). In certain embodiments, at least one of the N-glycosylation sites is engineered into the amino acid sequence of the bifunctional degrader. In certain embodiments, at least two of the N-glycosylation sites are engineered into the amino acid sequence of the bifunctional degrader. In certain embodiments, at least three of the N-glycosylation sites are engineered into the amino acid sequence of the bifunctional degrader. In certain embodiments, at least four of the N-glycosylation sites are engineered into the amino acid sequence of the bifunctional degrader. In certain embodiments, one or more of the N-glycosylation sites are engineered distal to a target-specific binding location of the bifunctional degrader. In certain embodiments, at least one or at least two N-glycosylation sites are engineered distal to the target-specific binding location. In certain embodiments, wherein all of the N-glycosylation sites are engineered distal to the target-specific binding location. In certain embodiments, the target-specific binding location is a variable region of an antibody or antigen-binding fragment (Fab), or an ectodomain of an Fc-fusion protein. In certain embodiments, one or more of the engineered N-glycosylation sites are glycotags fused to the N- and/or C-terminus of the the bifunctional degrader via a peptide linker. In certain embodiments, a glycotag is fused to the N-terminus of the bifunctional degrader. In certain embodiments, a glycotag is fused to the C-terminus of the bifunctional degrader. In certain embodiments, a glycotag is fused to the N- and the C-terminus of the bifunctional degrader. In certain embodiments, one or more of the N-glycosylation sites are natural N-glycosylation sites (i.e. one or more of the N-glycosylation sites are present in a wild-type, natural, synthetic, or commercial precursor of the bifunctional degrader). In certain embodiments, at least one of the N-glycosylation sites is a natural N-glycosylation site. In certain embodiments, at least two of the N-glycosylation sites are natural N-glycosylation sites. In certain embodiments, one or more natural N-glycosylation sites have been deleted, mutated, or functionally inactivated. In certain embodiments, at least one or at least two natural N-glycosylations sites have been deleted, mutated, or functionally inactivated. In certain embodiments, all natural N-glycosylation sites have been deleted, mutated, or functionally inactivated. In certain embodiments, the one or more natural N-glycosylation sites are located at or proximal to the target-specific binding location of the wild-type, natural, synthetic, or commercial precursor of the bifunctional degrader. In certain embodiments, one or more natural N-glycosylation sites have been deleted, mutated, or functionally inactivated and one or more glycoengineered N-glycosylation sites are present in the bifunctional degrader. In certain embodiments, one or more glycoengineered N-glycosylation sites are located distal to a target-specific binding location of the bifunctional degrader.

In certain embodiments, the 1, 2, 3, 4, 5, 6, 7, 8, 9, 10 or more N-glycosylation sites can be glycosylated by the N-glycan such that the resulting glycoengineered bifunctional degrader can engage with or bind to 1, 2, 3, 4, 5, 6, 7, 8, 9, 10 or more endocytic receptor molecules. In certain embodiments, the bifunctional degrader is glycosylated by the N-glycan at least 2, at least 3, at least 4, at least 5, at least 6, at least 7, at least 8, at least 9, or at least 10 of the N-glycosylation sites. In certain embodiments, the bifunctional degrader is glycosylated by the N-glycan at least 2 N-glycosylation sites. In certain embodiments, the bifunctional degrader is glycosylated by the N-glycan at least 3 N-glycosylation sites. In certain embodiments, the bifunctional degrader is glycosylated by the N-glycan at least 4 N-glycosylation sites. In certain embodiments, the bifunctional degrader is glycosylated by the N-glycan at least 5 N-glycosylation sites. In certain embodiments, the bifunctional degrader is glycosylated by the N-glycan at least 6 N-glycosylation sites. In certain embodiments, the bifunctional degrader is glycosylated by the N-glycan at least 7 N-glycosylation sites. In certain embodiments, the bifunctional degrader is glycosylated by the N-glycan at least 8 N-glycosylation sites. In certain embodiments, the bifunctional degrader is glycosylated by the N-glycan at least 9 N-glycosylation sites. In certain embodiments, the bifunctional degrader is glycosylated by the N-glycan at least 10 N-glycosylation sites. In certain embodiments, the bifunctional degrader is glycosylated by the N-glycan at 2 N-glycosylation sites. In certain embodiments, the bifunctional degrader is glycosylated by the N-glycan at 3 N-glycosylation sites. In certain embodiments, the bifunctional degrader is glycosylated by the N-glycan at 4 N-glycosylation sites. In certain embodiments, the bifunctional degrader is glycosylated by the N-glycan at 5 N-glycosylation sites. In certain embodiments, the bifunctional degrader is glycosylated by the N-glycan at 6 N-glycosylation sites. In certain embodiments, the bifunctional degrader is glycosylated by the N-glycan at 7 N-glycosylation sites. In certain embodiments, the bifunctional degrader is glycosylated by the N-glycan at 8 N-glycosylation sites. In certain embodiments, the bifunctional degrader is glycosylated by the N-glycan at 9 N-glycosylation sites. In certain embodiments, the bifunctional degrader is glycosylated by the N-glycan at 10 N-glycosylation sites. In certain embodiments, the bifunctional degrader is glycosylated at an Asn amino acid residue of the bifunctional degrader. In certain embodiments, the N-glycosylation site is an N-glycosylation consensus sequence. In certain embodiments, the N-glycosylation site comprises a consensus sequence of N-X-S/T or N-X-C, wherein X is any amino acid except proline.

In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan. In certain embodiments, at least 10%, at least 20%, at least 30%, at least 40%, at least 50%, at least 60%, at least 70%, at least 80%, at least 90%, at least 95, or at least 98% of the N-glycosylation sites are occupied by an N-glycan of the structure:

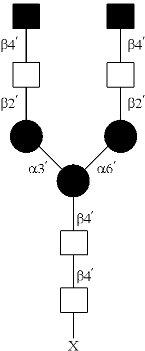

linked to the bifunctional degrader at one or more N-glycosylation sites, wherein the black square represents an N-acetyl galactosamine (GalNAc), the white square represents an N-acetylglucosamine (GlcNAc) residue the black circle represents a mannose (Man) residue, and X represents an amino acid residue of the bifunctional degrader. In certain embodiments, at least 10% of the N-glycosylation sites are occupied by the N-glycan. In certain embodiments, at least 20% of the N-glycosylation sites are occupied by the N-glycan. In certain embodiments, at least 30% of the N-glycosylation sites are occupied by the N-glycan. In certain embodiments, at least 40% of the N-glycosylation sites are occupied by the N-glycan. In certain embodiments, at least 50% of the N-glycosylation sites are occupied by the N-glycan. In certain embodiments, at least 60% of the N-glycosylation sites are occupied by the N-glycan. In certain embodiments, at least 70% of the N-glycosylation sites are occupied by the N-glycan. In certain embodiments, at least 80% of the N-glycosylation sites are occupied by the N-glycan. In certain embodiments, at least 90% of the N-glycosylation sites are occupied by the N-glycan. In certain embodiments, at least 95% of the N-glycosylation sites are occupied by the N-glycan. In certain embodiments, at least 98% of the N-glycosylation sites are occupied by the N-glycan.

In certain embodiments, the N-glycan is linked to the bifunctional degrader at least one N-glycosylation site. In certain embodiments, the N-glycan is linked to the bifunctional degrader at least two N-glycosylation sites. In certain embodiments, the N-glycan is linked to the bifunctional degrader at one, two, three, or four N-glycosylation sites. In certain embodiments, the N-glycan is linked to the bifunctional degrader at one N-glycosylation site. In certain embodiments, the N-glycan is linked to the bifunctional degrader at two N-glycosylation sites. In certain embodiments, the N-glycan is linked to the bifunctional degrader at three N-glycosylation sites. In certain embodiments, the N-glycan is linked to the bifunctional degrader at four N-glycosylation sites. In certain embodiments, the N-glycan is linked to the bifunctional degrader at an Asn amino acid residue of the bifunctional degrader. In certain embodiments, the N-glycan is linked to the bifunctional degrader at an N-glycosylation consensus sequence. In certain embodiments, the N-glycan is linked to the bifunctional degrader at a consensus sequence of N-X-S/T or N-X-C, wherein X is any amino acid except proline.

In certain embodiments, the one or more N-glycosylation sites are distal to a target-specific binding location of the bifunctional degrader. In certain embodiments, at least one, at least two, at least three, or at least four N-glycosylation sites are distal to the target-specific binding location. In certain embodiments, at least one N-glycosylation site is distal to the target-specific binding location. In certain embodiments, at least two N-glycosylation sites are distal to the target-specific binding location. In certain embodiments, at least three N-glycosylation sites are distal to the target-specific binding location. In certain embodiments, at least four N-glycosylation sites are distal to the target-specific binding location. In certain embodiments, one N-glycosylation site is distal to the target-specific binding location. In certain embodiments, two N-glycosylation sites are distal to the target-specific binding location. In certain embodiments, three N-glycosylation sites are distal to the target-specific binding location. In certain embodiments, four N-glycosylation sites are distal to the target-specific binding location. In certain embodiments, all of the N-glycosylation sites are distal to the target-specific binding location. In certain embodiments, the one or more N-glycosylation sites are not present in a wild-type, natural, synthetic, or commercial precursor of the bifunctional degrader. In certain embodiments, at least one, at least two, at least three, or at least four of the N-glycosylation sites are not present in a wild-type, natural, synthetic, or commercial precursor of the bifunctional degrader. In certain embodiments, at least one of the N-glycosylation sites is not present in a wild-type, natural, synthetic, or commercial precursor of the bifunctional degrader. In certain embodiments, at least two of the N-glycosylation sites are not present in a wild-type, natural, synthetic, or commercial precursor of the bifunctional degrader. In certain embodiments, at least three of the N-glycosylation sites are not present in a wild-type, natural, synthetic, or commercial precursor of the bifunctional degrader. In certain embodiments, at least four of the N-glycosylation sites are not present in a wild-type, natural, synthetic, or commercial precursor of the bifunctional degrader. In certain embodiments, one of the N-glycosylation sites is not present in a wild-type, natural, synthetic, or commercial precursor of the bifunctional degrader. In certain embodiments, two of the N-glycosylation sites are not present in a wild-type, natural, synthetic, or commercial precursor of the bifunctional degrader. In certain embodiments, three of the N-glycosylation sites are not present in a wild-type, natural, synthetic, or commercial precursor of the bifunctional degrader. In certain embodiments, four of the N-glycosylation sites are not present in a wild-type, natural, synthetic, or commercial precursor of the bifunctional degrader. In certain embodiments, the target-specific binding location is a variable region of an antibody or antigen-binding fragment (Fab), or an ectodomain of an Fc-fusion protein.

In certain embodiments, the distal N-glycosylation site(s) and the target-specific binding location are separated by at least 5, at least 10, at least 20, at least 30, at least 40, at least 50, at least 60, at least 70, at least 80, at least 90, or at least 100 amino acids. In certain embodiments, the distal N-glycosylation site(s) and the target-specific binding location separated by a distance of about 5-10, about 10-20, about 20-30, about 30-40, about 40-50, about 50-60, about 60-70, about 70-80, about 80-90, about 90-100, about 100-150, about 150-200, about 200-300, or about 300-400 amino acids. In certain embodiments, the amino acid separation between the distal N-glycosylation site(s) and the target-specific binding location is the number of amino acids between the terminal amino acids of the N-glycosylation consensus sequence. Without being bound by theory, the bifunctional degrader folds in space and, thus, has a three-dimensional geometry in addition to its primary amino acid structure. Also without being bound by theory, this three-dimensional geometry, including the position of the N-glycan is not static but dynamic (see, for example, Re, S., et al Biophysical Reviews, 4, 179-187 (2012)). Notwithstanding, in certain embodiments, the distance between the distal N-glycosylation site(s) and the target-specific binding location on a bifunctional degrader may be from an equilibrium geometry of the bifunctional degrader, as determined by any standard means known in the art, including for example computational modelling studies. In certain embodiments, the distal N-glycosylation site(s) and the target-specific binding location are separated by a distance of at least 4 nm, at least 5 nm, at least 6 nm, at least 7 nm, at least 8 nm, at least 9 nm, at least 10 nm, at least 11 nm, at least 12 nm, at least 13 nm, at least 14 nm, at least 15 nm, at least 16 nm, at least 17 nm, at least 18 nm, at least 19 nm, or at least 20 nm. In certain embodiments, the distal N-glycosylation site(s) and the target-specific binding location are separated by a distance of about 4 to 20 nm, about 5 to 20 nm, about 6 to 20 nm, about 7 to 20 nm, about 8 to 20 nm, about 9 to 20 nm, about 10 to 20 nm, about 11 to 20 nm, about 12 to 20 nm, about 13 to 20 nm, about 14 to 20 nm, about 15 to 20 nm, about 16 to 20 nm, about 17 to 20 nm, about 18 to 20 nm, about 4 to 6 nm, about 5 to 7 nm, about 7 to 9 nm, about 8 to 10 nm, about 9 to 11 nm, about 10 to 12 nm, about 11 to 13 nm, about 12 to 14 nm, about 13 to 15 nm, about 14 to 16 nm, about 15 to 17 nm, about 16 to 18 nm, or about 17 to 19 nm. In certain embodiments, the distance between the distal N-glycosylation site(s) and the target-specific binding location is chosen to minimize steric hinderance, for example between the bifunctional degrader(s), the target protein(s), and/or the ASGPR receptor(s), when the target protein is bound to the bifunctional degrader. In certain embodiments, the N-glycan is linked to the bifunctional degrader at an Asn amino acid residue of the bifunctional degrader. In certain embodiments, the N-glycan is linked to the bifunctional degrader at an N-glycosylation consensus sequence. In certain embodiments, the N-glycan is linked to the bifunctional degrader at a consensus sequence of N-X-S/T or N-X-C, wherein X is any amino acid except proline.

In certain embodiments, one or more N-glycosylation sites present in a wild-type, natural, synthetic, or commercial precursor of the bifunctional degrader (“natural N-glycosylation sites”) have been deleted, mutated, or functionally inactivated. In certain embodiments, at least one, at least two, at least three, or at least four natural N-glycosylation sites have been deleted, mutated, or functionally inactivated. In certain embodiments, at least one natural N-glycosylation site has been deleted, mutated, or functionally inactivated. In certain embodiments, at least two natural N-glycosylation sites have been deleted, mutated, or functionally inactivated. In certain embodiments, at least three natural N-glycosylation sites have been deleted, mutated, or functionally inactivated. In certain embodiments, at least four natural N-glycosylation sites have been deleted, mutated, or functionally inactivated. In certain embodiments, one natural N-glycosylation site has been deleted, mutated, or functionally inactivated. In certain embodiments, two natural N-glycosylation sites have been deleted, mutated, or functionally inactivated. In certain embodiments, three natural N-glycosylation sites have been deleted, mutated, or functionally inactivated. In certain embodiments, four natural N-glycosylation sites have been deleted, mutated, or functionally inactivated. In certain embodiments, all natural N-glycosylation sites have been deleted, mutated, or functionally inactivated. In certain embodiments, the one or more natural N-glycosylation sites are located at or proximal to the target-specific binding location of the wild-type, natural, synthetic, or commercial precursor of the bifunctional degrader. In certain embodiments, the target-specific binding location is a variable region of an antibody or antigen-binding fragment (Fab), or an ectodomain of an Fc-fusion protein.

In certain embodiments, the glycoengineered bifunctional degrader comprises two different N-glycans (i.e. a first and a second N-glycan), wherein each N-glycan is independently linked to the bifunctional degrader at 1, 2, 3, 4, 5, 6, 7, 8, 9, 10 or more N-glycosylation sites, and wherein one of the N-glycans (i.e. the first N-glycan) has the structure:

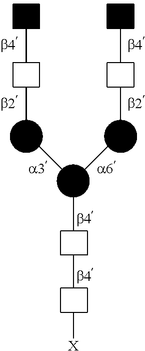

wherein the black square represents an N-acetyl galactosamine (GalNAc), the white square represents an N-acetylglucosamine (GlcNAc) residue the black circle represents a mannose (Man) residue, and X represents an amino acid residue of the bifunctional degrader. In certain embodiments, the different N-glycans specifically bind to different endocytic receptors. In certain embodiments, the first N-glycan specifically binds to ASGPR. In certain embodiments, the other N-glycan (i.e. the second N-glycan) is an N-glycan depicted in Section 5.3. In certain embodiments, the other N-glycan is an N-glycan described in PCT/EP2022/057556, which is incorporated herein by reference in its entirety. In certain embodiments, the first N-glycan is larger than the second N-glycan. In other embodiments, the first N-glycan is smaller than the second N-glycan. In certain embodiments, the N-glycosylation sites predominantly or exclusively occupied by the larger N-glycan are more sterically accessible than the N-glycosylation sites predominantly or exclusively occupied by the smaller N-glycan. In certain embodiments, the other N-glycan is A2. In certain embodiments, the other N-glycan is A1GalNAc1 or A2GalNAc1. In certain embodiments, the N-glycans are linked to the bifunctional degrader at an Asn amino acid residue of the bifunctional degrader. In certain embodiments, the N-glycans are linked to the bifunctional degrader at an N-glycosylation consensus sequence. In certain embodiments, the N-glycans are linked to the bifunctional degrader at a consensus sequence of N-X-S/T or N-X-C, wherein X is any amino acid except proline. In certain embodiments, the first N-glycan is linked to the bifunctional degrader at 1, 2, 3, 4, 5, 6, 7, 8, 9, 10 or more N-glycosylation sites, and the second N-glycan is linked to the bifunctional degrader at 1, 2, 3, 4, 5, 6, 7, 8, 9, 10 or more N-glycosylation sites.

In certain embodiments, the bifunctional degrader further comprises a third N-glycan, wherein the third N-glycan is linked to the bifunctional degrader at 1, 2, 3, 4, 5, 6, 7, 8, 9, 10 or more N-glycosylation sites. In certain embodiments, the third N-glycan specifically binds to a different endocytic receptor than the first and/or second N-glycan. In certain embodiments, the third N-glycan is an N-glycan depicted in Section 5.3. In certain embodiments, the third N-glycan is an N-glycan described in PCT/EP2022/057556, which is incorporated herein by reference in its entirety. In certain embodiments, the third N-glycan is A2. In certain embodiments, the third N-glycan is A1GalNAc1 or A2GalNAc1. In certain embodiments, the third N-glycan is linked to the bifunctional degrader at an Asn amino acid residue of the bifunctional degrader. In certain embodiments, the third N-glycan is linked to the bifunctional degrader at an N-glycosylation consensus sequence. In certain embodiments, the third N-glycan is linked to the bifunctional degrader at a consensus sequence of N-X-S/T or N-X-C, wherein X is any amino acid except proline.

In certain embodiments, the second and/or third N-glycan specifically bind to an endocytic lectin. In some embodiments, the endocytic lectin is a mannose binding receptor. In some embodiments, the endocytic lectin is a Cluster of Differentiation 206 (CD206) receptor. In some embodiments, the endocytic lectin is a DC-SIGN (Cluster of Differentiation 209 or CD209) receptor. In some embodiments, the endocytic lectin is a C-Type Lectin Domain Family 4 Member G (LSECTin) receptor. In some embodiments, the endocytic lectin is a macrophage inducible Ca²⁺-dependent lectin receptor (Mincle). In some embodiments, the endocytic receptor is L-SIGN CD209L. In some embodiments, the endocytic receptor is asialoglycoprotein (ASGPR). In some embodiments, the endocytic receptor is dectin-1. In some embodiments, the endocytic receptor is dectin-2. In some embodiments, the endocytic receptor is langerin. In some embodiments, the second and/or third N-glycan specifically bind to a receptor selected from the group consisting of macrophage mannose 2 receptor, BDCA-2, DCIR, MBL, MDL, MICL, CLEC2, DNGR1, CLEC12B, DEC-205, and mannose 6 phosphate receptor (M6PR).

CD206 is a C-type lectin and phagocytic/endocytic recycling and signaling receptor. CD206 is expressed primarily by M2 anti-inflammatory macrophages, dendritic cells, and live sinusoidal endothelial cells. DC-SIGN is a non-recycling, signaling receptor that targets both the ligand and receptor to the lysosome for degradation. LSECTin is expressed on liver sinusoidal endothelial cells.

In certain embodiments, the glycoengineered bifunctional degrader is glycosylated at two or more N-glycosylation sites by an N-glycan of the structure:

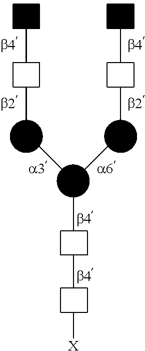

wherein the black square represents an N-acetyl galactosamine (GalNAc), the white square represents an N-acetylglucosamine (GlcNAc) residue the black circle represents a mannose (Man) residue, and X represents an amino acid residue of the bifunctional degrader, and wherein two of the N-glycosylation sites are separated by at least 5, at least 10, at least 20, at least 30, at least 40, at least 50, at least 60, at least 70, at least 80, at least 90, or at least 100 amino acids. In certain embodiments, the N-glycan is linked to the bifunctional degrader at two N-glycosylation sites separated by a distance of about 5-10, about 10-20, about 20-30, about 30-40, about 40-50, about 50-60, about 60-70, about 70-80, about 80-90, about 90-100, about 100-150, about 150-200, or about 200-300 amino acids. In certain embodiments, the amino acid separation between the N-glycosylation sites is the number of amino acids between the terminal amino acids of the N-glycosylation consensus sequence. Without being bound by theory, the bifunctional degrader folds in space and, thus, has a three-dimensional geometry in addition to its primary amino acid structure. Also without being bound by theory, this three-dimensional geometry, including the position of the N-glycan is not static but dynamic (see, for example, Re, S., et al Biophysical Reviews, 4, 179-187 (2012)). Notwithstanding, in certain embodiments, the distance between N-glycosylation sites and/or N-glycans on a bifunctional degrader may be from an equilibrium geometry of the bifunctional degrader, as determined by any standard means known in the art, including for example computational modelling studies. In certain embodiments, the N-glycan is linked to the bifunctional degrader at two N-glycosylation sites separated by a distance of at least 1.0 nm. In certain embodiments, the N-glycan is linked to the bifunctional degrader at two N-glycosylation sites separated by a distance of about 1.0-5.0 nm. In certain embodiments, the N-glycan is linked to the bifunctional degrader at two N-glycosylation sites separated by a distance of about 1.5-3.0 nm. In certain embodiments, the N-glycan is linked to the bifunctional degrader at two N-glycosylation sites separated by a distance of about 1.5-2.5 nm. In certain embodiments, the N-glycan is linked to the bifunctional degrader at three N-glycosylation sites each separated by a distance of about 1.0-5.0 nm. In certain embodiments, the N-glycan is linked to the bifunctional degrader at three N-glycosylation sites each separated by a distance of about 1.5-3.0 nm. In certain embodiments, the N-glycan is linked to the bifunctional degrader at three N-glycosylation sites each separated by a distance of about 1.5-2.5 nm. In certain embodiments, the N-glycans are separated by a distance of at least 1.0 nm. In certain embodiments, the N-glycans are separated by a distance of about 1.0 to about 5.0 nm. In certain embodiments, the N-glycans are separated by a distance of about 1.5 to about 2.5 nm. In certain embodiments, the distance between the N-glycosylation sites and/or N-glycans is chosen to minimize steric hinderance, for example between the bifunctional degrader(s), the target protein(s), and/or the ASGPR receptor(s). In certain embodiments, the distance between the N-glycosylation sites and/or N-glycans is chosen based on the separation of ASGPR receptors on a cell surface. In certain embodiments, the distance between the N-glycosylation sites and/or N-glycans is chosen to be similar (e.g. no more than twice, or no less than half) to the separation of ASGPR receptors on a cell surface. In certain embodiments, the N-glycan is linked to the bifunctional degrader at an Asn amino acid residue of the bifunctional degrader. In certain embodiments, the N-glycan is linked to the bifunctional degrader at an N-glycosylation consensus sequence. In certain embodiments, the N-glycan is linked to the bifunctional degrader at a consensus sequence of N-X-S/T or N-X-C, wherein X is any amino acid except proline.

In certain embodiments, the bifunctional degrader comprises 1, 2, 3, 4, 5, 6, 7, 8, 9, 10 or more polypeptide chains. In certain embodiments, each chain can be produced in a different cell line. In certain embodiments, the bifunctional degrader is a dimer comprising two polypeptide chains that have at least 80%, at least 85%, at least 90%, at least 95, at least 98%, or about 100% sequence identity. In certain embodiments, the dimer comprises two identical polypeptide chains. In certain embodiments, the bifunctional degrader comprises four polypeptide chains. In certain embodiments, two of the four polypeptides chains have at least 80%, at least 85%, at least 90%, at least 95, at least 98%, or about 100% sequence identity to each other. In certain embodiments, two of the four polypeptides chains are identical to each other.

In certain embodiments, the bifunctional degrader is an antibody or fragment thereof. In other embodiments, the antibody is a full length antibody, an Fab, an F(ab′)₂, an Scfv, or a sdAb. In certain embodiments, the bifunctional degrader is a Fab, scFv, Fc, Fv fragment of an antibody. In certain embodiments, the bifunctional degrader is an antibody. In certain embodiments, the antibody is isolated from a human subject. In certain embodiments, the antibody is a monoclonal or polyclonal antibody. In certain embodiments, the antibody is a recombinant antibody. In certain embodiments, the antibody is humanized, chimeric or fully human. In certain embodiments, the bifunctional degrader is an autoantigen. In certain embodiments, the bifunctional degrader is an autoantibody. In certain embodiments, the glycoengineered bifunctional degrader is a nanobody.

In certain embodiments, the glycoengineered bifunctional degrader is an antibody. In certain embodiments, the antibody has the amino acid sequence of adalimumab (Humira® (AbbVie Inc.)); Remicade® (Janssen Biotech, Inc.) (Infliximab); ReoPro® (Janssen Biotech, Inc.) (Abciximab); Rituxan® (Genentech, Inc.) (Rituximab); Simulect® (Novartis Pharmaceuticals Corporation) (Basiliximab); Synagis® (Medimmune, LLC) (Palivizumab); Herceptin® (Genentech, Inc.) (Trastuzumab); Mylotarg® (Pfizer) (Gemtuzumab ozogamicin); Campath® (Takeda Oncology Corporation) (Alemtuzumab); Zevalin® (Acrotech Biopharma Inc.) (Ibritumomab tiuxetan); Xolair® (Genentech, Inc.) (Omalizumab); Bexxar® (GlaxoSmithKline) (Tositumomab-I-131); Erbitux® (Lilly USA, Inc.) (Cetuximab); Avastin® (Genentech, Inc.) (Bevacizumab); Tysabri® (Biogen Idec Corporation) (Natalizumab); Actemra® (Genentech) (Tocilizumab); Vectibix® (Amgen, Inc.) (Panitumumab); Lucentis® (Genentech, Inc.) (Ranibizumab); Soliris® (Alexion Pharmaceuticals Inc.) (Eculizumab); Cimzia® (UCB Pharma Ltd.) (Certolizumab pegol); Simponi® (Janssen Biotech, Inc.) (Golimumab); Ilaris (Novartis Pharmaceuticals Corporation) (Canakinumab); Stelara® (Janssen Biotech, Inc.) (Ustekinumab); Arzerra® (GlaxoSmithKline) (Ofatumumab); Prolia® and Xgeva® (Amgen, Inc.) (Denosumab); Numax® (Medimmune, LLC) (Motavizumab); ABThrax® (GlaxoSmithKline) (Raxibacumab); Benlysta® (GlaxoSmithKline) (Belimumab); Yervoy® (Bristol-Myers Squibb) (Ipilimumab); Adcetris® (Seagen, Inc.) (Brentuximab Vedotin); Perjeta® (Genentech, Inc.) (Pertuzumab); Kadcyla® (Genentech, Inc.) (Ado-trastuzumab emtansine); or Gazyva® (Genentech, Inc.) (Obinutuzumab).

In certain embodiments, the bifunctional degrader is an antibody or fragment thereof, wherein the antibody comprises one or more N-glycosylation sites glycosylated by an N-glycan of structure:

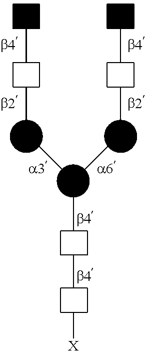

wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, the N-glycan is linked to an N-glycosylation site of the light chain of the antibody or fragment thereof. In certain embodiments, the N-glycan is linked to an N-glycosylation site of the heavy chain of the antibody or fragment thereof. In certain embodiments, one or more of the N-glycosylation sites are located in a constant domain of the antibody or fragment thereof. In certain embodiments, one or more of the N-glycosylation sites are located in a variable domain of the antibody or fragment thereof. In certain embodiments, one or more of the N-glycosylation sites are located in the Fab region of the antibody. In certain embodiments, one or more of the N-glycosylation sites are located in the Fc region of the antibody. In certain embodiments, one or more of the N-glycosylation sites are located in the hinge region of the antibody. In certain embodiments, the N-glycan is linked to the bifunctional degrader at an Asn amino acid residue of the antibody. In certain embodiments, the N-glycan is linked to the antibody at an N-glycosylation consensus sequence. In certain embodiments, the N-glycan is linked to the antibody at a consensus sequence of N-X-S/T or N-X-C, wherein X is any amino acid except proline. In certain embodiments, at least one of the N-glycosylation sites is not present in a wild-type form of the antibody. In certain embodiments, all but two of the N-glycosylation sites are not present in a wild-type form of the antibody.

In certain embodiments, the Fab region of the antibody has 1, 2, 3, 4, 5, 6, 7, 8, 9, 10 or more N-glycosylation sites. In certain embodiments, Fab region of the antibody has 2 N-glycosylation sites. In certain embodiments, the Fab region of the antibody has 4 N-glycosylation sites. In certain embodiments, the Fab region of the antibody has 6 N-glycosylation sites. In certain embodiments, 1, 2, 3, 4, 5, 6, 7, 8, 9, 10 or more N-glycosylation sites in the Fab region of the antibody are glycosylated by an N-glycan of structure:

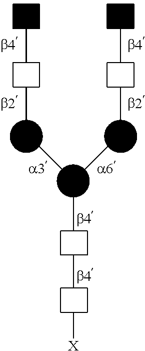

wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, at least two N-glycosylation sites of the Fab region of the antibody are glycosylated by the N-glycan. In certain embodiments, two N-glycosylation sites of the Fab region of the antibody are glycosylated by the N-glycan. In certain embodiments, one N-glycosylation site of the Fab region is located on each of the two heavy chain polypeptides of the antibody and each of said N-glycosylation sites is glycosylated by the N-glycan. In certain embodiments, four N-glycosylation sites of the Fab region of the antibody are glycosylated by the N-glycan. In certain embodiments, six N-glycosylation sites of the Fab region of the antibody are glycosylated by the N-glycan.

In certain embodiments, the Fc region of the antibody has 1, 2, 3, 4, 5, 6, 7, 8, 9, 10 or more N-glycosylation sites. In certain embodiments, Fc region of the antibody has 2 N-glycosylation sites. In certain embodiments, the Fc region of the antibody has 4 N-glycosylation sites. In certain embodiments, the Fc region of the antibody has 6 N-glycosylation sites. In certain embodiments, 1, 2, 3, 4, 5, 6, 7, 8, 9, 10 or more N-glycosylation sites in the Fc region of the antibody are glycosylated by an N-glycan of structure:

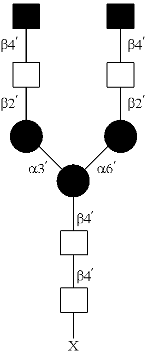

wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, at least two N-glycosylation sites of the Fc region of the antibody are glycosylated by the N-glycan. In certain embodiments, two N-glycosylation sites of the Fc region of the antibody are glycosylated by the N-glycan.

In other embodiments, 1, 2, 3, 4, 5, 6, 7, 8, 9, 10 or more N-glycosylation sites of the Fc region of the antibody are glycosylated by an N-glycan depicted in Section 5.3. In certain embodiments, 1, 2, 3, 4, 5, 6, 7, 8, 9, 10 or more N-glycosylation sites of the Fc region of the antibody are glycosylated by an N-glycan described in PCT/EP2022/057556, which is incorporated herein by reference in its entirety. In certain embodiments, at least two N-glycosylation sites of the Fc region of the antibody are glycosylated by an N-glycan having the structure of A2. In certain embodiments, two N-glycosylation sites of the Fc region of the antibody are glycosylated by an N-glycan having the structure of A2. In certain embodiments, four N-glycosylation sites of the Fc region of the antibody are glycosylated by an N-glycan having the structure of A2.

In certain embodiments, the Fc region comprises two different N-glycans (i.e. a first and a second N-glycan), wherein each N-glycan is independently linked to the Fc region at 1, 2, 3, 4, 5, 6, 7, 8, 9, 10 or more N-glycosylation sites, and wherein one of the N-glycans (i.e. the first N-glycan) has the structure of:

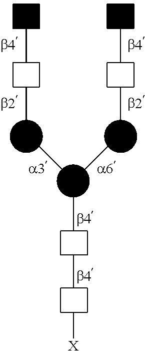

Wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, at least two N-glycosylation sites of the Fc region of the antibody are glycosylated by the first N-glycan. In certain embodiments, two N-glycosylation sites of the Fc region of the antibody are glycosylated by the first N-glycan. In certain embodiments, at least two N-glycosylation sites of the Fc region of the antibody are glycosylated by the second N-glycan. In certain embodiments, two N-glycosylation sites of the Fc region of the antibody are glycosylated by the second N-glycan. In certain embodiments, the different N-glycans specifically bind to different endocytic receptors. In certain embodiments, the first N-glycan specifically binds to ASGPR. In certain embodiments, the second N-glycan is an N-glycan depicted in Section 5.3. In certain embodiments, the second N-glycan is an N-glycan described in PCT/EP2022/057556, each of which is incorporated herein by reference in its entirety. In certain embodiments, the first N-glycan is larger than the second N-glycan. In other embodiments, the first N-glycan is smaller than the second N-glycan. In certain embodiments, the N-glycosylation sites predominantly or exclusively occupied by the larger N-glycan are more sterically accessible than the N-glycosylation sites predominantly or exclusively occupied by the smaller N-glycan. In certain embodiments, the other N-glycan is A2. In certain embodiments, the other N-glycan is A1GalNAc1 or A2GalNAc1.

In certain embodiments, only the Fc region and/or the hinge region of the antibody has one or more N-glycosylation sites. In other embodiments, the Fab region and the Fc region of the antibody each independently have 1, 2, 3, 4, 5, 6, 7, 8, 9, 10 or more N-glycosylation sites. In certain embodiments, the Fab region and the Fc region of the antibody each independently have 2, 4, or 6 N-glycosylation sites. In certain embodiments, the Fab region contains more N-glycosylation sites than the Fc region. In certain embodiments, the Fab region contains 1, 2, 3, 4, 5, 6, 7, 8, 9, or 10 more N-glycosylation sites than the Fc region. In certain embodiments, the Fab region contains 2 more N-glycosylation sites than the Fc region. In other embodiments, the Fc region contains more N-glycosylation sites than the Fab region. In certain embodiments, the Fc region contains 1, 2, 3, 4, 5, 6, 7, 8, 9, or 10 more N-glycosylation sites than the Fab region. In certain embodiments, the Fc region contains 2 or 4 more N-glycosylation sites than the Fab region. In still other embodiments, the Fab region and the Fc region contain the same number of N-glycosylation sites. In certain embodiments, the 1, 2, 3, 4, 5, 6, 7, 8, 9, 10 or more N-glycosylation sites of the Fab region are glycosylated by an N-glycan having a structure of:

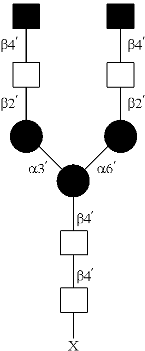

wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, 1, 2, 3, 4, 5, 6, 7, 8, 9, 10 or more N-glycosylation sites of the Fc region are glycosylated by an N-glycan having a structure of:

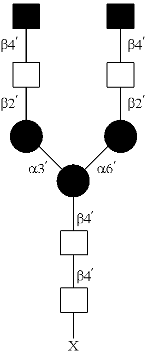

wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, 1, 2, 3, 4, 5, 6, 7, 8, 9, 10 or more N-glycosylation sites of the Fc region are glycosylated by an N-glycan depicted in Section 5.3. In certain embodiments, 1, 2, 3, 4, 5, 6, 7, 8, 9, 10 or more N-glycosylation sites of the Fc region are glycosylated by an N-glycan described in PCT/EP2022/057556, each of which is incorporated herein by reference in its entirety. In certain embodiments, 1, 2, 3, 4, 5, 6, 7, 8, 9, 10 or more N-glycosylation sites of the Fc region are glycosylated by an N-glycan having the structure of A2. In certain embodiments, 1, 2, 3, 4, 5, 6, 7, 8, 9, 10 or more N-glycosylation sites of the Fc region are glycosylated by two different N-glycans. In certain embodiments, the N-glycosylation sites of the Fab and/or Fc region that are distal to the hinge region of the antibody are glycosylated by the N-glycan having a structure of:

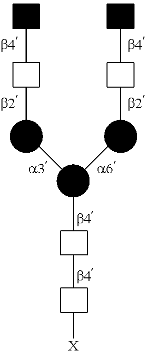

wherein the black square represents an N-acetyl galactosamine (GalNAc), the white square represents an N-acetylglucosamine (GlcNAc) residue the black circle represents a mannose (Man) residue, and X represents an amino acid residue of the antibody. In certain embodiments, the N-glycosylation sites of the Fab and/or Fc region that are proximal to the hinge region of the antibody are glycosylated by the N-glycan having the structure of A2.

In certain embodiments, the Fab region contains more N-glycans than the Fc region. In some embodiments, the Fab region contains 1, 2, 3, 4, 5, 6, 7, 8, 9, 10 or more N-glycans compared to the Fc region. In some embodiments, the Fab region contains 2 more N-glycans than the Fc region. In some embodiments, about 10% of the N-glycans are in the Fc region and about 90% of the N-glycans are in the Fab region. In some embodiments, about 20% of the N-glycans are in the Fc region and about 80% of the N-glycans are in the Fab region. In some embodiments, about 30% of the N-glycans are in the Fc region and about 70% of the N-glycans are in the Fab region. In some embodiments, about 40% of the N-glycans are in the Fc region and about 60% of the N-glycans are in the Fab region. In some embodiments, about 50% of the N-glycans are in the Fc region and about 50% of the N-glycans are in the Fab region. In some embodiments, the N-glycan structures in the Fab region and Fc region are identical (i.e., the same). In some embodiments, the N-glycan structures in the Fab region and Fc region are nonidentical (i.e., not the same).

In certain embodiments, the Fc region contains more N-glycans than the Fab region. In some embodiments, the Fc region contains 1, 2, 3, 4, 5, 6, 7, 8, 9, 10 or more N-glycans compared to the Fab region. In some embodiments, the Fc region contains 2 or 4 more N-glycans than the Fab region. In some embodiments, about 10% of the N-glycans are in the Fab region and about 90% of the N-glycans are in the Fc region. In some embodiments, about 20% of the N-glycans are in the Fab region and about 80% of the N-glycans are in the Fc region. In some embodiments, about 30% of the N-glycans are in the Fab region and about 70% of the N-glycans are in the Fc region. In some embodiments, about 40% of the N-glycans are in the Fab region and about 60% of the N-glycans are in the Fc region. In some embodiments, about 50% of the N-glycans are in the Fab region and about 50% of the N-glycans are in the Fc region. In some embodiments, the N-glycan structures in the Fab region and Fc region are identical (i.e., the same). In some embodiments, the N-glycan structures in the Fab region and Fc region are nonidentical (i.e., not the same).

In certain embodiments, wherein the target protein is a membrane protein, the Fc region and/or the hinge region contain more A2GalNAc2 glycans than the Fab region. In certain embodiments, wherein the target protein is a soluble protein, the Fab region contains more A2GalNAc2 glycans than the Fc region and/or the hinge region. In other embodiments, wherein the target protein is a soluble protein, the Fc region and/or the hinge region contain more A2GalNAc2 glycans than the Fab region.

In certain embodiments, the antibody has a N-glycan to protein ratio of 2 to 1, 4 to 1, 6 to 1, 8 to 1, or 10 to 1. In some embodiments, the antibody is glycosylated at a predetermined and specific residue. In other embodiments, the antibody is glycosylated at a random residue.

In certain embodiments, the glycoengineered bifunctional degrader binds to an autoantibody and comprises an autoantigen or immunogenic fragment thereof. In certain embodiments, the glycoengineered bifunctional degrader comprises a moiety that specifically binds to the target protein, and wherein the target protein is associated with a disease.

In certain embodiments, the glycoengineered bifunctional degrader a therapeutic polypeptide, i.e., a polypeptide used in the treatment of a disease or disorder. For example, the glycoengineered bifunctional degrader can be an enzyme, a cytokine, or an antibody. In certain embodiments, the glycoengineered bifunctional degrader is selected from the group consisting of adalimumab, rituximab and erythropoietin (EPO).

The glycoengineered bifunctional degrader can be any polypeptide (or peptide/polypeptide corresponding to the polypeptide) known in the art and used in accordance with the methods described herein. One of skill in the art will readily appreciate that the nucleic acid sequence of a known polypeptide, as well as a newly identified polypeptide, can easily be deduced using methods known in the art, and thus it would be well within the capacity of one of skill in the art to introduce a nucleic acid that encodes any bifunctional degrader into a host cell provided herein (e.g., via an expression vector, e.g., a plasmid, e.g., a site specific integration by homologous recombination).

In certain embodiments, the glycoengineered bifunctional degrader comprises the amino acid sequence of human Interferon-α (INF-α), Interferon-β (INF-β), Interferon-γ (INF-γ), Interleukin-2 (IL2), Chimeric diptheria toxin-IL-2 (Denileukin diftitox), Interleukin-1 (IL1), IL1B, IL3, IL4, IL11, IL21, IL22, IL1 receptor antagonist (anakinra), Tumor necrosis factor alpha (TNF-α), Insulin, Pramlintide, Growth hormone (GH), Insulin-like growth factor (IGF1), Human parathyroid hormone, Calcitonin, Glucagon-like peptide-1 agonist (GLP-1), Glucagon, Growth hormone-releasing hormone (GHRH), Secretin, Thyroid stimulating hormone (TSH), Human bone morphogenic protein 2 (hBMP2), Human bone morphogenic protein 7 (hBMP7), Gonadotropin releasing hormone (GnRH), Keratinocyte growth factor (KGF), Platelet-derived growth factor (PDGF), Fibroblast growth factor 7 (FGF7), Fibroblast growth factor 20 (FGF20), Fibroblast growth factor 21 (FGF21), Epidermal growth factor (EGF), Vascular endothelial growth factor (VEGF), Neurotrophin-3, Human follicle-stimulating hormone (FSH), Human chorionic gonadotropin (HCG), Lutropin-α, Erythropoietin, Granulocyte colony-stimulating factor (G-CSF), Granulocyte-macrophage colony-stimulating factor (GM-CSF), the extracellular domain of CTLA4 (e.g., an FC-fusion), or the extracellular domain of TNF receptor (e.g., an FC-fusion). In a specific embodiment, the glycoengineered bifunctional degrader is an enzyme or an inhibitor. Exemplary enzymes and inhibitors that can be used as a glycoengineered bifunctional degrader include, without limitation, Factor VII, Factor VIII, Factor IX, Factor X, Factor XIII, Factor VIIa, Antithrombin III (AT-III), Polypeptide C, Tissue plasminogen activator (tPA) and tPA variants, Urokinase, Hirudin, Streptokinase, Glucocerebrosidase, Alglucosidase-α, Laronidase (α-L-iduronidase), Idursulphase (Iduronate-2-sulphatase), Galsulphase, Agalsidase-β (human α-galactosidase A), Botulinum toxin, Collagenase, Human DNAse-I, Hyaluronidase, Papain, L-Asparaginase, Uricase (Urate oxidase), glutamate carboxypeptidase (glucarpidase), α1 Protease inhibitor (α1 antitrypsin), Lactase, Pancreatic enzymes (lipase, amylase, protease), and Adenosine deaminase.

In a specific embodiment, the glycoengineered bifunctional degrader is a cytokine. Exemplary cytokines that can be used as a glycoengineered bifunctional degrader include, without limitation, Interferon-α (INF-α), Interferon-β (INF-β), Interferon-γ (INF-γ), Interleukin-2 (IL2), Chimeric diptheria toxin-IL-2 (Denileukin diftitox), Interleukin-1 (IL1), IL1B, IL3, IL4, IL11, IL21, IL22, IL1 receptor antagonist (anakinra), and Tumor necrosis factor alpha (TNF-α).

In a specific embodiment, the glycoengineered bifunctional degrader is a hormone or growth factor. Exemplary hormones and growth factors that can be used as a glycoengineered bifunctional degrader include, without limitation, Insulin, Pramlintide, Growth hormone (GH), Insulin-like growth factor (IGF1), Human parathyroid hormone, Calcitonin, Glucagon-like peptide-1 agonist (GLP-1), Glucagon, Growth hormone-releasing hormone (GHRH), Secretin, Thyroid stimulating hormone (TSH), Human bone morphogenic protein 2 (hBMP2), Human bone morphogenic protein 7 (hBMP7), Gonadotropin releasing hormone (GnRH), Keratinocyte growth factor (KGF), Platelet-derived growth factor (PDGF), Fibroblast growth factor 7 (FGF7), Fibroblast growth factor 20 (FGF20), Fibroblast growth factor 21 (FGF21), Epidermal growth factor (EGF), Vascular endothelial growth factor (VEGF), Neurotrophin-3, Human follicle-stimulating hormone (FSH), Human chorionic gonadotropin (HCG), Lutropin-α, Erythropoietin, Granulocyte colony-stimulating factor (G-CSF), and Granulocyte-macrophage colony-stimulating factor (GM-CSF).

In a specific embodiment, the glycoengineered bifunctional degrader is a receptor. Exemplary receptors that can be used as a glycoengineered bifunctional degrader include, without limitation, the extracellular domain of human CTLA4 (e.g., fused to an Fc) and the soluble TNF receptor (e.g., fused to an Fc).

In other embodiments, the glycoengineered bifunctional degrader is a therapeutic polypeptide. In other embodiments, the glycoengineered bifunctional degrader is an approved biologic drug. In another embodiment, the therapeutic polypeptide comprises the amino acid sequence of Abatacept (e.g., Orencia® (Bristol-Myers Squibb)), Aducanumab-avwa (e.g., Aduhelm® (Biogen Corporation)), Aflibercept (e.g., Eylea® (Regeneron Corp.)), Agalsidase beta (e.g., Fabrazyme® (Genzyme Corp.)), Albiglutide (e.g., Eperzan® (GlaxoSmithKline Corp.)), Aldesleukin (e.g., Proleukin® (Clinigen, Inc.)), Alefacept (e.g., Amevive® (Astellas Pharma, Inc.)), Alglucerase (e.g., Ceredase® (Genzyme Corp.)), Alglucosidase alfa (e.g., Lumizyme® (Genzyme Corp.)), Aliskiren (e.g., Tekturna® (Noden Pharma)), Alpha-1-polypeptidease inhibitor (e.g., Aralast® (Takeda Pharmaceuticals, Inc.)), Alteplase (e.g., Activase® (Genentech)), Anakinra (e.g., Kineret® (Sobi, Inc.)), Anistreplase (e.g., Eminase® (SmithKlineBeecham)), Anthrax immune globulin human (e.g., Anthrasil® (Cangene Corp.)), Antihemophilic Factor (e.g., Advate® (Baxter Healthcare Corp.)), Antihemophelic Factor Fc-VWF-XTEN fusion protein (e.g., Altuviiio® (Bioverativ Therapeutics, Inc.)), Anti-inhibitor coagulant complex (e.g., Feiba Nf® (Takeda Pharmaceuticals, Inc.)), Antithrombin Alfa, Antithrombin III human, Antithymocyte globulin (e.g., Antithymocyte globulin), Anti-thymocyte Globulin (Equine) (e.g., ATGAM® (Pfizer)), Anti-thymocyte Globulin (Rabbit) (e.g., ATG-Fresenius), Aprotinin (e.g., Trasylol® (Bayer AG)), Asfotase Alfa (e.g., Strensiq® (AstraZeneca)), Asparaginase (e.g., Elspar® (Merck & Co., Inc.)), Asparaginase Erwinia chrysanthemi (e.g., Erwinaze® (EUSA Pharma, Inc.)), Becaplermin (e.g., Regranex® (Smith & Nephew, Inc.)), Belatacept (e.g., Nulojix® (Bristol-Myers Squibb)), Beractant, Bivalirudin (e.g., Angiomax® (The Medicines Co.)), Botulinum Toxin Type A (e.g., Botox® (Allergan, Inc)), Botulinum Toxin Type B (e.g., Myobloc® (Supernus Pharmaceuticals)), Brentuximab vedotin (e.g., Adcetris® (Seagen Inc.)), Buserelin (e.g., Suprecur® (Sanofi-Aventis)), C1 Esterase Inhibitor (Human) (e.g., Cinryze® (Takeda Corporation)), C1 Esterase Inhibitor (Recombinant) (e.g., Ruconest® (Salix Pharmaceuticals, Inc.)), Cerliponase alfa (e.g., Brineura® Biomarin Pharmaceutical, Inc.)), Certolizumab pegol (e.g., Cimzia® (UCB Pharma Ltd.)), Choriogonadotropin alfa (e.g., Choriogonadotropin alfa), Chorionic Gonadotropin (Human) (e.g., Ovidrel® (EMD Serono)), Chorionic Gonadotropin (Recombinant) (e.g., Ovitrelle® (Merck Serono)), Coagulation factor ix (e.g., Alprolix® (Bioverativ Therapeutics, Inc.)), Coagulation factor VIIa (e.g., NovoSeven® (Novo Nordisk A/S)), Coagulation factor X human (e.g., Coagadex® (Bio Products Laboratory, Ltd.)), Coagulation Factor XIII A-Subunit (Recombinant), Collagenase (e.g., Cordase® (Headway Pharma PVT Ltd.)), Conestat alfa, Corticotropin (e.g., H.P. Acthar® (Mallinckrodt Pharmaceuticals)), Cosyntropin (e.g., Cortrosyn® (Amphastar Pharmaceuticals, Inc.)), Darbepoetin alfa (e.g., Aranesp® (Amgen Inc.)), Defibrotide (e.g., Noravid® (Gentium S.p.A.)), Denileukin diftitox (e.g., Ontak® (Eisai Medical Research)), Desirudin, Digoxin Immune Fab (Ovine) (e.g., Digibind® (GlaxoSmithKline LLC)), Dornase alfa (e.g., Pulmozyme® (Genentech Inc.)), Drotrecogin alfa (e.g., Xigris® (Eli Lilly & Co.)), Dulaglutide (e.g., Trulicity® (Eli Lilly and Co.)), Efgartigimod alfa (e.g., Vyvgart® Hytrulo (Argenx, US, Inc.)), Ecallantide (e.g., Kalbitor® (Dyax Corp.)), Elapegademase (e.g., Revcovi® (Leadiant Biosciences, Inc.)), Efmoroctocog alfa (e.g., Elocta® (Swedish Orphan Biovitrum AB)), Elosulfase alfa (e.g., Vimizim® (Biomarin Pharmaceutical, Inc.)), Enfuvirtide (e.g., Fuzeon® (Genentech)), Eptinezumab (e.g., Vyepti® (Lundbeck Seattle Biopharmaceuticals, Inc.)), Epoetin alfa (e.g., Binocrit® (Sandoz GmbH)), Epoetin zeta (e.g., Retacrit® (Pfizer)), Eptifibatide (e.g., Integrilin® (COR Therapeutics, Inc.)), Etanercept (e.g., Enbrel® (Amgen Inc.)), Exenatide (e.g., Byetta® (AstraZeneca)), Factor IX Complex (Human) (e.g., AlphaNine® (Grifols Biologicals LLC)), Fibrinolysin aka plasmin (e.g., Elase® (Parke-Davis)), Filgrastim (e.g., N.A.), Filgrastim-sndz, Follitropin alfa (e.g., Gonal-F® (EMD Serono)), Follitropin beta (e.g., Follistim AQ® (Organon & Co.)), Galsulfase (e.g., Naglazyme® (BioMarin Pharmaceutical Inc.)), Gastric intrinsic factor, Gemtuzumab ozogamicin (e.g., Mylotarg® (Pfizer)), Glatiramer acetate (e.g., Copaxone® (Teva Neuroscience)), Glucagon recombinant (e.g., GlucaGen® (Novo Nordisk, Inc.)), Glucarpidase (e.g., Voraxaze® (BTG Pharmaceuticals)), Gramicidin D (e.g., Neosporin® (Johnson & Johnson Consumer, Inc.)), Hepatitis B immune globulin, Human calcitonin, Human Clostridium tetani toxoid immune globulin, Human rabies virus immune globulin (e.g., Hyperab Rabies Immune Globulin Human), Human Rho(D) immune globulin (e.g., Hyp Rho D Inj 16.5%), Human Serum Albumin (e.g., Albuminar® (CSL Behring LLC)), Human Varicella-Zoster Immune Globulin (e.g., Varizig® (Cangene Corporation)), Hyaluronidase (e.g., Hylenex® (Henry Schein, Inc.)), Hyaluronidase (Human Recombinant), Ibritumomab tiuxetan (e.g., Zevalin® (Acrotech Biopharma Inc.)), Idursulfase (e.g., Elaprase® (Takeda Pharmaceuticals, Inc.)), Imiglucerase (e.g., Cerezyme® (Genzyme Corporation)), Immune Globulin Human, Insulin aspart (e.g., NovoLog® (Novo Nordisk A/S)), Insulin Beef, Insulin Degludec (e.g., Tresiba® (Novo Nordisk A/S)), Insulin detemir (e.g., Levemir® (Novo Nordisk A/S)), Insulin Glargine (e.g., Lantus® (Sanofi-Aventis US LLC)), Insulin glulisine (e.g., Apidra® (Sanofi-Aventis US LLC)), Insulin Lispro (e.g., Humalog® (Eli Lilly Corp.)), Insulin Pork (e.g., Iletin II® (Eli Lilly Corporation)), Insulin Regular (e.g., Humulin R® (Eli Lilly Corp.)), Insulin, porcine (e.g., Vetsulin® (Merck & Co.)), Insulin, isophane (e.g., Novolin N)® (Novo Nordisk A/S), Interferon Alfa-2a, Recombinant (e.g., Roferon A® (Hoffman-LaRoche, Inc.)), Interferon alfa-2b (e.g., Intron A® (Merck & Co., Inc.)), Interferon alfacon-1 (e.g., Infergen® (Three Rivers Pharmaceuticals, LLC)), Interferon alfa-n1 (e.g., Wellferon® (GlaxoSmithKline)), Interferon alfa-n3 (e.g., Alferon® (AIM Immunotech Inc.)), Interferon beta-1a (e.g., Avonex® (Biogen-Idec Corporation)), Interferon beta-1b (e.g., Betaseron® (Bayer Healthcare Pharmaceuticals)), Interferon gamma-1b (e.g., Actimmune® (Horizon Pharma USA, Inc.)), Intravenous Immunoglobulin (e.g., Xembify® (Grifols Therapeutics LLC)), Laronidase (e.g., Aldurazyme® (Genzyme Corporation)), Lenograstim (e.g., Granocyte® (Chugai Pharmaceuticals, Inc.)), Lepirudin (e.g., Refludan® (Behring GmbH)), Leuprolide (e.g., Eligard® (Sanofi-Aventis US, LLC)), Liraglutide (e.g., Saxenda® (Novo-Nordisk, Inc.)), Lucinactant (e.g., Surfaxin), Lutropin alfa (e.g., Luveris® (EMD Serono)), Mecasermin (e.g., N.A.), Menotropins (e.g., Menopur® (Ferring Pharmaceuticals)), Methoxy polyethylene glycol-epoetin beta (e.g., Mircera® (Vifor Pharma)), Metreleptin (e.g., Myalept)® (AstraZeneca)), Natural alpha interferon OR multiferon (e.g., Intron/Roferon-A® (Merck & Co./Hoffman-LaRoche, Inc.)), Nesiritide (e.g., Natrecor® (Scios, Inc.))), Ocriplasmin (e.g., Jetrea® (ThromboGenics, Inc.)), Oprelvekin (e.g., Neumega® (Wyeth Pharmaceuticals, Inc.)), OspA lipopolypeptide (e.g., Lymerix® (GlaxoSmithKline)), Oxytocin (e.g., Pitocin® (Pfizer)), Palifermin (e.g., Kepivance® (Amgen, Inc.)), Pancrelipase (e.g., Creon® (AbbVie Inc.)), Pegademase bovine (e.g., Adagen® (Enzon Pharmaceuticals, Inc.)), Pegaspargase (e.g., Oncaspar® (Sigma-Tau Pharmaceuticals, Inc.)), Pegfilgrastim (e.g., Neulasta® (Amgen, Inc.)), Peginterferon alfa-2a (e.g., Pegasys® (Genentech USA, Inc.)), Peginterferon alfa-2b (e.g., PEG-Intron® (Merck & Co.)), Peginterferon beta-1a (e.g., Plegridy® (Biogen Corporation)), Pegloticase (e.g., (Krystexxa® (Horizon Therapeutics)), Pegvisomant (e.g., Somavert® (Pfizer)), Poractant alfa (e.g., Curosurf® (Chiesi USA, Inc/Cornerstone Therapeutics, Inc.)), Pramlintide (e.g., Symlin® (AstraZeneca Pharmaceuticals)), Preotact (e.g., Preotact® (Nycomed A/S)), Protamine sulfate (e.g., Protamine Sulfate Injection, USP), Polypeptide S human (e.g., Polypeptide S human), Prothrombin (e.g., Feiba Nf® (Takeda Pharmaceuticals, Inc.)), Prothrombin complex (e.g., Cofact® (Sanquin Plasma Products B.V.)), Prothrombin complex concentrate (e.g., Kcentra® (CSL Behring LLC)), Rasburicase (e.g., Elitek® (Sanofi-Aventis US, LLC)), Reteplase (e.g., Retavase® (Chiesi USA, Inc.)), Rilonacept (e.g., Arcalyst® (Kiniksa Pharmaceuticals, Ltd.)), Romiplostim (e.g., Nplate® (Amgen, Inc.)), Sacrosidase (e.g., Sucraid® (QOL Medical, LLC)), Salmon Calcitonin (e.g., Calcimar® (Sandoz GmbH))), Sargramostim (e.g., Leucomax® (Novartis)), Satumomab Pendetide (e.g., OncoScint® (Cytogen Corporation)), Sebelipase alfa (e.g., Kanuma® (Alexion Pharmaceuticals, Inc.)), Secretin (e.g., SecreFlo® (Repligen Corp.)), Sermorelin (e.g., Sermorelin acetate), Serum albumin (e.g., Albunex® (Mallinckrodt Medical, Inc.)), Serum albumin iodinated (e.g., Megatope® (Iso-Tex Diagnostics, Inc.)), Simoctocog Alfa (e.g., Nuwiq® (Octapharma USA, Inc.)), Sipuleucel-T (e.g., Provenge® (Dendreon Corporation)), Somatotropin Recombinant (e.g., NutropinAQ® (Genentech)), Somatropin recombinant (e.g., BioTropin® (Bio-Technology General)), Streptokinase (e.g., Streptase® (CSL Behring LLC)), Susoctocog alfa (e.g., Obizur® (Baxalta US, Inc.)), Taliglucerase alfa (e.g., Elelyso® (Pfizer, Inc.)), Teduglutide (e.g., Gattex® (NPS Pharmaceuticals, Inc.)), Tenecteplase (e.g., TNKase® (Genentech, Inc.)), Teriparatide (e.g., Forteo® (Lilly US, LLC)), Tesamorelin (e.g., Egrifta® (Theratechnologies, Inc.)), Thrombomodulin Alfa (e.g., Recomodulin® (Asahi Kasei Pharma)), Thymalfasin (e.g., Zadaxin® (SciClone Pharmaceuticals, Int'l)), Thyroglobulin, Thyrotropin Alfa (e.g., Thyrogen® (Genzyme Corporation)), Tuberculin Purified Polypeptide Derivative (e.g., Aplisol® (Par Pharmaceuticals)), Turoctocog alfa (e.g., Zonovate® (Novo Nordisk)), Urofollitropin (e.g., Bravelle® (Ferring Pharmaceuticals, Inc.)), Urokinase (e.g., Kinlytic® (ImaRx Therapeutics, Inc.)), Vasopressin (e.g., Pitressin® (JHP Pharmaceuticals, LLC)), Velaglucerase alfa (e.g., Vpriv® (Takeda Pharmaceuticals)), Abciximab (e.g., ReoPro® (Janssen Biotech, Inc.)), Adalimumab (e.g., Humira® (AbbVie, Inc.)), Alemtuzumab (e.g., Campath® (Takeda Oncology, Inc.)), Alirocumab (e.g., Praluent® (Regeneron/Sanofi)), Arcitumomab (e.g., CEA-Scan® (Immunomedics, Inc.)), Atezolizumab (e.g., Tecentriq® (Genentech)), Basiliximab (e.g., Simulect® (Novartis Pharmaceuticals Corporation)), Belimumab (e.g., Benlysta® (GlaxoSmithKline Inc.)), Benralizumab (e.g., Fasenra® (AstraZeneca)), Bevacizumab (e.g., Avastin® (Genentech, Inc.)), Bezlotoxumab (e.g., Zinplava® (Merck & Co., Inc.)), Blinatumomab (e.g., Blincyto® (Amgen, Inc.)), Brodalumab (e.g., Siliq® (Valeant Pharmaceuticals)), Brolucizumab (e.g., Beovu® (Novartis Pharmaceuticals Corporation)), Burosumab (e.g., Crysvita® (Ultragenyx, Inc.)), Calaspargase pegol (e.g., Asparlas® (Servier Pharmaceuticals, LLC)), Canakinumab (e.g., Ilaris® (Novartis Pharmaceuticals Corporation)), Caplacizumab (e.g., Cablivi® (Ablynx, N.V.)), Capromab (e.g., ProstaScint® (EUSA Pharma (USA), Inc.)), Cemiplimab (e.g., Libtayo® (Regeneron Pharmaceuticals, Inc.)), Cetuximab (e.g., Erbitux® (Lilly USA, LLC)), Crizanlizumab (e.g., Adakveo® (Novartis Pharmaceuticals Corporation)), Daclizumab (e.g., Zenapax® (Hoffmann-LaRoche, Inc.)), Daratumumab (e.g., Darzalex® (Janssen Biotech, Inc.)), Denosumab (e.g., Prolia® (Amgen, Inc., Xgeva® (Amgen, Inc.)), Dinutuximab (e.g., Unituxin® (United Therapeutics Corp.)), Dostarlimab (e.g., Jemperli® (GlaxoSmithKline, LLC)), Durvalumab (e.g., Imfinzi® (AstraZeneca)), Dupilimab (e.g., Dupixent® (Regeneron Pharmaceuticals, Inc.)), Eculizumab (e.g., Soliris® (Alexion Pharmaceuticals, Inc.)), Efalizumab (e.g., Raptiva® (Genentech, Inc.)), Elotuzumab (e.g., Empliciti® (Bristol-Myers Squibb)), Elranatamab (e.g., Elrexfio® (Pfizer, Inc.)), Emapalumab (e.g., Gamifant® (Sobi, Inc.)), Emicizumab (e.g., Hemlibra® (Genentech, Inc.)), Erenumab (e.g., Aimovig® (Amgen, Inc.)), Evinacumab (e.g., Evkeeza® (Regeneron Pharmaceuticals, Inc.)), Evolocumab (e.g., Repatha® (Amgen, Inc.)), fam-trastuzumab deruxtecan-nxki (e.g., Enhertu® (Daiichi Sankyo, Inc.)), Fremanezumab (e.g., Ajovy® (Teva Pharmaceuticals, Inc.)), Galcanezumab (e.g., Emgality® (Eli Lilly and Company)), Golimumab (e.g., Simponi® (Janssen Biotech, Inc.)), Guselkumab (e.g., Tremfya® (Janssen Biotech, Inc.)), Ibalizumab (e.g., Trogarzo® (Theratechnologies, Inc.)), Ibritumomab (e.g., Zevalin® (Acrotech Biopharma Inc.)), Idarucizumab (e.g., Praxbind® (Boehringer Ingelheim Pharmaceuticals, Inc.)), Infliximab (e.g., Remicade® (Janssen Biotech, Inc.)), Ipilimumab (e.g., Yervoy® (Bristol-Myers Squibb)), Isatuximab (e.g., Sarclisa® (Sanofi-Aventis, US, LLC)), Ixekizumab (e.g., Taltz® (Eli Lilly & Co.)), Lanadelumab (e.g., Takhzyro® (Takeda Pharmaceuticals, USA, Inc.)), Magrolimab (Gilead Sciences, Inc.), Margetuximab (e.g., Margenza® (Macrogenics, Inc.)), Mepolizumab (e.g., Nucala® (GlaxoSmithKline)), Muromonab (e.g., Orthoclone OKT3® (Centocor Ortho Biotech Products, LP)), Natalizumab (e.g., Tysabri® (Biogen Idec Corporation)), Necitumumab (e.g., Portrazza® (Eli Lilly and Company)), Nivolumab (e.g., Opdivo® (Bristol-Myers Squibb)), Obiltoxaximab (e.g., Anthim® (Elysys Therapeutics, Inc.)), Obinutuzumab (e.g., Gazyva® (Genentech, Inc.)), Ofatumumab (e.g., Arzerra® (GlaxoSmithKline)), Omalizumab (e.g., Xolair® (Genentech, Inc.)), Palivizumab (e.g., Synagis® (Medimmune, LLC)), Panitumumab (e.g., Vectibix® (Amgen, Inc.)), Pembrolizumab (e.g., Keytruda® (Merck & Co.)), Pertuzumab (e.g., Perjeta® (Genentech, Inc.)), Polatuzumab (e.g., Polivy® (Genentech, Inc.)), Pozelimab® (e.g., Veopoz (Regeneron Pharmaceuticals, Inc.)), Ramucirumab (e.g., Cyramza® (Eli Lilly and Company)), Ranibizumab (e.g., Lucentis® (Genentech, Inc.)), Ravulizumab-cwvz (e.g., Ultomoris® (AstraZeneca)), Raxibacumab (GlaxoSmithKline), Risankizumab (e.g., Risanizumab-rzaa, Skyrizi® (AbbVie Inc.)), Rituximab (e.g., Rituxan® (Genentech, Inc.)), Rozanolixizumab (e.g., Rystiggo® (UCB, Inc.)), Sarilumab (e.g., Kevzara® (Sanofi-Aventis, US, LLC)), Satrilizumab® (e.g., Enspryng (Genentech, Inc.)), Secukinumab (e.g., Cosentyx® (Novartis Pharmaceuticals Corporation)), Siltuximab (e.g., Sylvant® (Janssen Biotech, Inc.)), Tildrakizumab (e.g., Ilumya® (Merck & Co.)), Talquetamab (e.g., Talvey® (Janssen Biotech, Inc.)), Teclistamab (e.g., Tecvayli® (Janssen Biotech, Inc.)), Tocilizumab (e.g., Actemra® (Genentech, Inc.)), Tositumomab (e.g., Bexxar® (GlaxoSmithKline)), Trastuzumab (e.g., Herceptin® (Genentech, Inc.)), Ustekinumab (e.g., Stelara® (Janssen Biotech, Inc.)), or Vedolizumab (e.g., Entyvio® (Takeda Pharmaceuticals, USA, Inc.)).

In other embodiments, the glycoengineered bifunctional degrader comprises the amino acid sequence of an enzyme or an inhibitor thereof. In another embodiment, the glycoengineered bifunctional degrader comprises the amino acid sequence of Factor VII, Factor VIII, Factor IX, Factor X, Factor XIII, Factor VIIa, Antithrombin III (AT-III), Polypeptide C, Tissue plasminogen activator (tPA) and tPA variants, Urokinase, Hirudin, Streptokinase, Glucocerebrosidase, Alglucosidase-α, Laronidase (α-L-iduronidase), Idursulphase (Iduronate-2-sulphatase), Galsulphase, Agalsidase-β (human α-galactosidase A), Botulinum toxin, Collagenase, Human DNAse-I, Hyaluronidase, Papain, L-Asparaginase, Uricase (Urate oxidase), glutamate carboxypeptidase (glucarpidase), α1 Protease inhibitor (α1 antitrypsin), Lactase, Pancreatic enzymes (lipase, amylase, protease), and Adenosine deaminase.

In a specific embodiment, the glycoengineered bifunctional degrader is a receptor. Exemplary receptors that can be used as a glycoengineered bifunctional degrader include, without limitation, the extracellular domain of human CTLA4 (e.g., fused to an Fc) and the soluble TNF receptor (e.g., fused to an Fc).

In another embodiment, the glycoengineered bifunctional degrader is secreted into the culture media. In certain embodiments, the glycoengineered bifunctional degrader is purified from the culture media. In another embodiment, the glycoengineered bifunctional degrader is purified from the culture media via affinity purification or ion exchange chromatography. In another embodiment, the glycoengineered bifunctional degrader contains an FC domain and is affinity purified from the culture media via polypeptide-A. In another embodiment, the glycoengineered bifunctional degrader contains an affinity tag and is affinity purified.

In certain embodiments, the glycoengineered bifunctional degrader can be a full length polypeptide, a truncation, a polypeptide domain, a region, a motif or a peptide thereof.

In certain embodiments, the glycoengineered bifunctional degrader is a soluble receptor. In certain embodiments, the glycoengineered bifunctional degrader is an Fc-fusion polypeptide.

In certain embodiments, the glycoengineered bifunctional degrader is a biologic comprising an Fc domain of an IgG.

In certain embodiments, the glycoengineered bifunctional degrader is a ligand to a receptor.

In certain embodiments, the glycoengineered bifunctional degrader is localized in the secretory pathway. Without being bound by theory, localization within the secretory pathway includes, but is not limited to, localization to one or more of the following sub-cellular compartments: the endoplasmic reticulum, the Golgi apparatus, lysosomes, intracellular membrane proteins, cell surface anchored proteins, and membrane proteins. In certain embodiments, localization in the secretory pathway comprises localization to one or more of said sub-cellular compartments.

In certain embodiments, the glycoengineered bifunctional degrader comprises a signal peptide localizing the glycoengineered bifunctional degrader in the secretory pathway. In certain embodiments, the signal peptide is derived from the same source as the glycoengineered bifunctional degrader (i.e. the signal peptide is not added to the glycoengineered bifunctional degrader, but is one fused to the glycoengineered bifunctional degrader when naturally expressed in the source). In certain embodiments, the glycoengineered bifunctional degrader is localized in the secretory pathway without adding a Leishmania signal peptide to the glycoengineered bifunctional degrader. In other embodiments, the signal peptide is added to the glycoengineered bifunctional degrader. In certain embodiments, the signal peptide is derived from a Leishmania species. In certain embodiments, the signal peptide is derived from Leishmania tarentolae. In certain embodiments, the signal peptide is an invertase signal peptide from derived from Leishmania tarentolae. In certain embodiments, the signal peptide comprises an amino acid sequence of SEQ ID NO: 11. In certain embodiments, the signal peptide comprises an amino acid sequence of SEQ ID NO: 12. In certain embodiments, the signal peptide is processed and removed from the glycoengineered bifunctional degrader.

In another embodiment, the glycoengineered bifunctional degrader has been engineered to comprise one or more tag(s). In other embodiments, the tag is processed and removed from the glycoengineered bifunctional degrader.

In certain embodiments, the glycoengineered bifunctional degrader is expressed from a Leishmania host cell described in Section 7.3. In certain embodiments, the Leishmania host cells used to make the glycoengineered bifunctional degraders provided herein are genetically engineered using the methods described in Section 7.4. In certain embodiments, the Leishmania host cells used to make the glycoengineered bifunctional degraders provided herein are cultured according to the methods described in Section 7.5.

### 7.1.1 Pharmaceutical Formulations Comprising the Glycoengineered Bifunctional Degrader

In another aspect, provided herein are pharmaceutical compositions comprising the glycoengineered bifunctional degrader described herein. The compositions described herein are useful in the treatment and/or prevention of diseases/disorders in subjects (e.g., human subjects) (see Section 7.8).

In certain embodiments, in addition to comprising a glycoengineered bifunctional degrader described herein, the pharmaceutical described herein comprise a pharmaceutically acceptable carrier. As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In certain embodiments, the pharmaceutical compositions described herein are formulated to be suitable for the intended route of administration to a subject. For example, the pharmaceutical compositions described herein may be formulated to be suitable for subcutaneous, parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration.

In certain embodiments, the pharmaceutical compositions described herein additionally comprise one or more buffers, e.g., phosphate buffer and sucrose phosphate glutamate buffer. In other embodiments, the pharmaceutical compositions described herein do not comprise buffers.

In certain embodiments, the pharmaceutical compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the pharmaceutical compositions described herein do not comprise salts.

In some embodiments, the pharmaceutical compositions described herein can be administered in a single dosage form, for example a single dosage form of a glycoengineered bifunctional degrader described here.

The pharmaceutical compositions described herein can be included in a kit, container, pack, or dispenser together with instructions for administration. In some embodiments, provided herein is a kit comprising the glycoengineered bifunctional degrader of the present disclosure is provided herein. In some embodiments, the kit further provides instructions for administering the bifunctional molecule or pharmaceutical composition to an individual in need thereof.

The pharmaceutical compositions described herein can be stored before use, e.g., the compositions can be stored frozen (e.g., at about −20 °C or at about −70 °C); stored in refrigerated conditions (e.g., at about 4 °C); or stored at room temperature.

### 7.2 Population of Glycoengineered Bifunctional Degraders

Also provided herein is a composition comprising a population of bifunctional degraders described in Section 7.1, wherein the population of bifunctional degraders has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s). In certain embodiment, the N-glycan profile is at least 30%, at least 40%, at least 50%, at least 60%, at least 70%, at least 80%, at least 90%, at least 95%, at least 98%, or about 100% homogenous at one of the N-glycosylation sites. In certain embodiments, the N-glycan profile is at least 30%, at least 40%, at least 50%, at least 60%, at least 70%, at least 80%, at least 90%, at least 95%, at least 98%, or about 100% homogeneous at two of the N-glycosylation sites. In certain embodiments, the N-glycan profile is at least 50% homogeneous at one or more of the N-glycosylation site(s). In certain embodiments, the N-glycan profile is at least 60% homogeneous at one or more of the N-glycosylation site(s). In certain embodiments, the N-glycan profile is at least 70% homogenous at one or more of the N-glycosylation site(s). In certain embodiments, the N-glycan profile is at least 80% homogeneous at one or more of the N-glycosylation site(s). In certain embodiments, the N-glycan profile is at least 90% homogeneous at one or more of the N-glycosylation site(s). In certain embodiments, the N-glycan profile is at least 95% homogenous at one or more of the N-glycosylation site(s). In certain embodiments, the N-glycan profile is at least 98% homogenous at one or more of the N-glycosylation site(s). In certain embodiments, the homogeneity of the N-glycan profile at one or more of the N-glycosylation sites is determined according to any standard means known in the art. In certain embodiments, the homogeneity of the N-glycan profile at one or more of the N-glycosylation sites is determined by N-glycan analysis, glycopeptide analysis or intact protein analysis. In certain embodiments, the homogeneity of the N-glycan profile at one or more of the N-glycosylation sites is determined according to one or more assays described in Section 7.9.3.

Also provided herein is a composition comprising a population of bifunctional degraders described in Section 7.1, wherein the population of bifunctional degraders has an N-glycan profile comprising about 30% to 40%, about 40% to about 50%, about 50% to about 60%, about 60% to about 70%, about 70% to about 80%, about 80% to about 90%, or about 90% to about 100% of an N-glycan of the structure:

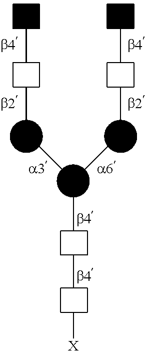

at one or more of the N-glycosylation site(s), wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In certain embodiments, the N-glycan profile comprises about 30% to about 40%, about 40% to about 50%, about 50% to about 60%, about 60% to about 70%, about 70% to about 80%, about 80% to about 90%, or about 90% to about 100% of the N-glycan at one N-glycosylation site. In certain embodiments, the N-glycan profile comprises about 30% to about 40%, about 40% to about 50%, about 50% to about 60%, about 60% to about 70%, about 70% to about 80%, about 80% to about 90%, or about 90% to about 100% of the N-glycan at two N-glycosylation sites, respectively. In certain embodiments, the N-glycan profile comprises about 30% to about 40% of the N-glycan of the structure at one or more of the N-glycosylation site(s). In certain embodiments, the N-glycan profile comprises about 40% to about 50% of the N-glycan of the structure at one or more of the N-glycosylation site(s). In certain embodiments, the N-glycan profile comprises about 50% to about 60% of the N-glycan of the structure at one or more of the N-glycosylation site(s). In certain embodiments, the N-glycan profile comprises about 60% to about 70% of the N-glycan at one or more of the N-glycosylation site(s). In certain embodiments, the N-glycan profile comprises about 70% to about 80% of the N-glycan at one or more of the N-glycosylation site(s). In certain embodiments, the N-glycan profile comprises about 80% to about 90% of the N-glycan at one or more of the N-glycosylation site(s). In certain embodiments, the N-glycan profile comprises about 90% to about 100% of the N-glycan at one or more of the N-glycosylation site(s). In certain embodiments, the relative amount of the N-glycan at one or more of the N-glycosylation sites is determined according to any standard means known in the art. In certain embodiments, the relative amount of the N-glycan at one or more of the N-glycosylation sites is determined by N-glycan analysis or glycopeptide analysis. In certain embodiments, the relative amount of the N-glycan at one or more of the N-glycosylation sites is determined according to one or more assays described in Section 7.9.3.

Without being bound by theory, N-glycan structures that are not fully capped with GalNAc (for example, A1GalNAc1 or A2GalNAc1 as compared to A2GalNAc2) do not engage ASGPR. In certain preferred embodiments, the N-glycan profile comprises about 90% to about 100% of the N-glycan of structure:

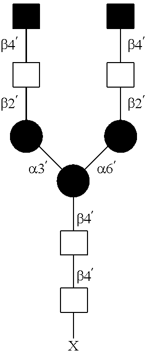

at one or more of the N-glycosylation site(s), wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In one embodiment, the N-glycan profile comprises about 90% to about 100% of the N-glycan at one N-glycosylation site. In one embodiment, the N-glycan profile comprises about 95% to about 100% of the N-glycan at one N-glycosylation site. In one embodiment, the N-glycan profile comprises about 90% to about 100% of the N-glycan at two N-glycosylation sites, respectively. In one embodiment, the N-glycan profile comprises about 95% to about 100% of the N-glycan at two N-glycosylation sites, respectively. In one embodiment, the N-glycan profile comprises about 80% to about 90% of the N-glycan at two N-glycosylation sites, collectively. In one embodiment, the N-glycan profile comprises about 90% to about 100% of the N-glycan at two N-glycosylation sites, collectively. In certain embodiments, the relative amount of the N-glycan at one or more of the N-glycosylation sites is determined according to any standard means known in the art. In certain embodiments, the relative amount of the N-glycan at one or more of the N-glycosylation sites is determined by N-glycan analysis or glycopeptide analysis. In certain embodiments, the relative amount of the N-glycan at one or more of the N-glycosylation sites is determined according to one or more assays described in Section 7.9.3.

Also provided herein is a composition comprising a population of bifunctional degraders described in Section 7.1, wherein the population of bifunctional degraders has an N-glycan profile comprising at least 30%, at least 40%, at least 50%, at least 60%, at least 70%, at least 80%, at least 90%, at least 95%, at least 98%, or about 100% of an N-glycan of the structure:

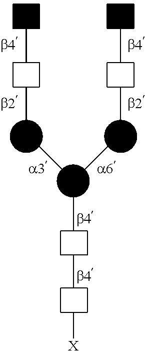

among all glycans in the N-glycan profile, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In certain embodiments, the population of bifunctional degraders has an N-glycan profile comprising at least 30% of the N-glycan among all glycans in the N-glycan profile. In certain embodiments, the population of bifunctional degraders has an N-glycan profile comprising at least 40% of the N-glycan among all glycans in the N-glycan profile. In certain embodiments, the population of bifunctional degraders has an N-glycan profile comprising at least 50% of the N-glycan among all glycans in the N-glycan profile. In certain embodiments, the population of bifunctional degraders has an N-glycan profile comprising at least 60% of the N-glycan among all glycans in the N-glycan profile. In certain embodiments, the population of bifunctional degraders has an N-glycan profile comprising at least 70% of the N-glycan among all glycans in the N-glycan profile. In certain embodiments, the population of bifunctional degraders has an N-glycan profile comprising at least 80% of the N-glycan among all glycans in the N-glycan profile. In certain embodiments, the population of bifunctional degraders has an N-glycan profile comprising at least 90% of the N-glycan among all glycans in the N-glycan profile. In certain embodiments, the population of bifunctional degraders has an N-glycan profile comprising at least 95% of the N-glycan among all glycans in the N-glycan profile. In certain embodiments, the population of bifunctional degraders has an N-glycan profile comprising at least 98% of the N-glycan among all glycans in the N-glycan profile.

Also provided herein is a composition comprising a population of bifunctional degraders described in Section 7.1, wherein the population of bifunctional degraders has an N-glycan profile comprising about 30% to about 40%, about 40% to about 50%, about 50% to about 60%, about 60% to about 70%, about 70% to about 80%, about 80% to about 90%, or about 90% to about 100% of an N-glycan of the structure:

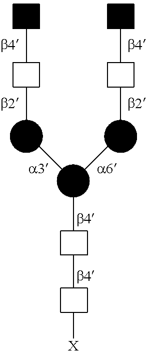

among all glycans in the N-glycan profile, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader. In certain embodiments, the population of bifunctional degraders has an N-glycan profile comprising about 30% to about 40% of the N-glycan among all glycans in the N-glycan profile. In certain embodiments, the population of bifunctional degraders has an N-glycan profile comprising about 40% to about 50% of the N-glycan among all glycans in the N-glycan profile. In certain embodiments, the population of bifunctional degraders has an N-glycan profile comprising about 50% to about 60% of the N-glycan among all glycans in the N-glycan profile. In certain embodiments, the population of bifunctional degraders has an N-glycan profile comprising about 60% to about 70% of the N-glycan among all glycans in the N-glycan profile. In certain embodiments, the population of bifunctional degraders has an N-glycan profile comprising about 70% to about 80% of the N-glycan among all glycans in the N-glycan profile. In certain embodiments, the population of bifunctional degraders has an N-glycan profile comprising about 80% to about 90% of the N-glycan among all glycans in the N-glycan profile. In certain embodiments, the population of bifunctional degraders has an N-glycan profile comprising about 90% to about 100% of the N-glycan among all glycans in the N-glycan profile. In certain embodiments, the relative amount of the N-glycan among all glycans in the N-glycan profile is determined according to any standard means known in the art. In certain embodiments, the relative amount of the N-glycan among all glycans in the N-glycan profile is determined by N-glycan analysis, glycopeptide analysis or intact protein analysis. In certain embodiments, the relative amount of the N-glycan among all glycans in the N-glycan profile is determined according to one or more assays described in Section 7.9.3.

Also provided herein is a composition comprising a population of bifunctional degraders described in Section 7.1, wherein the population of bifunctional degraders has an N-glycan profile that is at least 60%, at least 70%, at least 80%, at least 90%, at least 95%, at least 98%, or about 100% homogeneous. In certain embodiments, the population has an N-glycan profile that is at least 60% homogeneous. In certain embodiments, the population has an N-glycan profile that is at least 70% homogeneous. In certain embodiments, the population has an N-glycan profile that is at least 80% homogeneous. In certain embodiments, the population has an N-glycan profile that is at least 90% homogeneous. In certain embodiments, the population has an N-glycan profile that is at least 95% homogeneous. In certain embodiments, the population has an N-glycan profile that is at least 98% homogeneous. In certain embodiments, the population has an N-glycan profile that is about 100% homogeneous. In certain embodiments, the homogeneity of the N-glycan profile is determined according to any standard means known in the art. In certain embodiments, the homogeneity of the N-glycan profile is determined by N-glycan analysis, glycopeptide analysis or intact protein analysis. In certain embodiments, the homogeneity of the N-glycan profile is determined according to one or more assays described in Section 7.9.3.

Also provided herein is a composition comprising a population of bifunctional degraders described in Section 7.1, wherein the population of bifunctional degraders has an N-glycan profile that is about 60% to about 70%, about 70% to about 80%, about 80% to about 90%, or about 90% to about 100% homogeneous. In certain embodiments, the population has an N-glycan profile that is about 30% to about 40% homogeneous. In certain embodiments, the population has an N-glycan profile that is about 40% to about 50% homogeneous. In certain embodiments, the population has an N-glycan profile that is about 50% to about 60% homogeneous. In certain embodiments, the population has an N-glycan profile that is about 60% to about 70% homogeneous. In certain embodiments, the population has an N-glycan profile that is about 70% to about 80% homogeneous. In certain embodiments, the population has an N-glycan profile that is about 80% to about 90% homogeneous. In certain embodiments, the population has an N-glycan profile that is about 90% to about 100% homogeneous. In certain embodiments, the homogeneity of the N-glycan profile is determined according to any standard means known in the art. In certain embodiments, the homogeneity of the N-glycan profile is determined by N-glycan analysis, glycopeptide analysis or intact protein analysis. In certain embodiments, the homogeneity of the N-glycan profile is determined according to one or more assays described in Section 7.9.3.

In certain embodiments, the population of bifunctional degraders described in this Section is produced by Leishmania host cells described in Section 7.3. In certain embodiments, the Leishmania host cells used to produce the population of glycoengineered bifunctional degraders described in this Section aree genetically engineered using the methods described in Section 7.4. In certain embodiments, the Leishmania host cell used to produce the population of glycoengineered bifunctional degraders described in this Section are cultured according to the methods described in Section 7.5.

### 7.2.1 Pharmaceutical Compositions Comprising a Population of Bifunctional Degraders

In another aspect, provided herein are pharmaceutical compositions comprising a population of glycoengineered bifunctional degraders described herein. The compositions described herein are useful in the treatment and/or prevention of diseases/disorders in subjects (e.g., human subjects) (see Section 7.8).

In certain embodiments, in addition to comprising a population of glycoengineered bifunctional degraders described herein, the pharmaceutical described herein comprise a pharmaceutically acceptable carrier. As used herein, the term “pharmaceutically acceptable” means approved by a regulatory agency of the Federal or a state government or listed in the U.S. Pharmacopeia or other generally recognized pharmacopeiae for use in animals, and more particularly in humans. The term “carrier,” as used herein in the context of a pharmaceutically acceptable carrier, refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin.

In certain embodiments, the pharmaceutical compositions described herein are formulated to be suitable for the intended route of administration to a subject. For example, the pharmaceutical compositions described herein may be formulated to be suitable for subcutaneous, parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration.

In certain embodiments, the pharmaceutical compositions described herein additionally comprise one or more buffers, e.g., phosphate buffer and sucrose phosphate glutamate buffer. In other embodiments, the pharmaceutical compositions described herein do not comprise buffers.

In certain embodiments, the pharmaceutical compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the pharmaceutical compositions described herein do not comprise salts.

In some embodiments, the pharmaceutical compositions described herein can be administered in a single dosage form, for example a single dosage form of a population of glycoengineered bifunctional degraders described here.

The pharmaceutical compositions described herein can be included in a kit, container, pack, or dispenser together with instructions for administration. In some embodiments, provided herein is a kit comprising the population of glycoengineered bifunctional degraders of the present disclosure is provided herein. In some embodiments, the kit further provides instructions for administering the population of glycoengineered bifunctional degraders or pharmaceutical composition to an individual in need thereof.

The pharmaceutical compositions described herein can be stored before use, e.g., the compositions can be stored frozen (e.g., at about −20 °C or at about −70 °C); stored in refrigerated conditions (e.g., at about 4 °C); or stored at room temperature.

### 7.3 Leishmania Host Cells

Provided herein are Leishmania host cells for the production of glycoengineered bifunctional degraders described in Section 7.1 or a population of glycoengineered bifunctional degraders described in Section 7.2, wherein the Leishmania host cells comprise: (a) a recombinant nucleic acid encoding a glycoengineered bifunctional degrader; and (b) a recombinant nucleic acid encoding one or more recombinant N-acetylgalactosamine (GalNAc) transferases. In certain embodiments, the Leishmania host cells provided herein are capable of producing glycoengineered bifunctional degraders comprising a biantennary, GalNAc-terminated N-glycan. In particular, the Leishmania host cells provided herein are capable of producing glycoengineered bifunctional degraders comprising an N-glycan of the following structure:

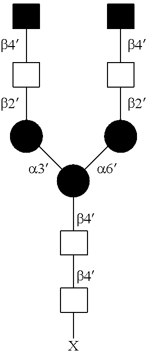

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered bifunctional degrader.

In certain embodiments, the Leishmania host cells provided herein comprise a recombinant nucleic acid encoding one or more recombinant N-acetylgalactosamine (GalNAc) transferases described in Section 7.3.1. In certain embodiments, the Leishmania host cells provided herein comprise a recombinant nucleic acid encoding one or more additional recombinant glycosyltransferases described in Section 7.3.2. In certain embodiments, one or more endogenous enzymes described in Section 7.3.3 from the glycan biosynthesis pathway of the the Leishmania host cells provided herein have been deleted, mutated and/or functionally inactivated. In certain embodiments, the Leishmania host cells provided herein further comprise a recombinant nucleic acid encoding heterologous UDP-GalNAc biosynthetic pathway proteins as described in Section 7.3.4 capable of generating UDP-GalNAc. In certain embodiments, the Leishmania host cells provided herein comprise a recombinant nucleic acid encoding a heterologous UDP-GalNAc transporter protein as described in Section 7.3.5 capable of transporting UDP-GalNAc to the secretory pathway. In certain embodiments, the strain of the Leishmania host cells provided herein is described in Section 7.3.6.

In certain embodiments, the Leishmania host cells provided herein below are genetically engineered using the methods described in Section 7.4. In certain embodiments, the Leishmania host cells provided herein below are cultured according to the methods described in Section 7.5.

Notwithstanding anything in this Section, other suitable host cells comprise liver cells, myeloid cells, immune cells, endothelial cells, parenchymal cells or epithelial cells. In some embodiments, the immune cell is a dendritic cell, a macrophage, a monocyte, a microglia cell, a granulocyte or a B lymphocyte.

### 7.3.1 N-Acetylgalactosamine (GalNAc) Transferases

The Leishmania host cells provided herein comprise a recombinant nucleic acid encoding one or more recombinant N-acetylgalactosamine (GalNAc) transferases. In certain embodiments, the GalNAc transferase, or a functionally active variant thereof, is capable of catalyzing the addition of a GalNAc to a N-acetyl glucosamine-terminated glycan.

In certain embodiments, the GalNAc transferase is heterologous to the Leishmania host cell. In certain embodiments, the GalNAc transferase is derived from Homo sapiens, Caenorhabditis elegans, Parasteatoda tepidariorum, Salmo trutta, or Hucho hucho. In certain embodiments, the GalNAc transferase is derived from a mammalian source. In certain embodiments, the mammalian source is Homo sapiens.

In certain embodiments, the GalNAc transferase is selected from the group consisting of β4-GalNAcT3, β4-GalNAcT4, Ceβ4GalNAcT, Ptβ4GalNAcT, and Stβ4GalNAcT, or functionally active variants thereof. In certain embodiments, the GalNAc transferase is selected from the group consisting of β4-GalNAcT3, β4-GalNAcT4, Ceβ4GalNAcT, Ptβ4GalNAcT, and Stβ4GalNAcT, or variants that are at least 80%, at least 85%, at least 90%, at least 95%, at least 98%, or at least 99% homologous thereof.

In certain embodiments, the GalNAc transferase comprises β4-GalNAcT3, or a functionally active variant thereof. In certain embodiments, the GalNAc transferase comprises β4-GalNAcT3. In certain embodiments, the GalNAc transferase comprises a variant that is at least 80%, at least 85%, at least 90%, at least 95%, at least 98%, or at least 99% homologous to β4-GalNAcT3. In certain embodiments, the GalNAc transferase comprises an N-terminally truncated variant of β4-GalNAcT3. In certain embodiments, the β4-GalNAcT3 comprises β4-GalNAcT3 of Homo sapiens, or a functionally active variant thereof. In certain embodiments, the GalNAc transferase comprises β4-GalNAcT3 of Homo sapiens. In certain embodiments, the GalNAc transferase comprises an amino acid sequence of SEQ ID NO: 1. In certain embodiments, the GalNAc transferase comprises one that is homologous to β4-GalNAcT3 of Homo sapiens. In certain embodiments, the GalNAc transferase comprises a variant that is at least 80%, at least 85%, at least 90%, at least 95%, at least 98%, or at least 99% homologous to β4-GalNAcT3 of Homo sapiens. In certain embodiments, the GalNAc transferase comprises an N-terminally truncated variant of β4-GalNAcT3 of Homo sapiens comprising an amino acid sequence of SEQ ID NO: 2.

In certain embodiments, the GalNAc transferase comprises β4-GalNAcT4, or a functionally active variant thereof. In certain embodiments, the GalNAc transferase comprises β4-GalNAcT4. In certain embodiments, the GalNAc transferase comprises a variant that is at least 80%, at least 85%, at least 90%, at least 95%, at least 98%, or at least 99% homologous to β4-GalNAcT4. In certain embodiments, the GalNAc transferase comprises an N-terminally truncated variant of β4-GalNAcT4. In certain embodiments, the β4-GalNAcT4 comprises β4-GalNAcT4 of Homo sapiens, or a functionally active variant thereof. In certain embodiments, the GalNAc transferase comprises β4-GalNAcT4 of Homo sapiens. In certain embodiments, the GalNAc transferase comprises an amino acid sequence of SEQ ID NO: 3. In certain embodiments, the GalNAc transferase comprises one that is homologous to β4-GalNAcT4 of Homo sapiens. In certain embodiments, the GalNAc transferase comprises a variant that is at least 80%, at least 85%, at least 90%, at least 95%, at least 98%, or at least 99% homologous to β4-GalNAcT4 of Homo sapiens. In certain embodiments, the GalNAc transferase comprises an N-terminally truncated variant of β4-GalNAcT4 of Homo sapiens comprising an amino acid sequence of SEQ ID NO: 4.

In certain embodiments, the GalNAc transferases comprise β4-GalNAcT3 and β-GalNAcT4, or functionally active variants thereof. In certain embodiments, the GalNAc transferases comprise β4-GalNAcT3 and β4-GalNAcT4. In certain embodiments, the GalNAc transferases comprise variants that are at least 80%, at least 85%, at least 90%, at least 95%, at least 98%, or at least 99% homologous to β4-GalNAcT3 and β-GalNAcT4, respectively. In certain embodiments, the GalNAc transferases comprise N-terminally truncated variants of β4-GalNAcT3 and/or β4-GalNAcT4. In certain embodiments, the GalNAc transferases comprise β4-GalNAcT3 and β4-GalNAcT4 of Homo sapiens, or functionally active variants thereof. In certain embodiments, the GalNAc transferases comprise β4-GalNAcT3 and β4-GalNAcT4 of Homo sapiens. In certain embodiments, the GalNAc transferases comprise amino acid sequences of SEQ ID NO: 1 and SEQ ID NO: 3. In certain embodiments, the GalNAc transferases comprise ones that are homologous to β4-GalNAcT3 and β4-GalNAcT4 of Homo sapiens. In certain embodiments, the GalNAc transferases comprise variants that are at least 80%, at least 85%, at least 90%, at least 95%, at least 98%, or at least 99% homologous to β4-GalNAcT3 and β4-GalNAcT4 of Homo sapiens, respectively. In certain embodiments, the GalNAc transferases comprise N-terminally truncated variants of β4-GalNAcT3 and/or β4-GalNAcT4 of Homo sapiens comprising amino acid sequences of SEQ ID NO: 2 and SEQ ID NO: 4, respectively.

In certain embodiments, the GalNAc transferase is Ceβ4GalNAcT, or a functionally active variant thereof. In certain embodiments, the GalNAc transferase is Ceβ4GalNAcT. In certain embodiments, the GalNAc transferase is a variant that is at least 80%, at least 85%, at least 90%, at least 95%, at least 98%, or at least 99% homologous to Ceβ4GalNAcT. In certain embodiments, the GalNAc transferase is an N-terminally truncated variant of Ceβ4GalNAcT. In certain embodiments, the Ceβ4GalNAcT is a β4GalNAcT of Caenorhabditis elegans, or a functionally active variant thereof. In certain embodiments, the GalNAc transferase is a β4GalNAcT of Caenorhabditis elegans. In certain embodiments, the GalNAc transferase comprises an amino acid sequence of SEQ ID NO: 5. In certain embodiments, the GalNAc transferase is one that is homologous to a Ceβ4GalNAcT of Caenorhabditis elegans. In certain embodiments, the GalNAc transferase is a variant that is at least 80%, at least 85%, at least 90%, at least 95%, at least 98%, or at least 99% homologous to a Ceβ4GalNAcT of Caenorhabditis elegans. In certain embodiments, the GalNAc transferase comprises an N-terminally truncated variant of Ceβ4GalNAcT of Caenorhabditis elegans comprising an amino acid sequence of SEQ ID NO: 6.

In certain embodiments, the GalNAc transferase is Ptβ4GalNAcT, or a functionally active variant thereof. In certain embodiments, the GalNAc transferase is Ptβ4GalNAcT. In certain embodiments, the GalNAc transferase is a variant that is at least 80%, at least 85%, at least 90%, at least 95%, at least 98%, or at least 99% homologous to Ptβ4GalNAcT. In certain embodiments, the GalNAc transferase is an N-terminally truncated variant of Ptβ4GalNAcT. In certain embodiments, the Ptβ4GalNAcT is a β4GalNAcT of Parasteatoda tepidariorum, or a functionally active variant thereof. In certain embodiments, the GalNAc transferase is a Ptβ4GalNAcT of Parasteatoda tepidariorum. In certain embodiments, the GalNAc transferase comprises an amino acid sequence of SEQ ID NO: 7. In certain embodiments, the GalNAc transferase is one that is homologous to a Ptβ4GalNAcT of Parasteatoda tepidariorum. In certain embodiments, the GalNAc transferase is a variant that is at least 80%, at least 85%, at least 90%, at least 95%, at least 98%, or at least 99% homologous to a Ptβ4GalNAcT of Parasteatoda tepidariorum. In certain embodiments, the GalNAc transferase comprises an N-terminally truncated variant of Ptβ4GalNAcT of Parasteatoda tepidariorum comprising an amino acid sequence of SEQ ID NO: 8.

In certain embodiments, the GalNAc transferase is Stβ4GalNAcT, or a functionally active variant thereof. In certain embodiments, the GalNAc transferase is Stβ4GalNAcT. In certain embodiments, the GalNAc transferase is a variant that is at least 80%, at least 85%, at least 90%, at least 95%, at least 98%, or at least 99% homologous to Stβ4GalNAcT. In certain embodiments, the GalNAc transferase is an N-terminally truncated variant of Stβ4GalNAcT. In certain embodiments, the Stβ4GalNAcT is a β4GalNAcT of Salmo trutta, or a functionally active variant thereof. In certain embodiments, the GalNAc transferase is a β4GalNAcT of Salmo trutta. In certain embodiments, the GalNAc transferase comprises an amino acid sequence of SEQ ID NO: 9. In certain embodiments, the GalNAc transferase is one that is homologous to a β4GalNAcT of Salmo trutta. In certain embodiments, the GalNAc transferase is a variant that is at least 80%, at least 85%, at least 90%, at least 95%, at least 98%, or at least 99% homologous to a β4GalNAcT of Salmo trutta.

In certain embodiments, the GalNAc transferase is Hhβ4GalNAcT, or a functionally active variant thereof. In certain embodiments, the GalNAc transferase is Hhβ4GalNAcT. In certain embodiments, the GalNAc transferase is a variant that is at least 80%, at least 85%, at least 90%, at least 95%, at least 98%, or at least 99% homologous to Hhβ4GalNAcT. In certain embodiments, the GalNAc transferase is an N-terminally truncated variant of Hhβ4GalNAcT. In certain embodiments, the Hhβ4GalNAcT is a β4GalNAcT of Hucho hucho, or a functionally active variant thereof. In certain embodiments, the GalNAc transferase is a β4GalNAcT of Hucho hucho. In certain embodiments, the GalNAc transferase comprises an amino acid sequence of SEQ ID NO: 10. In certain embodiments, the GalNAc transferase is one that is homologous to a β4GalNAcT of Hucho hucho. In certain embodiments, the GalNAc transferase is a variant that is at least 80%, at least 85%, at least 90%, at least 95%, at least 98%, or at least 99% homologous to a β4GalNAcT of Hucho hucho.

In certain embodiments, the GalNAc transferase is localized in the secretory pathway. Without being bound by theory, localization within the secretory pathway includes, but is not limited to, localization to one or more of the following sub-cellular compartments: the endoplasmic reticulum, the Golgi apparatus, lysosomes, intracellular membrane proteins, cell surface anchored proteins, and membrane proteins. In certain embodiments, localization in the secretory pathway comprises localization to one or more of said sub-cellular compartments.

In certain embodiments, the GalNAc transferase comprises a signal peptide localizing the GalNAc transferase in the secretory pathway. In certain embodiments, the signal peptide is derived from the same source as the GalNAc transferase (i.e. the signal peptide is not added to the GalNAc transferase, but is one contained in the GalNAc transferase when naturally expressed in the source). In certain embodiments, the GalNAc transferase is localized in the secretory pathway without adding a Leishmania signal peptide to the GalNAc transferase. In other embodiments, the signal peptide is added to the GalNAc transferase. In certain embodiments, the signal peptide is fused to the C-terminus of the GalNAc transferase. In certain embodiments, the signal peptide is fused to the N-terminus of the GalNAc transferase. In certain embodiments, the signal peptides is fused to one or more amino acids within the polypeptide of the GalNAc transferase. In certain embodiments, the signal peptide is fused to the N-terminus of an N-terminally truncated variant of the GalNAc transferase. In certain embodiments, the signal peptide is fused to one or more amino acids within the polypeptide of an N-terminally truncated variant of the GalNAc transferase. In certain embodiments, the signal peptide is derived from a Leishmania species. In certain embodiments, the signal peptide is derived from Leishmania tarentolae. In certain embodiments, the signal peptide is an invertase signal peptide from derived from Leishmania tarentolae. In certain embodiments, the signal peptide comprises an amino acid sequence of SEQ ID NO: 11. In certain embodiments, the signal peptide comprises an amino acid sequence of SEQ ID NO: 12. In certain embodiments, the signal peptide is processed and removed from the GalNAc transferase.

In certain embodiments, the GalNAc transferase and the additional recombinant glycosyltransferase described in Section 7.3.2 are co-localized in the secretory pathway. In certain embodiments, the GalNAc transferase and the glycoengineered bifunctional degrader described in Section 7.1 are co-localized in the secretory pathway.

### 7.3.2 Additional Recombinant Glycosyltransferases

In certain embodiments, the Leishmania host cells provided herein comprise a recombinant nucleic acid encoding one or more additional recombinant glycosyltransferases. In certain embodiments, the additional recombinant glycosyltransferase, or a functionally active variant thereof, is capable of catalyzing the addition of a first glycan to a second glycan. In certain embodiments, the additional recombinant glycosyltransferase is a N-acetyl glucosamine transferase, or a functionally active variant thereof, capable of catalyzing the addition of a N-acetyl glucosamine (GlcNAc) to a mannose-terminated glycan, for example, a Man3GlcNAc2 glycan (Man3, see Section 5.3).

In certain embodiments, the additional recombinant glycosyltransferase comprises one or more N-acetyl glucosamine transferases. In certain embodiments, the N-acetyl glucosamine transferase is heterologous to the host cell. In certain embodiments, the N-acetyl glucosamine transferase is derived from Homo sapiens, Spodoptera frugiperda, Trypanosoma brucei, Pan troglodytes, Macaca mulatta, Mus musculus, Rattus norvegicus, Danio rerio A, Drosophila melanogaster, Anopheles gambiae, Caenorhabditis elegans, Arabidopsis thaliana, Oryza sativa japonica, Xenopus tropicalis, Canis lupus, Bos taurus, Danio rerio B, or Gekko japonicus. In certain embodiments, the additional recombinant glycosyltransferase is derived from a mammalian source. In certain embodiments, the mammalian source is Homo sapiens.

In certain embodiments, the N-acetyl glucosamine transferase is selected from the group consisting of MGAT1 (alpha-1,3-mannosyl-glycoprotein 2-beta-N-acetylglucosaminyltransferase) and MGAT2 (alpha-1,6-mannosylglycoprotein 2-beta-N-acetylglucosaminyltransferase), or functionally active variants thereof. In certain embodiments, the additional recombinant glycosyltransferases comprise MGAT1 and MGAT2.

In certain embodiments, the N-acetyl glucosamine transferase comprises MGAT1, or a functionally active variant thereof. In certain embodiments, the N-acetyl glucosamine transferase comprises MGAT1. In certain embodiments, the N-acetyl glucosamine transferase comprises a variant that is at least 80%, at least 85%, at least 90%, at least 95%, at least 98%, or at least 99% homologous to MGAT1. In certain embodiments, the N-acetyl glucosamine transferase comprises MGAT1 of Homo sapiens (accession number P26572), or a functionally active variant thereof. In certain embodiments, the N-acetyl glucosamine transferase comprises MGAT1 of Homo sapiens. In certain embodiments, the N-acetyl glucosamine transferase comprises an amino acid sequence of SEQ ID NO: 13. In certain embodiments, the N-acetyl glucosamine transferase comprises one that is homologous to MGAT1 of Homo sapiens. In certain embodiments, the N-acetyl glucosamine transferase comprises a variant that is at least 80%, at least 85%, at least 90%, at least 95%, at least 98%, or at least 99% homologous to MGAT1 of Homo sapiens.

In certain embodiments, the N-acetyl glucosamine transferase comprises MGAT2, or a functionally active variant thereof. In certain embodiments, the N-acetyl glucosamine transferase comprises MGAT2. In certain embodiments, the N-acetyl glucosamine transferase comprises a variant that is at least 80%, at least 85%, at least 90%, at least 95%, at least 98%, or at least 99% homologous to MGAT2. In certain embodiments, the N-acetyl glucosamine transferase comprises MGAT2 of Homo sapiens (accession number: Q10469.1), or a functionally active variant thereof. In certain embodiments, the N-acetyl glucosamine transferase comprises MGAT2 of Homo sapiens. In certain embodiments, the N-acetyl glucosamine transferase comprises an amino acid sequence of SEQ ID NO: 14. In certain embodiments, the N-acetyl glucosamine transferase comprises one that is homologous to MGAT2 of Homo sapiens. In certain embodiments, the N-acetyl glucosamine transferase comprises a variant that is at least 80%, at least 85%, at least 90%, at least 95%, at least 98%, or at least 99% homologous to MGAT2 of Homo sapiens.

In certain embodiments, the N-acetyl glucosamine transferases comprise MGAT1 and MGAT2, or functionally active variants thereof. In certain embodiments, the N-acetyl glucosamine transferases comprise MGAT1 and MGAT2. In certain embodiments, the N-acetyl glucosamine transferases are variants that are at least 80%, at least 85%, at least 90%, at least 95%, at least 98%, or at least 99% homologous to MGAT1 and MGAT2, respectively. In certain embodiments, the N-acetyl glucosamine transferases are MGAT1 and MGAT2 of Homo sapiens, or functionally active variants thereof. In certain embodiments, the N-acetyl glucosamine transferases are MGAT1 and MGAT2 of Homo sapiens. In certain embodiments, the N-acetyl glucosamine transferases comprise amino acid sequences of SEQ ID NO: 13 and SEQ ID NO: 14, respectively. In certain embodiments, the N-acetyl glucosamine transferases are ones that are homologous to MGAT1 and MGAT2 of Homo sapiens. In certain embodiments, the N-acetyl glucosamine transferases are variants that are at least 80%, at least 85%, at least 90%, at least 95%, at least 98%, or at least 99% homologous to MGAT1 and MGAT2 of Homo sapiens, respectively.

In certain embodiments, the N-acetyl glucosamine transferase comprises MGAT1 of Spodoptera frugiperda (SfGnT-I, accession number: AEX00082), or a functionally active variant thereof. In certain embodiments, the N-acetyl glucosamine transferase comprises MGAT1 of Spodoptera frugiperda. In certain embodiments, the N-acetyl glucosamine transferase comprises one that is homologous to MGAT1 of Spodoptera frugiperda. In certain embodiments, the N-acetyl glucosamine transferase comprises a variant that is at least 80%, at least 85%, at least 90%, at least 95%, at least 98%, or at least 99% homologous to MGAT1 of Spodoptera frugiperda.

In certain embodiments, the N-acetyl glucosamine transferase comprises MGAT1 of Trypanosoma brucei (TbGnT-I, accession number: XP_844156), or a functionally active variant thereof. In certain embodiments, the N-acetyl glucosamine transferase comprises MGAT1 of Trypanosoma brucei. In certain embodiments, the N-acetyl glucosamine transferase comprises one that is homologous to MGAT1 of Trypanosoma brucei. In certain embodiments, the N-acetyl glucosamine transferase comprises a variant that is at least 80%, at least 85%, at least 90%, at least 95%, at least 98%, or at least 99% homologous to MGAT1 of Trypanosoma brucei.

In certain embodiments, the N-acetyl glucosamine transferase comprises MGAT1 of Pan troglodytes (PtMGAT1, accession number: XP_001155433.2), or a functionally active variant thereof. In certain embodiments, the N-acetyl glucosamine transferase comprises MGAT1 of Pan troglodytes. In certain embodiments, the N-acetyl glucosamine transferase comprises one that is homologous to MGAT1 of Pan troglodytes. In certain embodiments, the N-acetyl glucosamine transferase comprises a variant that is at least 80%, at least 85%, at least 90%, at least 95%, at least 98%, or at least 99% homologous to MGAT1 of Pan troglodytes.

In certain embodiments, the N-acetyl glucosamine transferase comprises MGAT1 of Macaca mulatta (MaMGAT1, accession number: NP_001244759), or a functionally active variant thereof. In certain embodiments, the N-acetyl glucosamine transferase comprises MGAT1 of Macaca mulatta. In certain embodiments, the N-acetyl glucosamine transferase comprises one that is homologous to MGAT1 of Macaca mulatta. In certain embodiments, the N-acetyl glucosamine transferase comprises a variant that is at least 80%, at least 85%, at least 90%, at least 95%, at least 98%, or at least 99% homologous to MGAT1 of Macaca mulatta.

In certain embodiments, the N-acetyl glucosamine transferase comprises MGAT1 of Mus musculus (MuMGAT1, accession number: NP_001103620.1), or a functionally active variant thereof. In certain embodiments, the N-acetyl glucosamine transferase comprises MGAT1 of Mus musculus. In certain embodiments, the N-acetyl glucosamine transferase comprises one that is homologous to MGAT1 of Mus musculus. In certain embodiments, the N-acetyl glucosamine transferase comprises a variant that is at least 80%, at least 85%, at least 90%, at least 95%, at least 98%, or at least 99% homologous to MGAT1 of Mus musculus.

In certain embodiments, the N-acetyl glucosamine transferase comprises MGAT1 of Rattus norvegicus (RnMGAT1, accession number: NP_110488.1), or a functionally active variant thereof. In certain embodiments, the N-acetyl glucosamine transferase comprises MGAT1 of Rattus norvegicus. In certain embodiments, the N-acetyl glucosamine transferase comprises one that is homologous to MGAT1 of Rattus norvegicus. In certain embodiments, the N-acetyl glucosamine transferase comprises a variant that is at least 80%, at least 85%, at least 90%, at least 95%, at least 98%, or at least 99% homologous to MGAT1 of Rattus norvegicus.

In certain embodiments, the N-acetyl glucosamine transferase comprises MGAT1 of Danio rerio A (DrMGAT1a, accession number: NP_956970.1), or a functionally active variant thereof. In certain embodiments, the N-acetyl glucosamine transferase comprises MGAT1 of Danio rerio A. In certain embodiments, the N-acetyl glucosamine transferase comprises one that is homologous to MGAT1 of Danio rerio A. In certain embodiments, the N-acetyl glucosamine transferase comprises a variant that is at least 80%, at least 85%, at least 90%, at least 95%, at least 98%, or at least 99% homologous to MGAT1 of Danio rerio A.

In certain embodiments, the N-acetyl glucosamine transferase comprises an MGAT1 of Caenorhabditis elegans (Ce14MGAT1, accession number: NP_497719.1 or Ce13MGAT1, accession number: NP_509566.1), or a functionally active variant thereof. In certain embodiments, the N-acetyl glucosamine transferase comprises MGAT1 of Caenorhabditis elegans. In certain embodiments, the N-acetyl glucosamine transferase comprises one that is homologous to MGAT1 of Caenorhabditis elegans. In certain embodiments, the N-acetyl glucosamine transferase comprises a variant that is at least 80%, at least 85%, at least 90%, at least 95%, at least 98%, or at least 99% homologous to MGAT1 of Caenorhabditis elegans.

In certain embodiments, the N-acetyl glucosamine transferase comprises MGAT1 of Arabidopsis thaliana (AtMGAT1, accession number: NP_195537.2), or a functionally active variant thereof. In certain embodiments, the N-acetyl glucosamine transferase comprises MGAT1 of Arabidopsis thaliana. In certain embodiments, the N-acetyl glucosamine transferase comprises one that is homologous to MGAT1 of Arabidopsis thaliana. In certain embodiments, the N-acetyl glucosamine transferase comprises a variant that is at least 80%, at least 85%, at least 90%, at least 95%, at least 98%, or at least 99% homologous to MGAT1 of Arabidopsis thaliana.

In certain embodiments, the N-acetyl glucosamine transferase comprises MGAT1 of Oryza sativa japonica (OsJMGAT1, accession number: XP_015624616.1), or a functionally active variant thereof. In certain embodiments, the N-acetyl glucosamine transferase comprises MGAT1 of Oryza sativa japonica. In certain embodiments, the N-acetyl glucosamine transferase comprises one that is homologous to MGAT1 Oryza sativa japonica. In certain embodiments, the N-acetyl glucosamine transferase comprises a variant that is at least 80%, at least 85%, at least 90%, at least 95%, at least 98%, or at least 99% homologous to MGAT1 of Oryza sativa japonica.

In certain embodiments, the N-acetyl glucosamine transferase comprises MGAT1 of Xenopus tropicalis (XtMGAT1, accession number: NP_001011350.1), or a functionally active variant thereof. In certain embodiments, the N-acetyl glucosamine transferase comprises MGAT1 of Xenopus tropicalis. In certain embodiments, the N-acetyl glucosamine transferase comprises one that is homologous to MGAT1 Xenopus tropicalis. In certain embodiments, the N-acetyl glucosamine transferase comprises a variant that is at least 80%, at least 85%, at least 90%, at least 95%, at least 98%, or at least 99% homologous to MGAT1 of Xenopus tropicalis.

In certain embodiments, the N-acetyl glucosamine transferase comprises MGAT1 of Canis lupus (ClMGAT1, accession number: XP_855658.1), or a functionally active variant thereof. In certain embodiments, the N-acetyl glucosamine transferase comprises MGAT1 of Canis lupus. In certain embodiments, the N-acetyl glucosamine transferase comprises one that is homologous to MGAT1 Canis lupus. In certain embodiments, the N-acetyl glucosamine transferase comprises a variant that is at least 80%, at least 85%, at least 90%, at least 95%, at least 98%, or at least 99% homologous to MGAT1 of Canis lupus.

In certain embodiments, the N-acetyl glucosamine transferase comprises MGAT1 of Bos taurus (BtMGAT1, accession number: NP_001015653.1), or a functionally active variant thereof. In certain embodiments, the N-acetyl glucosamine transferase comprises MGAT1 of Bos taurus. In certain embodiments, the N-acetyl glucosamine transferase comprises one that is homologous to MGAT1 Bos taurus. In certain embodiments, the N-acetyl glucosamine transferase comprises a variant that is at least 80%, at least 85%, at least 90%, at least 95%, at least 98%, or at least 99% homologous to MGAT1 of Bos taurus.

In certain embodiments, the N-acetyl glucosamine transferase comprises MGAT1 of Danio rerio B (DrMGAT1b, accession number: NP_001073440.1), or a functionally active variant thereof. In certain embodiments, the N-acetyl glucosamine transferase comprises MGAT1 of Danio rerio B. In certain embodiments, the N-acetyl glucosamine transferase comprises one that is homologous to MGAT1 Danio rerio B. In certain embodiments, the N-acetyl glucosamine transferase comprises a variant that is at least 80%, at least 85%, at least 90%, at least 95%, at least 98%, or at least 99% homologous to MGAT1 of Danio rerio B.

In certain embodiments, the N-acetyl glucosamine transferase comprises MGAT1 of Gekko japonicus (GjMGAT1, accession number: XP_015280466.1), or a functionally active variant thereof. In certain embodiments, the N-acetyl glucosamine transferase comprises MGAT1 of Gekko japonicus. In certain embodiments, the N-acetyl glucosamine transferase comprises one that is homologous to MGAT1 Gekko japonicus. In certain embodiments, the N-acetyl glucosamine transferase comprises a variant that is at least 80%, at least 85%, at least 90%, at least 95%, at least 98%, or at least 99% homologous to MGAT1 of Gekko japonicus.

In certain embodiments, the N-acetyl glucosamine transferase comprises MGAT2 of Rattus norvegicus (rMGAT2, accession number: NP_446056), or a functionally active variant thereof. In certain embodiments, the N-acetyl glucosamine transferase comprises MGAT2 of Rattus norvegicus. In certain embodiments, the N-acetyl glucosamine transferase comprises one that is homologous to MGAT2 of Rattus norvegicus. In certain embodiments, the N-acetyl glucosamine transferase comprises a variant that is at least 80%, at least 85%, at least 90%, at least 95%, at least 98%, or at least 99% homologous to MGAT2 of Rattus norvegicus.

In certain embodiments, the N-acetyl glucosamine transferase comprises MGAT2 of Spodoptera frugiperda (SfGnT-II, accession number: AEX00083), or a functionally active variant thereof. In certain embodiments, the N-acetyl glucosamine transferase comprises MGAT2 of Spodoptera frugiperda. In certain embodiments, the N-acetyl glucosamine transferase comprises one that is homologous to MGAT2 of Spodoptera frugiperda. In certain embodiments, the N-acetyl glucosamine transferase comprises a variant that is at least 80%, at least 85%, at least 90%, at least 95%, at least 98%, or at least 99% homologous to MGAT2 of Spodoptera frugiperda.

In certain embodiments, the N-acetyl glucosamine transferase comprises MGAT2 of Trypanosoma brucei (TbGnT-II, accession number: XP_845654), or a functionally active variant thereof. In certain embodiments, the N-acetyl glucosamine transferase comprises MGAT2 of Trypanosoma brucei. In certain embodiments, the N-acetyl glucosamine transferase comprises one that is homologous to MGAT2 of Trypanosoma brucei. In certain embodiments, the N-acetyl glucosamine transferase comprises a variant that is at least 80%, at least 85%, at least 90%, at least 95%, at least 98%, or at least 99% homologous to MGAT2 of Trypanosoma brucei.

In certain embodiments, the additional recombinant glycosyltransferase is localized in the secretory pathway. Without being bound by theory, localization within the secretory pathway includes, but is not limited to, localization to one or more of the following sub-cellular compartments: the endoplasmic reticulum, the Golgi apparatus, lysosomes, intracellular membrane proteins, cell surface anchored proteins, and membrane proteins. In certain embodiments, localization in the secretory pathway comprises localization to one or more of said sub-cellular compartments.

In certain embodiments, the additional recombinant glycosyltransferase comprises a signal peptide localizing the additional recombinant glycosyltransferase in the secretory pathway. In certain embodiments, the signal peptide is derived from the same source as the additional recombinant glycosyltransferase (i.e. the signal peptide is not added to additional recombinant glycosyltransferase, but is one fused to the additional recombinant glycosyltransferase when naturally expressed in the source). In certain embodiments, the additional recombinant glycosyltransferase is localized in the secretory pathway without adding a Leishmania signal peptide to the additional recombinant glycosyltransferase. In other embodiments, the signal peptide is added to the additional recombinant glycosyltransferase. In certain embodiments, the signal peptide is fused to the C-terminus of the additional recombinant glycosyltransferase. In certain embodiments, the signal peptide is fused to the N-terminus of the additional recombinant glycosyltransferase. In certain embodiments, the signal peptides is fused to one or more amino acids within the polypeptide of the additional recombinant glycosyltransferase. In certain embodiments, the signal peptide is derived from a Leishmania species. In certain embodiments, the signal peptide is derived from Leishmania tarentolae. In certain embodiments, the signal peptide is an invertase signal peptide from derived from Leishmania tarentolae. In certain embodiments, the signal peptide comprises an amino acid sequence of SEQ ID NO: 11. In certain embodiments, the signal peptide comprises an amino acid sequence of SEQ ID NO: 12. In certain embodiments, the signal peptide is processed and removed from the additional recombinant glycosyltransferase.

In certain embodiments, the GalNAc transferase described in Section 7.3.1 and the additional recombinant glycosyltransferase are co-localized in the secretory pathway.

7.3.3 Deletion, Mutation and/or Functionally Inactivation of Endogenous Enzymes from the Glycan Biosynthesis Pathway

In certain embodiments, the Leishmania host cells provided herein are characterized in that one or more endogenous enzymes from the glycan biosynthesis pathway have been deleted, mutated and/or functionally inactivated. In certain embodiments, the Leishmania host cell does not have endogenous N-glycan elongation. In certain embodiments, the Leishmania host cells do not have endogenous N-glycan elongation as described in WO 2019/002512, which is incorporated herein by reference in its entirety. In certain embodiments, the Leishmania host cell has been genetically engineered such that the formation of an O-linked GlcNAc on a polypeptide in the host cell is reduced or eliminated. In certain embodiments, the formation of O-linked GlcNAc in the Leishmania host cell prior to genetic engineering is catalyzed by at least one N-acetylglucosamine (GlcNAc)-transferase. In certain embodiments, the gene encoding the at least one GlcNAc-transferase in the Leishmania host cell is functionally inactivated, downregulated, deleted, and/or mutated.

In certain embodiments, the enzyme that catalyzes the formation of O-linked GlcNAc is an N-acetylglucosamine (GlcNAc)-transferase. In certain embodiments, the GlcNAc-transferase is selected from the group consisting of OGNT1, OGNT2, OGNTL, and homologous GlcNAc-transferases thereof. Without being bound by theory, OGNT1, OGNT2, OGNTL were identified based on homology to Trypanosoma enzymes and not mammalian (e.g. human) enzymes (Heise, N., et al Glycobiology, 19(8), 918-933 (2009) and Chiribao, M. L. et al Gene, 498(2), 147-154 (2012), each of which is incorporated herein by reference in its entirety). In certain embodiments, the GlcNAc-transferase is OGNT1. In other embodiments, the GlcNAc-transferase is OGNT2. In yet other embodiments, the GlcNAc-transferase is OGNTL. In certain embodiments, the GlcNAc-transferase is a GlcNAc-transferase that is homologous to OGNT1. In certain embodiments, the GlcNAc-transferase is a GlcNAc-transferase that is homologous to OGNT2. In certain embodiments, the GlcNAc-transferase is a GlcNAc-transferase that is homologous to OGNTL. In certain embodiments, the GlcNAc-transferase is derived from Leishmania tarentolae. In certain embodiments, the GlcNAc-transferase is derived from other Trypanosomatida species. Nonlimiting examples of GlcNAc-transferases in Trypanosomatida are listed in Table 1, in which one representative genome per species is listed.

TABLE 1
Exemplary GlcNAc-transferases in Trypanosomatida.

| | Strain/TriTrypDB | | | |
|---|---|---|---|---|
| Species | Genome accession | OGNT1 | OGNT2 | OGNTL |
| Blechomonas | TriTrypDB- | rna_Baya_228_ | rna_Baya_014 | rna_Baya_228_ |
| ayalai | 44_BayalaiB08- | 0070-1-p1 | 0750-1-p1 | 0080-1-p1 |
| | 376_AnnotatedProteins | | | |
| Bodo | TriTrypDB- | none | BSAL_53670- | None |
| saltans | 44_BsaltansLakeKonstanz_ | | t42_1-p1 | |
| | AnnotatedProteins | | | |
| Crithidia | TriTrypDB- | CFAC1_ | CFAC1_ | CFAC1_ |
| fasciculata | 44_CfasciculataCfCl_ | 160013000.1- | 090018200.1- | 160013300.1- |
| | AnnotatedProteins | p1 | p1 | p1 |
| Endotrypanum | TriTrypDB- | EMOLV88_ | EMOLV88_ | EMOLV88_ |
| monterogeii | 44_EmonterogeiiLV88_ | 020005500.1- | 170014900.1- | 020005600.1- |
| | AnnotatedProteins | p1 | p1 | p1 |
| Leishmania | TriTrypDB- | LAEL147_ | LAEL147_ | LAEL147_ |
| aethiopica | 44_LaethiopicaL147_ | 000030200.1- | 000253000.1- | 000030300.1- |
| | AnnotatedProteins | p1 | p1 | p1 |
| Leishmania | TriTrypDB- | LAMA_ | LAMA_ | LAMA_ |
| amazonensis | 44_LamazonensisMHOM | 000035900.1- | 000268900.1- | 000036200.1- |
| | BR71973M2269_ | p1 | p1 | p1 |
| | AnnotatedProteins | | | |
| Leishmania | TriTrypDB- | LARLEM1108_ | LARLEM1108_ | LARLEM1108_ |
| arabica | 44_LarabicaLEM1108_ | 020007100.1- | 170015900.1- | 020007 |
| | AnnotatedProteins | p1 | p1 | 200.1-p1 |
| Leishmania | TriTrypDB- | LbrM.02.0270: | LbrM.17.1130: | LbrM.02.0280: |
| braziliensis | 44_LbraziliensisMHOMB | mRNA-p1 | mRNA-p1 | mRNA- |
| | R75M2904_ | | | p1 |
| | AnnotatedProteins | | | |
| Leishmania | TriTrypDB- | LdCL_ | LdCL_ | LdCL_ |
| donovani | 44_LdonovaniCL- | 020007500- | 170017200-_ | 020007600-_ |
| | SL_AnnotatedProteins | t42_1-p1 | t42_1-p1 | t42_1-p1 |
| Leishmania | TriTrypDB- | LENLEM3045_ | LENLEM3045_ | None |
| enrietti | 44_LenriettiiLEM3045_ | 000029600.1- | 170016400.1- | |
| | AnnotatedProteins | p1 | p1 | |
| Leishmania | TriTrypDB- | LGELEM452_ | LGELEM452_ | LGELEM452_ |
| gerbilli | 44_LgerbilliLEM452_ | 020006800.1- | 170012100.1- | 02000690 |
| | AnnotatedProteins | p1 | p1 | 0.1-p1 |
| Leishmania | TriTrypDB- | LINF_ | LINF_ | LINF_ |
| infantum | 44_LinfantumJPCM5_ | 020007400- | 170017300- | 020007500- |
| | AnnotatedProteins | T1-p1 | T1-p1 | T1-p1 |
| Leishmania | TriTrypDB- | LmjF.02.0240: | LmjF.17.1020: | LmjF.02.050:2 |
| major | 44_LmajorFriedlin_ | mRNA-p1 | mRNA-pl | mRNA- |
| | AnnotatedProteins | | | p1 |
| Leishmania | TriTrypDB- | LmxM.02.0240.1- | LmxM.17.1020.1- | LmxM.02.0250.1- |
| mexicana | 44_LmexicanaMHOMGT | p1 | p1 | p1 |
| | 2001U1103_ | | | |
| | AnnotatedProteins | | | |
| Leishmania | TriTrypDB- | LPMP_ | LPMP_ | LPMP_ |
| panamensis | 44_LpanamensisMHOMP | 020160- | 170990- | 020170- |
| | A94PSC1_ | t37_1-p1 | t37_1-p1 | t37_1-p1 |
| | AnnotatedProteins | | | |
| Leishmania | TriTrypDB- | rna_LpyrH10 | none | rna_LpyrH10_ |
| pyrrhocoris | 44_LpyrrhocorisH10_ | 27_0530-p1 | | 27_0550- |
| | AnnotatedProteins | | | pl |
| Leishmania | TriTrypDB- | rna_Lsey_ | none | rna_Lsey_ |
| seymouri | 44_LseymouriATCC30220_ | 03790030-1-p1 | | 0379_0020- |
| | AnnotatedProteins | | | 1-p1 |
| Leishmania | TriTrypDB- | none | LMARLEM2494_ | LMARLEM2494_ |
| sp. MAR | 44_LspMARLEM2494_ | | 1700155001- | 020006500.1- |
| | AnnotatedProteins | | p1 | p1 |
| Leishmania | TriTrypDB- | LTRL590_ | LTRL590_ | LTRL590_ |
| tropica | 44_LtropicaL590_ | 020007500.1-p1 | 000019100.1-p1 | 020007600.1- |
| | AnnotatedProteins | | | p1 |
| Leishmania | TriTrypDB- | LTULEM423 | LTULEM423 | LTULEM423_ |
| turanica | 44_LturanicaLEM423_ | 020006600.1- | 170015700.1- | 020006700.1- |
| | AnnotatedProteins | p1 | p1 | p1 |
| Para- | TriTrypDB- | none | PCON_0046770. | None |
| trypanosoma | 44_PconfusumCUL13_ | | mRNA-p1 | |
| confusum | AnnotatedProteins | | | |
| Trypanosoma | TriTrypDB- | Tbg.972.2.950: | Tbg972.5.3320: | Tbg.972.2.930: |
| brucei | 44_TbruceigambienseDA | mRNA-p1 | mRNA-p1 | mRNA- |
| | L972_AnnotatedProteins | | | p1 |
| Trypanosoma | TriTrypDB- | TcIL3000_0_ | TcIL3000_5_ | TcIL3000_2_ |
| congolense | 44_TcongolenseIL3000 | 43290.1-p1 | 2160.1-p1 | 200.1-p1 |
| | AnnotatedProteins | | | |
| Trypanosoma | TriTrypDB- | TevSTIB805.2. | TevSTIB805.5. | TevSTIB80 |
| evansi | 44_TevansiSTIB805_ | 1330-t26_1-p1 | 2740-t26_1-p1 | 5.2.1310- |
| | AnnotatedProteins | | | t26_1-p1 |
| Trypanosoma | TriTrypDB- | DQ04_09771010- | DQ04_01951010- | DQ04_0429 |
| grayi | 44_TgrayiANR4_ | t26_1-p1 | t26_1-p1 | 1000-t26_1- |
| | AnnotatedProteins | | | p1 |
| Trypanosoma | TriTrypDB- | TRSC58_0008 | TRSC58_05317- | TRSC58_ |
| rangeli | 44_TrangeliSC58_ | 3-t26_1-p1 | t26_1-p1 | 03864-t26_1- |
| | AnnotatedProteins | | | p1 |
| Trypanosoma | TriTrypDB- | TM35_000391720- | TM35_ | TM35_ |
| theileri | 44_TtheileriEdinburgh_ | t36_1-p1 | 000201600- | 000391740- |
| | AnnotatedProteins | | t36_1-p1 | t36_1-p1 |
| Trypanosoma | TriTrypDB- | TvY486_0200430: | TvY486_ | TvY486_ |
| vivax | 44_TvivaxY486_ | mRNA-p1 | 0501730: | 0200410: |
| | AnnotatedProteins | | mRNA-p1 | mRNA-p1 |
| | | | | (Pseudogene) |

In certain embodiments, the enzyme that catalyzes the formation of O-linked GlcNAc is derived from species other than Trypanosomatida species. In certain embodiments, the enzyme is a human O-GlcNAc transferase (OGT, Uniprot: O15294) and homologous enzymes thereof. In certain embodiments, the O-GlcNAc transferase (OGT; uridine diphospho-N-acetylglucosamine:polypeptide β-N-acetylglucosaminyltransferase; EC 2.4.1.255) can catalyze the transfer of a single N-acetylglucosamine from UDP-GlcNAc to a serine or threonine residue in cytoplasmic and nuclear proteins resulting in their modification with a beta-linked N-acetylglucosamine (O-GlcNAc). In certain embodiments, the enzyme that catalyzes the formation of O-linked GlcNAc may be different isoforms of OGT. Exemplary isoforms of OGT include but are not limited to: (1) the nucleocytoplasmic or full-length variant (ncOGT), which may be 110 kDa; (2) a short isoform of OGT (sOGT), which may be 78 kDa; and (3) a variant of OGT that is targeted to the mitochondria (mOGT; which may be 90 kDa). In certain embodiments, OGT may appear to form multimers in the nucleus and cytoplasm, consisting of one or more 110-kDa subunits and 78-kDa subunits (Varki, Ajit, et al. (Eds.) (2015): Essentials of Glycobiology. Cold Spring Harbor Laboratory Press. 3rd. Cold Spring Harbor (NY)). In certain embodiments, the enzyme that catalyzes the formation of O-linked GlcNAc is human EOGT (Uniprot: Q5NDL2). In certain embodiments, the enzyme catalyzes the transfer of a single N-acetylglucosamine from UDP-GlcNAc to a serine or threonine residue in extracellular proteins resulting in their modification with a beta-linked N-acetylglucosamine (O-GlcNAc). In certain embodiments, the enzyme catalyzes Specific glycosylation of the Thr residue located between the fifth and sixth conserved cysteines of folded EGF-like domains.

In certain embodiments, the enzyme that catalyzes the formation of O-linked GlcNAc may transfer in alpha-linkage. In other embodiments, the enzyme that catalyzes the formation of O-linked GlcNAc may transfer in beta-linkage.

In certain embodiments, the formation of O-linked GlcNAc in the Leishmania host cell prior to genetic engineering is catalyzed by at least one enzyme as described in this Section, for example one, two, three, four, five, six, seven, eight, nine or ten enzymes as described in this Section.

In certain embodiments, the formation of O-linked GlcNAc in the Leishmania host cell prior to genetic engineering is catalyzed by at least one GlcNAc-transferase derived from Trypanosomatida species, for example Leishmania tarentolae. In certain embodiments, the formation of O-linked GlcNAc in the Leishmania host cell prior to genetic engineering is catalyzed by at least one GlcNAc-transferase, for example one, two, three, four, five, six, seven, eight, nine or ten GlcNAc-transferases, one or more of which is derived from Trypanosomatida species. In certain embodiments, the number of the at least one GlcNAc-transferase is one, two or three. In certain embodiments, the at least one GlcNAc-transferase is selected from the group consisting of OGNT1, OGNT2, OGNTL and homologous GlcNAc-transferases thereof. In certain embodiments, at least one GlcNAc-transferase is a GlcNAc-transferase that is homologous to OGNT1, OGNT2 and/or OGNTL.

In certain embodiments, the formation of O-linked GlcNAc in the Leishmania host cell prior to genetic engineering is catalyzed by at least one GlcNAc-transferase derived from species that is other than Trypanosomatida species, for example human. In certain embodiments, the formation of O-linked GlcNAc in the Leishmania host cell prior to genetic engineering is catalyzed by at least one GlcNAc-transferase, for example one, two, three, four, five, six, seven, eight, nine or ten GlcNAc-transferases, one or more of which is derived from human. In certain embodiments, the number of the at least one GlcNAc-transferase is one, two or three. In certain embodiments, the at least one GlcNAc-transferase is selected from the group consisting of human O-GlcNAc transferase and human EOGT and homologous enzymes thereof. In certain embodiments, at least one GlcNAc-transferase is an enzyme that is homologous to human O-GlcNAc transferase and/or human EOGT.

In certain embodiments, the enzyme catalyzes the formation of O-linked GlcNAc prior to the genetic engineering of the Leishmania host cell. In certain embodiments, the enzyme still catalyzes the formation of O-linked GlcNAc after the genetic engineering of the Leishmania host cell. In certain embodiments, the enzyme does not catalyze the formation of O-linked GlcNAc after the genetic engineering of the Leishmania host cell.

In certain embodiments, the gene encoding the at least one GlcNAc-transferase in the Leishmania host cell is functionally inactivated. In certain embodiments, the gene encoding the at least one GlcNAc-transferase in the Leishmania host cell is downregulated. In certain embodiments, the gene encoding the at least one GlcNAc-transferase in the Leishmania host cell is overexpressed.

In certain embodiments, the Leishmania host cell provided herein comprises at least one gene deletion. In certain embodiments, the gene encoding the at least one GlcNAc-transferase is deleted. In certain embodiments, the gene encoding the at least one GlcNAc-transferase is mutated. In certain embodiments, the gene encoding the at least one GlcNAc-transferase is overexpressed. In certain embodiments, additional modifications may be introduced (e.g., using recombinant techniques) into the Leishmania host cell described herein.

In certain embodiments, the genes encoding at least 1, 2, 3, 4, 5, 6, 7, 8, 9, 10, 11, 12, 13, 14, 15, 16, 17, 18, 19, or at least 20 enzymes that each catalyzes the formation of O-linked GlcNAc may be functionally inactivated. In certain embodiments, the genes encoding 1, 2, 3, 4, 5, 6, 7, 8, 9, 10, 11, 12, 13, 14, 15, 16, 17, 18, 19, or 20 enzymes that each catalyzes the formation of O-linked GlcNAc may be functionally inactivated. In certain embodiments, the genes encoding three enzymes that each catalyzes the formation of O-linked GlcNAc may be functionally inactivated. In certain embodiments, the genes and/or genetic loci that may be functionally inactivated include but are not limited to OGNT1, OGNT2, and OGNTL.

In certain embodiments, the genes encoding at least 1, 2, 3, 4, 5, 6, 7, 8, 9, 10, 11, 12, 13, 14, 15, 16, 17, 18, 19, or at least 20 GlcNAc-transferase that each catalyzes the formation of O-linked GlcNAc may be functionally inactivated. In certain embodiments, the genes encoding 1, 2, 3, 4, 5, 6, 7, 8, 9, 10, 11, 12, 13, 14, 15, 16, 17, 18, 19, or 20 GlcNAc-transferase that each catalyzes the formation of O-linked GlcNAc may be functionally inactivated. In certain embodiments, the genes encoding three GlcNAc-transferases that each catalyzes the formation of O-linked GlcNAc may be functionally inactivated.

In certain embodiments, the genes encoding at least 5%, 10%, 20%, 30%, 40%, 50%, 60%, 70%, 80%, 90%, or 100% of all enzymes that each catalyzes the formation of 0-linked GlcNAc may be functionally inactivated. In certain embodiments, the genes encoding 5%, 10%, 20%, 30%, 40%, 50%, 60%, 70%, 80%, 90%, or 100% of all enzymes that each catalyzes the formation of O-linked GlcNAc may be functionally inactivated.

In certain embodiments, the genes encoding at least 5%, 10%, 20%, 30%, 40%, 50%, 60%, 70%, 80%, 90%, or 100% of all GlcNAc-transferases that each catalyzes the formation of O-linked GlcNAc may be functionally inactivated. In certain embodiments, the genes encoding 5%, 10%, 20%, 30%, 40%, 50%, 60%, 70%, 80%, 90%, or 100% of all GlcNAc-transferases that each catalyzes the formation of O-linked GlcNAc may be functionally inactivated.

In certain embodiments, the at least one GlcNAc-transferase is selected from the group consisting of OGNT1, OGNT2 and OGNTL, and homologous GlcNAc-transferases thereof. In certain embodiments, the Leishmania host cell is a OGNT1, OGNT2 and OGNTL triple knockout.

In certain embodiments, in the Leishmania host cell, the formation of the O-linked GlcNAc is reduced by at least 5%, 7%, 10%, 12%, 15%, 18%, 20%, 25%, 30%, 35%, 40%, 45%, 50%, 55%, 60%, 65%, 70%, 75%, 80%, 85%, 90%, 95%, or 100% from the formation of the O-linked GlcNAc in a reference Leishmania cell. In certain embodiments, in the Leishmania host cell, the formation of the O-linked GlcNAc is reduced by 5%, 7%, 10%, 12%, 15%, 18%, 20%, 25%, 30%, 35%, 40%, 45%, 50%, 55%, 60%, 65%, 70%, 75%, 80%, 85%, 90%, 95%, or 100% from the formation of the O-linked GlcNAc in a reference Leishmania cell. In certain embodiments, the reference Leishmania cell is wild-type. In certain embodiments, the reference Leishmania cell is genetically engineered differently as the genetically engineered Leishmania cells described herein. In certain embodiments, some of the engineering of the reference Leishmania cell may be the same of the engineering of the genetically engineered Leishmania cells described herein, for example the deletion of one or more enzymes that catalyze the formation of O-linked GlcNAc. In certain embodiments, the reference Leishmania cell may comprise a recombinant nucleic acid encoding a heterologous glycosyltransferase, for example the Leishmania cells described in International Publication No. WO2019/002512 A2, incorporated by reference in its entirety herein. In certain embodiments, the formation of the O-linked GlcNAc is reduced by at least 5%, 7%, 10%, 12%, 15%, 18%, 20%, 25%, 30%, 35%, 40%, 45%, 50%, 55%, 60%, 65%, 70%, 75%, 80%, 85%, 90%, 95%, or 100% from the formation of the O-linked GlcNAc in a wild-type Leishmania cell. In certain embodiments, the formation of the O-linked GlcNAc is reduced by 5%, 7% %, 12%, 15%, 18%, 20%, 25%, 30%, 35%, 40%, 45%, 50%, 55%, 60%, 65%, 70%, 75%, 80%, 85%, 90%, 95%, or 100% from the formation of the O-linked GlcNAc in a wild-type Leishmania cell. In certain embodiments, the formation of the O-linked GlcNAc is reduced by at least 5%, 7%, 10%, 12%, 15%, 18%, 20%, 25%, 30%, 35%, 40%, 45%, 50%, 55%, 60%, 65%, 70%, 75%, 80%, 85%, 90%, 95%, or 100% from the formation of the O-linked GlcNAc in a Leishmania cell that comprises a recombinant nucleic acid encoding a heterologous glycosyltransferase. In certain embodiments, the formation of the O-linked GlcNAc is reduced by 5%, 7%, %, 12%, 15%, 18%, 20%, 25%, 30%, 35%, 40%, 45%, 50%, 55%, 60%, 65%, 70%, 75%, 80%, 85%, 90%, 95%, or 100% from the formation of the O-linked GlcNAc in a Leishmania cell that comprises a recombinant nucleic acid encoding a heterologous glycosyltransferase.

In certain embodiments, the growth rate of the Leishmania host cell described herein is at least 60%, 65%, 70%, 75%, 80%, 85%, 90%, 95%, or 100% of the growth rate of a reference Leishmania cell. In certain embodiments, the growth rate of the Leishmania host cell is 60%, 65%, 70%, 75%, 80%, 85%, 90%, 95%, or 100% of the growth rate of a reference Leishmania cell. In certain embodiments, the reference Leishmania cell is wild-type. In certain embodiments, the reference Leishmania cell is genetically engineered differently as the genetically engineered Leishmania cells described herein. In certain embodiments, some of the engineering of the reference Leishmania cell may be the same of the engineering of the genetically engineered Leishmania cells described herein, for example the deletion of one or more enzymes that catalyze the formation of O-linked GlcNAc. In certain embodiments, the growth rate of the Leishmania cell is at least 60%, 65%, 70%, 75%, 80%, 85%, 90%, 95%, or 100% of the growth rate of a wild-type Leishmania cell. In certain embodiments, the growth rate of the Leishmania cell is 60%, 65%, 70%, 75%, 80%, 85%, 90%, 95%, or 100% of the growth rate of a wild-type Leishmania cell.

### 7.3.4 Heterologous UDP-GalNAc Biosynthetic Pathway Proteins

In certain embodiments, the Leishmania host cells provided herein comprise a recombinant nucleic acid encoding UDP-GalNAc biosynthetic pathway proteins capable of generating UDP-GalNAc. In certain embodiments, the recombinant UDP-GalNAc biosynthetic pathway proteins are heterologous to the Leishmania host cell.

In certain embodiments, the heterologous UDP-GalNAc biosynthetic pathway proteins are capable of converting GalNAc to UDP-GalNAc. In certain embodiments, the heterologous UDP-GalNAc biosynthetic pathway proteins capable of converting GalNAc to UDP-GalNAc are derived from a mammalian source. In certain embodiments, the mammalian source is Homo sapiens.

In certain embodiments, the heterologous UDP-GalNAc biosynthetic pathway proteins are capable of converting N-Acetyl galactosamine 1-phosphate (GalNAc-1-P) to UDP-GalNAc. In certain embodiments, the heterologous UDP-GalNAc biosynthetic pathway proteins are capable of converting GalNAc-1-P and UTP to UDP-GalNAc. In certain embodiments, the heterologous UDP-GalNAc biosynthetic pathway proteins comprise UDP-N-acetyl hexosamine pyrophosphorylase (UAP1), or a functionally active variant thereof. In certain embodiments, the heterologous UDP-GalNAc biosynthetic pathway proteins comprise UDP-N-acetyl hexosamine pyrophosphorylase (UAP1) of Homo sapiens, or a functionally active variant thereof. In certain embodiments, the heterologous UDP-GalNAc biosynthetic pathway proteins comprise UDP-N-acetyl hexosamine pyrophosphorylase (UAP1) of Homo sapiens, or a variant that is at least 80%, at least 85%, at least 90%, at least 95%, at least 98%, or at least 99% homologous thereof. In certain embodiments, the heterologous UDP-GalNAc biosynthetic pathway proteins comprise UDP-N-acetyl hexosamine pyrophosphorylase (UAP1) of Homo sapiens. In certain embodiments, the heterologous UDP-GalNAc biosynthetic pathway proteins comprise a variant of UDP-N-acetyl hexosamine pyrophosphorylase (UAP1) of Homo sapiens that is at least 80%, at least 85%, at least 90%, at least 95%, at least 98%, or at least 99% homologous thereof. Without being bound by theory, the AGX1 isoform of UDP-N-acetyl hexosamine pyrophosphorylase is about two to three times more active towards GalNAc-1-P than GlcNAc-1-P, whereas the AGX2 isoform is about eight times more active towards GlcNAc-1-P than GalNAc-1-P. In certain embodiments, the UDP-N-acetyl hexosamine pyrophosphorylase (UAP1) is the AGX1 isoform of UAP1. In other embodiments, the UDP-N-acetyl hexosamine pyrophosphorylase (UAP1) is the AGX2 isoform of UAP1. In certain embodiments, the UDP-N-acetyl hexosamine pyrophosphorylase (UAP1) comprises an amino acid sequence of SEQ ID NO: 15.

In certain embodiments, the heterologous UDP-GalNAc biosynthetic pathway proteins are capable of converting GalNAc to GalNAc-1-P. In certain embodiments, the heterologous UDP-GalNAc biosynthetic pathway proteins comprise N-acetyl galactosamine kinase (GALK2), or a functionally active variant thereof. In certain embodiments, the heterologous UDP-GalNAc biosynthetic pathway proteins comprise N-acetyl galactosamine kinase (GALK2) of Homo sapiens, or a functionally active variant thereof. In certain embodiments, the heterologous UDP-GalNAc biosynthetic pathway proteins comprise N-acetyl galactosamine kinase (GALK2) of Homo sapiens, or a variant that is at least 80%, at least 85%, at least 90%, at least 95%, at least 98%, or at least 99% homologous thereof. In certain embodiments, the heterologous UDP-GalNAc biosynthetic pathway proteins comprise N-acetyl galactosamine kinase (GALK2) of Homo sapiens. In certain embodiments, the heterologous UDP-GalNAc biosynthetic pathway proteins comprise a variant of N-acetyl galactosamine kinase (GALK2) of Homo sapiens that is at least 80%, at least 85%, at least 90%, at least 95%, at least 98%, or at least 99% homologous thereof. In certain embodiments, the N-acetyl galactosamine kinase (GALK2) comprises an amino acid sequence of SEQ ID NO: 15.

In certain embodiments, the heterologous UDP-GalNAc biosynthetic pathway proteins are capable of converting UDP-GlcNAc to UDP-GalNAc. In certain embodiments, the heterologous UDP-GalNAc biosynthetic pathway protein capable of converting UDP-GlcNAc to UDP-GalNAc comprises a NAD-dependent epimerase that converts UDP-GlcNAc to UDP-GalNAc. In certain embodiments, the heterologous UDP-GalNAc biosynthetic pathway proteins capable of converting UDP-GlcNAc to UDP-GalNAc is derived from a mammalian source. In certain embodiments, the mammalian source is Homo sapiens. In certain embodiments, the heterologous UDP-GalNAc biosynthetic pathway proteins comprise UDP-galactose 4-epimerase (GalE), or a functionally active variant thereof. In certain embodiments, the heterologous UDP-GalNAc biosynthetic pathway proteins comprise UDP-galactose 4-epimerase (GalE) of Homo sapiens (hGalE), or a functionally active variant thereof. In certain embodiments, the heterologous UDP-GalNAc biosynthetic pathway protein comprise hGalE, or a variant that is at least 80%, at least 85%, at least 90%, at least 95%, at least 98%, or at least 99% homologous thereof. In certain embodiments, the heterologous UDP-GalNAc biosynthetic pathway proteins comprise hGalE. In certain embodiments, the heterologous UDP-GalNAc biosynthetic pathway proteins comprise a variant of hGalE that is at least 80%, at least 85%, at least 90%, at least 95%, at least 98%, or at least 99% homologous thereof. In certain embodiments, the hGalE comprises an amino acid sequence of SEQ ID NO: 17. In certain embodiments, the heterologous UDP-GalNAc biosynthetic pathway proteins capable of converting UDP-GlcNAc to UDP-GalNAc are derived from a bacterial source. In certain embodiments, the bacterial source is Campylobacter jejuni. In certain embodiments, the heterologous UDP-GalNAc biosynthetic pathway proteins comprise UDP-GlcNAc/Glc 4-epimerase of Campylobacter jejuni (CjGne), or a functionally active variant thereof. In certain embodiments, the heterologous UDP-GalNAc biosynthetic pathway proteins comprise CjGne, or a variant that is at least 80%, at least 85%, at least 90%, at least 95%, at least 98%, or at least 99% homologous thereof. In certain embodiments, the heterologous UDP-GalNAc biosynthetic pathway proteins comprise CjGne. In certain embodiments, the heterologous UDP-GalNAc biosynthetic pathway proteins comprise comprise a variant of CjGne that is at least 80%, at least 85%, at least 90%, at least 95%, at least 98%, or at least 99% homologous thereof. In certain embodiments, the CjGne comprises an amino acid sequence of SEQ ID NO: 18.

In certain embodiments, the Leishmania host cell comprises (a) a recombinant nucleic acid encoding recombinant UDP-GalNAc biosynthetic pathway proteins capable of converting GalNAc to UDP-GalNAc as described in this Section; and (b) a recombinant nucleic acid encoding a recombinant UDP-GalNAc transporter protein capable of transporting UDP-GalNAc to the secretory pathway as described in Section 7.3.5. In certain embodiments, the recombinant nucleic acid in (a) encodes UDP-GalNAc biosynthetic pathway proteins capable of converting GalNAc-1-P to UDP-GalNAc and UDP-GalNAc biosynthetic pathway proteins capable of converting GalNAc to GalNAc-1-P, as described in this Section.

In certain embodiments, the Leishmania host cell comprises (a) a recombinant nucleic acid encoding a recombinant UDP-GalNAc biosynthetic pathway protein capable of converting UDP-GlcNAc to UDP-GalNAc as described in this Section; and (b) a recombinant nucleic acid encoding a recombinant UDP-GalNAc transporter protein capable of transporting UDP-GalNAc to the secretory pathway as described in Section 7.3.5.

In certain embodiments, the Leishmania host cell comprises (a) a recombinant nucleic acid encoding recombinant UDP-GalNAc biosynthetic pathway proteins capable of converting GalNAc to UDP-GalNAc as described in this Section; and (b) a recombinant nucleic acid encoding a recombinant UDP-GalNAc biosynthetic pathway protein capable of converting UDP-GlcNAc to UDP-GalNAc as described in this Section. In certain embodiments, the recombinant nucleic acid in (a) encodes UDP-GalNAc biosynthetic pathway proteins capable of converting GalNAc-1-P to UDP-GalNAc and UDP-GalNAc biosynthetic pathway proteins capable of converting GalNAc to GalNAc-1-P, as described in this Section.

In certain embodiments, the Leishmania host cell comprises (a) a recombinant nucleic acid encoding recombinant UDP-GalNAc biosynthetic pathway proteins capable of converting GalNAc to UDP-GalNAc as described in this Section; and (b) a recombinant nucleic acid encoding a recombinant UDP-GalNAc biosynthetic pathway protein capable of converting UDP-GlcNAc to UDP-GalNAc as described in this Section; and (c) a recombinant nucleic acid encoding a recombinant UDP-GalNAc transporter protein capable of transporting UDP-GalNAc to the secretory pathway as described in Section 7.3.5. In certain embodiments, the recombinant nucleic acid in (a) encodes UDP-GalNAc biosynthetic pathway proteins capable of converting GalNAc-1-P to UDP-GalNAc and UDP-GalNAc biosynthetic pathway proteins capable of converting GalNAc to GalNAc-1-P, as described in this Section.

### 7.3.5 Heterologous UDP-GalNAc Transporter Proteins

In certain embodiments, the Leishmania host cells provided herein comprise a recombinant nucleic acid encoding a UDP-GalNAc transporter protein capable of transporting UDP-GalNAc to the secretory pathway. In certain embodiments, the UDP-GalNAc transporter protein is heterologous to the Leishmania host cell.

In certain embodiments, the UDP-GalNAc transporter protein capable of transporting UDP-GalNAc to the secretory pathway is derived from a nematode source. In certain embodiments, the nematode source is C. elegans.

In certain embodiments, the UDP-GalNAc transporter protein capable of transporting UDP-GalNAc to the secretory pathway comprises UDP-GalNAc transporter of C. elegans (CeC03H5.2), or a functionally active variant thereof. In certain embodiments, the UDP-GalNAc transporter protein capable of transporting UDP-GalNAc to the secretory pathway comprises CeC03H5.2, or a variant that is at least 80%, at least 85%, at least 90%, at least 95%, at least 98%, or at least 99% homologous thereof. In certain embodiments, the UDP-GalNAc transporter protein capable of transporting UDP-GalNAc to the secretory pathway comprises CeC03H5.2. In certain embodiments, the UDP-GalNAc transporter protein capable of transporting UDP-GalNAc to the secretory pathway is CeC03H5.2. In certain embodiments, the UDP-GalNAc transporter protein capable of transporting UDP-GalNAc to the secretory pathway comprises a variant of CeC03H5.2 that is at least 80%, at least 85%, at least 90%, at least 95%, at least 98%, or at least 99% homologous thereof. In certain embodiments, the UDP-GalNAc transporter protein capable of transporting UDP-GalNAc to the secretory pathway is a variant of CeC03H5.2 that is at least 80%, at least 85%, at least 90%, at least 95%, at least 98%, or at least 99% homologous thereof. In certain embodiments, the CeC03H5.2 has an amino acid sequence of SEQ ID NO: 19.

### 7.3.6 Strains of the Leishmania Host Cell

In certain embodiments, the Leishmania host cell is a Leishmania tarentolae cell. In certain embodiments, the Leishmania host cell is a Leishmania aethiopica cell. In certain embodiments, the Leishmania host cell is part of the Leishmania aethiopica species complex. In certain embodiments, the Leishmania host cell is a Leishmania aristidesi cell. In certain embodiments, the Leishmania host cell is a Leishmania deanei cell. In certain embodiments, the Leishmania host cell is part of the Leishmania donovani species complex. In certain embodiments, the Leishmania host cell is a Leishmania donovani cell. In certain embodiments, the Leishmania host cell is a Leishmania chagasi cell. In certain embodiments, the Leishmania host cell is a Leishmania infantum cell. In certain embodiments, the Leishmania host cell is a Leishmania hertigi cell. In certain embodiments, the Leishmania host cell is part of the Leishmania major species complex. In certain embodiments, the Leishmania host cell is a Leishmania major cell. In certain embodiments, the Leishmania host cell is a Leishmania martiniquensis cell. In certain embodiments, the Leishmania host cell is part of the Leishmania mexicana species complex. In certain embodiments, the Leishmania host cell is a Leishmania mexicana cell. In certain embodiments, the Leishmania host cell is a Leishmania pifanoi cell. In certain embodiments, the Leishmania host cell is part of the Leishmania tropica species complex. In certain embodiments, the Leishmania host cell is a Leishmania tropica cell.

### 7.4 Methods of Genetically Engineering a Leishmania Cell

Also provided herein are methods of genetically engineering a Leishmania host cell as described in Section 7.3. In certain embodiments, the method may be used to accomplish the introduction of one or more genes encoding a GalNAc transferase as described in Section 7.3.1. In certain embodiments, the method may be used to accomplish the introduction of one or more genes encoding an additional recombinant glycosyltransferase as described in Section 7.3.2. In certain embodiments, the method may be used to accomplish the functional inactivation of one or more genes encoding an enzyme that catalyzes the formation of O-linked GlcNAc as described in Section 7.3.3. In certain embodiments, the method may be used to accomplish the introduction of one or more genes encoding a heterologous UDP-GalNAc biosynthetic pathway proteins as described in Section 7.3.4. In certain embodiments, the method may be used to accomplish the introduction of one or more genes encoding a heterologous UDP-GalNAc transporter protein as described in Section 7.3.5. In certain embodiments, the strain of the engineered Leishmania host cell is described in Section 7.3.6.

Any method known in the art can be used to engineer the Leishmania host cell, e.g., Leishmania tarentolae. In certain embodiments, nucleic acids are introduced into the host cells described herein using a plasmid, e.g., the heterologous nucleic acids are expressed in the host cells by a plasmid (e.g., an expression vector), and the plasmid is introduced into the modified host cells by transfection, infection, or electroporation, chemical transformation by heat shock, natural transformation, phage transduction, or conjugation. In a specific embodiment, said plasmid is introduced into the modified host cells by stable transfection.

In specific embodiments, linearized nucleic acids are introduced into the host cells described herein using transfection, infection, or electroporation, chemical transformation by heat shock, natural transformation, phage transduction, or conjugation. In a further embodiment, heterologous nucleic acids are integrated site-specifically into the host cell genome by homologous recombination.

In certain embodiments, the method of engineering the Leishmania host cell comprises introducing one or more genes encoding a GalNAc transferase as described in Section 7.3.1.

In certain embodiments, the method of engineering the Leishmania host cell comprises introducing one or more genes encoding an additional recombinant glycosyltransferase as described in Section 7.3.2.

In certain embodiments, the method of engineering the Leishmania host cell comprises functionally inactivating one or more genes encoding an enzyme that catalyzes the formation of O-linked GlcNAc as described in Section 7.3.3. In certain embodiments, the method comprises downregulating the gene encoding the at least one GlcNAc-transferase. In certain embodiments, the method comprises deleting the gene encoding the at least one GlcNAc-transferase. In certain embodiments, the method comprises mutating the gene encoding the at least one GlcNAc-transferase. In certain embodiments, the method comprises overexpressing the gene encoding the at least one GlcNAc-transferase. In certain embodiments, the method comprises functionally inactivating the gene encoding an enzyme that catalyzes the formation of O-linked GlcNAc using the methods described in the Assay or Example Sections (Sections 7.7 and 8, respectively). In certain embodiments, the method comprises functionally inactivating the gene encoding an enzyme that catalyzes the formation of O-linked GlcNAc using any method known in the art, for example methods described in International Publication No. WO2019/002512 A2, incorporated by reference in its entirety herein.

Non-limiting exemplary mutagenesis approaches include site directed mutagenesis using targeted gene editing techniques such as TALENs, ZFNs, CRISPR/Cas9; in combination with a repair scaffold for directed, homologous recombination mediated repair (Zhang, W et al. (2017) mSphere 2 (1); Gupta, R. and Musunuru, K. (2014) The Journal of clinical investigation 124 (10):4154-4161), transposon mutagenesis (Damasceno, J. et al. (2015) Christopher Peacock (Ed.): Parasite Genomics Protocols, vol. 1201. New York, NY: Springer New York (Methods in Molecular Biology), pp. 235-245); replacing the endogenous copy in situ with selectional markers, potentially in combination with a mutated gene version, that are integrated by homologous recombination (Roberts, S. (2011) Bioeng Bugs 2 (6): 320-326); RNA interference (RNAi) (Lye, L. et al. (2010) PLoS Pathog 6 (10), e1001161), conditional knock-down using Cre/LoxP or FRT/FLP (Duncan, S. (2017) Molecular and Biochemical Parasitology 216: 30-38).

Overexpression may be accomplished by the following non-limiting exemplary approaches, such as gene copy number increase by introduction of additional copies into separate loci (Beverley, S. (1991): Gene amplification in Leishmania. In Annu. Rev. Microbiol. 45, pp. 417-444), high expression loci (ribosomal DNA loci) or episomal constructs (Lodes, M. et al. (1995) Mol Cell Biol 15 (12), pp. 6845-6853. DOI: 10.1128/mcb.15.12.6845; Boucher, N. (2004) Nucleic Acids Res 32 (9): 2925-2936), modification of the native UTRs flanking the coding sequence; introduction of additional promoter regions such as the endogenous Poll promoter or a T7 promoter in combination with expression of bacterial T7 polymerase to increase the expression levels (Boucher, N. et al. (2002) Molecular and Biochemical Parasitology 119 (1): 153-158; Gu, P. et al. (2015) Scientific reports 5, p. 9684), use of transposable elements or recombinase based systems such as FRT-FLP or Cre/LoxP to introduce multiple copies of an expression construct (Duncan, S. et al. (2017) Molecular and Biochemical Parasitology 216, pp. 30-38), minichromosome integration (Zomerdijk, J. et al. (1992) Nucleic acids research 20 (11): 2725-2734), and forced chromosomal translocation by CRISPR (Zhang, W. et al. (2017) mSphere 2 (1). DOI: 10.1128/mSphere.00340-16).

In certain embodiments, the method of engineering the Leishmania host cell comprises introducing one or more genes encoding a heterologous UDP-GalNAc biosynthetic pathway proteins as described in Section 7.3.4.

In certain embodiments, the method of engineering the Leishmania host cell comprises introducing one or more genes encoding a heterologous UDP-GalNAc transporter protein as described in Section 7.3.5.

In certain embodiments, the method of engineering the Leishmania host cell comprises (i) functionally inactivating one or more genes encoding an enzyme that catalyzes the formation of O-linked GlcNAc as described in Section 7.3.3; (ii) introducing one or more genes encoding a heterologous UDP-GalNAc biosynthetic pathway proteins as described in Section 7.3.4; (iii) introducing one or more genes encoding a heterologous UDP-GalNAc transporter protein as described in Section 7.3.5; (iv) introducing one or more genes encoding a GalNAc transferase as described in Section 7.3.1; and (v) introducing one or more genes encoding an additional recombinant glycosyltransferase as described in Section 7.3.2.

In certain embodiments, the method comprises conducting steps (i)-(v) in sequential order. In other embodiments, steps (i)-(v) are conducted in a different order. For example, in certain embodiments, steps (ii) and (iii) are conducted before step (i). In other embodiments, step (iv) and/or (v) are conducted before step (i). In certain embodiments, step (v) is conducted before step (iv). In certain embodiments, step (v) is conducted first.

In some embodiments, one or more of steps (i)-(v) may be conducted simultaneously, for example by introducing the genes in a single module. For example, in certain embodiments, steps (ii) and (iii) are conducted simultaneously. In certain embodiments, step (v) is conducted before step (iv). In certain embodiments, step (v) is conducted first.

In a specific embodiment, step (i) is conducted, followed by steps (ii) and (iii), which are conducted simultaneously, and then step (iv) separately. In another specific embodiment, steps (ii) and (iii) are conducted simultaneously and before step (iv), and step (iv) is conducted before step (i). In yet another specific embodiment, step (i) is conducted, and steps (ii), (iii), and (iv) are conducted simultaneously after step (i). In certain embodiments, step (v) is conducted before step (iv). In certain embodiments, step (v) is conducted first.

In certain embodiments, the Leishmania host cells may be engineered using the methods described in the Assay and Examples Sections (Sections 7.7 and 8, respectively).

### 7.5 Methods of Culturing Leishmania Host Cells

Provided herein are methods for culturing Leishmania host cells described in Section 7.3.

In one embodiment, the Leishmania host cells are cultured using any of the standard culturing techniques known in the art. For example, cells are routinely grown in rich media like Brain Heart Infusion, Trypticase Soy Broth or Yeast Extract, all containing 5 μg/ml Hemin. Additionally, incubation is done at 26 °C in the dark as static or shaking cultures for 2-3 days. In some embodiments, cultures of recombinant cell lines contain the appropriate selective agents. Non-limiting exemplary selective agents are provided in Table 2.

In certain embodiments, the Leishmania host cells are cultured in a growth medium comprising GalNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GalNAc. In certain embodiments, the growth medium comprises about 1 mM to about 5 mM, about 5 mM to about 10 mM, about 10 mM to about 15 mM, or about 15 mM to about 20 mM GalNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GalNAc. In certain embodiments, the growth medium comprises about about 10 mM GalNAc.

In certain embodiments, the Leishmania host cells are cultured in a growth medium comprising GlcNAc. In certain embodiments, the growth medium comprises at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, or at least 20 mM GlcNAc. In certain embodiments, the growth medium comprises about 1 mM to about 5 mM, about 5 mM to about 10 mM, about 10 mM to about 15 mM, or about 15 mM to about 20 mM GlcNAc. In certain embodiments, the growth medium comprises about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM GlcNAc.

In certain embodiments, the Leishmania host cells may be cultured using the methods described in the Assay and Examples Sections (Sections 7.7 and 8, respectively).

### 7.6 Uses of the Leishmania Host Cell as an Expression System

In certain embodiments, a Leishmania host cell described in Section 7.3 may be used as an expression system for making a glycoengineered bifunctional degrader described in Section 7.1 or a population of glycoengineered bifunctional degraders described in Section 7.2. In certain embodiments, the glycoengineered bifunctional degrader may be a heterologous, non-Leishmania protein, such as a therapeutic protein (e.g., an antibody). The Leishmania host cells may be engineered as described in Sections 7.4 and cultured as described in Section 7.5. Other methods of producing Leishmania host cells for use as expression systems are known and may also be used, for example, see WO 2019/002512, WO 2021/140144 and WO 2021/140143, each of which are incorporated herein by reference in their entirety. Use of Leishmania host cells to make monoclonal antibodies are also known. Exemplary methods are described in WO 2022/053673, which is incorporated herein by reference in its entirety.

In certain embodiments, the Leishmania host cells may be used as an expression system for producing a glycoengineered bifunctional degrader according to the methods described in the Assay and Examples Sections (Sections 7.7 and 8, respectively).

### 7.6.1 Compositions Comprising Host Cells

In one aspect, provided herein are compositions comprising the Leishmania host cells described in Section 7.3. Such compositions can be used in methods for generating a glycoengineered bifunctional degrader as described in Section 7.1 or a population of glycoengineered bifunctional degraders as described in Section 7.2. In certain embodiments, the compositions comprising Leishmania host cells can be cultured under conditions suitable for the production of glycoengineered bifunctional degraders. Subsequently, the glycoengineered bifunctional degrader can be isolated from said compositions comprising Leishmania host cells using methods known in the art.

The compositions comprising the Leishmania host cells can comprise additional components suitable for maintenance and survival of the Leishmania host cells, and can additionally comprise additional components required or beneficial to the production of glycoengineered bifunctional degraders by the Leishmania host cells, e.g., inducers for inducible promoters, such as arabinose, IPTG.

### 7.6.2 Methods of Making Glycoengineered Bifunctional Degraders

In one aspect, provided herein are methods for making a glycoengineered bifunctional degrader, for example, one described in Section 7.1. In one embodiment, provided herein is a method of producing a glycoengineered bifunctional degrader in vivo, using a Leishmania host cell described in Section 7.3. In a specific embodiment, provided herein is a method for producing a glycoengineered bifunctional degrader, said method comprising (i) culturing a Leishmania host cell described in Section 7.3 under conditions suitable for polypeptide production and (ii) isolating said glycoengineered bifunctional degrader. In a specific embodiment, the Leishmania host cell comprises: (a) a recombinant nucleic acid encoding a glycoengineered bifunctional degrader; and (b) a recombinant nucleic acid encoding one or more recombinant N-acetylgalactosamine (GalNAc) transferases. In certain embodiments, the Leishmania host cell is capable of producing glycoengineered bifunctional degraders comprising a biantennary, GalNAc-terminated N-glycan. In particular, the Leishmania host cells provided herein is capable of producing glycoengineered bifunctional degraders comprising an N-glycan of the following structure:

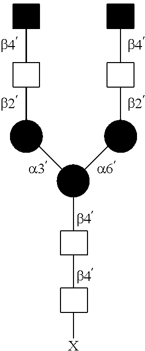

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered bifunctional degrader.

In certain embodiments, the glycoengineered bifunctional degraders described herein are made according to methods described in ‘GLYCOENGINEERING USING LEISHMANIA CELLS’, filed Sep. 27, 2023 as an international application with the European Receiving Office, which claims priority to U.S. Provisional Application Nos. 63/410,936 and 63/410,955, and which is incorporated herein in its entirety.

In certain embodiments, the glycoengineered bifunctional degrader produced by the Leishmania host cell is a therapeutic polypeptide, i.e., a polypeptide used in the treatment of a disease or disorder. For example, the glycoengineered bifunctional degrader produced by the Leishmania host cell can be an enzyme, a cytokine, or an antibody. A list of non-limiting exemplary polypeptides of interest is provided in Section 7.1.

One of skill in the art will readily appreciate that the nucleic acid sequence of a known protein (e.g., a monoclonal antibody), as well as a newly identified protein (e.g., a monoclonal antibody), can easily be deduced using methods known in the art, and thus it would be well within the capacity of one of skill in the art to introduce a nucleic acid that encodes any glycoengineered bifunctional degrader into a host cell provided herein (e.g., via an expression vector, e.g., a plasmid, e.g., a site specific integration by homologous recombination).

Other methods to generate the glycoengineered bifunctional degraders provided herein can be used. For example, chemical conjugation or chemo-enzymatic modifications can be used to generate the bifunctional degraders provided herein

### 7.7 Target Proteins

In some embodiments, the target protein is a cell surface molecule or a non-cell surface molecule. In some embodiments, the cell surface molecule is a receptor. In some embodiments, the non-cell surface receptor is an extracellular protein. In some embodiments, the extracellular protein is an autoantibody, a hormone, a cytokine, a chemokine, a blood protein, or a protein expressed in the central nervous system (CNS).

In some embodiments, the target protein associated with a disease is upregulated in the disease compared to a non-disease state. In some embodiments, the target protein associated with a disease is expressed in the disease compared to a non-disease state. In some embodiments, the target protein associated with a disease is involved in the progression of the disease. In some embodiments, the disease is a cancer or tumor. In some embodiments, the target protein is involved in cancer progression. In some embodiments the disease is an autoimmune disease. In some embodiments, the disease is neurodegenerative disease.

In some embodiments, the disease is Graves' disease. Graves' disease is the most common cause of hyperthyroidism. Prevalence in the US is 1.2% (1), with lifetime risk in women as high as 3%. Production of agonistic anti-TSH Receptor (TSHR) antibodies (TRAb) leading to over production of thyroxine hormone (>90% of patients are TRAb+) (2). Current treatments have not advanced in the 50 years and are limited by high risk of recurrence or severe side effects such as hypothyroidism. In some embodiments, the target protein associated with Graves' disease is an autoantibody binding TSHR. In other embodiments, the target protein associated with Graves' disease is TSHR.

In some embodiments, the target protein comprises a protein selected from the group consisting of TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2 Frizzled receptors, Wnt, LRP5/6, CSF-1R, SIRPα, CD38, CD73, TGF-β, TSHRα, AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin and gangliosides GM1, GD3, and GQ1B. In other embodiments, the target protein comprises an antibody that binds to TSHRα, MOG, AChR-α1, noncollagen domain 1 of the α3 chain of type IV collagen (α3NC1), ADAMTS13, Desmoglein-1/3, GPIb/IX, GPIIb/IIIa, GPIa/IIa, NMDA receptor, glutamic acid decarboxylase (GAD), amphiphysin and gangliosides GM1, GD3, and GQ1B.

In some embodiments, the target protein is a protein that is upregulated in cancer. In some embodiments, the target protein is a protein that is involved in cancer progression. Examples of target proteins that are upregulated in cancer or involved in cancer progression that can be bound by a glycoengineered bifunctional degrader provided herein include, but are not limited to TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2 Frizzled receptors, Wnt, LRP5/6, CSF-1R, SIRPα, CD38, CD73, TGF-β, Bombesin R, CAIX, CD13, CD44v6, Emmprin, Endoglin, EpCAM, EphA2, FAP-α, Folate R, GRP78, IGF-1R, Matriptase, Mesothelin, sMET/HGFR, MT1-MMP, MT6-MMP, PSCA, PSMA, Tn antigen, and uPAR.

In some embodiments, the target protein is an autoantibody, such as those associated with an autoimmune disease. Examples of an autoantibody that can be bound by a glycoengineered bifunctional degrader provided herein include, but are not limited to, autoantibodies directed against TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B.

In some embodiments, the target protein comprises a protein that is upregulated or expressed in tumor associated macrophages (TAMs). In some embodiments, the target protein is upregulated or expressed in pro-tumor TAMs. Examples of target proteins that are upregulated or expressed in TAMs comprise SIRPα, CCR2, CSF-1R, LILRB1, LILRB2, VEGF-R, or CXCR4 (9*). In other embodiments, the target proteins comprise CCL2, CXCL12, CSF-1 or CD47 (9*). These targets play a role in promoting pro-tumor TAMs particularly by promoting TAM recruitment and programming.

In certain embodiments, the target protein is a protein that is upregulated or expressed in a neurodegenerative disease. Examples of target proteins that are upregulated or expressed in neurodegenerative diseases comprise alpha-synuclein, amyloid beta or complement cascade component.

In certain embodiments, the target protein is a protein that is upregulated or expressed in systemic amyloidosis or localized amyloidosis. In some embodiments, the target protein that is upregulated or expressed in systemic amyloidosis is transthyretin.

### 7.8 Methods of Use of the Glycoengineered Bifunctional Degrader

In one aspect, provided herein are methods of preventing or treating a disease or disorder in a subject comprising administering to the subject a glycoengineered bifunctional degrader described in Section 7.1 (including pharmaceutical compositions thereof) or a population of glycoengineered bifunctional degraders described in Section 7.2 (including pharmaceutical compositions thereof). Further provided herein are methods of preventing a disease or disorder in a subject comprising administering to the subject a glycoengineered bifunctional degrader or a population thereof.

In one aspect, provided herein are methods of treating a disease or disorder in a subject comprising administering to the subject a glycoengineered bifunctional degrader described herein or a population thereof. In another aspect, provided herein are methods of preventing a disease or disorder in a subject comprising administering to the subject a glycoengineered bifunctional degrader described herein or a population thereof. In a specific embodiment, provided herein is a method for treating or preventing a disease or disorder in a subject comprising administering to the subject a glycoengineered bifunctional degrader produced according to the methods described herein, wherein the glycoengineered bifunctional degrader is glycosylated with an N-glycan of the following structure:

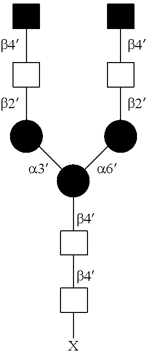

wherein the black square represents an N-acetyl galactosamine (GalNAc), the white square represents an N-acetylglucosamine (GlcNAc) residue and the black circle represents a mannose (Man) residue, and wherein X represents an amino acid residue of the glycoengineered bifunctional degrader.

In certain embodiments, the disease or disorder may be caused by the presence of a defective version of a glycoengineered bifunctional degrader in a subject, the absence of a glycoengineered bifunctional degrader in a subject, diminished expression of a glycoengineered bifunctional degrader in a subject can be treated or prevented using the glycoengineered bifunctional degrader produced using the methods described herein. In certain embodiments, the diseases or disorder may be mediated by a receptor that is bound by a glycoengineered bifunctional degrader produced using the methods described herein, or mediated by a ligand that is bound by a glycoengineered bifunctional degrader produced using the methods described herein (e.g., where the glycoengineered bifunctional degrader is a receptor for the ligand).

In certain embodiments, the methods of preventing or treating a disease or disorder in a subject comprise administering to the subject an effective amount of a glycoengineered bifunctional degrader described herein or a population thereof. In certain embodiments, the effective amount is the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a disease/disorder or symptom associated therewith; (ii) reduce the duration of a disease/disorder or symptom associated therewith; (iii) prevent the progression of a disease/disorder or symptom associated therewith; (iv) cause regression of a disease/disorder or symptom associated therewith; (v) prevent the development or onset of a disease/disorder, or symptom associated therewith; (vi) prevent the recurrence of a disease/disorder or symptom associated therewith; (vii) reduce organ failure associated with a disease/disorder; (viii) reduce hospitalization of a subject having a disease/disorder; (ix) reduce hospitalization length of a subject having a disease/disorder; (x) increase the survival of a subject with a disease/disorder; (xi) eliminate a disease/disorder in a subject; and/or (xii) enhance or improve the prophylactic or therapeutic effect(s) of another therapy.

In some embodiments, provided herein is a method of treating or preventing a disease in a patient comprising administering to the patient a glycoengineered bifunctional degrader described herein or a population described herein. In some embodiments, the disease is an autoimmune disease, a cancer or tumor, a liver disease, an inflammatory disorder, or a blood disorder. In some embodiments, the autoimmune disease is selected from Graves' Disease, Myasthenia Gravis, Anti-GBM Disease, Immune Thrombotic Thrombocytopenic Purpura, Acquired Pemphigus Vulgaris, Immune Thrombocytopenia, Guillain-Barre Syndrome, and Membranous Nephropathy. In some embodiments, the cancer or tumor is selected from breast cancer, colorectal cancer, pancreatic cancer, non-small cell lung cancer, hepatocellular carcinoma, and hematological T cell and B cell malignancies.

In some embodiments, a method of treating or preventing a disease provided herein includes an administration step that comprises intravenous injection, intraperitoneal injection, subcutaneous injection, transdermal injection, or intramuscular injection of a glycoengineered bifunctional degrader described herein or a population described herein.

In some embodiments, a method of treating or preventing a disease provided herein requires a lower dose and/or lower administration frequency to achieve the same effect as compared to the same antibody having a different glycosylation profile; and/or can be administered for an extended period of time (at least 1, 2, 3, 4, 5, 6, 7, 8, 9, 10, 11 or at least 12 months, at least 2, 3, 4, 5, 6, 7, 8, 9, or at least 10 years); and/or does not trigger an immune response against the glycoengineered bifunctional degrader in the patient.

In some embodiments, a suitable dose of a glycoengineered bifunctional degrader described herein is the amount corresponding to the lowest dose effective to produce a therapeutic effect. For example, an effective amount of an anti-TSH receptor antibody can be an amount that inhibits TSH activity in a subject suffering from a Graves' disease.

In some embodiments, the amount of glycoengineered bifunctional degrader described herein administered to a patient is less than the amount listed in the label of a drug product of the same glycoengineered bifunctional degrader having a different glycosylation profile from that of the glycoengineered bifunctional degrader described herein.

In some embodiments, the accumulated amount of a glycoengineered bifunctional degrader described herein administered to a patient over a period of time is less than the accumulated amount indicated in the label of a drug product of the same glycoengineered bifunctional degrader having a different glycosylation profile from that of the glycoengineered bifunctional degrader described herein. In some embodiments, the reduced accumulated amount could be administered in reduced doses on a reduced frequency. In some embodiments, the reduced accumulated amount could be administered in one or more doses that are the same or higher than the dose in the label on a reduced frequency. In some embodiments, the reduced accumulated amount could be administered in one or more reduced doses on a frequency that is the same or higher than the frequency in the label. In some embodiments, the reduced accumulated amount could be administered over a shorter period of time than the period of time for the drug product to achieve the same level of effect in treatment or prevention.

In some embodiments, the amount of the glycoengineered bifunctional degrader described herein in a single dose administered to a patient can be from about 1 to 150 mg, about 5 to 145 mg, about 10 to 140 mg, about 15 to 135 mg, about 20 to 130 mg, about 25 to 125 mg, about 30 to 120 mg, about 35 to 115 mg, about 40 to 110 mg, about 45 to 105 mg, about 50 to 100 mg, about 55 to 95 mg, about 60 to 90 mg, about 65 to 5 mg, about 70 to 80 mg, or about 75 mg. In some embodiments, the amount of glycoengineered bifunctional degrader described herein in a single dose administered to a patient can be from about 5 to about 80 mg. In some embodiments, the amount of glycoengineered bifunctional degrader described herein in a single dose administered to a patient can be from about 25 to about 50 mg. In some embodiments, the amount of a glycoengineered bifunctional degrader described herein in a single dose administered to a patient can from about 15 mg to about 35 mg.

In some embodiments, the amount of a glycoengineered bifunctional degrader described herein in a single dose administered to a patient can be no more than 40 mg, for example 40 mg, 35 mg, 30 mg, 25 mg, 20 mg, 18 mg, 15 mg, 12 mg, 10 mg, 7 mg, 5 mg, and 2 mg. In some embodiments, the amount of a glycoengineered bifunctional degrader described herein in a single dose administered to a patient can be no more than 80 mg, for example 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a glycoengineered bifunctional degrader described herein in a single dose administered to a patient can be no more than 160 mg, for example 150 mg, 140 mg, 130 mg, 120 mg, 110 mg, 100 mg, 90 mg, 80 mg, 75 mg, 70 mg, 65 mg, 60 mg, 55 mg, 50 mg, 45 mg, 40 mg, 35 mg, 30 mg, 20 mg, 15 mg, 10 mg, 5 mg and 2 mg. In some embodiments, the amount of a glycoengineered bifunctional degrader described herein in a single dose administered to a patient can be equal to or more than 160 mg, for example 170 mg, 180 mg, 200 mg, 250 mg, and 300 mg.

In some embodiments, a glycoengineered bifunctional degrader of the disclosure can be administered on a frequency that is every other week, namely every 14 days. In some embodiments, a glycoengineered bifunctional degrader of the disclosure can be administered on a frequency that is lower than every 14 days, for example, every half a month, every 21 days, monthly, every 8 weeks, bimonthly, every 12 weeks, every 3 months, every 4 months, every 5 months, or every 6 months. In some embodiments, a glycoengineered bifunctional degrader of the disclosure can be administered on a frequency that is the same or higher than every 14 days, for example, every 14 days, every 10 days, every 7 days, every 5 days, every other day, or daily.

In some embodiments, the administration of a glycoengineered bifunctional degrader of the disclosure can comprise an induction dose that is higher than the following doses, for example the following maintenance doses. In some embodiments, the administration of a glycoengineered bifunctional degrader of the disclosure can comprise a second dose that is lower than the induction dose and higher than the following maintenance doses. In some embodiments, the administration of a glycoengineered bifunctional degrader of the disclosure can comprise the same amount of the glycoengineered bifunctional degrader in all the doses throughout the treatment period.

In some embodiments, provided herein is a method of treating an acute condition associated with increased levels of a target protein, wherein the method comprises administering to a patient in need thereof a glycoengineered bifunctional degrader described herein, wherein the method results in a half-life that is at least 50%, 60%, 70%, 80%, 90% or 99% of the bifunctional degrader without any glycosylation. In some embodiments, the half-life of the target protein in the presence of a bifunctional degrader provided herein in a patient is 5 minutes, 10 minutes, 15 minutes, 30 minutes, 45 minutes, 1 hour, 1.5 hours, 2 hours, 2.5 hours, or 3 hours.

In some embodiments, provided herein is a method of treating a chronic condition associated with increased levels of a target protein, wherein the method comprises administering to a patient in need thereof a bifunctional degrader described herein, wherein the method results in a half-life that is at least 50%, 60%, 70%, 80%, 90% or 99% of the bifunctional degrader without any glycosylation in the patient. In some embodiments, the half-life of the target protein is at least 6 hours, 12 hours, 18 hours, 1 day, 2 days, 3 days, 4 days, 5 days, 6 days or 7 days.

In some embodiments, provided herein is a method of treating a chronic condition associated with increased levels of a target protein, wherein the method comprises administering to a patient in need thereof a bifunctional degrader described herein, wherein the bifunctional degrader (i) specifically binds to the target protein and (ii) comprises an N-glycan of the following structure:

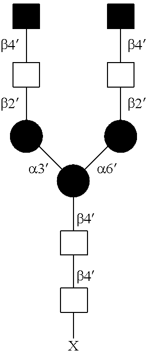

wherein the black square represents an N-acetyl galactosamine (GalNAc), the white square represents an N-acetylglucosamine (GlcNAc) residue and the black circle represents a mannose (Man) residue, and X represents an amino acid residue of the glycoengineered bifunctional degrader, and wherein the N-glycan is linked to the bifunctional degrader at one, two or more N-glycosylation sites such that the half-life is at most 20%, 30%, 40%, 50%, 60%, 70%, 80%, 90% or 99% of the half-life of the target protein in the patient in the absence of the bifunctional degrader or in the absence of any treatment.

In some embodiments, the chronic condition is an autoimmune disease, a cancer or tumor, a liver disease, an inflammatory disorder, or a blood coagulation disorder.

In some embodiments, the autoimmune disease is selected from Graves' Disease, Myasthenia Gravis, Anti-GBM Disease, Immune Thrombotic Thrombocytopenic Purpura, Acquired Pemphigus Vulgaris, Immune Thrombocytopenia, autoimmune encephalitis, Guillain-Barré Syndrome, and Membranous Nephropathy.

In some embodiments, the cancer comprises a solid tumor. In some embodiments, the cancer comprises a blood-borne cancer or tumor. In some embodiments, the cancer may be a carcinoma or a sarcoma. In some embodiments, the cancer is selected from lung cancer (small cell or non-small cell), breast cancer, gastric cancer, colorectal cancer, bladder cancer, malignant melanoma, brain cancer (e.g., astrocytoma, glioma, meningioma, neuroblastoma, or others), bone cancer (e.g., osteosarcoma), cervical cancer, cholangiocarcinoma, digestive tract cancer (e.g., oral, esophageal, stomach, colon or rectal cancer), head and neck cancer, leiomyosarcoma, liposarcoma, liver cancer (e.g., hepatocellular carcinoma), mesothelioma, nasopharyngeal cancer, neuroendocrine cancer, ovarian cancer, pancreatic cancer, prostate cancer, renal cancer, rhabdomyosarcoma, salivary gland cancer, skin cancer, spindle cell carcinoma, testicular cancer, thyroid cancer, or uterine cancer (e.g., endometrial cancer). In certain embodiments, the cancer can be relapsed following a previous therapy, or refractory to conventional therapy. In certain embodiments, the cancer can be disseminated or metastatic. In some embodiments, the blood-borne cancer or tumor is selected from leukemia, myeloma (e.g., multiple myeloma) lymphoma (e.g., Hodgkin's lymphoma or non-Hodgkin's lymphoma). In certain embodiments, the leukemia is chronic lymphocytic leukemia, chronic myeloid leukemia, acute lymphocytic leukemia, acute myelogenous leukemia and acute myeloblastic leukemia.

In some embodiments, treatment comprises reprogramming tumor associated macrophages (TAMs) by administering the bifunctional degrader under conditions to mediate endocytosis of a target protein. In some embodiments, the target protein is upregulated or expressed in TAMs. In some embodiments, the target protein upregulated or expressed in TAMs comprises SIRPα, CCR2, CSF-1R, LILRB1, LILRB2, VEGF-R, CXCR4, CCL2, CXCL12, CSF-1 or CD47.

In some embodiments, the administration step comprises intravenous injection, intraperitoneal injection, subcutaneous injection, transdermal injection, or intramuscular injection.

In some embodiments, a method of delivering a target protein to a hepatocyte endosome is provided herein. In some embodiments, the method of delivering a target protein to a hepatocyte comprises contacting the target protein with any of the glycoengineered bifunctional degraders disclosed herein under conditions to mediate endocytosis of any of the target proteins disclosed herein. In some embodiments, the method of delivering the target protein to a hepatocyte endosome occurs in vivo. In some embodiments, the mode of delivering a target protein to a hepatocyte endosome in vivo comprises intravenous injection, intraperitoneal injection, subcutaneous injection, transdermal injection or intramuscular injection. In some embodiments, the method of delivering the target protein to a hepatocyte endosome occurs ex vivo.

In some embodiments, the rate of delivery can be increased based on the number of N-glycan structures present on the glycoengineered bifunctional degrader. In some embodiments, increasing the number of N-glycan structures on the glycoengineered bifunctional degrader increases the rate of delivery. In some embodiments, the glycoengineered bifunctional degrader can comprise 1 or more, 2 or more, 3 or more, 4 or more, 5 or more, 6 or more, 7 or more, 8 or more, 9 or more or 10 or more N-glycans of the following structure:

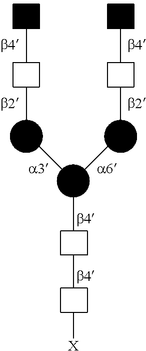

wherein the black square represents an N-acetyl galactosamine (GalNAc), the white square represents an N-acetylglucosamine (GlcNAc) residue and the black circle represents a mannose (Man) residue, and X represents an amino acid residue of the glycoengineered bifunctional degrader.

In some embodiments, a method of degrading a target protein is provided herein. In some embodiments, the method of degrading a target protein comprises contacting the target protein with any of the glycoengineered bifunctional degraders disclosed herein under conditions to mediate degradation of any of the target proteins disclosed herein by a host cell. In some embodiments, degradation is lysosomal degradation. In some embodiments, degradation is mediated by endocytosis or phagocytosis. In certain embodiments, the method comprises degrading a structure comprising at least one target protein bound to at least one glycoengineered bifunctional degrader. In certain embodiments, the structure has a size of about 50 kDa or more, about 75 kDa or more, about 100 kDa or more, about 150 kDa or more, about 200 kDa or more, about 250 kDa or more, about 300 kDa or more, about 400 kDa or more, about 500 kDa or more, about 600 kDa or more, about 700 kDa or more, about 800 kDa or more, about 900 kDa or more, about 1000 kDa or more, about 1100 kDa or more, about 1200 kDa or more, or about 1300 kDa or more. In some embodiments, degradation is at least 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than degradation mediated by a glycoengineered bifunctional degrader not comprising an at least one or at least two N-glycans of the following structure:

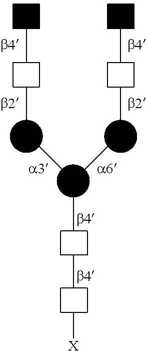

wherein the black square represents an N-acetyl galactosamine (GalNAc), the white square represents an N-acetylglucosamine (GlcNAc) residue and the black circle represents a mannose (Man) residue, and X represents an amino acid residue of the glycoengineered bifunctional degrader. In other embodiments, the glycoengineered bifunctional degrader enhances degradation of any of the disclosed target proteins relative to degradation of the target protein in the presence of a glycoengineered bifunctional degrader not comprising at least one or at least two of the N-glycans.

Without being bound by theory, increasing the rate and/or efficiency of internalization of the glycoengineered bifunctional degrader (mediated, for example, by ASGPR) increases the rate and/or activity of lysosomal degradation. In certain embodiments, the rate and/or efficiency of internalization of the glycoengineered bifunctional degrader (mediated, for example, by ASGPR) is proportional to the rate and/or activity of lysosomal degradation. In certain embodiments, relative changes in the rate and/or efficiency of internalization of the glycoengineered bifunctional degrader (mediated, for example, by ASGPR) are determined from relative changes in the rate and/or activity of lysosomal degradation. In some embodiments, the rate and/or efficiency of internalization can be regulated through glycoengineering. In some embodiments, the rate and/or efficiency of internalization can be regulated based on the number of N-glycan structures present on the glycoengineered bifunctional degrader. In some embodiments, the rate and/or efficiency of internalization can be increased based on the number of N-glycan structures present on the glycoengineered bifunctional degrader. In some embodiments, increasing the number of N-glycan structures on the glycoengineered bifunctional degrader increases the rate and/or efficiency of internalization. In some embodiments, increasing the number of N-glycan structures on the glycoengineered bifunctional degrader by one N-glycan increases the rate and/or efficiency of internalization as compared to a glycoengineered bifunctional degrader comprising one fewer N-glycan. In some embodiments, the increase in rate and/or efficiency of internalization resulting from increasing the number of N-glycan structures on the glycoengineered bifunctional degrader by one N-glycan is more than additive. In certain embodiments, the increase in rate and/or efficiency of internalization resulting from increasing the number of N-glycan structures on the glycoengineered bifunctional degrader by one N-glycan is about 10% or more, about 20% or more, about 30% or more, about 40% or more, about 50% or more, about 60% or more, about 70% or more, about 80% or more, about 90% or more, or about 100% or more than the rate and/or efficiency of internalization per N-glycan for a glycoengineered bifunctional degrader comprising one less, two less, three less, or four less N-glycans. In some embodiments, the glycoengineered bifunctional degrader can comprise 1 or more, 2 or more, 3 or more, 4 or more, 5 or more, 6 or more, 7 or more, 8 or more, 9 or more or 10 or more N-glycans of the following structure:

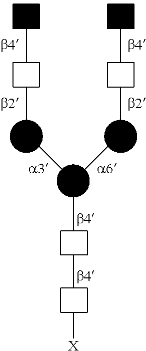

wherein the black square represents an N-acetyl galactosamine (GalNAc), the white square represents an N-acetylglucosamine (GlcNAc) residue and the black circle represents a mannose (Man) residue, and X represents an amino acid residue of the glycoengineered bifunctional degrader. In some embodiments, the presence of two or more of the N-glycans on the glycoengineered bifunctional degrader can increase the rate and/or efficiency of internalization relative to a glycoengineered bifunctional degrader comprising only one of the N-glycan. In some embodiments, the rate and/or efficiency of internalization can be fine-tuned. That is, the rate and/or efficiency of internalization can be increased by increasing the number of N-glycan structures present. Depending on the condition to be treated, different internalization rates are desired. For the treatment of an acute condition, rapid internalization of the complex between a bifunctional degrader provided herein bound to its target protein(s) would be desired. To accomplish that at least 1, 2, 3, 4, 5, 6, 7, 8, 9, or 10 N-glycosylation sites can be introduced and linked to the N-glycan, which in turn results in a rapid internalization and low half lifes of the target protein of less than 5 minutes, 10 minutes, 15 minutes, 30 minutes, 45 minutes, 60 minutes, 90 minutes, two hours, three hours, or less than four hours. For the treatment of an acute condition associated with increased levels of a target protein, the method comprises administering to a patient in need thereof a bifunctional degrader, wherein the bifunctional degrader (i) specifically binds to the target protein and (ii) comprises the N-glycan, wherein the N-glycan is linked to the bifunctional degrader at one, two or more N-glycosylation sites such that the half-life of the target protein is at most 0.1% 0.5%, 1%, 10% 20%, 30%, 40%, 50%, 60%, 70%, 80%, 90% or 99% of the half-life of the target protein in the patient in the absence of the bifunctional degrader or in the absence of any treatment.

In some embodiments, the rate and/or efficiency of internalization of the bifunctional degrader can be regulated based on location of the N-glycan(s) on the bifunctional degrader. In some embodiments, the rate and/or efficiency of internalization can be increased based on the location of the N-glycans(s) on the bifunctional degrader. In certain embodiments, the rate and/or efficiency of internalization of the bifunctional degrader is enhanced by engineering the bifunctional degrader so as to comprise one or more N-glycosylation sites distal to a target-specific binding location of the bifunctional degrader. In certain embodiments, the rate and/or efficiency of internalization of the bifunctional degrader is enhanced by engineering the bifunctional degrader so as to comprise at least one or at least two N-glycosylation sites distal to the target-specific binding location. In certain embodiments, the rate and/or efficiency of internalization of the bifunctional degrader is enhanced by engineering the bifunctional degrader so as to comprise all N-glycosylation sites distal to the target-specific binding location. In certain embodiments, the efficiency of target engagement and internalization by a bifunctional degrader is enhanced relative to an unmutated form of the bifunctional degrader by engineering the bifunctional degrader so as to delete, mutate, or functionally inactivate N-glycosylation sites present in a wild-type, natural, synthetic, or commercial precursor of the bifunctional degrader (“natural N-glycosylation sites”). In certain embodiments, the efficiency of target engagement and internalization by the bifunctional degrader is enhanced relative to an unmutated form of the bifunctional degrader by engineering the bifunctional degrader so as to delete, mutate, or functionally inactivate at least one or at least two natural N-glycosylation sites. In certain embodiments, the efficiency of target engagement and internalization by the bifunctional degrader is enhanced relative to an unmutated form of the bifunctional degrader by engineering the bifunctional degrader so as to delete, mutate, or functionally inactivate all natural N-glycosylation sites. In certain embodiments, efficiency of target engagement and internalization by the bifunctional degrader is enhanced relative to an unmutated form of the bifunctional degrader by engineering the bifunctional degrader so as to delete, mutate, or functionally inactivate one or more natural N-glycosylation sites are located at or proximal to the target-specific binding location of the wild-type, natural, synthetic, or commercial precursor of the bifunctional degrader. In certain embodiments, the target-specific binding location is a variable region of an antibody or antigen-binding fragment (Fab), or an ectodomain of an Fc-fusion protein. In certain embodiments, the efficiency of target degradation by a mutated form of the bifunctional degrader is enhanced relative to the unmutated form of the bifunctional degrader by engineering the bifunctional degrader so as to delete, mutate, or functionally inactivate one or more N-glycosylation sites present in a wild-type, natural, synthetic, or commercial precursor of the bifunctional degrader (“natural N-glycosylation sites”). In certain embodiments, the efficiency of target degradation is enhanced by eliminating or decreasing the number of N-glycosylation sites located at or proximal to the target-specific binding location. Without being bound by theory, eliminating or decreasing the number of A2GalNAc2 glycans located at or proximal to the target-specific binding location can reduce the rate of internalization and clearance of the bifunctional degrader before target binding (i.e. of an unbound form of the degrader) as compared to after target binding (i.e. to a bound form of the degrader). Thus, without being bound by theory, eliminating or decreasing the number of A2GalNAc2 glycans located at or proximal to the target-specific binding location can increase the probability that the bifunctional degrader binds to its target before being internalized via ASGPR. In certain embodiments, the number of N-glycosylation sites located at or proximal to the target-specific binding location is eliminated or reduced by deleting, mutating, or functionally inactivating one or more natural N-glycosylation sites. In certain embodiments, all glycoengineered N-glycosylation sites are distal to the target-specific binding location of the bifunctional degrader. In certain embodiments, the rate and/or efficiency of internalization of the bifunctional degrader, wherein the bifunctional degrader is an antibody, is enhanced by engineering the bifunctional degrader so as to comprise two N-glycosylation sites in the Fab region of the antibody, wherein one N-glycosylation site is located on each of the two heavy chain polypeptides of the antibody and each of said heavy chain N-glycosylation sites is glycosylated by the N-glycan, as compared to engineering the bifunctional degrader so as to comprise two N-glycosylation sites in the Fab region of the antibody, wherein one N-glycosylation site is located on each of the two light chain polypeptides of the antibody and each of said light chain N-glycosylation sites is glycosylated by the N-glycan. In certain embodiments, rate and/or efficiency of internalization of the bifunctional degrader, wherein the bifunctional degrader is an antibody, is enhanced by engineering the bifunctional degrader so as to comprise at least two N-glycosylation sites distal (in terms of the quaternary structure of the antibody) to the hinge region of the antibody, and wherein at least two of the distal N-glycosylation sites are glycosylated by the N-glycan, as compared to a bifunctional degrader comprising N-glycosylation sites proximal (in terms of the quaternary structure of the antibody) to the hinge region of the antibody, and wherein only the proximal N-glycosylation sites are glycosylated by the N-glycan. In certain embodiments, the bifunctional degrader has been engineered as described in Section 7.1.

In some embodiments, a method of degrading a target protein comprises GalNAc mediated degradation. In some embodiments, GalNAc degradation is optimal due to engagement of endocytic receptors. In some embodiments, the method of degrading a target protein via GalNAc mediated degradation is selective. In some embodiments, GalNAc degradation removes inflammatory cytokines from circulation, removes unwanted blood factors, removes autoantibodies, removes pathogenic antibodies, removes cell surface receptors, removes protein aggregates and removes extracellular soluble proteins.

### 7.9 Assay

### 7.9.1 Strains, Growth and Genetic Methods

Provided herein are methods for culturing Leishmania host cells described in Section 7.3 for the production of bifunctional degraders.

Host cells are cultured using any of the standard culturing techniques known in the art. For example, cells are routinely grown in rich media like Brain Heart Infusion, Trypticase Soy Broth or Yeast Extract, all containing 5 μg/ml Hemin. In some embodiments, incubation is done at 26 °C in the dark as static or shaking cultures for 2-3 days. In some embodiments, cultures of recombinant cell lines contain the appropriate selective agents. In some embodiments, cultures contain Biopterin at a final concentration of 10 μM to support growth.

A non-limiting list of selective agents is provided in Table 2.

TABLE 2
Selective agents used during transfection (50% concentration for
preselection and 100% concentration for main selection) and standard
culturing of L. tarentolae. Double amounts of the selective agents
could be used if higher selection pressure was intended.

| | | Concentration | |
|---|---|---|---|
| | Resistance | (100%) | Concentration |
| Selective | conferring | main selection/ | (50%) |
| agent | gene | standard culturing | preselection |
| Nourseothricin | sat | 50 μg/ml | 25 μg/ml |
| Geneticin | neo | 50 μg/ml | 25 μg/ml |
| Paromomycin | neo | 300 μg/ml | 150 μg/ml |
| Zeocin | ble | 150 μg/ml | 75 μg/ml |
| Hygromycin | hyg | 50 μg/ml | 25 μg/ml |
| Blasticidin | bsd | 5 μg/ml | 2.5 μg/ml |
| Puromycin | pac | 5 μg/ml | 2.5 μg/ml |

TABLE 3
Summary of exemplary strains. Some of the strains were produced by
several rounds of transfection building on top of each other.

| | Parental strain | Engineering module_selection | | |
|---|---|---|---|---|
| Strain number | number | marker [integration locus] | Glycan | Description Target Protein |
| StLMTB10569 | / | / | Man3 | / |
| StLMTB18407 | StLMTB10569 | G0 long_hyg(A) [Pfr] | A2 | / |
| StLMTB19462 | StLMTB18407 | G0 3.2_pac(D) [ssu-PolI] | A2 | / |
| StLMTB19855 | StLMTB19462 | triple OGNT KO_ble(F) | A2 | / |
| StLMTB19915 | StLMTB19855 | Adalimumab (K84N/D86N) | A2 | Adalimumab (K84N/D86N) |
| | | ntc(E) [ssu] | | |
| StLMTB20097 | StLMTB19462 | triple OGNT KO_neo(B) | A2 | / |
| StCGP2801 | StLMTB19915 | GalNAc synth: T + S + E | A2 | Adalimumab (K84N/D86N) |
| | | (IR variant J, AC) bsd(C) [Pfr] | | |
| StCGP2803 | StLMTB19915 | GalNAc synth: T + S + E | A2 | Adalimumab (K84N/D86N) |
| | | (IR variant J, AC) bsd(C) [aTub] | | |
| StCGP2805 | StLMTB19915 | GalNAc synth; T + S | A2 | Adalimumab (K84N/D86N) |
| | | (IR variant Y, Z) bsd(C) [aTub] | | |
| StCGP2807 | StLMTB19915 | GalNAc synth: T + E bsd(C) | A2 | Adalimumab (K84N/D86N) |
| | | [aTub] | | |
| StCGP2847 | StLMTB19915 | GalNAc synth T + S + E | A2 | Adalimumab (K84N/D86N) |
| | | (IR variant Y, Z) bsd(C) [aTub] | | |
| StCGP2853 | StCGP2801 | GalNAc T3_ neo(B) [Pfr] | A2GalNAc2 | Adalimumab (K84N/D86N) |
| StCGP2877 | StCGP2801 | GalNAc T3/T4_ neo(B) [Pfr] | A2GalNAc2 | Adalimumab (K84N/D86N) |
| StCGP2855 | StCGP2803 | GalNAc T3_ neo(B) [Pfr] | A2GalNAc2 | Adalimumab (K84N/D86N) |
| StCGP2879 | StCGP2803 | GalNAc T3/T4_ neo(B) [Pfr] | A2GalNAc2 | Adalimumab (K84N/D86N) |
| StCGP2857 | StCGP2805 | GalNAc T3_ neo(B) [Pfr] | A2GalNAc2 | Adalimumab (K84N/D86N) |
| StCGP2881 | StCGP2805 | GalNAc T3/T4_ neo(B) [Pfr] | A2GalNAc2 | Adalimumab (K84N/D86N) |
| StCGP2860 | StCGP2807 | GalNAc T3_ neo(B) [Pfr] | A2GalNAc2 | Adalimumab (K84N/D86N) |
| StCGP2861 | StCGP2847 | GalNAc T3_ neo(B) [Pfr] | A2GalNAc2 | Adalimumab (K84N/D86N) |
| StCGP3023 | StLMTB19462 | GalNAc synth_ ble(F) [Pfr] | A2 | / |
| StCGP3053 | StCGP3023 | GalNAcT_ bsd(C) [ssuPolI] | A2GalNAc2 | / |
| StCGP3169 | StCGP3053 | +triple OGNT KO_neo(B) | A2GalNAc2 | / |
| StCGP3558 | StLMTB20097 | GalNAc synth GalNAcT | A2GalNAc2 | / |
| | | bsd(C) [ssuPoll] | | |
| StCGP3127 | StCGP3053 | Adalimumab (K84N/D86N) | A2GalNAc2 | Adalimumab (K84N/D86N) |
| | | ntc(E) [ssu] | | |
| StCGP3220 | StCGP3169 | Adalimumab (K84N/D86N) | A2GalNAc2 | Adalimumab (K84N/D86N) |
| | | ntc(E) [ssu] | | |
| SCGP3270 | StCGP3169 | 5C9 (D86N) ntc(E) [ssu] | A2GalNAc2 | 5C9 (D86N), |
| | | | | IgG4 mAb |
| StCGP3264 | StCGP3169 | 11k2 (D86N) ntc(E) [ssu] | A2GalNAc2 | 11k2 (D86N), |
| | | | | IgG4 mAb |

### (i) Plasmids

Plasmids were derived from a pUC57 vector backbone for E. coli propagation and contained an ampicillin or kanamycin section marker. The expression cassettes are flanked by restriction sites suitable for excision. The composition of the cassettes depends on the intended use and is described in the respective methods and examples. The genes of interest are included as ORFs that were codon usage optimized for L. tarentolae. Optimized sequences were manually curated for avoidance of restriction sites and deletion of repeats or homopolymer stretches. The plasmids were generated and sequenced by a gene synthesis provider. Plasmids and descriptions are found in the sequence listings.

For codon usage optimization, protein sequences were back-translated to nucleotide sequences using a custom Python3 script that stochastically selects codons based on the L. tarentolae codon usage frequency while excluding rare codons (frequency <10%). The codon usage has been calculated using cusp (Rice, et al. (2000) Trends in genetics: TIG 16 (6), pp. 276-277) on all annotated L. tarentolae nucleotide coding sequences.

### (ii) Transfection Method

### (A) Preparation of DNA

Restriction digest (12 μg DNA in total volume of 240 μL) was performed using standard restriction enzymes (ThermoFisher, preferably FastDigest) according to the manufacturer's instructions. The restriction digest was performed until completion or o/n at 30 °C and DNA was purified by EtOH precipitation (2 volume 100% ice cold EtOH was added to 1 volume digested DNA, incubated 30 min on ice, centrifuged for 30 min 17'500×g at 4 °C. Pellet was washed with 70% EtOH and subsequently dried for maximum 15 min and resuspended in ddH20. For optimized removal of circularized plasmid, 1 or 2 restriction enzymes with recognition sites in the vector backbones were chosen and digest was done for 1 h at 37 °C and purified by EtOH as described above. The digest was analyzed by agarose gel electrophoresis in 0.7-2% agarose gels (TAE buffer). Optionally, gel extraction was performed with the NucleoSpin® Gel and PCR Clean-up kit (Macherey&Nagel) according to manufacturer's instructions to remove undigested plasmid from the preparation.

(B) Transfection with Nucleofector

One day before transfection, a densely grown culture of the parental strain was diluted 1:10 into fresh media (Brain Heart Infusion plus Hemin, “BHIH”; or Yeast Extract plus Hemin, “YEH”) containing all antibiotics for which selection markers were previously integrated and cultured overnight at 26 °C.

The linear DNA fragments for integration are mixed for transfection in the needed combinations for multiple DNA fragment-homologous recombination at 1 μg per fragment. The volume of the mix was reduced to approximately 2 μl per transfection in a vacuum concentrator at 30 °C. For episomal transfection of plasmids, 0.1-1 μg of plasmid DNA were directly used for transfection.

Transfection was performed using the 4D-Nucleofector™_Core_X with the P3 Primary Cell 4D-Nucleofector™ X Kit (Lonza). For this, DNA as prepared above was mixed with 16.4 μl P3 Primary Cell solution and 3.6 μl Supplement Solution. The equivalent culture volume of 10⁷ cells (OD should be around 0.3-1.0/ml, cell shape round to drop-like) was pelleted by centrifugation at 1800 g for 5 min and the supernatant was removed. The cell pellet was resuspended in the DNA mix and transfected using a 16-well electroporation strip with pulse FI-158 (in some examples alternative pulses FP167, CM150, EO115, DN100, FP158, FB158 were used). As negative control, an additional culture was transfected with ddH2O only.

80 μl of fresh media (BHIH or YEH plus parental selection markers) was added to each well and 2×45 μl of the mix were transferred to individual wells of a 96 well culture plate that were prefilled with 200 μl of fresh medium. After incubation for 24 h at 26 °C in the dark (recovery), the new selection marker was added at 50% concentration (preselection; see Table 2). After further incubation for 1-2 days, the selection marker was topped up to 100% (main selection, see Table 2) and several dilutions between 1:2 and 1:10 were performed in 96 well format (final volume 250 μl). Cultures were further incubated at 26 °C in the dark for up to 7 days. If no growth was observed, the culture medium was replaced (centrifugation at 1800 g, 10 min, RT) and cultures were again incubated for up to 7 days. This step was repeated if necessary. Growing cultures were expanded in to higher culture volumes by dilutions in the range of 1:5 and 1:20 before analysis.

For clonal selection, cells were streaked on BHIH or YEH plates (containing 1.4% agar and the appropriate 100% selective agent) as soon as the liquid culture turned turbid. Plates were sealed with parafilm and incubated 7-10 days upside down in dark at 26 °C. Single colonies (1-2 mm size) were transferred into 24-well plates containing 1 ml BHIH or YEH, sealed with parafilm and incubated in dark at 26 °C for around 7-10 days. 1 ml culture was then transferred from 24-well plate into 10 ml BHI or YEH in a flask and further grown statically as usual.

(iii) Method of Engineering Leishmania tarentolae Cells Devoid of O-glycosylation

Without being bound by theory, one of the most effective methods to control O-linked GlcNAc modification in L. tarentolae has been found to be an RNP transfection for CRISPR/Cas9 mediated replacement of all three OGNT genes with a single selection marker. See WO 2021/140143. However, other methods have been reported and may be employed, See WO 2021/140143 which is incorporated herein by reference in its entirety. In one embodiment, a ribonucleoprotein complex formed of the endonuclease SpCas9 and bipartite guideRNAs (gRNA) are transfected into L. tarentolae to introduce double-strand breaks in the 5′ and 3′ regions of the open reading frames encoding OGNT1, OGNT2 and OGNTL. The gRNAs are formed by a scaffold RNA (tracrRNA) and one of the six sequence specific targeting RNAs (crRNA), used in this method. Along with the RNP complexes a selection marker expression construct consisting of two linear DNA fragments is transfected into the cells. During double-strand break repair in L. tarentolae the linear DNA pieces are integrated at the former OGNT expression sites by homologous recombination with each other and the 5′ and 3′ untranslated regions of the OGNT gene. In the setup described here, the selection marker expression construct does not introduce additional flanking untranslated regions and thus results in transcription of the marker by endogenous PolII.

### (A) Preparation of Ribonucleoprotein (RNP) Complexes for Transfection

gRNA for CRISPR/Cas9 mediated genome editing was assembled from equimolar amounts of tracrRNA and crRNA (Microsynth) as above by denaturation for 5 min at 95 °C and subsequent slow cool down at 0.1 °C/s in a thermo cycler. This was done separately for every crRNA used before the different gRNAs were subsequently mixed in equimolar amounts. Next, 122 pmol recombinantly expressed Cas9 protein (i.e. Alt-R® S.p. HiFi Cas9 Nuclease V3 (IDT, #1081061) were added to 360 pmol of the gRNA mix and incubated for 15 min at RT to allow RNP formation. The final volume used for a transfection by Nucleofector (as described in this Section) should not exceed 6 μl. Lastly, the RNP mix was added to the repair DNA containing transfection solution described below along with 1 μl of Alt-R® Cas9 Electroporation Enhancer (IDT, #1081072).

### (B) DNA Preparation for Transfection

The linear DNA fragments for integration are mixed for transfection in the needed combinations at 1 μg per fragment and the gRNA was prepared as described above and mixed with the integration fragments. The volume of the mix was reduced to maximum 2 μl per transfection in a vacuum concentrator at 30 °C.

(C) Transfection with Nucleofector

One day before transfection, a densely grown culture of the parental strain was diluted 1:10 into fresh media (BHIH) containing all antibiotics for which selection markers were previously integrated and cultured over night at 26 °C.

Transfection was performed using the 4D-Nucleofector™_Core_X with the P3 Primary Cell 4D-Nucleofector™ X Kit (Lonza). For this, DNA (or DNA/RNA or DNA/RNP) as prepared above was mixed with 16.4 μl P3 Primary Cell solution and 3.6 μl Supplement Solution. The equivalent culture volume of 10⁷ cells (OD should be around 0.3-1.0/ml, cell shape round to drop-like) was pelleted by centrifugation at 1800 g for 5 min and the supernatant was removed. The cell pellet was resuspended in 20 μl of the DNA (or DNA/RNA or DNA/RNP) mix and transfected using a 16-well electroporation strip with pulse FI-158 (in some examples alternative pulses FP167, CM150, EO115, DN100, FP158, FB158 were used). As negative control, an additional culture was transfected with ddH2O only.

80 μl of fresh media (containing selection markers for the parental cell line) was added to each well and 2×45 μl of the mix were transferred to individual wells of a 96 well culture plate that were prefilled with 200 μl of fresh medium. After incubation for 24 h at 26 °C in the dark (recovery), the new selection marker was added at 50% concentration (preselection; see Table 2). After further incubation for 1-2 days, the selection marker was topped up to 100% (main selection) and several dilutions between 1:2 and 1:10 were performed in 96-well format (final volume 250 μl). Cultures were further incubated at 26 °C in the dark for up to 7 days. If no growth was observed, the culture medium was replaced (centrifugation at 1800 g, 10 min, RT) and cultures were again incubated for up to 7 days. This step was repeated if necessary. Growing cultures were expanded into higher culture volumes by dilutions in the range of 1:5 and 1:20 before analysis.

(D) crRNA Design

crRNAs were designed based on the target regions (usually coding sequences of OGNT genes) for use with SpCas9 (PAM=NGG) by EuPaGDT (http://grna.ctegd.uga.edu/) and were counterchecked for on-/off-target effects by blast against the whole genome of L. tarentolae. crRNAs were then selected such that they are ideally targeting the extremities of the coding sequence to be replaced.

TABLE 4
crRNAs designed for knockout of OGNT genes. Information about stabilizing
modifications of tracrRNA and crRNA ordered at Microsynth.

| RNA Oligo Name | RNA Sequence (5′ to 3′) | SEQ ID NO: |
|---|---|---|
| tracrRNA | 5*7*6 585 765 AGU UAA AAU AAG GCU | 20 |
| | AGU CCG UUA UCA A68 875 555 578 776 | |
| | 566 757 867 787 68*8 *8 | |
| OGNT1_3prime | 7*8*8 *688 676 GAA AUC CGA UGG UUU | 21 |
| | UAG AGC UAU *7*6*8 | |
| OGNT2_3prime | 7*5*8 *777 656 GGA ACG GCC UGG UUU | 22 |
| | UAG AGC UAU *7*6*8 | |
| OGNT2_5prime | 5*7*8 *778 865 CAA CCG AAU CCG UUU | 23 |
| | UAG AGC UAU *7*6*8 | |
| OGNTL_5prime | 5*6*7 *787 567 CGA ACU CGC AGG UUU | 24 |
| | UAG AGC UAU *7*6*8 | |
| OGNT1_5prime | 5*6*5 *676 587 CGG GUG AGG UGG UUU | 25 |
| | UAG AGC UAU *7*6*8 | |
| OGNTL_3prime | 6*8*8 *786 867 GUG GCG CCA AGG UUU | 26 |
| | UAG AGC UAU *7*6*8 | |

*= PTO bond (Phosphorothioate bond); 5 = 2′-O-Methyl-RNA-A; 6 = 2′-O-Methyl-RNA-C, 7 = 2′-O-Methyl-RNA-G; 8 = 2′-O-Methyl-RNA-U; HPL = IEX-HPLC purified.

### (iv) PCR and Sequence Analysis of Deletion Strains

(A) Preparation of gDNA—Genomic DNA Isolation by Tissue Kit

2 ml of densely grown L. tarentolae culture were pelleted at 1800 g and the supernatant was discarded. The pellet was used for preparation of genomic DNA by the NucleoSpin® Tissue Kit (Macherey-Nagel). For this, the pellet is resuspended in 200 μl of Buffer T1 and further treated according to the manufacturer's instructions until elution. For efficient elution, 50 μl of prewarmed (50 °C) Buffer BE are added to the column and incubated at RT for 3 min. The eluate is collected by centrifugation for 1 min at 11000 g. Repetition of this step as well as reloading of the eluate can be used to increase the yield.

### (B) Preparation of Crude Cell Extracts for PCR Analysis

50 μl of culture were washed in 1 ml PBS and pelleted at 1800 g for 5 min. The supernatant was removed and the pellet was resuspended in 50 μl of PBS and boiled at 95 °C for 5 min with intermitted vortexing. 1 μl was used instead of template DNA in the PCR reaction.

### (C) PCR Analysis of OGNT KOs

PCR confirmation of OGNT knock-outs was performed by either amplification of the complete locus (OGNT1, OGNT2, OGNTL or OGNT1+L, where OGNT1+L comprises OGNT1 and OGNTL in tandem on the chromosome) or by amplification of the shorter fragments covering the integration sites.

Usually the successful replacement of the wildtype OGNT sequence (OGNT1=3.4 kbp, OGNT2=1.9 kbp and OGNTL=3.4 kbp) by the selection marker coding sequences (0.4-1.0 kbp) with or without the additional intergenic regions (together approx. 0.9 kbp) could be easily confirmed by the size of the amplicon resulting from the PCR targeting the whole native locus, since correct replacement of the wt gene would lead to a much shorter amplicon. For these PCRs LATaq DNA polymerase (TaKaRa) was used in combination with a buffer for amplification of GC rich sequences that allowed the amplification of the long wildtype regions (see Table 5). In some cases, amplification of shorter regions with primer binding within the OGNT coding sequence was preferred to test for presence of remaining wt genes (see Table 6). For these, the DreamTaq DNA polymerase (Thermo Fisher Scientific) was used. Alternatively, the correct integration of the selection marker gene into the respective OGNT locus could be tested by combinations of a primer binding in the genome with one primer binding to the selection marker CDS or the intergenic regions of the integrated construct and the other one targeting the genome.

TABLE 5
PCRs for analysis of OGNT deletions. PCR primers
used for confirmation OGNT knock-outs by absence of
the respective OGNT wt gene and the expected
amplicon sizes are summarized.

| | Target | | wt | KO |
|---|---|---|---|---|
| Target gene | region | Primers | amplicon | amplicon |
| OGNT2 | 5′ end | 360, 363 | 942 bp | none |
| OGNT2 | 5′ end | 362, 361 | 950 bp | none |
| OGNT1 | 3′ end | 290, 291 | 2572 bp | none |
| OGNTL | 5′ end | 292, 293 | 2645 bp | none |
| OGNT2* | Whole locus | 127, 128 | 3.3 kbp | shorter |
| OGNT1* + L | Whole locus | 161, 160 | 8.1 kbp | shorter |

*KO amplicon length for whole locus PCRs depends on the combination of selection marker and intergenic regions used.

TABLE 6
Listing of primer sequences used in the
described examples.

| | | SEQ |
|---|---|---|
| | | ID |
| Primers | Sequence | NO: |
| 360 | CCATGGCCAGATGCCAAGCTTACTC | 27 |
| 363 | CGGAACACAGACCTGCCATCTGTGG | 28 |
| 362 | CCACAGATGGCAGGTCTGTGTTCCG | 29 |
| 361 | CATCCACCTCTGCTCGTCGTCCTTC | 30 |
| 290 | CGCACCTCGGCTTCAACTGCCAATG | 31 |
| 291 | AACGTGCCTGAAGCCCGTAGCTTTG | 32 |
| 292 | GGCGACGGATGATGCCCACCATTTG | 33 |
| 293 | GGAGGCAACCAAGAAGGCGTCAGAG | 34 |
| 127 | GCTCAGCTGCGAGAGCGTAAACCTCCGTAACAATG | 35 |
| 128 | AGCTAAACTCTGCACTCATCTGCGCTTCCGCAAGG | 36 |
| 161 | CGACCGCGATATCTGCTTTCCATTCTGCAGTTCTC | 37 |
| 160 | CGCACAGGGTGACGAGGAAAGCAGTATCGAAACAG | 38 |

### (v) Expression Analysis

(A) Sample Preparation from Leishmania tarentolae

Cells were grown for 2-3 days at 26 °C, static (e.g. in 3 ml in a 6-well plates). Whole cell extract (WCE) cell free culture supernatants were analyzed by Western blot. For supernatant analysis, grown culture was centrifuged at 1800 g at RT for 5 min and cell free supernatant was transferred to a new tube and mixed with Laemmli dye under reducing or non-reducing conditions. Cell pellets for WCE were washed with 1×PBS, centrifuged again at 1800 g at RT for 5 min and frozen at −80 °C for minimally 30 min. After thawing it again at RT pellet was then resolved in Leammli (reducing) buffer, boiled again at 95 °C for 10 min and vortexed intensively.

### (B) Expression Analysis by Western Blot

Samples were run on 4-12% Bis-Tris SDS PAGE, using a MOPS running buffer with 200 V for 60 min. Gels were blotted using an Iblot device for 7 min on PVDF membranes. Membranes were blocked for at least 30 min at RT in 10% milk. Primary antibodies (i.e. goat anti-Human IgG-HRP (A6029, Sigma) 1:2000 diluted, mouse anti-Human Kappa Light Chain (K4377, Sigma) 1:5000 diluted were used diluted in 1% milk, 1×PBST for o/n incubation at 4 °C. Afterwards, the blot was washed with 1×PBST three times for 5 min before detection with horse reddish peroxidase (HRP) coupled secondary antibodies (anti-mouse polyvalent-HRP (A0412, Sigma) 1:2000 diluted or anti-rabbit-RP conjugate (Jackson ImmunoResearch #111-035-008) 1:2000 diluted) in 1% milk, 1×PBST for 3 h rotating at 30 °C, followed by three washes for 5 min in 1×PBST and one component 3,3′,5,5′-tetramethylbenzidine (TMB) substrate staining for colorimetric detection (TMBM-1000-01, Surmodics).

### 7.9.2 Small Scale Expression and Purification of Monoclonal Antibodies

Host cells were routinely grown in 50 ml culture in BHIH or YEH for 48 h at 26 °C shaking at 140 rpm. Cultures were harvested and centrifuged for 10 min at 1800×g at RT. Media SN was filtered through 0.22 μm filter (Steriflip, SCGP00525) and EDTA (0.5 M pH8) was added to each load in a 1:100 dilution. Media SNs of each strain were subjected to 4 h incubation with 100 μl of proteinA resin (ProteinA-Sepharose 4B Fast Flow, Sigma Aldrich, P9424) per Falcon tube in batch while rotating at RT. After treatment with ProteinA resin, the samples were centrifuged at 500×g for 5 min, the FT was discarded and the resin was transferred to spin columns. Washes were performed with 3×5 CV using Buffer A (pH 7.2, 20 mM Na₂HPO₄, 150 mM NaCl, pH was adjusted with HCl to 7.20) using 500 μl for 100 μl resin; with centrifugation at 1000×g, RT, 1 min between each step. Elution was performed with several CV of Buffer B (0.1 M acetic acid, 100 mM NaCl, pH was adjusted with 1 M NaOH to 3.20) using 100 μl for 100 μl resin, with centrifugation at 1000×g, RT, 1 min between each step (e.g. 3×1CV and 1×0.5 CV). Elution fractions were pooled and immediately neutralized by adding 100 mM Tris-HCl (1 M pH8). Afterwards, the pooled elutions were buffer exchanged to PBS pH 6 using 2 ml 7K ZebaSpin desalting columns and optionally concentrated using Amicon 0.5 ml 30 K concentrators.

Elution fractions were pooled and immediately neutralized by adding 100 mM Tris-HCl (1 M pH8). Afterwards, the pooled elutions were buffer exchanged to PBS pH 6 using 2 ml 7K ZebaSpin desalting columns and optionally concentrated using Amicon 0.5 ml 30 K concentrators.

Samples were then subjected to analyses such as HILIC-UPLC-MS as described below.

### 7.9.3 Analysis

### (i) SDS PAGE and Capillary Gel Electrophoresis

SDS PAGE was performed under reduced or non-reducing conditions using 10 μg for Coomassie, 2.5 μg for WB, separated on 4-12% Gel with MOPS buffer for 55 minutes. Determination of Protein purity was done by Coomassie Stained SDS-PAGE with 10 μg protein sample and compared to a BSA standard curve. Impurities were quantified by ImageQuant. Capillary Gel Electrophoresis (CGE) was performed using an Agilent Protein 230 Kit (5067-1518), according to protocol.

### (ii) Analytical SEC

MAbPac SEC-1 (4×300 mm) is a size exclusion chromatography (SEC) column specifically designed for separation and characterization of monoclonal antibodies (mAbs) and was used according to manufacturer's recommendation (Temperature: 30 °C; Eluent: PBS 50 mM NaPO4, 300 mM NaCl pH 6.8; Elution: isocratic, 30 minutes; Flow: 0.2 mL/minute; Detection: 215 nm; Injection V: 5 μL corresponding to 5 μg protein).

(iii) O-HexNAc Identification

Intact monoclonal antibodies were analyzed by mass spectrometry employing the state-of-the-art instrumentation (Orbitrap FTMS), data processing and data analysis (bioinformatics) tools by SpectroSwiss. In addition to the intact measurement the antibody was reduced with TCEP or enzymatically cleaved (IdeS) to generate Fd, LC and Fc/2 subunits, which were analyzed using the same instrumentation.

### (iv) Sample Preparation for IdeS Derived Subunit Analysis

To generate mAb subunits of about 25 kDa each, IdeS (FabRICATOR, Genovis, Lund, Sweden) digestion of mAbs was performed in formulation buffers. One unit of IdeS was added to each μg of mAbs and left to react for 30 minutes at 37 °C. Then, mAbs were denatured and reduced by incubation with 6 M GdnCl and 30 mM TCEP at room temperature for 30 minutes. Finally, the reaction was quenched by acidifying the solution to 1% TFA. For the analysis samples were diluted with 0.1% FA in water to a final concentration of 1 μg/μl.

### (v) Mass Spectrometry for Intact and Subunit Analysis, Bioinformatics and Data Processing.

A standard Orbitrap-based intact protein mass measurement set-up (a triplicate LC-MS experiment) was employed using:
Analytical HPLC (Dionex) with 30 min run duration,
Analytical column (Waters), C4 Aquity, BEH, 90 μl/min @60 C,
Q Exactive HF FTMS (Thermo),
FTMS Booster (Spectroswiss), advanced data acquisition system
Full MS resolution: 30'000 @m/z 200 for intact mAb and 60'000 @m/z 200 for subunit analysis
Charge target number (ion number): AGC of 3e6

Intact Mass (Protein Metrics) was employed for mass spectra deconvolution of intact mAbs. MASH Suite software tool (open access, Wisconsin University, Ying Ge Group) was used for deconvolution of the mass spectra of the subunit analysis. The employed resolution enabled obtaining isotopically-resolved data. Peak-by-Peak software tool (Spectroswiss) was employed for time domain signals and mass spectra processing. The employed methods enabled obtaining high spectral dynamic range data.

(vi) Analysis of N-Glycans Released from Purified Proteins and Cells Surfaces by HILIC-UPLC-MS

Enzymatic release of N-glycans from cell surfaces was performed using PNGase F (New England Biolabs). Cells (grown for 48 or 72 h at 26 °C shaking at 140 rpm) were harvested and washed with PBS by centrifugation for 10 min at 1800×g at RT. 50 mg of cell pellet were re-suspended in Glyco Buffer 2 and incubated with 1 μl PNGase F for 1 h at 37 °C and 650 rpm. Cells were again pelleted by centrifugation and 75 μl of the supernatant was dried down in a SpeedVac concentrator. Glycans were resuspended in 10 μl of water. Following release, glycans were directly labeled with procainamide as described previously (Behrens, et al. (2018) Glycobiology 28 (11), pp. 825-831). Briefly, released glycans were mixed with 1 μl acetic acid, 8 μl of a procainamide stock solution (550 mg/ml in DMSO) and 12 μl of a sodium cyanoborohydride stock solution (200 mg/ml in H₂O). Samples were incubated for 60 min at 65 °C and cleaned up using LC-PROC-96 clean up plates (Ludger Ltd) according to the manufacturer's instructions.

Enzymatic release of N-glycans from purified proteins was performed using Rapid PNGase F (New England Biolabs) as recommended by the supplier. 8 μl of sample (15 μg of protein) were mixed with 2 μl Rapid Buffer and 1 μl of Rapid PNGase F. The mixture was incubated at 50 °C for 10 min followed by 1 min at 90 °C.

For site specific N-glycan analysis of monoclonal antibody subunits, IgG1 mAb was either cleaved with IdeZ to F(ab′)2 and Fc/2, or heavy and light chains were reduced before separation on SDS PAGE. Bands were excised and enzymatic release of N-glycans from the monoclonal antibody was performed using PNGase F. Following release, glycans were directly labeled with procainamide (PC).

Procainamide-labeled N-glycans were analyzed by hydrophilic interaction chromatography-ultra performance liquid chromatography-mass spectrometry (HILIC-UPLC-MS) using am Acquity UPLC System (Waters) with fluorescence detection coupled to a Synapt G2-Si mass spectrometer (Waters). Glycans were separated using an Acquity BEH Amide column (130 Å, 1.7 μm, 2.1 mM×150 mM; Waters) with 50 mM ammonium formate, pH 4.4 as solvent A and acetonitrile as solvent B. The separation was performed using a linear gradient of 72-55% solvent B at 0.5 ml/min for 40 min. Fluorescence was detected at an excitation wavelength of 310 nm and a detection wavelength of 370 nm. The Synapt G2-Si mass spectrometer fitted with a Zspray electrospray source was used for mass detection in positive resolution mode using the following parameters: Scan range: m/z 300-3500; scan time: 1 sec; capillary: 2.2 kV; source temperature: 120 °C and sampling cone: 75 V. MassLynx 4.2 (Waters) was used for data acquisition. Data processing and analysis was performed using Unifi 1.9.4.053 (Waters). Glucose units were assigned using a fifth-order polynomial distribution curve based on the retention times of a procainamide-labeled dextran ladder (Ludger Ltd). Glycan structures were assigned based on their m/z values and their retention times and matched against a previously constructed N-glycan library. For individual samples the UPLC was coupled to a Synapt HDMS mass spectrometer using comparable settings.

7.9.4 Analysis of UDP-GalNAc by High Performance Anion Exchange Chromatography Coupled with Pulsed Amperometric Detection (HPAEC-PAD)

MeOH/Chloroform extraction procedure for L. tarentolae cell pellets was performed on 2 OD of each sample, which were harvested by centrifugation and washed 2× with 1×PBS (2200 g, 10 min, RT) and frozen. For extraction, pellets were thawed, resuspended in 480 μl MeOH, supplemented with 20 μl water and sonicated in a water bath at RT for 15 min. The samples were spun in a table-top centrifuge at 18000 g and 4 °C for 10 min. The SN was transferred into a glass vial, supplemented with 268 μl chloroform and vortexed. Next, 500 μl H2O (MS grade) was added and the sample was vortexed again. The MeOH/chloroform/H2O (1/0.54/1) mixture was spun at 2200 g and RT for 20 min to remove proteins, lipids and DNA in the CHCl3 phase. Approximately half (525 μl) of the upper MeOH/H2O phase was collected and transferred into Eppendorf tubes, corresponding to extracted material from 0.5 OD pellet. The samples were dried in a speed-vac and stored at −20 °C until analysis. Thawed samples were resuspended in H2O and amounts corresponding to 0.5 OD analyzed by High performance anion exchange chromatography coupled with pulsed amperometric detection (HPAEC-PAD) using CarboPac PA1, 4×250 mm (Thermo), based on Tomiya et al. 2001: Determination of Nucleotides and Sugar Nucleotides Involved in Protein Glycosylation by High-Performance Anion-Exchange Chromatography: Sugar Nucleotide Contents in Cultured Insect Celles and Mammalian Cells. Analytical Biochemistry 293, p. 129-137, with an adjusted gradient. A standard of UDP-GalNAc/-GlcNAc mix (each 25 ug/mL) in H2O was prepared in addition to the cell extract samples->1000 ug/mL UDP-GlcNAc and -GlcNAc solutions were pre-diluted 1:10 to 100 ug/mL solutions. 50 uL of each 100 ug/mL solution was added to 100 uL H2O for a solution containing 25 ug/mL from each component (corresponds to 38.4 uM from each component). Spiked UDP-GalNAc was detectable in cell extract samples as set for another control. Variability for three independent replicates of 0.5 OD injection is at 17.2% CV (n=3). Average recovery lies at 69%. For the 1.2 OD injection, spike recovery lies at 62% (n=1).

### 8. EXAMPLES

### 8.1 Example 1—Generation and Characterization of Glycosylated Bifunctional Antibodies

Antibodies were prepared as described in the Section 7.9.1, in particular Section 7.9.1(v)(A).

Quality and glycosylation of antibodies (Table 9) was analyzed using the method described in the Section 7.9.3. The Table 7 shows the antibody structure and N-Glycan structure on the engineered glycosites.

TABLE 7
Structure of Antibodies and Glycoengineering

| | | Engineered | | Main N- |
|---|---|---|---|---|
| | | Glycosites (Eu | | glycan structure on |
| Antibody | Format | numbering) | Glycotags | engineered glycosites |
| H-A2F | Human | None | None | NA |
| | IgG1 | | | |
| A-84.86-A2 | Human | LC: D86N | None | A2 |
| | IgG1 | HC: K84N | | |
| A-84.86-A2G2 | Human | LC: D86N | None | A2G2 |
| | IgG1 | HC: K84N | | |
| A-84.86-M3 | Human | LC: D86N | None | M3 |
| | IgG1 | HC: K84N | | |
| A-84.86- | Human | LC: D86N | None | A2GalNAc2 |
| A2GalNAc2 | IgG1 | HC: K84N | | |
| A-84-A2G2 | Human | HC: K84N | None | A2G2 |
| | IgG1 | | | |
| A-84.86-A2G2S2 | Human | LC: D86N | None | A2G2S2 |
| | IgG1 | HC: K84N | | |
| 11K2-IgG4-A2F | Chimeric | None | None | NA |
| | mouse | | | |
| 11K2-84.86- | Variable- | LC: D86N | None | A2GalNAc2 |
| A2GalNAc2 | | HC: K84N | | |
| 11K2-86- | Human | LC: D86N | None | A2GalNAc2 |
| A2GalNAc2 | | | | |
| 11K2-gt1- | IgG4-PAA | None | C-Terminal HC | A2GalNAc2 |
| A2GalNAc2 | | | | |
| 11K2-84.gt1- | | HC: K84N | C-Terminal HC | A2GalNAc2 |
| A2GalNAc2 | | | | |
| 11K2-LCLgt1- | | None | C-terminal LC | A2GalNAc2 |
| A2GalNAC2 | | | | |
| 11K2- | | None | C-Terminal HC | A2GalNAc2 |
| LCLgt1.gt1- | | | C-terminal LC | |
| A2GalNAc2 | | | | |
| 11K2-84.86- | | LC: D86N | None | A1GalNAcl |
| A1GalNAcl | | HC: K84N | | |
| 11K2-86- | | LC: D86N | None | A1GalNAcl |
| A1GalNAcl | | | | |
| Ptz-A2F | Human | None | None | NA |
| | IgG1 | | | |
| Ptz-86- | Human | LC: D86N | None | A2GalNAc2 |
| A2GalNAc2 | IgG1 | | | |
| Ptz-gt1- | Human | None | C-Terminal HC | A2GalNAc2 |
| A2GalNAc2 | IgG1 | | | |
| Ptz-hgt- | Human | None | Inserted in hinge | A2GalNAc2 |
| A2GalNAc2 | IgG1 | | of HC | |

Table 7 shows the protein structure and the main N-glycoform displayed at engineered glycosites for indicated antibodies. The glycan nomenclature is described in Section 5.3. HC: Heavy chain. LC: light chain.

Table 8 shows the formats and N-Glycan structure on the engineered glycosites of antibody fragments or non-antibody molecules.

TABLE 8
Structure of Antibody fragments or non-antibodies and glycoengineering.

| | | Engineered | | Main N-glycan |
|---|---|---|---|---|
| | | Glycosites (Eu | | structure on |
| Antibody | Format | numbering) | Glycotags | engineered glycosites |
| Fab-Fent- | Fab Fragment. | None | C-terminal LC | A2 |
| LCLgt1-A2 | Chimeric | | | |
| Fab-Fent- | mouse variable- | None | C-terminal LC | A2GalNAC2 |
| LCLgt1- | human IgG4 | | | |
| A2GalNAc2 | HC | | | |
| Fab-Fent- | | LC: D86N | C-terminal LC | A2GalNAC2 |
| 86.LCLgt1- | | | | |
| A2GalNAc2 | | | | |
| MOG-Fc- | Fusion protein | None | None | A2GalNAc2 |
| A2GalNAC2 | MOG-EDC | | | |
| | and human | | | |
| | IgG1 Fc | | | |
| MOG-Fc-gt1- | Fusion protein | None | C-terminal Fc | A2GalNAc2 |
| A2GalNAc2 | MOG-EDC | | | |
| | and human | | | |
| | IgG1 Fc | | | |
| MOG-Fc-N60Q- | Fusion protein | None | C-terminal Fc | A2GalNAc2 |
| gt1-A2GalNAc2 | MOG-EDC | | | |
| | with | | | |
| | endogenous | | | |
| | glycosite | | | |
| | mutated and | | | |
| | human IgG1 | | | |
| | Fc | | | |
| Fab-A-FLGT4- | Fab fragment | None | C-terminal LC | A2GalNAc2 |
| A2GalNAc2 | human IgG1 | | | |

Table 8 shows the protein structure and the main N-glycoform displayed at engineered glycosites for indicated antibody fragment or non antibody scaffold. The glycan nomenclature is described in Section 5.3. HC: Heavy chain. LC: light chain. MOG: myelin oligodendrocyte protein. EDC: Extracellular domain. Fc: Fragment crystallizable.

Table 9 shows the quality profile of the main antibodies and fragments described herein.

TABLE 9
Quality profile of Antibodies.

| | | Aggregation/ | | | | |
|---|---|---|---|---|---|---|
| Antibody | N-glycan | Degradation | Endotoxin | | Unoccupancy | Target binding |
| A-84- | Fab: 69% | 85.8% | <0.5 | EU/mg | ND | 50-200% |
| A2GalNAc2 | A2GalNAc2 | monomer | | | | |
| | Fc: 92% A2 | 11.9% | | | | |
| | | degradation | | | | |
| | | 2.1% | | | | |
| | | aggregates | | | | |
| A-84.86- | Fab: 71% | 88.9% | <0.5 | EU/mg | 71% fully occupied, | 50-200% |
| A2GalNAc2 | A2GalNAc2 | monomer | | | 25% 1 site | |
| | Fc: 95% A2 | 9.5% | | | unoccupied, 4% 2 | |
| | | degradation | | | sites unoccupied | |
| | | 1.9% | | | | |
| | | aggregates | | | | |
| A-84.86- | Fab: 48% | 97.1% | <0.5 | EU/mg | ND | 108% |
| A2G2S2 | A2G2S2/ | monomer | | | | |
| | 27% A2G2S1 | 2.3% | | | | |
| | Fc: 52% A2/ | degradation | | | | |
| | 2% M3 | 0.6% | | | | |
| | | aggregates | | | | |
| 11K2-gt1- | mAb: 57% | 86.9% | <1.00 | EU/mg | HC(Fc + tag): 5% 1 | IND |
| A2GalNAc2 | A2GalNac2 | monomer | | | site unoccupied | |
| | (in solution) | 7.8% | | | | |
| | | degradation | | | | |
| | | 5.3% | | | | |
| | | aggregates | | | | |
| 11K2-gt1- | mAb: 52% | 93.5% | 1.28 | EU/mg | HC (84 + Fc): 3% | ND |
| A2GalNAc2 | A2GalNac2 | monomer | | | unoccupied | |
| | (in solution) | 4.6% | | | | |
| | | degradation | | | | |
| | | 1.7% | | | | |
| | | aggregates | | | | |
| 11K2-84.86- | HC (Fc + 84): | 87% monomer | 1.43 | EU/mg | LC (86): fully | IND |
| A2GalNAc2 | 45% | 7.7% | | | occupied | |
| | A2GalNac2 | degradation | | | HC (Fc + 84): 3% | |
| | LC: 72% | 3.5% | | | | |
| | A2GalNac2 | aggregates | | | | |
| 11K2-84.gt1- | mAb: 55.27% | 80.9% | 1.68 | EU/mg | HC: (Fc + 84 + gt1): | ND |
| A2GalNAc2 | A2GalNac2 | monomer | | | approx. 4% | |
| | (in-solution) | 9.9% | | | | |
| | | degradation | | | | |
| | | 6.9% | | | | |
| | | aggregates | | | | |
| 11K2- | mAb: 42.5% | 89.3% | 1.72 | EU/mg | LC: fully occupied | ND |
| LCLgt1.gt1- | A2GalNAc2 | monomer | | | HC (Fc + gt1): 2% | |
| A2GalNAc2 | (in-solution) | 5.8% | | | | |
| | | degradation | | | | |
| | | 4.6% | | | | |
| | | aggregates | | | | |
| 11K2- | HC(Fc site + | 80.8% | <1 | EU/mg | LC: fully occupied | IND |
| LCLgt1.gt1- | tag): 54.8% | monomer | | | HC (Fc + gt1): 2% | |
| A2GalNAc2 | A2GalNAc2; | 13.1% | | | | |
| | LC(tag): 92% | degradation | | | | |
| | A2GalNAc2 | 6.2% | | | | |
| | | aggregates | | | | |
| 11K2-84.86- | LC: 90.1% | 84.4% | <1 | EU/mg | HC(Fc site + 84): | ND |
| A1GalNAcl | A1GalNAc1; | monomer | | | 6%; LC: 0% | |
| | HC: 88.1% | 13.5% | | | | |
| | A1GalNAc1 | degradation | | | | |
| | | 2.1% | | | | |
| | | aggregation | | | | |
| Ptz-86- | Fc: 24% | 71.6% | <1.0 | EU/mg | HC (Fc site): 11% | 50-200% |
| GalNAc2 | A2GalNAc2, | monomer | | | unoccupied; LC(86): | |
| | D86N: 76% | 26.8% | | | 3% unoccupied | |
| | A2GalNAc2 | degradation | | | | |
| | | 1.1% | | | | |
| | | aggregates | | | | |
| Ptz-gt1- | 72% | 72.3% | <1.0 | EU/mg | HC (2 sites): 11% 1 | 50-200% |
| GalNAc2 | A2GalNAc2 | monomer | | | site unoccupied | |
| | (in solution) | 26.2% | | | | |
| | | degradation | | | | |
| | | 1.1% | | | | |
| | | aggregates | | | | |
| Ptz-hgt- | 77% | 72.2% | <1.0 | EU/mg | HC (2 sites): 61% 1 | 50-200% |
| GalNAc2 | A2GalNAc2 | monomer | | | site unoccupied; 3% | |
| | (in solution); | 26.0% | | | 2 sites unoccupied | |
| | Fab: 94% | degradation | | | | |
| | A2GalNAc2 | 1.4% | | | | |
| | Fc: 61% | aggregates | | | | |
| | A2GalNAc2 | | | | | |
| Fab-Fent- | 99% A2 (in | 94.9% | <1.0 | EU/mg | fully occupied | Reference molecule |
| LCLgt1-A2 | solution) | monomer | | | | |
| | | 1.9% | | | | |
| | | degradation | | | | |
| | | 3.2% | | | | |
| | | aggregates | | | | |
| Fab-Fent- | 97.34% | 96.2% | <1.0 | EU/mg | <2% unoccupied | 50-200% |
| LCLgt1- | A2GalNAc2 | monomer | | | | |
| A2GalNAc2 | (in solution) | 1.8% | | | | |
| | | degradation | | | | |
| | | 2.0% | | | | |
| | | aggregates | | | | |
| Fab-Fent- | 62% | 96.1% | <1.2 | EU/mg | fully occupied | 50-200% |
| LCLgt1- | A2GalNAc2 | monomer | | | | |
| A2GalNAc2 | | 1.1% | | | | |
| | | degradation | | | | |
| | | 2.8% | | | | |
| | | aggregates | | | | |
| Fab-Fent- | 97.7% | 92.3% | <1.0 | EU/mg | fully occupied | 50-200% |
| 86.LCLgt1- | A2GalNAc2 | monomer | | | | |
| A2GalNAc2 | | 5.1% | | | | |
| | | degradation | | | | |
| | | 2.6% | | | | |
| | | aggregates | | | | |
| MOG(ECD)- | 21% | 83.8% | <1.1 | EU/mg | fully occupied | ND |
| Fc- | A2GalNAc2 | monomer | | | | |
| A2GalNAc2 | (in-solution); | 12.2% | | | | |
| | 28-45% | degradation | | | | |
| | A2GalNAc2 | 0.6% | | | | |
| | on MOG | aggregates | | | | |
| | (IdeS vs | | | | | |
| | EndoS) | | | | | |

Table Legend: N-Glycan analysis was performed using the method described in Section 7.9.3. Aggregation and degradation data were generated using size-exclusion HPLC as further described in section 5.9.3. Unoccupancy means the relative amount of glycosylation sites not carrying a N-glycan as determined using the in-house IP-MS method (Section 5.9.3). The target binding (HCA202 binding for adalimumab variants and BSA-fentanyl for Fab-Fent variants) was considered conserved if the binding was within 50-200% of the reference molecule determined by ELISA EC50 values. The reference molecule was H-A2F for adalimumab glycovariants and was fab-Fent-LCLgt1-A2 for the anti-fentanyl Fab compounds.
ND: Not done.

Except for some 11K2 variants (11K2-LCLgt1.gt1-A2GalNAc2; 11K2-84.gt1-A2GalNAc2 and 11K2-gt1-A2GalNAc2), all antibodies showed aggregate levels below 5% as assessed by size exclusion HPLC method. All adalimumab glycoengineered antibodies conserved high binding to HCA202 an anti-adalimumab idiotype Fab Fragment which was used as antigen for adalimumab experiments. Fab-Fentanyl variant showed strong binding to BSA-fentanyl antigen.

### 8.2 Example 2—A2GalNAc2 Glycosylated Antibody Binds to ASGPR

### 8.2.1 SPR Analysis

Multi-cycle kinetics were measured using a Biacore 8 k+ with 10 mM HEPES pH 7.4, 150 mM NaCl, 40 mM CaCl2 and at a temperature of 25 °C. Steady state and 1:1 binding analysis was performed using Biacore Insights Software.

Briefly, Cytiva Series S CM5 sensor chips (Cat #29104988) were prepared for amine-based capture using the Cytiva Amine Coupling kit (Cat #BR100050). The surface was activated by 0.2M 1-ethyl-3-(3-dimethylaminopropyl)carbodiimide hydrochloride (EDC) and 0.05M N-hydroxysuccinimide (NHS) prior to injection of anti-human Fc from the Cytiva Human Antibody Capture Kit, type 2 (Cat #29234600). Antibody was immobilized at a final concentration of 10 μg/ml with final resonance units (RU) reaching approximately 2000. Unbound activated groups were blocked by 1M ethanolamine. ASGPR1-Fc fusion and ASGPR2-Fc fusion were immobilized on 4 channels each at a final concentration of 1.175 μM (500-1000 RU) in 10 mM HEPES pH 7.4, 150 mM NaCl.

### 8.2.2 A2GalNAc2 Binding to ASGPR1 and ASGPR2

Analytes were prepared by diluting stocks of A84.86-A2GalNAc2 to 2 μm. A 5-fold dilution series was performed for a total of 8 points. Binding to either ASGPR1 or ASGPR2 was measured for each analyte. Association and dissociation time was measured for 180 seconds with a flow rate of 20 μl/min. Channels were regenerated between each injection with 20 mM EDTA. Blanks were included before, during, and after cycle

Data are shown in Table 10.

TABLE 10
Kinetic Parameters of A-84.86-A2GalNAc2 for ASGPR Binding

| | ka (1/Ms) | kd (1/s) | Kinetic KD (M) |
|---|---|---|---|
| ASGPR1 | 35615 | 0.002633 | 7.4E−08 |
| ASGPR2 | 118395 | 0.007175 | 6.1E−08 |

A similar experiment was done in reverse orientation. Avi-Tag ASGPR1 (Acro Biosystems #GS1-H82Q3) was captured on series S SA chip (Cytiva 29699621) at 2.0 μg/mL in HEPES pH 7.4, 150 mM NaCl, 3 mM EDTA, 0.05% TW20 (HBS-EP+). The Set Target Level was 250RU. A-84.86-A2GalNAc2 and H-A2F were prepared in running buffer: HEPES pH 7.4, 150 mM NaCl, 2 mM CaCl2, TW20 0.05% (HBS-N, 2mMCaCl2 and TW20 0.05%). A multicycle experiment with a 12-point titration with a 2-fold dilution was run. The contact time was 300 s and the dissociation time was 900 s at 30 μL/min. The regeneration solution was HBS-N+, 50 mM EDTA, TW20 0.05% with a contact time of 60 s at a flow rate of 30 μL/min. The data are shown in Table 10.B. Detectable binding and kinetic parameters were obtained only with A-84.86-A2GalNAc2.

TABLE 10.B
Kinetic parameters of A-84.86-A2GalNAc2 and H-A2F binding
to immobilized ASGPR1

| | Quality kinetics | Steady state |
|---|---|---|
| | chi² (RU²) | affinity KD (nM) |
| A-84.86- | 6.46e+02 | 907 |
| A2GalNAc2 | | |
| H-A2F | NA | NA |

### 8.3 Example 3—A2GalNAc2 Glycosylated Antibody Leads to Efficient Internalization Via ASGPR in Hepatocyte Cells

HepG2 hepatocarcinoma cells (ATCC #HB-8065) express ASGPR and were maintained in a low glucose DMEM medium (Sigma, Ref. D5546) supplemented with 10% FBS. Adalimumab antibody glycovariants were labeled with pHrodo dye (pHrodo iFL Red STP Ester [amine-reactive], ThermoFisher, ref P36011) according to manufacturer instructions. The fluorescence of pHrodo is activated at low pH and therefore will allow for the visualization of protein internalization and targeting to the lysosomal pathway. The pHrodo Degree of Labeling (DOL) for each antibody was determined as follows. Antibodies were diluted 1:2 in denaturing buffer and analyzed with Nanodrop at 280 nm and 560 nm wavelength (A280 and A560). Protein concentration and pHrodo DOL were calculated as follows.

$$Protein conc . [ M ] = \frac{protein conc . [ \frac{mg}{ml} ]}{MW [ g / mol ]} = \frac{protein conc . [ \frac{mg}{ml} ]}{144000}$$

$$D O L = \frac{{λ}_{max} * dilution factor}{{ε}_{dye} [ M - 1 cm - 1 ] * protein conc . [ M ]} = \frac{A 560 * 2}{65000 * protein conc . [ M ]}$$

MW is the molecular weight of the antibody used: 144000 g/Mol. λmax is the absorbance measured at 560 nm. εdye is the Extinction coefficient: 65000 M⁻¹ cm⁻¹. Dilution factor is 2.

HepG2 cell monolayers were incubated for 3 to 4 hours or 24 hours with pHrodo-antibodies (3 μg/ml)+IVIg (1 mg/ml) (Hizentra, obtained from pharmacy), at 37 °C. In some conditions, cells were also treated with the following reagents: fetuin (Sigma, Ref F3385) at 2 μM; asialofetuin (Sigma, Ref A4781) at 2 μM; Chloroquine (Sigma, Ref. C6628) at 50 μM; Bafilomycin (Millipore, Ref 19148) at 10 nM; cytochalasin D (Sigma, Ref. C2618) at 50 μM. Cell cultures were then washed, and harvested using Accutase (Sigma/Merck, Ref. SCR005) and immediately acquired on a flow cytometer. Mean fluorescence intensity (MFI) of pHrodo for gated single and live (DAPI+ cells excluded) cell population was analyzed using standard flow cytometry software. MFI values were adjusted to pHrodo DOL.

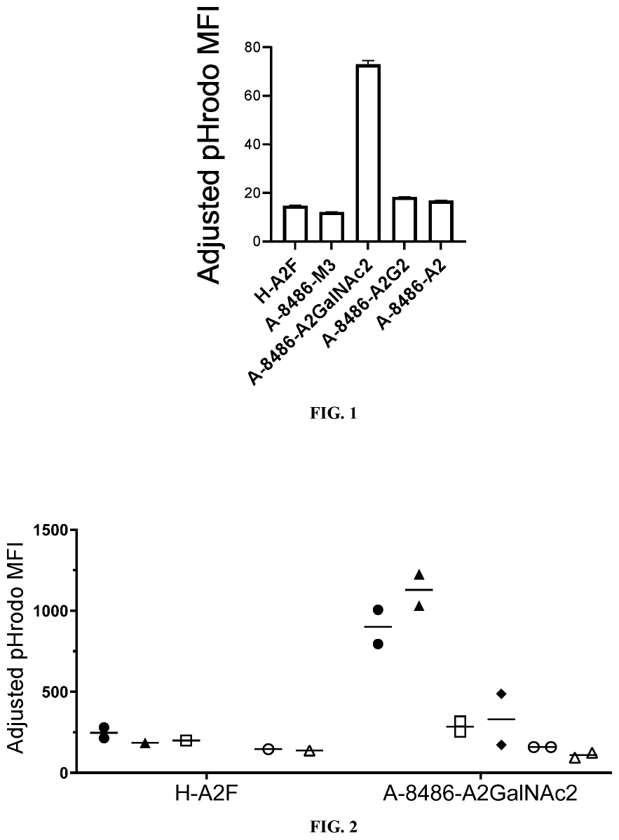

FIG. 1 shows the data obtained comparing H-A2F (Adalimumab; Humira, obtained from pharmacy), A-84.86-A2, A-84.86-A2G2, A-84.86-A2GalNAc2 and A-84.86-M3 antibodies. After 4 hours of incubation, only GalNAc2 displaying antibodies were internalized in HepG2 cells, indicating that GalNAc2 is a potent glycan for recognition and internalization by hepatocyte cells.

Another experiment to assess whether the uptake of GalNAc2 antibody is mediated by ASGPR was performed by using different inhibitors. FIG. 2 shows the data obtained in this inhibition experiment. The internalization of A-84.86-A2GalNAc2 was inhibited by EGTA, a calcium ion chelator, indicating that uptake of the antibody is likely mediated by a calcium-dependent C-type lectin receptor. In addition, internalization was selectively inhibited by asialofetuin (ligand for ASGPR) but not by fetuin (not a ligand for ASGPR), indicating that the recognition and internalization of A2GalNAc2-antibodies is mediated by ASGPR (Braun et al. J Biol Chem, 271(35):21160-6 (1996). The pHrodo signal produced by A-84.86-A2GalNAc2 was completely blunted by chloroquine and bafilomycin that disrupt clathrin-mediated endocytosis by blocking endosomal acidification (Ippoliti et al. Cell Mol Life Sci, 56:866-875 (1998). Endosomal acidification is a key step to achieve lysosomal protein degradation.

The pattern of inhibition observed in these experiments is consistent with an endocytic mechanism that is mediated by ASGPR, relies on clathrin, but not other endocytic pathways and that directs the endocytosed material to lysosomal compartment. Therefore, these data support the use of CGP-produced GalNAc2 displaying proteins to target compounds to ASGPR-mediated internalization and degradation.

To characterize the relative apparent affinity of A-84.86-A2GalNAc2 and asialofetuin, a competition experiment was conducted. HepG2 cells were plated in a 96-well flat bottom plate at a concentration of 50'000 cells/well in 100 μl complete HepG2 medium containing increasing concentrations (0.002-4 μM) of asialofetuin (Sigma, A4781). After 30 minutes incubation at 37 °C, 3 μg/ml of pHRodo labeled H-A2F or A-84.86-A2GalNac2 were added, and cells were then incubated for 3 hours at 37 °C.

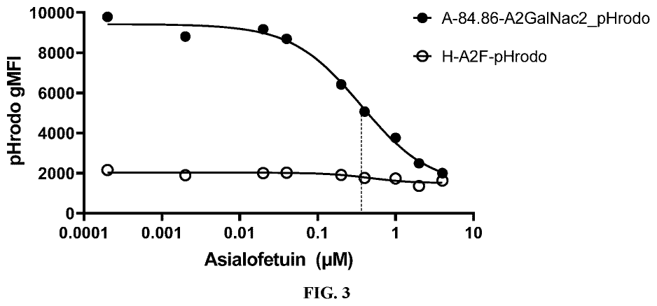

FIG. 3 shows that a 50-fold molar excess of asialofetuin is required to reach 50% blocking of A-84.86-A2GalNAc2 internalization response. These data suggest that the potency of engagement of ASGPR by A2GalNAc2 antibody is significantly stronger than the potency of asialofetuin, an ASGPR ligand displaying Galactose terminated glycans.

### 8.4 Example 4—A2GalNAc2 Antibody is not Internalized by HepG2 Cells in Absence of ASGPR

To verify that internalization of antibodies displaying A2GalNac2 structures is dependent on ASGPR engagement, HepG2 cells (Sigma, 85011430) were treated with siRNA targeting ASGPR1 or ASGPR2 mRNA to knock-down their expression. For each well to be transfected, 6 pmol of ASGPR1 Silencer Select Pre-designed (Ambion, 4392420-s1663), ASGPR2 Silencer Select Pre-designed (Ambion, 4392420-s1665) or Silencer Select Negative Control No. 1 (Ambion, 4390843) were incubated with 1 μl Lipofectamine RNAiMAX (Thermo Fisher Scientific, 13778100) in 100 μl of OptiMEM I Reduced Serum Medium (Thermo Fisher Scientific, 31985062) for 20 minutes at room temperature. HepG2 cells were plated in a 24-well plate at a concentration of 50000 cells/well in 500 μl of antibiotic free DMEM low glucose medium (Sigma, D5546) supplemented with 10% heat inactivated FBS (Pan-Biotech, P30-5500), 2 mM L-Glutamine (Sigma, G7513) and 100 μl of the siRNA-lipofectamine complexes. After 24 hours, HepG2 cells were washed once, and the medium changed with complete HepG2 cells medium containing 10 Units/ml penicillin and 100 μg/ml Streptomycin. HepG2 cells were used for pHRodo internalization assays 48-72 hours post transfection.

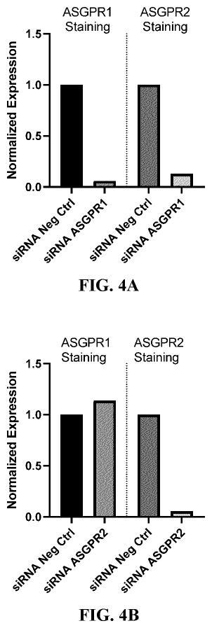

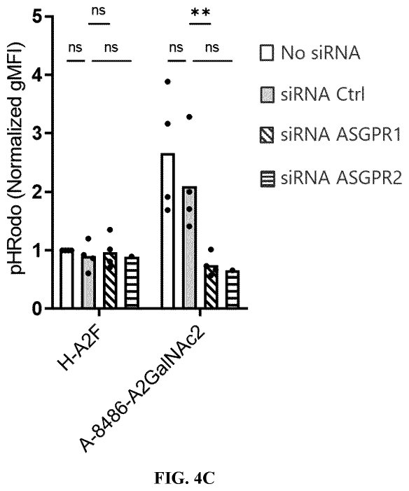

FIGS. 4A-C show that ASGPR1 siRNA treated HepG2 cells do not internalize A-84.86-A2GalNAc2 antibody. Similarly, ASGPR2 siRNA treated cells do no internalized A-84.86-A2GalNAc2. The ASGPR1 siRNA induced a complete reduction of ASGPR1 and ASGPR2 protein on cell surface, as probed by specific antibody staining by flow cytometry. In contrast ASGPR2 siRNA induced only reduction of ASGPR2 protein from cell surface.

HepG2 cells knock-out for ASGR1 were constructed using Crispr-Cas9 technique. Specific guide RNA targeting exon 4 of ASGR1 were designed and complexed together with the sp Cas9 to form a ribonucleoprotein (RNP). RNPs were then delivered to HepG2 cells via electroporation. To verify the editing efficiency, the edited site was PCR-amplified, and the amplicons were Sanger sequenced. As control, HepG2 cells were submitted to mock transfection in absence of RNP. Cells were expanded and maintained in DMEM low glucose medium (Sigma, D5546) supplemented with 10% heat inactivated FBS (Pan-Biotech, P30-5500), 2 mM L-Glutamine (Sigma, G7513), 10 Units/ml penicillin and 100 μg/ml Streptomycin

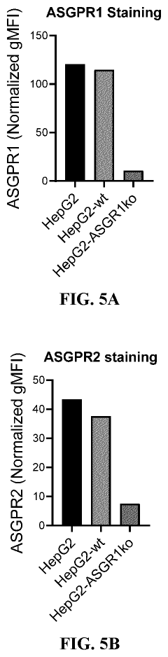

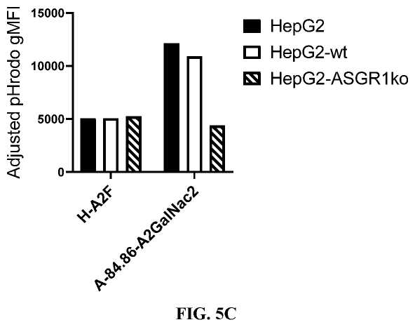

FIGS. 5A-5C show the data obtained on ASGPR1 knock-out HepG2. These data confirm the siRNA data and show that internalization of a A2GalNAc2 displaying antibody is abrogated in the absence of ASGPR1. The ASGPR knockout and siRNA experiment therefore demonstrate that the internalization of an antibody displaying A2GalNAc2 structure by hepatocyte cells is totally dependent on ASGPR1 and ASGPR2 expression.

### 8.5 Example 5—A2GalNAc2 Antibody is Directed to Lysosomal Vesicles in HepG2 Cells

To verify that internalized antibody was directed to lysosomal vesicles in HepG2 cells, a microscopy experiment using pHrodo-labeled antibodies and a lysosomal tracker dye was performed.

8.5.1 Labeling A-84.86-A2GalNAc2 with pHrodo (Deep Red) Amine Reactive Dye Kit

To label A-84.86-A2GalNAc2 with pHrodo Deep Red (ThermoFisher, Cat #P35356) the manufacturers recommendations was followed. In brief, 0.5 mg was diluted with sterile PBS (Component B) to a concentration of 2 mg/mL. Sodium Bicarbonate (Component C, 10×) was added to the A-84.86-A2GalNAc2 solution to increase the pH to ~8.3 for the amine reaction. The reaction mixture was added to one of the vials containing the amine reactive pHrodo dye and incubated for 2 hours at room temperature with gentle agitation. Afterward the provided pre-prepared spin columns (3500 MWCO, Component D) were spun at 1000 g for 2 min to remove storage buffer, then 0.5 mL of sterile PBS was added to the column and spun again for 1000 g for 2 min to equilibrate the column. Finally, the reaction mixture was added to the column and spun for a final time and the flow through was collected.

To analyze the degree of labeling (DOL) the labeled compound was analyzed with a Nanodrop at A280 and A647 under acidic conditions. The concentration of the A-84.86-A2GalNAc2 was analyzed with the provided correction factors outlined in ThermoFisher's protocol as well as the degree of labeling. The labeled conjugates were stored at 4 °C until use.

### 8.5.2 HepG2 Preparation for Internalization Confocal Experiment

On day 0 HepG2 cells were detached from their flask with Accutase (ThermoFisher, Cat #00-4555-56) according to the manufacturer's instructions. The cells were spun and counted and plated in a 384-well PhenoPlate (Perkin Elmer, Cat #6057300) at 2000 cells/well in complete media with 1% Pen/Strep (ThermoFisher, Cat #15140122). The plate was returned to the incubator at 37 °C and 5% CO₂ to recover overnight.

On day 1 a 2× dose plate was prepared. A-84.86-A2GalNAc2-pHrodo starting concentration was 200 nM, for A-84.86-A2GalNAc2-pHrodo inhibition by unlabeled A-84.86-A2GalNAc2, the top concentration of unlabeled A-84.86-A2GalNAc2 was also 200 nM while the A-84.86-A2GalNAc2-pHrodo across the concentration series was held at 1 nM, and finally for A-84.86-A2GalNAc2 inhibition by asialofetuin, the top concentration asialofetuin was 200 μM while A-84.86-A2GalNAc2-pHrodo across the concentration series was held at 1 nM. From this top concentration a 5-fold serial dilution was carried out for a total of five concentrations along with a set of wells with just background media. The background media for each condition is as follows: A-84.86-A2GalNAc2-pHrodo uptake, OptiMEM (Thermofisher, Cat #11058021); A-84.86-A2GalNAc2-pHrodo inhibition by A-84.86-A2GalNAc2, 1 nM A-84.86-A2GalNAc2-pHrodo and OptiMEM; and A-84.86-A2GalNAc2-pHrodo inhibition by asialofetuin (Sigma, A4781-1G), 1 nM A-84.86-A2GalNAc2-pHrodo and OptiMEM. For each condition across the concentration series there were three replicate wells.

After preparing the dose plate, the HepG2 cells are counter stained. First the media is decanted from the 384-well PhenoPlate. The cells are washed once with warmed OptiMEM, then the staining media is added. The staining media is OptiMEM with CellMask Orange (1:20000) (ThermoFisher, Cat #C10045) and Hoechst (1:20000) (ThermoFisher, Cat #H3570). The cells are then placed back in the incubator for 5 min. After incubation the media is decanted and the cells are washed once with warmed OptiMEM. Then 25 μL of OptiMEM with 1:20000 Lysotracker Green (ThermoFisher, Cat #L7526) was added followed by 25 μL of the A-84.86-A2GalNAc2 solutions described above.

Immediately the plate is loaded onto the Phenix Opera (Perkin Elmer), which has previously been set in equilibrate at 37 °C and 5% CO₂. The channels are set to TRITC for CellMask Orange, Hoechst for Hoechst, AlexaFluor 647 for A-84.86-A2GalNAc2-pHrodo, and CellTracker Green for the lysosomes. For each well 12 fields are imaged, and the plate is scanned once an hour for a total of 8 hours with the 40× water objective.

### 8.5.3 Image Analysis

After imaging the data was analyzed with the Harmony software (Perkin Elmer). In brief each image was subjective to a flatfield correction. The cells region of interest (ROI) were found by first finding and selecting the nuclei (Hoechst channel) and then the cytoplasm (TRITC channel). The boarder objects, the cells that are cut-off at the edge of the image, were also removed. Within the cell ROI, A-84.86-A2GalNAc2-pHrodo spot objects were searched for in the AlexaFluor 647 channel (DeepRed channel) and lysosomes spot objects were searched for in the CellMask Green channel. To determine colocalization of A-84.86-A2GalNAc2-pHrodo spots and lysosomes, A-84.86-A2GalNAc2-pHrodo spots were searched for in the lysosome ROI. The statistics of each of these three object types, namely, object count, intensity and size were evaluated for each well. These data were exported to PRISM to generate graphs and statistical analysis.

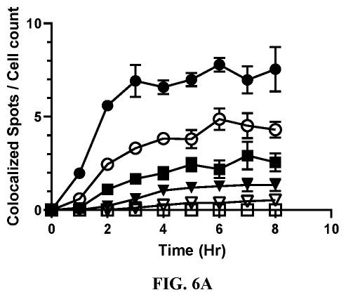

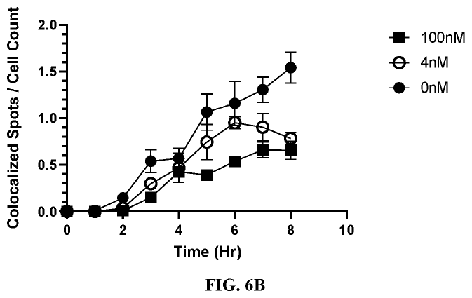

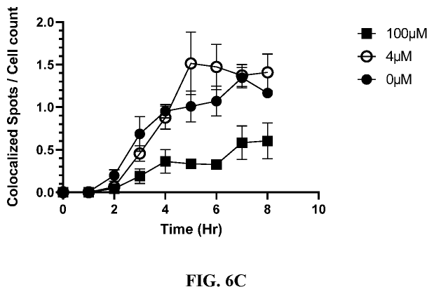

pHrodo labeled A-84.86-A2GalNAc2 shows a dose and time dependent increase in internalization and lysosomal localization (FIGS. 6A-6C). This process is biphasic with a rapid initial step observed in the first 4 hours following which equilibrium is reached between 4-8 hours. There was no saturation in the concentration range tested (0-100 nM) suggesting additional capacity of this in-vitro experimental system. The internalization and colocalization of A-84.86-A2GalNAc2 was competitively inhibited by increasing concentrations of either unlabeled A-84.86-A2GalNAc2 or asialofetuin indicating specificity and ASGPR dependency of this process respectively.

### 8.6 Example 6—A2GalNAc2 Antibody Targets its Antigen to Degradation in HepG2 Cells

HepG2 were plated at 0.8 million cell/well in 1 ml complete medium (DMEM low glucose supplemented with 10% FBS, 2 mM L-Glutamine and Penicillin-Streptomycin) in a 12-well plate and incubated overnight at 37 °C in a cell culture incubator. Cell culture medium was aspirated and 0.3 ml of complete medium with Fc block at 1:25 dilution (Thermofisher #14-9161-73) was added and cells were incubated 15 minutes at 37 °C. Adalimumab antibodies were added to achieve a final concentration of 0.1 mg/ml. Cells were incubated for various time (1.5 to 24 hours) at 37 °C in a cell culture incubator. The cells were then washed with PBS1× and detached with accutase, collected in a 1.5 ml tube and pelleted by centrifugation (3 min 400 g). The supernatant was aspirated and the cells were washed with 1 ml PBS and pelleted by centrifugation. The PBS supernatant was aspirated and the cell pellet was resuspended in 0.2 ml of RIPA lysis buffer complemented with protease inhibitor cocktail (Complete Protease Inhibitor Cocktail tablets, Roche). To lyse the cells, the eppendorf was kept on ice and vortexed vigorously (least 30 seconds) 3-4 times during this period. The cells were then sonicated in an ice cold bath at 35 kHz. The lysate was frozen at −80 °C during at least 15 hours. The lysates were thawed and centrifuged 20 min at >15'000 g to pellet the cell debris. The supernatant was transferred in a new 1.5 ml Eppendorf tube. The protein content was quantified using BCA protein assay kit (Thermofisher ##23225), according to the supplier instructions. The cell protein extracts were adjusted to similar concentration and stored at −80 °C until analysis. The protein extracts (approximately 20 μg/lane) were run on a SDS-PAGE using a 4-12% Bis-Tris pre-casted gel (Thermofisher). The proteins were transferred to nitrocellulose membrane using dry blotting system (iBlot 2, Thermofisher). The membrane was blotted using anti-human IgG H+L (Jackson Immuno Research #709-035-149), used at dilution 1:2500. After membrane stripping, the membrane was reblotted for anti-Beta Actin (Thermofisher #MA1-140) used at dilution 1:4000, detected with Goat anti-Mouse IgG H+L (HRP) (Thermofisher #31430), used at dilution 1:20'000.

Quantification of the western blot signal was performed using the iBright system (Thermofisher) and ibright analysis tool which reports the sum of pixel intensities in an identified lane with background correction (=adjusted total lane volume). Then, the relative signal intensity for a lane is obtained after normalization to the beta-actin signal for this lane.

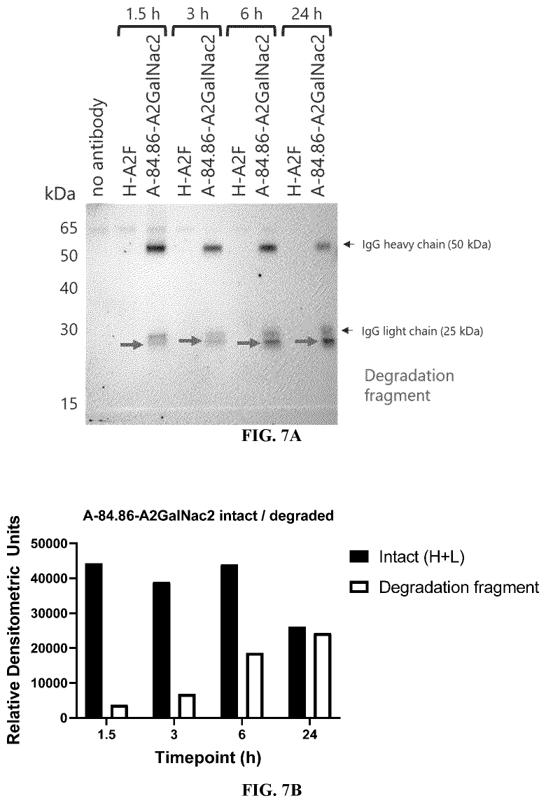

FIGS. 7A-7B show that A-84.86-A2GalNAc2 is internalized specifically in HepG2 cells, while the control H-A2F is not detectably internalized. A degradation fragment is visible and is increasing in intensity overtime while intact heavy chain and intact light chain signal are decreasing overtime, indicating that the internalized A-84.86-A2GalNAc2 antibody is degraded overtime.

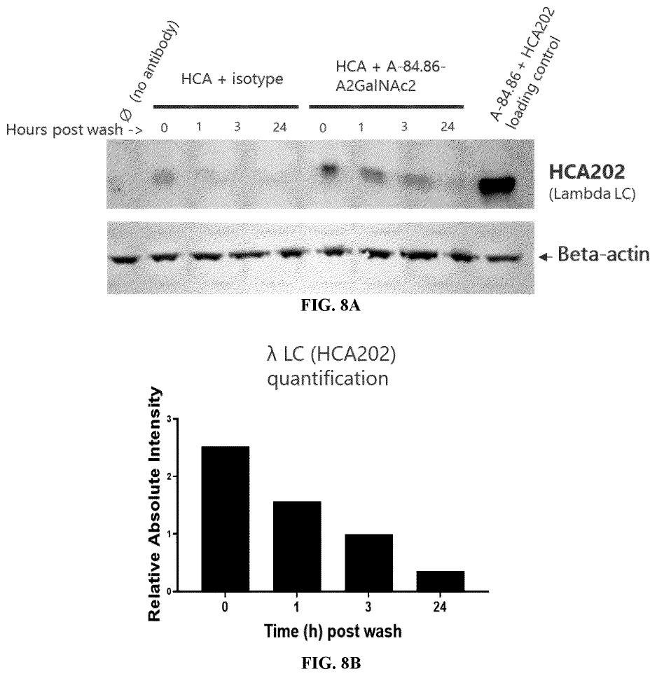

To verify that an antigen is also internalized by A2GalNAc2 displaying antibody a similar experiment was performed. Immune complexes between a human IgG1 control (isotype control) or A-84.86-A2GalNAc2 with anti-adalimumab Fab fragment HCA202 (Biorad, ref HCA202) (the antigen) were formed by mixing antibodies during 30 min in complete HepG2 medium at a ratio of 1 antigen for 1 adalimumab antibody in molarity. The HepG2 cells were incubated with the immune complexes added in the cell culture media at a final concentration corresponding to 0.1 mg/ml of adalimumab, for 3 hours at 37 °C in a cell culture incubator. The cells were then washed to remove immune complexes and the cells were then further incubated with new cell culture media for 1, 3 or 24 hours, at 37 °C in a cell culture incubator. The cells were washed and processed for protein extract as described above. The protein extracts were submitted to SDS-PAGE and western blot for anti-human lambda light chain with same method as described above. The anti-lambda light chain detects only the HCA202 antigen as adalimumab is a kappa light chain antibody. FIGS. 8A-8B show that the antigen is well internalized when complexed with A-84.86-A2GalNAc2 and not with an isotype control. When HCA202 was complexed to H-A2F same minimal internalization of HCA202 than with the isotype control was observed (not shown). Importantly, the signal corresponding to the antigen is decreasing overtime during the washout period. At 3 hours following the washout, the signal has decreased by 75% and is reduced to background level at 24 hours. These data indicate that an antigen for an antibody can be internalized by hepatocyte cells if the antibody displays A2GalNAc2 and that the antigen of the antibody is rapidly degraded after internalization.

### 8.7 Example 7—A2GalNAc2 Antibody is Depleted From Cell Culture Supernatant by HepG2 Cells

To study the ability of human hepatocytes to remove antibodies displaying A2GalNAc2 structure from the environment, an in vitro study was designed where the depletion of antibodies from the supernatants of cultured HepG2 cells was tested by ELISA. One million HepG2 cells (Sigma, 85011430) were plated in a 12-well plate in 600 μl of DMEM low glucose medium (Sigma, D5546) supplemented with 10% heat inactivated FBS (Pan-Biotech, P30-5500), 2 mM L-Glutamine (Sigma, G7513), 10 Units/ml penicillin and 100 μg/ml Streptomycin. Cells were allowed to adhere to the plate overnight and then treated with 5 ng/ml of H-A2F or A-84.86-A2GalNac2 antibodies. Supernatants were collected 24, 48 and 72 hours after treatment and the antibodies content was evaluated by ELISA. To this end, 96-well ELISA plates were coated with 1 μg/ml recombinant human TNF-α in PBS overnight at 4 °C. Plates were washed 3 times with wash buffer (PBST=PBS with 0.05% v/v Tween-20) and blocked with blocking buffer (2% (w/v) bovine serum albumin (BSA) in PBST) for at least 1 hour at room temperature. A 9-point standard curve ranging from 1000 ng/ml to 0.15 ng/ml in 1:3 dilutions was prepared in PBST. After washing the plates 3 times with wash buffer, standards and undiluted samples were added in duplicate and incubated for 1 hour at room temperature. Plates were washed 3 times with wash buffer and the detection antibody (Goat anti-human IgG, γ-specific, HRP, Sigma, A6029) diluted 10000× in PBST was added and incubated for 1 hour at room temperature. ELISA plates were then washed 3 times with wash buffer and revealed by addition of TMB substrate followed by addition of stop solution (H2SO4). ELISA plates were read at 450 and 650 nm on BioTek Synergy H1 instrument using Gen5 Software. H-A2F and A-84.86-A2GalNAc2 concentration in the supernatant was calculated using the standard curve fitting with 4PL and blank reduction.

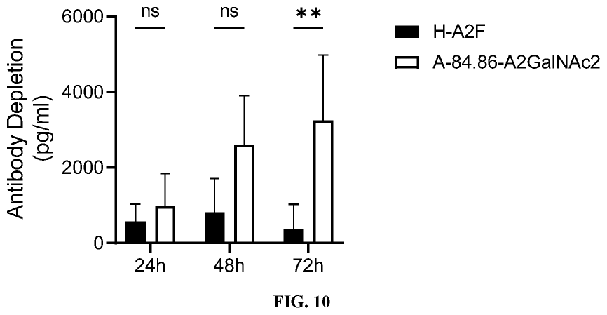

FIG. 10 shows the amount of H-A2F or A-84.86-A2GalNAc2 removed from the supernatants by HepG2 cells at 24, 48 and 72 hours. HepG2 cells depleted up to 3000 μg/ml of A-84.86-A2GalNAc2 after 72 hours of incubation. No significant depletion of H-A2F was observed at any of the timepoint tested.

### 8.8 Example 8—Glycotag-A2GalNAc2 Antibody is Internalized by HepG2 Cells

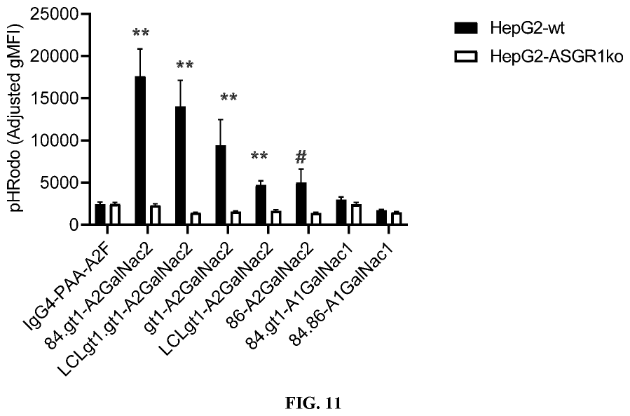

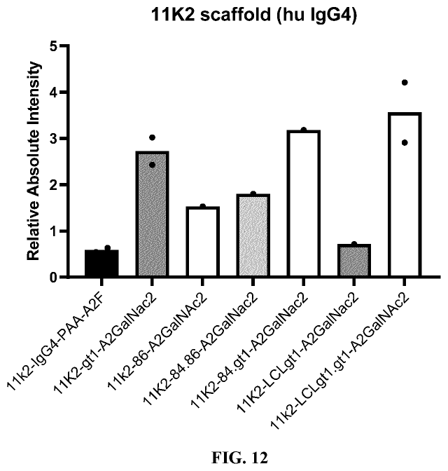

Previous examples show that an antibody engineered to display N-glycosites in the variable regions and presenting GalNAc2 glycan leads to potent internalization and lysosomal degradation by an ASGPR-specific mechanism in hepatocyte cells. A study was designed to assess whether an antibody engineered to display a glycotag attached to C-terminus of heavy or light chain could drive efficient internalization by HepG2 normal cells (HepG2-wt) or HepG2 knock-out for ASGPR1 (HepG2-ASGR1ko). 11K2 antibody, designed as a chimeric mouse-Human IgG4 antibody was used (11K2-IgG4-PAA format). 11K2 antibodies were labeled with pHrodo as described in example 3 and processed through same flow cytometry assay. FIG. 11 shows that 11K2-gt1-A2GalNAc2, with a glycotag added on C-terminus of HC was efficiently internalized, as compared to the non-ASGPR engaging antibody 11K2-IgG4-PAA-A2F. The internalization is ASGPR-specific as ASGPRko HepG2 cells did not internalize the variant. Similarly, a variant displaying a glycotag on C-terminus of LC (11K2-LCLgt1-A2GalNAc2) showed some internalization, albeit lower than the HC glycotag variant. A variant displaying with glycotags on C-terminus of HC and C-terminus of LC (11K2-LCLgt1.gt1-A2GalNAc2) showed a slightly better internalization than 11K2-gt1-A2GalNAc2. Similarly, the variant 11K2-84.gt1-A2GalNAc2 with a glycotag on HC plus a glycosite inserted at position 84 in the variable domain showed a higher internalization than the gt1 variant. These data were confirmed by western blot as shown on FIG. 12 For the western blot, a similar method was applied as described in Example 6, using 11K2 antibodies instead of adalimumab antibodies. These data show that ASGPR-mediated internalization of A2GalNAc2 displaying antibodies function similarly independently of antibody IgG1 or IgG4 isotype. The data also show that a single glycotag on C-terminus of HC drives efficient internalization, but that the position of the glycotag in an antibody can affect the potency of internalization as the glycotag on LC is less efficient that the glycotag on HC. In addition, the data show that increasing the A2GalNAc2 load by adding a second glycotag or a glycosylation site in variable domain increases the potency of internalization. The observation that addition of the 84 mutation, on top of a glycotag on the C-terminus of HC indicates that long spacing glycosites (distal sites) can still function to increase recognition by ASGPR.

Of note the data also show that A1GalNAc1 glycan is not a ligand for ASGPR in vitro as neither the 11K2-84.86-A1GalNAc1 nor 84.gt1-A1GalNAc1 variants were internalized.

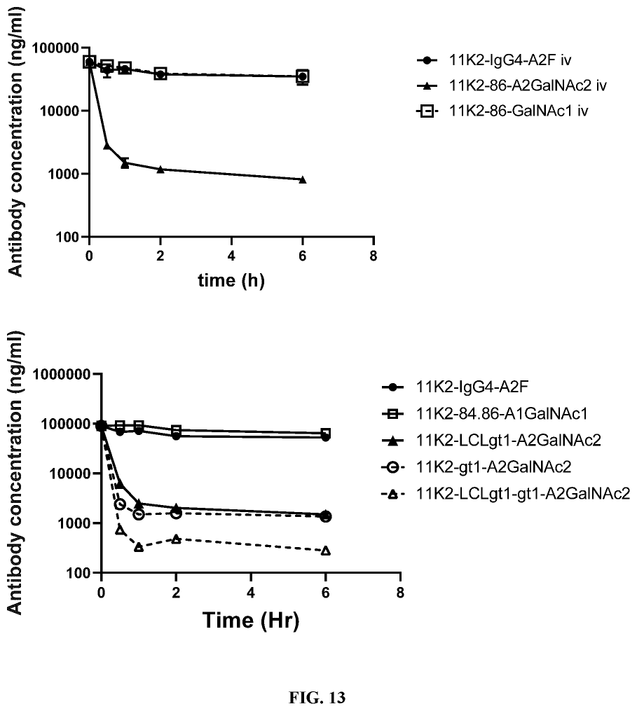

The ASGPR engagement activity of these 11K2 antibody variants was tested in vivo in mouse. It was reasoned that antibody PK profile and particularly the clearance rate of the antibody is directly correlated to ASGPR engagement potency. Mice were injected with the antibodies i.v at 2.5 mg/kg and the PK profile of the antibodies was measured in serum using an ELISA method. The data in FIG. 13 show that all the antibody variants displaying A2GalNAC2 either on light chain variable domain (11K2-86-A2GalNAc2) or on a glycotag on C-terminus of heavy chain (11K2-gt1-A2GalNAc2), C-terminus of light chain (11K2-LCLgt1-A2GalNAc2), or a glycotag on both heavy and light chain, were cleared very rapidly as compared to non glycoengineered A2F mAb, indicating strong ASGPR engagement in vivo, driving their rapid depletion from circulation. The data further highlight that a single glycotag is sufficient to drive this efficient clearance. In contrast the data confirm that A1GalNAc1 glycan does not engage ASGPR in vivo as these variants show overlapping profile with the non glycoengineered antibody, even if 2 glycan per Fab portion were present (A-84.86-A1GalNAc1).

### 8.9 Example 9—Glycotag-A2GalNAc2 Fab Fragment is Internalized and Degraded by HepG2 Cells

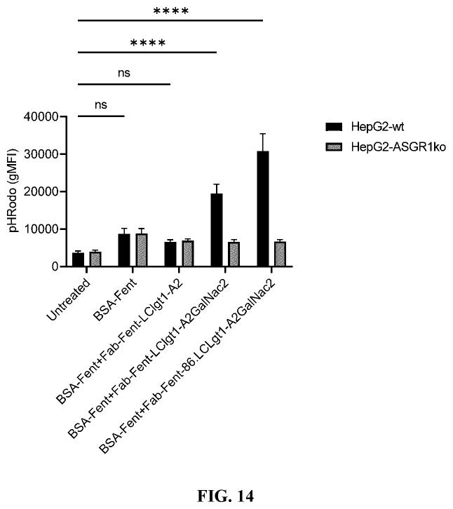

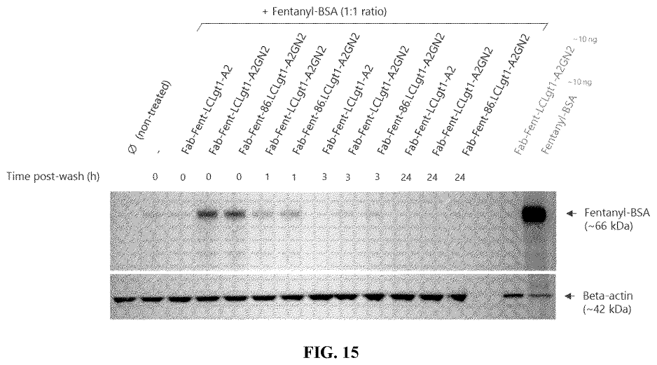

Glycovariants of a Fab fragment of a fentanyl specific antibody were produced (Fab-Fent variants) (See Tables 7-9). BSA-fentanyl (biorbyt #orb738533) was used as the antigen. BSA-fentanyl was pHrodo labeled using method described in Example 3. HepG2-wt and HepG2-ASGPR1ko cells were incubated with 3 μg/ml of pHrodo-BSA-Fentanyl×Fab-Fent complexes for 4 h. The pHrodo fluorescence intensity was analyzed as described in example 3. FIG. 14 shows that the control Fab-Fent-LCLgt1-A2, displaying a non ASGPR engaging glycan on a glycotag did not lead to significant internalization of BSA-fentanyl antigen. In contrast, Fab-Fent-LCLgt1-A2GalNAc2, which displays on its glycotag a single A2GalNAc2 glycan, induced a clear internalization of the antigen. This internalization is ASGPR dependent as it is abrogated in HepG2 knock out for ASGPR. Similarly the variant Fab-Fent-86.LCLgt1-A2GalNAc2 induced an even stronger ASGPR-dependent internalization of the antigen, as comparted to the LCLgt1 variant. These observations were confirmed by western blot visualization of degradation of the BSA-Fentanyl antigen after internalization. A similar experiment was performed as the one described in example 6. HepG2 cells were incubated with complexes between Fab-Fent variants and BSA-fentanyl antigen, for 3 hours, washed, and then further incubated for 0, 1, 3 and 24 hours and processed for protein cell extract. The cell extracts were submitted to SDS-PAGE and western blot using the anti-fentanyl antibody as a probe and for Beta-Actin. FIG. 15 shows the western blot. The BSA-fentanyl was internalized only when A2GalNAc2 displaying Fab-Fent variants were used. In addition, the internalized BSA-fentanyl was rapidly degraded as shown by the fact that the signal was almost completely lost 3 hours after the washout.

These data show that a Fab Fragment displaying a single A2GalNAc2 glycan can induce a potent ASGPR-mediated internalization of its antigen. When the Fab Fragments displays 2 units of A2GalNAc2 glycan, the internalization is increased. The antigen internalized is rapidly processed through lysosomal degradation.

### 8.10 Example 10—A2GalNAc2-MOG-Fc Fusion Protein is Internalized by HepG2 Cells

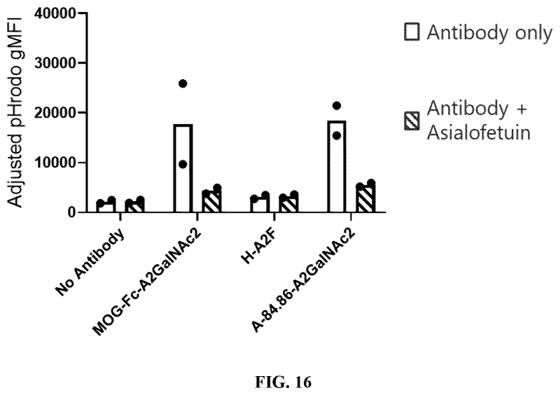

In order to test whether a fusion protein, displaying natural glycosite can be recognized and internalized by HepG2 cells, a MOG-Fc fusion protein was constructing, by fusing the extracellular domain of human MOG to the CH2 and CH3 domain of human IgG. The MOG extracellular domain displays a natural glycosite at position N60 (considering the entire sequence including the signal peptide). A MOG-Fc-A2GalNAc2 was produced. The construct was verified to be a homodimer. The MOG-Fc-A2GalNAc2 was labeled with pHrodo using method described in example 3. HepG2 cells were pre-incubated with HepG2 media or media+2 μM asialofetuin for 30 min at 3/°C and then pHrodo-labeled MOG-Fc or antibodies were added at 3 μg/ml final concentration and the cells were further incubated for 3 hours at 37 °C in a cell culture incubator. FIG. 16 shows that the MOG-Fc-A2GalNAc2 construct was efficiently internalized by HepG2 cells, to a similar extent than the A-84.86-A2GalNAc2 antibody. The internalization was ASGPR specific as it was block by competition with asialofetuin.

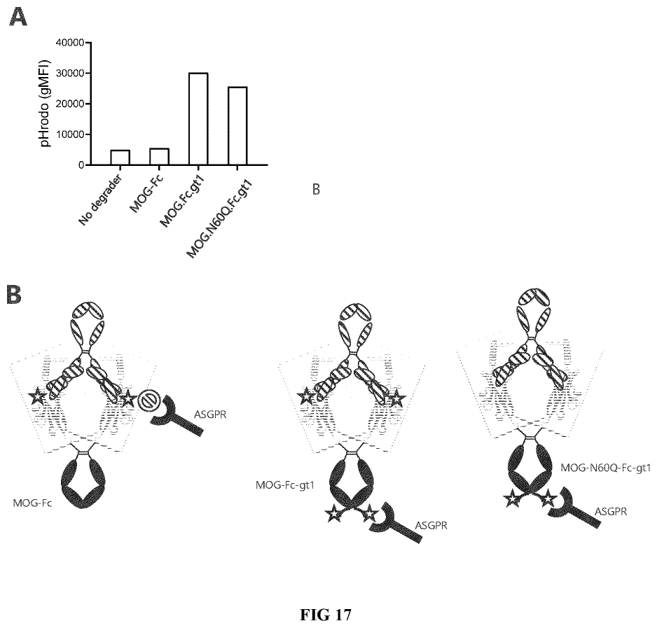

MOG extracellular domain (ECD) contains an endogenous N-glycosite at position N60, which displays A2GalNAc2 when produced in accordance to the methods described herein and drives efficient ASGPR-mediated internalization (FIG. 16). Target internalization by glycoengineered MOG constructs was tested, using 8-18C5 mAb as a model anti-MOG autoantibody (Sun et al. (2021) Mol. Ther. 29:1312-1323). Two additional MOG constructs were generated. The MOG-Fc-gt1 construct has an added glycotag at the C-terminus of the Fc. The MOG-N60Q-gt1 construct has the endogenous MOG ECD glycosite (N60) mutated and a Fc glycotag added at the C-terminus. The 8-18C5 mAb was labeled with pHrodo (same method than in Example 3) and incubated with each of the 3 different MOG constructs on HepG2 cells, similar to the procedures described above. The binding of pHrodo-8-18C5 was verified to be equivalent on the 3 constructs, by ELISA (data not shown). FIG. 17 shows the internationalization data. Interestingly the MOG-Fc-A2GalNAc2 construct was unable to drive internalization in the presence of the 8-18C5 mAb anti-MOG autoantibody. In contrast, each of the MOG constructs with a glycotag at the C-terminus of the Fc were able to drive efficient target internalization. These data indicate that binding of the anti-MOG autoantibody to the MOG-Fc construct may prevent recognition of the A2GalNAc2 at the endogenous MOG glycosite by ASGPR. The presence of a glycotag distal from the target binding site (e.g. at the C-terminus of the Fc) can restore ASGPR-mediated internalization of the MOG construct-anti-MOG autoantibody complex. More generally, the data suggest that placement of the A2GalNAc2 glycan distal to the target engagement epitope (e.g. an ECD or antigen-binding region) may be important to maintain efficient ASGPR mediated internalization in the presence of the target (e.g. an autoantibody or antigen, respectively).

In addition, these findings indicate that A2GalNAc2 displayed by a glycotag can replace A2GalNAc2 displayed by an endogenous glycosite (such as the N60 position in MOG ECD). Without being bound by theory, the endogenous glycosite of Fc, located at amino acid 297, is generally considered to be inaccessible, and therefore largely inactive. If desired, it is possible to mutate endogenous N-glycosites (such as that present in the Fc sequence at amino acid 297, or that present in MOG ECD at position N60) to eliminate the possibility of A2GalNAc2 display at these sites. In this manner, greater control of A2GalNAc2 display may be gained by addition of one or more glycotags situated at other positions and/or the deletion of endogenous glycosites, allowing more precise control of the total A2GalNAc2 load displayed by the degrader. The data presented here indicate that a single A2GalNAc2 glycan per molecule of degrader is sufficient to drive efficient ASGPR-mediated internalization. Without being bound by theory, avoiding too high an A2GalNAc2 load per degrader molecule may be beneficial to prevent too strong or unphysiological interaction with ASGPR and/or fine-tune the relative internalization rates of the degrader in the presence and absence of a target molecule.

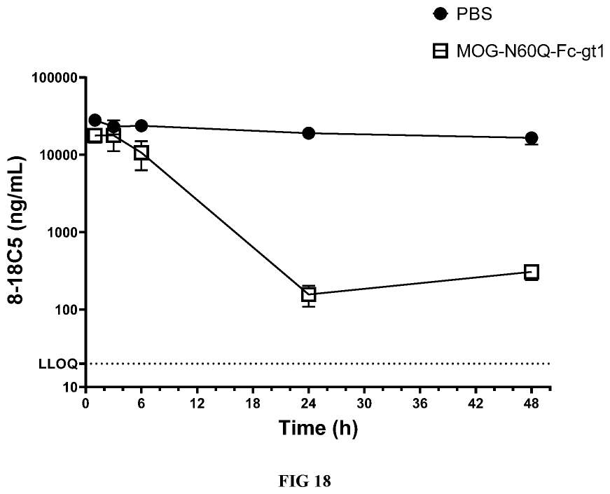

The in vivo target depletion potency of MOG-Fc-N60Q-gt1-A2GalNAc2 was tested in rats. Animals were injected with the 8-18C5 target antibody i.p. at 2 mg/kg followed 24 hours later by MOG-Fc-N60Q-gt1-A2GalNAc2 injection s.c. at 5 mg/kg, or PBS. The total (free+bound) levels of 8-18C5 mAb were measured by ELISA in serum. The data in FIG. 18 show that treatment with MOG-Fc-N60Q-gt1-A2GalNAc2 led to a strong depletion of the target antibody. The maximal depletion was reached at 24 h (>99% depletion as compared to PBS treated group). This timing of depletion is in agreement with slow distribution of s.c. injected biologic compound to the circulating blood compartment.

### 8.11 Example 11—A2GalNAc2 Antibodies Lead to Potent In Vivo Depletion of a Blood Circulating Antigen

To assess the potency of antibodies produced in accordance to the methods described herein (the glycan-modified antibodies and the glycoengineered host cells that produce them are elements of the inventors' “Custom Glycan Platform”, which is also referred to as “CGP”) and displaying GalNAc terminated glycans (e.g., an A2GalNAc2 structure) to deplete a circulating antigen, an experiment in rat was designed. Rats were injected with an antigen (i.e. a target) and with antibodies displaying A2GalNAc2 glycan structure on their Fab or control antibodies, specific for the antigen. The level of the circulating antigen in the serum of treated animals was quantified along time to measure the extend of depletion of the antigen from peripheral blood compartment. All antibodies showed aggregate levels below 5% as assessed by size exclusion HPLC method and higher than 94% purity as assessed by reducing polyacrylamide gel electrophoresis analysis and had an endotoxin level lower than 1 EU/mg (LAL assay). All glycoengineered antibodies (Table 9) conserved high binding to HCA202.

Wistar female rats (Janvier Labs, St Berthevin, France, ref. RjHan:WI) 180-220 g at start of experiment were injected i.v. bolus with anti-adalimumab Fab fragment HCA202 (Biorad, ref HCA202) (the antigen or target) at 0.5 mg/kg dose, 0.5 ml/rat. HCA202 compound was submitted prior to injection to an endotoxin removal step using Pierce™ High Capacity Endotoxin Removal Spin Columns (Thermofisher, ref. 88274). Fifteen (15) min. later rats were injected with antibodies or PBS. Blood samples were taken from jugular vein by puncture at different time points. Terminal blood samples were collected from abdominal aorta. Blood samples were left for clotting 30 min. at room temperature followed by centrifugation to collect serum.

Total HCA202 levels (antibody bound+free HCA202) were measured by ELISA method. Anti-Penta-His antibody (Qiagen, Ref. 34660) was coated on 96-well ELISA plates at 5 μg/ml in coating buffer (PBS pH 7.4, final composition: 8 mM Na-Phosphate; 8 mM K-Phosphate, 0.15 M NaCl, 10 mM KCl) overnight at 4 °C. This antibody allows the capture of antibody bound and free HCA202 as HCA202 has a histidine tag at C terminus of heavy chain. Plates were washed 3 times with wash buffer (PBST=PBS with 0.05% v/v Tween-20). Blocking buffer (2% (w/v) Bovine serum albumin (BSA) in PBST) was added to each well and plates were incubated for 1-3 hours at room temperature. A 7-point calibration curve from 500 ng/ml to 0.7 ng/ml in 1:3 dilutions was made on separate dilution plates. For that, undiluted normal rat wistar serum was spiked with 5 μg/ml HCA202 and 3-fold molar excess of adalimumab (Humira). Spiked serum was incubated 10 min. at room temperature to allow adalimumab/HCA202 immune complex formation. The spiked serum was diluted 10-fold (MRD10) by adding diluent B (2% (w/v) Bovine serum albumin (BSA) in PBST). A serial 1:3 dilution of the immune complex standard curve was performed using diluent B. Study samples were processed similarly. Study serum samples were diluted 10-fold in dilution plates (to achieve MRD10 samples) using diluent B. MRD10 samples were further diluted if needed in diluent A (1/10 wistar rat pooled serum diluted in 2% BSA+0.05% PBST) to achieve a signal within the linear range of the calibration standard curve. After blocking step, ELISA plates ware washed 3 times with wash buffer. Calibration standards and diluted study samples were added in duplicates to ELISA plates and incubated at room temperature for 1 h. ELISA plates were washed 3 times with wash buffer and a Humira solution at 1000 ng/ml was added to each sample. ELISA plates were incubated for 1 h at room temperature. Plates were washed 3 times with wash buffer. A detection antibody solution was prepared by diluting goat anti-human kappa LC-HRP (Thermofisher, ref. A18853) 1:5000 in diluent B. The detection antibody solution was added to the ELISA plates and incubated for 1 h at room temperature, protected from light. ELISA plates were then washed 3 times with wash buffer and revealed by addition of TMB substrate followed by quenching with H₂SO₄. ELISA plates were read at 450 and 650 nM on a plate reader such as BioTek Synergy H1. Data analysis was made using standard software such as Gen5 (Biotek).

Antibody levels in serum sample can be quantified by ELISA method. The assay consists of a coating step with human TNFα to capture adalimumab and adalimumab variants present in the sample. Detection can be performed via an anti-human gamma HC specific HRP-tagged detection antibody. The assay therefore quantifies only free antibodies (having at least one Fab arm not bound to HCA202). Briefly, recombinant Human TNF-α (Peprotech, ref. AF-300-01A) is coated on 96-well ELISA plates, typically at 1 μg/ml in PBS pH 7.4 at 4 °C overnight. Blocking buffer, dilution buffer A and B and wash buffer are the same than used for the HCA202 ELISA. Plates are washed 3 times and blocked with blocking buffer as described in the HCA202 ELISA. Typically, a 7-point calibration curve, for example from 333.3 ng/ml to 0.5 ng/ml in 1:3 dilutions is prepared by spiking pooled wistar rat serum diluted 10-fold (minimal required 10-fold dilution, MRD10) in dilution buffer B with 1 μg/ml adalimumab. Study serum samples are also diluted in dilution buffer B (MRD10 samples minimum). Diluted study samples and standard calibration curve samples are then transferred to the ELISA plate, after blocking step and incubated 1 h at room temperature. Plates are then washed 3 times and solution of detection antibody is added. Solution of detection antibody can be prepared by diluting for example a Goat Anti-Human IgG (γ-chain specific)-HRP (Sigma, ref. A6029) antibody (typical dilution 1:10'000) in dilution buffer B. ELISA plates are incubated with detection antibody typically 1 h at room temperature, protected from light. Plates are then washed 3 times and revealed by adding TMB substrate as described in the HCA202 ELISA.

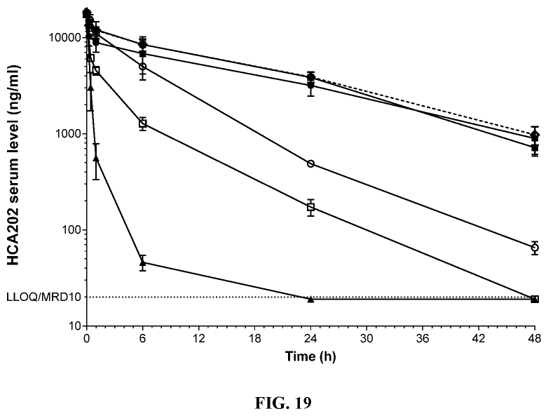

FIG. 19 shows the data obtained for HCA202 levels. Table 11 shows the HCA202 depletion numbers. When no antibody is injected (PBS condition), HCA202 decays slowly over period of 48 h, as expected for a Fab fragment. H-A2F (non-engineered adalimumab) treatment led to increase levels of HCA202 at 24 hours and 48 hours time point. Treatment with A-M3 and A-84.86-A2G2S2 also led to increased HCA levels as compared to PBS treatment (72% depletion from C_{zero} with PBS vs 52-63% depletion with H-A2F, A-M3 and A-84.86-A2G2S2 at 6 h), showing that these antibodies have no depleting potency. C_{zero} is the theoretical concentration (of HCA) in serum that would have been achieved immediately post injection, considering immediate homogeneous whole blood distribution. In contrast, injection of A-84-A2GalNAc2 led to a significant depletion of HCA202 as compared to non-depleting antibodies and PBS at 1 hour (74% depletion) and 6 hour (93% depletion). A-84.86-A2GalNAc2 treatment led to a more extensive and faster HCA202 depletion (97% depletion at 1 h, 100% at 6 h).

TABLE 11
HCA202 depletion by A2GalNAc2 glycosylated Fab antibodies

| | | H- | A- | A-84.86- | A-84- | A-84.86- |
|---|---|---|---|---|---|---|
| | PBS | A2F | M3 | A2G2S2 | A2GalNAc2 | A2GalNAc2 |
| % depletion | 39 | 51 | 34 | 31 | 74 | 97 |
| at 1 h | | | | | | |
| % depletion | 72 | 63 | 53 | 52 | 93 | 100 |
| at 6 h | | | | | | |

Table 11 shows the % of HCA202 depletion from C_{zero}.

These data highlight that antibodies displaying A2GalNAc2 glycans on their Fab fragment have a high potency to eliminate a circulating antigen from blood circulation in a very short time. These data also show that the glycan load displayed per molecule modulates the potency of antigen elimination, as the A-84.86-A2GalNAc2 antibody with 2 engineered glycosites per Fab showed a higher depletion potency as compared to A-84-A2GalNAc2 antibody with a single glycosite per Fab.

### 8.12 Example 12—A2GalNAc2 Antibody is Targeted to the Liver In Vivo

To study the in vivo distribution of antibodies displaying GalNAc terminated glycans (A2GalNAc2 structure), a study in mouse was designed with fluorescently-labeled antibodies displaying A2GalNAc2 or control glycans and using in vivo and ex-vivo tomography imaging. Antibodies were labeled with CF750 labeling kit (Biotium, ref 92221) for a volume of at least 1 mL at 1 mg/mL, following manufacturer's instructions. After labeling, the degree of labeling (DOL) was measured. The DOL ranged from 2.7 to 5.2 so antibodies were considered to be similarly labeled. SKH1 immunocompetent hairless mice (Charles River Laboratories, ref Crl:SKH1-hr) 5-6 weeks at experiment start were injected (intravenous bolus) with the CF750 labeled antibodies at 5 mg/kg dose. Mice were imaged using the FMT 2500™ fluorescence tomography in vivo imaging in the system (PerkinElmer), which collected both 2D surface fluorescence reflectance images (FRI) as well as 3D fluorescence tomographic (FMT) imaging datasets. For each animal 2 scans in decubitus dorsal position were performed (thorax area+abdomen area) with the FMT (NIR excitation laser 745 nm/emission 770-800 nm) at the following time points: 0.25 h, 1 h, 3 h, 6 h, 24 h and 48 h. The collected fluorescence data were reconstructed by FMT 2500 system software (TrueQuant V2.0, PerkinElmer) for the quantification of the three-dimensional fluorescence signal in whole animal body. Three-dimensional regions of interest (ROI) were drawn encompassing the relevant biology on the thorax, abdomen and liver areas. The quantity and percentage of dose of labeled antibodies in each ROI was determined for each time point. At time point 6 hour and 48 hour, thyroids (with trachea), lungs, heart, liver, spleen, kidneys were harvested and submitted to FMT imaging.

Table 12 shows the antibody characteristics that were included in the study.

TABLE 12
Antibody characteristics.

| | Engineered | Main N- | Main N-glycan |
|---|---|---|---|
| | glycosites | glycan | structure on |
| | (Eu | structure on | engineered |
| Antibody | numbering) | N297 (Fc) | glycosites |
| H-A2F | None | A2F | NA |
| A-M3 | None | M3 | NA |
| A-84.86- | LC: D86N | A2 | A2GalNAc2 |
| A2GalNAc2 | HC: K84N | | |
| A-84.86- | LC: D86N | A2 | A2G2S2 |
| A2G2S2 | HC: K84N | | |
| Ptz-A2F | None | A2F | NA |

NA: not applicable.
Main N-glycan structure on Fc is on the canonical N-297 position. Main N-glycan structure on Fab means on the engineered glycosite by point mutations (Eu numbering). LC is light chain, HC is heavy chain. Black striped circle represents mannose (Man), white square is N-acetyl glucosamine (GlcNAc), white circle is galactose (Gal), black square is N-acetyl galactosamine (GalNAc), white diamond is sialic acid, N-acetyl neuraminic acid (Neu5Ac), and white triangle is fucose (Fuc).

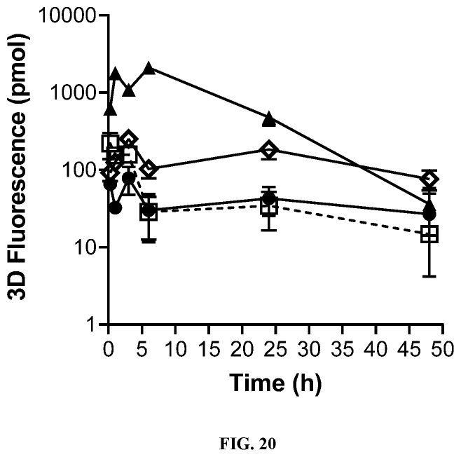

FIG. 20 shows the FMT imaging data for thorax and liver ROI along time for each antibody. Table 13 shows the FMT imaging data obtained on harvested organs at 6 hours. The two non-engineered control antibodies (H-A2F=adalimumab; Ptz-A2F=pertuzumab) showed a very similar distribution profile with an overall similar and low level of signal distributed in thorax or liver area. Signal did not increase over time in the liver area (FIG. 20). At 6 hour time point, less than 8% of injected dose of Ptz-A2F and H-A2F was present in the liver (Table 13) while these 2 antibodies could be detected in most other organs. This distribution pattern is consistent with normal human IgG and characteristics of antibodies that are broadly distributed and still mainly present in the blood. The antibodies A-84.86-A2G2S2 and A-M3 showed a distribution profile similar to H-A2F and Ptz-A2F control antibodies, with broad organ distribution and relatively low liver distribution. At 6 hour, 20% of the injected dose for A-84.86-A2G2S2 and A-M3 was present in the liver (Table 13). In contrast, A-84.86-A2GalNAc2 antibody showed a rapid and essentially exclusive distribution to the liver area, followed by a decline after 6 hours (FIG. 20). The decline observed after 6 hours is likely due to degradation of the antibody into peptides by the lysosomal machinery and excretion of the product of degradation (including the fluorophore-couple peptides) from the liver cells. At 6 hour time point, 85% of the injected dose of A-84.86-A2GalNAc2 was present in the liver (Table 13). A-84.86-A2GalNAc2 antibody was absent from Thyroid, lungs, heart and spleen and detectable at low level in the kidneys. This pattern of distribution is characteristics of an antibody that is essentially exclusively distributed to the liver and therefore not present in the blood and other organs. These data show that an antibody displaying A2GalNAc2 structure on its Fab fragment is directed almost exclusively to the liver.

TABLE 13
Organ distribution data at 6 hour time point.

| | H- | Ptz- | A- | A-84.86- | A-84.86- |
|---|---|---|---|---|---|
| | A2F | A2F | M3 | A2G2S2 | A2GalNAc2 |
| Liver | 6.1 | 7.8 | 16.4 | 20.2 | 85.3 |
| Lungs | 1.1 | 2.1 | 2.0 | 1.3 | 0.0 |
| Thyroid/ | 0.5 | 0.9 | 0.4 | 0.1 | 0.0 |
| Trachea | | | | | |
| Heart | 0.7 | 1.3 | 0.8 | 0.9 | 0.0 |
| Kidneys | 2.0 | 3.4 | 2.3 | 1.4 | 1.3 |
| Spleen | 0.3 | 0.2 | 0.7 | 0.5 | 0.1 |

Table 13 shows the average (N=3) % of injected fluorophore dose in indicated harvested organs at 6 hour time point.

### 8.13 Example 13—A2GalNAc2 Antibody and its Target are Directed to the Liver and Targeted to Degradation

The presence of injected CF750-labeled antibodies in the liver of animals from the experiment described in Example 12, was analyzed by western blot. At timepoint 6 hour and 48 hour the livers were harvested and submitted to total protein extraction using RIPA buffer. Fifty to hundred mg of liver tissue was homogenized using two microscope glasses. The homogenate was transferred to a 15 ml tube containing RIPA buffer supplemented with protease inhibitors and incubated on ice for 30 minutes. Samples were vortexed every 5 minutes and then spun for 3 minutes at 3000×g to remove debris. Supernatants were transferred in a new tube and further homogenized using a syringe and a 25 G needle. Extracts were then sonicated for 20 minutes in an ice bath and spun to remove debris. Protein content in each sample was quantify using the BCA Assay Kit (Pierce). The protein extracts were loaded on polyacrylamide gel electrophoresis, transferred to a nitrocellulose membrane using the iBlot dry transfer system and CF750 fluorescence was read using an iBright™ system (Invitrogen). Fluorescence densitometric signal was quantified using the iBright software. The CF750 fluorescence signals directly indicate the quantity of labeled antibody (intact or fragments of) present in the liver extract. Relative densitometric units were determined by normalizing the fluorescent signal to the signal density of the loading control β-actin

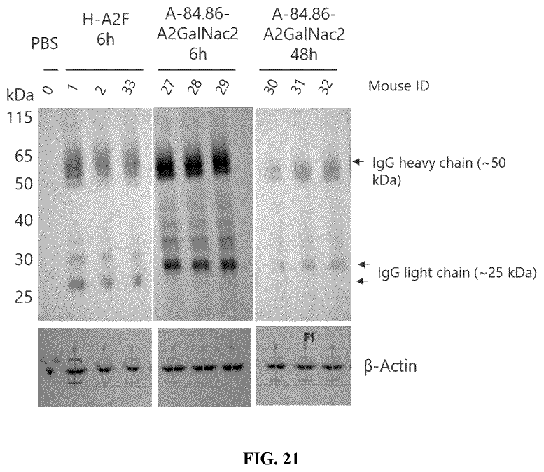

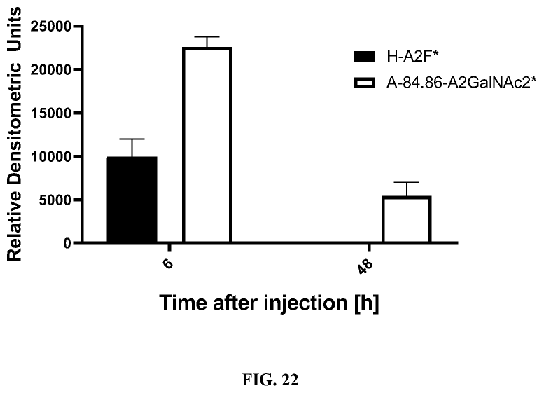

FIG. 21 shows the gel fluorescence blot. FIG. 22 shows the quantification of antibody signal from the blot. Livers from A-84.86-A2GalNAc2 injected animals, collected at 6 h after injection contained significantly higher amount of antibody (227% more) as compared to livers from H-A2F treated animals confirming that A-84.86-A2GalNAc2 is more directed to the liver as compared to the non-ASGPR engaging H-A2F control, as shown by the biodistribution data. Importantly the blot shows clearly bands corresponding to fragmentation of the heavy chain of the H-A2F and A-84.86-A2GalNAc2 antibodies, providing strong evidence that the liver targeted A2GalNAc2 antibody is submitted to lysosomal degradation. At 48 hour, the level of A-84.86-A2GalNAc2 antibody in liver cells has dramatically decreased to 25% compared to the 6 hour timepoint, further highlighting that A-84.86-A2GalNAC2 is degraded. These data are in agreement with the biodistribution data showing that the A-84.86-A2GalNAc2 signal is near maximal in the liver at 1 hour and decreases at 48 hour (Example 12).

To verify that the target antigen of the A2GalNAC2 antibody is also directed to the liver and degraded an experiment was setup. Mice (N=3/group) were injected i.v. with HCA202 at 0.25 mg/kg, followed by treatment i.v. with control antibody H-A2F, or PBS or A-84.86-A2GalNAc2. The liver of each animal was harvested at different timepoints, rinsed with PBS and dry frozen at −80 °C. The livers were then processed for total protein extract. A small chuck, ~30 mg, was chipped off in a pre-weighed petri dish on wet ice with a sterile, ice-cold, razor blade. The rest of the liver was returned to the −80 °C. The liver tissue was transferred to a 2 mL microcentrifuge tube with round bottom and rinsed with 1 ml ice-cold PBS. Then ice-cold RIPA (with 50×PI, 100×PMSF, and 100×Na3VO4) at 500 μL/30 mg of tissue was added to the livers. A single 5 mm grinding ball (OPS Diagnostics) was added to each sample. The samples were then placed in the TissueLyserII (Qiagen) at 4 °C. The livers were processed for 5 min at 20 Hz and then allowed to rest for an additional 15 min. The samples were then spun at Max Speed in a pre-cooled microcentrifuge for 15 min at 4 °C. The supernatant was collected, and a BCA was performed on a 1:5 dilution of the stock liver lysates. The liver extracts were processed using NuPAGE™ 12%, Bis-Tris, 1.0 mm, 17 wells Mini Protein Gels (NP0349BOX). The gel was analyzed by western blot using Goat Anti-Human Lambda LC IgG (Milipore Sigma #AP506P) at 1:2000 in 5% Milk-TBST. HCA202 is a human Lambda Fab antibody while H-A2F and A-84.86 are Kappa light chain antibodies. Therefore anti-Lambda LC western blot detects specifically HCA202. Total protein was imaged in AF680 fluorescent channel right after protein transfer. The signal density of the antibody band and total protein quantitation was performed using the iBright FL1500 analysis tool. Ratio of antibody band to corresponding total protein was normalized to HCA202+A-84.86-A2GalNAC2 at 0.5 h timepoint.

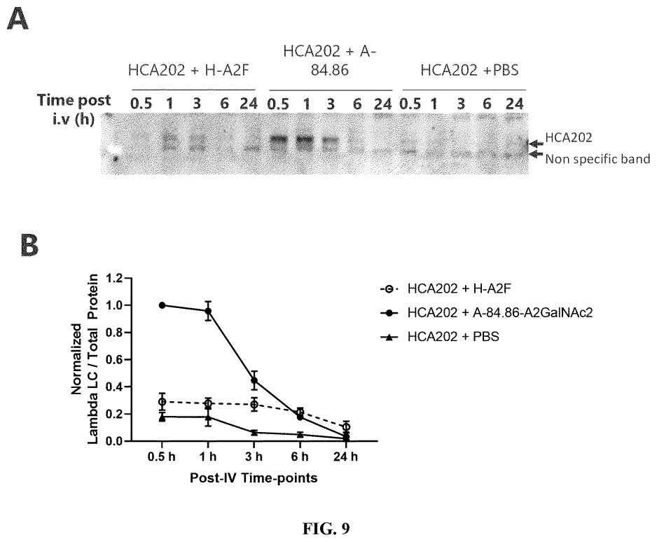

Low levels of HCA202 were observed at any timepoint in the livers of animals treated with H-A2F or PBS (FIG. 9). In contrast, treatment with A-84.86-A2GalNAc2 led to a strong accumulation of HCA202 in the liver at early timepoints 0.5 and 1 h. In addition, the HCA202 level then gradually decreased and was minimal at 6 hours post treatment. These data show that the HCA202 antigen is effectively and very rapidly directed to the liver by A-84.86-A2GalNAc2 antibody and is rapidly degraded in liver cells following internalization.

### 8.14 Example 14—Route of Injection Prolongs the Effect of GalNAc2 Antibody-Mediated Antigen Depletion

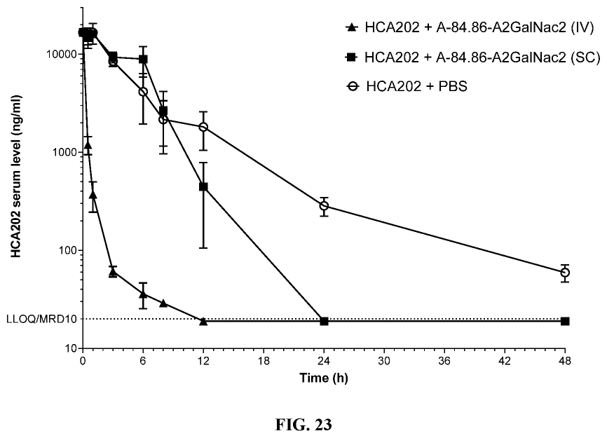

Intravenous (i.v.) injection of A-84.86-A2GalNAc2 led to a fast depletion of HCA202 antigen (97% depletion at 1 h; Example 11). It was rationalized that a treatment enabling slow release of the antibody into the blood would prolong the depletion effect. For this purpose, an experiment injecting A-84.86-A2GalNAc2 antibody via subcutaneous (s.c.) route was designed. Rats were injected i.v. with HCA202 antigen (0.5 mg/kg), followed 15 minutes later by a single s.c. or i.v. injection of A-84.86-A2GalNAc2 at 10 mg/kg and 5 mg/kg respectively or PBS injection. The level of HCA202 in the serum of treated animals was quantified along time. The method for HCA202 quantification is described in Example 11. FIG. 23 shows the data obtained. As in the previous experiments (Example 11), when no antibody is injected (PBS condition), HCA202 decays slowly over period of 48 h, as expected for a Fab fragment. Injection of A-84.86-A2GalNAc2 i.v. led to a fast and strong decrease of HCA202 levels. Injection of A-84.86-A2GalNAc2 s.c. led to a delayed HC202 depletion reaching complete depletion at 24 hours.

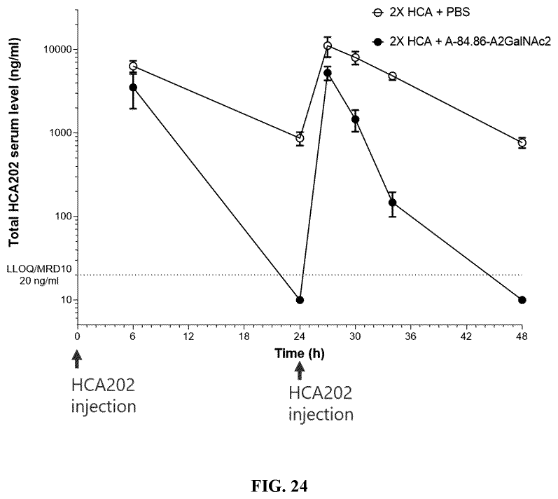

Another experiment was conducted to assess whether a single s.c. administration of A-84.86-A2GalNAc2 can lead to depletion of a second dose of HCA202 at 24 hours. Rats were injected i.v. with HCA202 (0.5 mg/kg) at timepoint zero, followed by a single s.c dose of A-84.86-A2GalNAc2 at 10 mg/kg (or PBS). A second i.v. dose of HCA202 at 24 hours. The serum levels of HCA202 were measured. The data are shown in FIG. 24. The data show that a single s.c. dose of A-84.86-A2GalNAC2 is able to prolong depletion of HCA202 antigen up to 48 hours. These data show that s.c. injection of an ASGPR targeting A2GalNAc2 antibody via a slow release route enables to significantly prolong the depletion effect of an antigen.

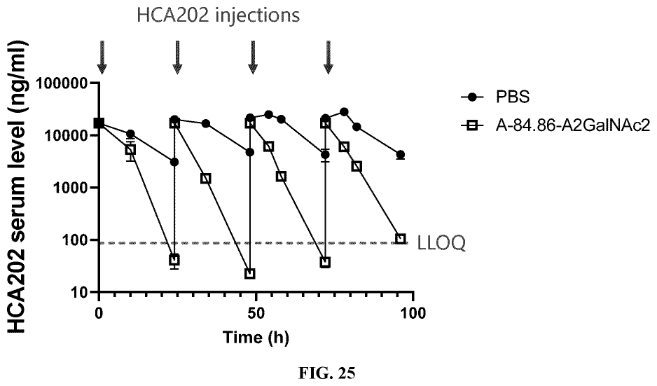

Another experiment was conducted to assess whether a single s.c administration of A-84.86-A2GalNAc2 can lead to depletion of a 4 consecutive doses of HCA202 given 24 hours apart. Rats were injected i.v. with HCA202 (0.5 mg/kg) at timepoint zero, 24 h, 48 h, and 72 h. A single s.c. dose of A-84.86-A2GalNAc2 at 10 mg/kg (or PBS) was given 15 min following initial HCA202 injection. The serum levels of HCA202 were measured. The data are shown in FIG. 25. The data show that a single s.c. administration of A-84.86-A2GalNAc2 at 10 mg/kg was able to deplete 4 consecutive HCA202 doses (FIG. 25), indicating that s.c dosing can maintain depletion efficiency for up to 96 hours. The calculated molar degrader (A-84.86-A2GalNAC2) to target (HCA202) ratio in this experiment is 1.67 further highlighting that low degrader:target ratio are sufficient to drive efficient target depletion by a mAb displaying A2GalNAc2.

### 8.15 Example 15—A2GalNAc2 Glycosylated Antibody Leads to Target Receptor Degradation on Surface of ASGPR-Expressing Cells

To assess whether an antibody specific for a target receptor, and displaying GalNAc2 terminated glycan (A2GalNAC2 structure) can lead to degradation of the target surface receptor on surface of cells expressing ASGPR, an experiment using CGP-produced glycovariants of the pertuzumab (Ptz) anti-HER2 antibody was performed. HepG2 cells co-express HER2 and ASGPR. The hypothesis tested was that A2GalNAc2 displaying Ptz CGP-produced variant is able to co-engage HER2 and ASGPR on surface of HepG2 cells and trigger internalization and degradation of the formed complex, leading to reduction of HER2 levels on HepG2 cells. Pertuzumab antibodies (Ptz-A2, Ptz-86-A2GalNAc2, Ptz-gt1-A2GalNAc2, Ptz-hgt-A2GalNAc2) were purified from cell culture supernatant with Protein A HiTrap Mabselect PrismA, (Cytiva) and formulated in PBS buffer pH 7, by using Amicon Concentrator, (4 ml, 30K MWCO). Due to lower levels of A2GalNAc2 (<70%) on the purified antibodies, the material was further polished by in-vitro glycoengineering to increase abundance of A2GalNAc2 glycan on Ptz. The GalNAc addition was performed using in vitro glycosylation (IVGE) in a reaction using 10 mM UDP-GalNAc, 2% (w/w) GalT1(Y285L), 100 mM MnCl2 in 25 mM Tris, pH 8 at 30 °C under mild rotation. The glycosylated mAb was purified from the reaction mixture with ProteinA sepharose (HiTrap MabSelect PrismA column GE Healthcare) according to manufacturer's recommendation using FPLC (Bio-Rad NGC, Germany). Thereafter, a desalting procedure using PD-10 (Sephadex 25, Sigma, Switzerland) was carried out for a buffer exchange to PBS pH 7. Sample was then treated with a Pierce High-Capacity Endotoxin Removal Spin Column, 0.25 mL (cat: 88273, Thermo), sterile filtrated, and diluted to 0.05 mg/ml with PBS pH 7. Ptz-gt1 antibody comprises a glycotag at the C-terminal part of the heavy chain (ANSTMMS addition with C-terminal lysine replaced by Alanine of the glycotag sequence). Ptz-hgt antibody comprises an inserted glycosite in the upper hinge region (LNLSS insertion after T223 position).

HepG2 Hepatocarcinoma cells (ATCC #HB-8065) were maintained in a low glucose DMEM medium (Sigma, Ref. D5546) supplemented with 10% FBS and 2 mM Glutamine. HepG2 cells were harvested using Accutase (Sigma/Merck, SCR005) and plated in flat-bottom 24-well plates at 0.1 million cells/well. Cells were left to recover for 72 hours at 37 °C in a cell culture incubator. Cells were then incubated for 24 hours with non-engineered pertuzumab (Ptz-A2F; Perjeta, obtained from pharmacy) or CGP-produced pertuzumab glycovariant antibodies at 1 μg/ml+IVIg (Hizentra, obtained from pharmacy) at 1 mg/ml, in cell culture medium, at 37 °C in cell culture incubator. Cells were then harvested and stained with anti-HER2 antibody (Human ErbB2/HER2 antibody, R&D, Ref. MAB 1129) at 1 μg/ml and anti-mouse IgG-fluorochrome secondary antibody (Goat anti-Mouse IgG (H+L) Cross-Adsorbed Secondary Antibody (PE), ThermoFisher, P-852), used at 2 μg/ml, according to standard flow cytometry staining protocol. Antibody MAB 1129 was shown to be non-competitive with pertuzumab for binding to HER2, ensuring that MAB 1129 can bind to HER2 even if pertuzumab is bound. Cells were immediately acquired on a flow cytometer and analyzed using FlowJo (Tri Star) software. The geometric mean fluorescence intensity (MFI) of HER2 was extracted for each condition. The HER2 MFI were adjusted to the pHrodo DOL (adjusted MFI). The adjusted MFI values were expressed as % of Ptz-A2F (normalized MFI).

Table 14 presents the adjusted and normalized HER2 MFI. Treatment with the control antibody Ptz-A2, which has no engineered glycosite and displays an A2 structure on the N297 Fc site did not reduce HER2 levels as compared to Ptz-A2F (104% of normalized HER2 MFI after treatment). In contrast, treatment with Ptz-gt1-A2GalNAc2 reduced normalized HER2 MFI to 55% and treatment with Ptz-hgt-A2GalNAc2 reduced normalized HER2 MFI to 68%. The reduction of HER2 by Ptz-gt1-A2GalNAc2 and Ptz-hgt-A2GalNAc2 was statistically significant compared to Ptz-A2F group (ratio paired t-test on adjusted MFI data) (Table 14). Interestingly, treatment with Ptz-86-A2GalNAc2 did not trigger HER2 degradation as compared to Ptz-A2F (96% normalized HER2 MFI). All the antibodies showed a similar HER2 binding capacity (Table 9), ruling out that different HER2 degradation capacity is linked to a reduced HER2 binding potency. Moreover, A2GalNAc2 antibodies containing the equivalent engineered glycosite at position 86 in the Fab fragment did show efficient uptake by HepG2 cells (Example 3, Example 5, Example 8), and high depletion potency of a circulating antigen, when injected in animals indicating efficient ASGPR engagement (Example 11), indicating that position 86 displays accessible, active glycans. This indicates that the position of glycan displayed on the antibody is important to enable efficient engagement of ASGPR when the antibody is bound on HER2.

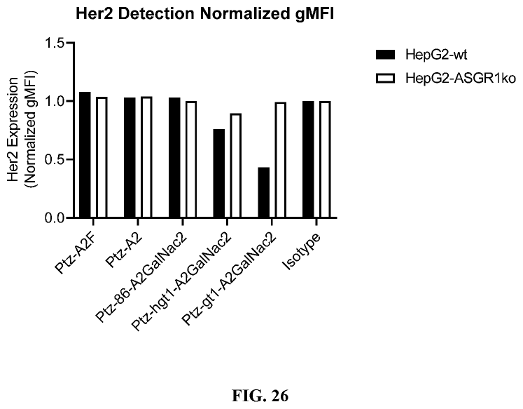

The ASGPR specificity of the HER2 reduction was verified using HepG2 cells knock-out for ASGPR1 (see Example 4). FIG. 26 shows that when HepG2 knock out for ASGPR1 (HepG2-ASGR1ko) are treated with Ptz-gt1-A2GalNAc2 or Ptz-hgt-A2GalNAc2 variants, no HER2 reduction is observed, as compared to isotype control or Ptz-A2F treated cells. Therefore, the HER2 reduction observed is mediated by an ASGPR-dependent mechanism. Altogether this study shows that A2GalNAc2 displaying antibodies can be used to remove a target molecule from a cell surface, by leveraging the ASGPR endocytic and lysosomal degradation pathway.

TABLE 14
HER2 degradation data on HepG2

| | | | Ptz-86- | Ptz-hgt- | Ptz-gt1- |
|---|---|---|---|---|---|
| | Ptz-A2F | Ptz-A2 | A2GalNAc2 | A2GalNAc2 | A2GalNAc2 |
| Mean | 100.0 | 103.8 | 95.70 | 67.79 | 55.40 |
| normalized | | (±2.478) | (±3.94) | (±4.77) | (±2.35) |
| HER2 MFI | | | | | |
| (±SEM) | | | | | |
| 95% CI of the | 100-100 | 98.08-109.6 | 89.63-101.8 | 58.15-77.43 | 43.73-67.06 |
| Mean | | | | | |
| normalized | | | | | |
| Mean | 14614 | 15146 | 14340 | 9121 | 7146 |
| adjusted MFI | (±5106) | (±5296) | (±3022) | (±2421) | (±7308) |
| (±SEM) | | | | | |
| p value vs | | NS | NS | <0.001 | <0.001 |
| Ptz-A2F | | | | | |

SEM: standard error of the mean.
CI: confidence interval.
P value was calculated with a ratio paired t-test on adjusted MFI data. The data are average from N= 7 independent experiments.
NS: not significant (>0.05).

### 8.16 Example 16—A2GalNAc2 mAb Internalises Large Immune Complexes

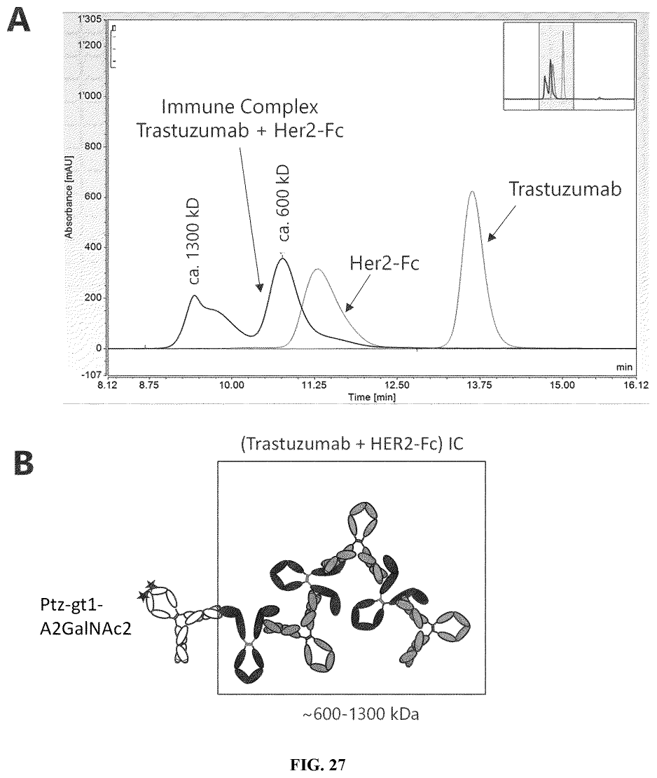

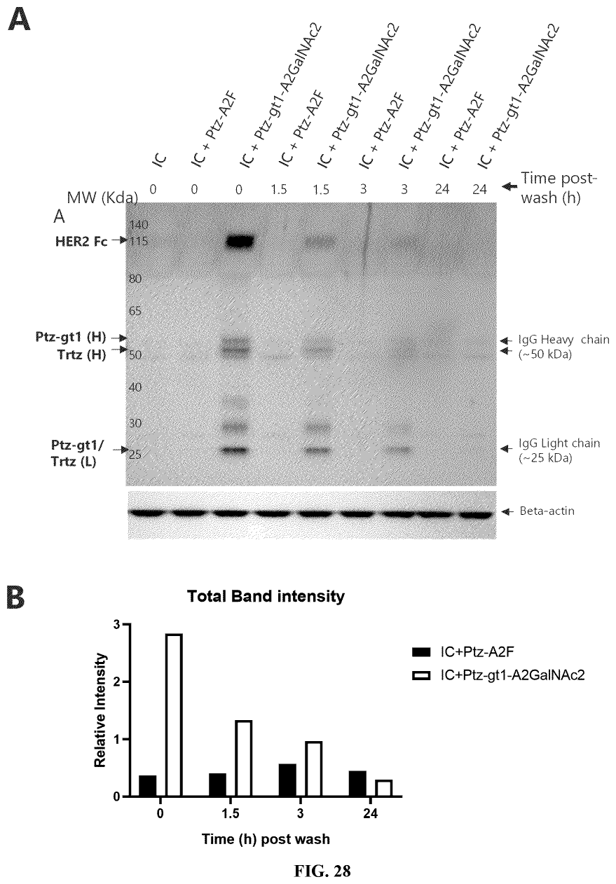

To assess whether A2GalNAC2 glycan displayed by a mAb can lead to internalization and degradation of large size structures, an experiment using immune complexes (ICs) was performed. ICs were preformed by mixing HER2-Fc (AcroBiosystems #HE2-H5253) at 0.1 mg/ml and trastuzumab (Herceptin©, obtained from local pharmacy), an anti-HER2 antibody at 0.055 mg/ml in PBS for 30 min at room temperature. The molar ratio of HER2-Fc and trastuzumab is approximately 1. The formation of the ICs was assessed using HPLC-SEC. In the IC solution no traces of monomeric trastuzumab and minimal levels of monomeric HER2-Fc were observed, indicating that all trastuzumab and HER2-Fc were engaged into large size complexes (FIG. 27). Two main complexes were observed at a size of ~600 and ~1300 kDa. The preformed ICs were incubated with pertuzumab (Ptz-A2F) or Ptz-gt1-GalNAc2, at 0.05 mg/ml, or PBS, for 30 min at room temperature. Ptz-gt1-A2GalNAc2 is a pertuzumab glycoengineered variant (Table 7, Table 9). Pertuzumab does not compete with trastuzumab for binding to HER2 and therefore can bind to HER2-Fc engaged in immune complexes with trastuzumab (FIG. 27, Panel B). HepG2 cells were then treated with IC ±Pertuzumab-A2F or gt1-A2GalNAc2 or PBS. After 3 hours, non-internalized ICs were washed out (time 0=pulse) and HepG2 cells were further cultured for 1.5/3/24 hours to allow lysosomal degradation of internalized complexes (chase). Whole cell extracts from HepG2 were processed, and IC internalization/degradation was assessed by western blot using anti-human IgG H+L detection. This detection allows to visualize specifically Her2-Fc internalization (MW ~120 KDa), trastuzumab and Ptz-gt1 internalization. The heavy chain of Ptz-gt1 contains a glycotag and has therefore a higher molecular weight than heavy chain of trastuzumab. IC alone were not internalized. Similarly, non-glycoengineered Ptz-A2F was unable to drive significant internalization of IC. However Ptz-gt1-A2GalNAc2 led to significant HER2-Fc and trastuzumab internalization (FIG. 28, Panel A), indicating that Her2-Fc/trastuzumab ICs were internalized. In addition, the internalized complexes were rapidly degraded as the band intensities disappeared gradually during the chase period (FIG. 28, Panel A and FIG. 28, Panel B). These data therefore indicate that large size immune complexes (600-1300 kDa) can be efficiently internalized by a A2GalNAc2 displaying mAb.

ICs and large size protein aggregates can cause severe pathologies such as IgA nephropathy; systemic lupus erythematosus; amyloidosis; COVID; iTTP; cutaneous necrotizing vasculitis. Therefore, these data indicate that biologics compounds displaying A2GalNAc2 and able to bind a component of large size immune complexes or protein aggregates can be used to remove these pathogenic units from circulating and cause their degradation in hepatocytes via the lysosomal pathway.

### Example 17—Depletion of Target by A2GalNAc2 mAb and Fab Requires Low Degrader:Target Ratio

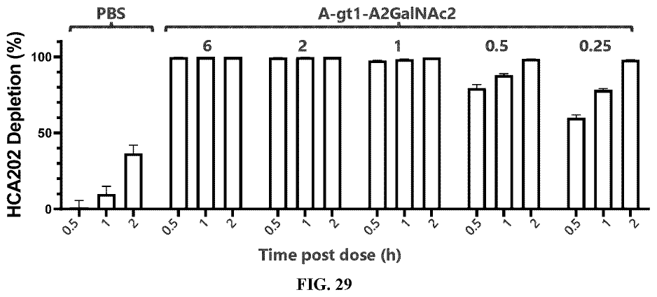

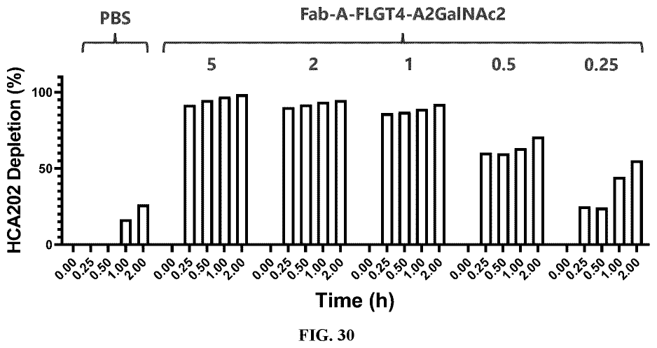

An experiment was setup to study the dose response pharmacodynamic of target depletion with a mAb or Fab compound, displaying A2GalNAc2 on a glycotag. Addition of a glycotag on mAb, Fab or MOG-Fc fusion protein was shown to drive efficient ASGPR mediated internalization and degradation in vitro and in vivo (Examples 8, 9 and 10). The experiment setup was very similar to the setup described in Example 11. Briefly, Rats were injected i.v with HCA202 target, followed by PBS or different doses of A-gt1-A2GalNAC2 or Fab-A-FLGT4-A2GalNAc2. The levels of HCA202 target were quantified in serum and expressed as % of HCA202 depletion from theoretical C_{zero} concentration. FIG. 29 shows the data with A-gt1 mAb, FIG. 30 shows the data with Fab-A-FLGT4 Fab compound. The depletion of HCA202 by A-gt1-A2GalNAc2 followed a dose response. When the degrader:target ratio fell below 1, the depletion of HCA202 at early timepoints was incomplete. The data highlight that a low degrader:target ratio of 1 (i.e 1 A-gt1 for 1 HCA202 target in molarity) still drives rapid and complete depletion of the target. Similarly the depletion of HCA202 by Fab-A-FLGT4-A2GalNAC2 followed a clear dose response, with incomplete depletion of the target observed at degrader:target lower than 1. At an degrader:target of 0.5, only 50% of the target was specifically depleted, as compared to PBS group and similarly at degrader:target of 0.25, only 25% of depletion was observed. This is in agreement with the monovalent interaction of Fab-A-FLGT4 with HCA202 target. The data also highlight that at degrader:target of 1 the target depletion by the Fab A2GalNAc2 compound is rapid and complete.

Altogether, these data show that target depletion with mAb or Fab compound displaying A2GalNAC2 on a glycotag leads to expected dose response pharmacology and that the mechanism is very fast and efficient at low degrader to target ratio.

### 9. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated herein by reference in their entireties.

### 10. SEQUENCE LISTING

| SEQ | | |
|---|---|---|
| ID | | |
| NO. | Description | Sequence |
| 1 | β4GalNAcT3 of | MGSPRAARPPLLLRPVKLLRRRFRLLLALAVVSVGLWTL |
| | Homo sapiens | YLELVASAQVGGNPLNRRYGSWRELAKALASRNIPAVD |
| | | PHLQFYHPQRLSLEDHDIDQGVSSNSSYLKWNKPVPWLS |
| | | EFRGRANLHVFEDWCGSSIQQLRRNLHFPLYPHIRTTLR |
| | | KLAVSPKWTNYGLRIFGYLHPFTDGKIQFAIAADDNAEF |
| | | WLSLDDQVSGLQLLASVGKTGKEWTAPGEFGKFRSQIS |
| | | KPVSLSASHRYYFEVLHKQNEEGTDHVEVAWRRNDPGA |
| | | KFTIIDSLSLSLFTNETFLQMDEVGHIPQTAASHVDSSNAL |
| | | PRDEQPPADMLRPDPRDTLYRVPLIPKSHLRHVLPDCPY |
| | | KPSYLVDGLPLQRYQGLRFVHLSFVYPNDYTRLSHMET |
| | | HNKCFYQENAYYQDRFSFQEYIKIDQPEKQGLEQPGFEE |
| | | NLLEESQYGEVAEETPASNNQNARMLEGRQTPASTLEQ |
| | | DATDYRLRSLRKLLAQPREGLLAPFSKRNSTASFPGRTS |
| | | HIPVQQPEKRKQKPSPEPSQDSPHSDKWPPGHPVKNLPQ |
| | | MRGPRPRPAGDSPRKTQWLNQVESYIAEQRRGDRMRPQ |
| | | APGRGWHGEEEVVAAAGQEGQVEGEEEGEEEEEEEDM |
| | | SEVFEYVPVFDPVVNWDQTFSARNLDFQALRTDWIDLS |
| | | CNTSGNLLLPEQEALEVTRVFLKKLNQRSRGRYQLQRIV |
| | | NVEKRQDQLRGGRYLLELELLEQGQRVVRLSEYVSARG |
| | | WQGIDPAGGEEVEARNLQGLVWDPHNRRRQVLNTRAQ |
| | | EPKLCWPQGFSWSHRAVVHFVVPVKNQARWVQQFIKD |
| | | MENLFQVTGDPHFNIVITDYSSEDMDVEMALKRSKLRSY |
| | | QYVKLSGNFERSAGLQAGIDLVKDPHSIIFLCDLHIHFPA |
| | | GVIDAIRKHCVEGKMAFAPMVMRLHCGATPQWPEGYW |
| | | EVNGFGLLGIYKSDLDRIGGMNTKEFRDRWGGEDWELL |
| | | DRILQAGLDVERLSLRNFFHHFHSKRGMWSRRQMKTL |
| 2 | N-terminally | MIASSVRHAVILLLVAVAMMAAVIAMDYKDDDDKRYG |
| | truncated variant | SWRELAKALASRNIPAVDPHLQFYHPQRLSLEDHDIDQG |
| | of β4GalNAcT3 | VSSNSSYLKWNKPVPWLSEFRGRANLHVFEDWCGSSIQ |
| | of Homo sapiens | QLRRNLHFPLYPHIRTTLRKLAVSPKWTNYGLRIFGYLH |
| | (L. tarentolae | PFTDGKIQFAIAADDNAEFWLSLDDQVSGLQLLASVGKT |
| | signal peptide | GKEWTAPGEFGKFRSQISKPVSLSASHRYYFEVLHKQNE |
| | and Flag tag are | EGTDHVEVAWRRNDPGAKFTIIDSLSLSLFTNETFLQMD |
| | fused to the | EVGHIPQTAASHVDSSNALPRDEQPPADMLRPDPRDTLY |
| | putative | RVPLIPKSHLRHVLPDCPYKPSYLVDGLPLQRYQGLRFV |
| | catalytic domain | HLSFVYPNDYTRLSHMETHNKCFYQENAYYQDRFSFQE |
| | of human | YIKIDQPEKQGLEQPGFEENLLEESQYGEVAEETPASNNQ |
| | β4GalNAc-T3 | NARMLEGRQTPASTLEQDATDYRLRSLRKLLAQPREGL |
| | (amino acids | LAPFSKRNSTASFPGRTSHIPVQQPEKRKQKPSPEPSQDSP |
| | 57-999)) | HSDKWPPGHPVKNLPQMRGPRPRPAGDSPRKTQWLNQ |
| | | VESYIAEQRRGDRMRPQAPGRGWHGEEEVVAAAGQEG |
| | | QVEGEEEGEEEEEEEDMSEVFEYVPVFDPVVNWDQTFS |
| | | ARNLDFQALRTDWIDLSCNTSGNLLLPEQEALEVTRVFL |
| | | KKLNQRSRGRYQLQRIVNVEKRQDQLRGGRYLLELELL |
| | | EQGQRVVRLSEYVSARGWQGIDPAGGEEVEARNLQGLV |
| | | WDPHNRRRQVLNTRAQEPKLCWPQGFSWSHRAVVHFV |
| | | VPVKNQARWVQQFIKDMENLFQVTGDPHFNIVITDYSSE |
| | | DMDVEMALKRSKLRSYQYVKLSGNFERSAGLQAGIDLV |
| | | KDPHSIIFLCDLHIHFPAGVIDAIRKHCVEGKMAFAPMV |
| | | MRLHCGATPQWPEGYWEVNGFGLLGIYKSDLDRIGGM |
| | | NTKEFRDRWGGEDWELLDRILQAGLDVERLSLRNFFHH |
| | | FHSKRGMWSRRQMKTL |
| 3 | β4GalNAcT4 of | MPRLPVKKIRKQMKLLLLLLLLSCAAWLTYVHLGLVRQ |
| | Homo sapiens | GRALRQRLGYGRDGEKLTSETDGRGVHAAPSTQRAEDS |
| | | SESREEEQAPEGRDLDMLFPGGAGRLPLNFTHQTPPWRE |
| | | EYKGQVNLHVFEDWCGGAVGHLRRNLHFPLFPHTRTTV |
| | | KKLAVSPKWKNYGLRIFGFIHPARDGDVQFSVASDDNSE |
| | | FWLSLDESPAAAQLVAFVGKTGSEWTAPGEFTKFSSQVS |
| | | KPRRLMASRRYYFELLHKQDDRGSDHVEVGWRAFLPGL |
| | | KFEVISSAHISLYTDESALKMDHVAHVPQSPASHVGGRP |
| | | PQEETSADMLRPDPRDTFFLTPRMESSSLENVLEPCAYAP |
| | | TYVVKDFPIARYQGLQFVYLSFVYPNDYTRLTHMETDN |
| | | KCFYRESPLYLERFGFYKYMKMDKEEGDEDEEDEVQRR |
| | | AFLFLNPDDFLDDEDEGELLDSLEPTEAAPPRSGPQSPAP |
| | | AAPAQPGATLAPPTPPRPRDGGTPRHSRALSWAARAARP |
| | | LPLFLGRAPPPRPAVEQPPPKVYVTRVRPGQRASPRAPAP |
| | | RAPWPPFPGVFLHPRPLPRVQLRAPPRPPRPHGRRTGGPQ |
| | | ATQPRPPARAQATQGGREGQARTLGPAAPTVDSNLSSEA |
| | | RPVTSFLSLSQVSGPQLPGEGEEEEEGEDDGAPGDEAASE |
| | | DSEEAAGPALGRWREDAIDWQRTFSVGAVDFELLRSDW |
| | | NDLRCNVSGNLQLPEAEAVDVTAQYMERLNARHGGRF |
| | | ALLRIVNVEKRRDSARGSRFLLELELQERGGGRLRLSEY |
| | | VFLRLPGARVGDADGESPEPAPAASVRPDGRPELCRPLR |
| | | LAWRQDVMVHFIVPVKNQARWVAQFLADMAALHART |
| | | GDSRFSVVLVDFESEDMDVERALRAARLPRYQYLRRTG |
| | | NFERSAGLQAGVDAVEDASSIVFLCDLHIHFPPNILDGIR |
| | | KHCVEGRLAFAPVVMRLSCGSSPRDPHGYWEVNGFGLF |
| | | GIYKSDFDRVGGMNTEEFRDQWGGEDWELLDRVLQAG |
| | | LEVERLRLRNFYHHYHSKRGMWSVRSRKGSRTGAS |
| 4 | N-terminally | MIASSVRHAVILLLVAVAMMAAVIAMDYKDDDDKRGV |
| | truncated variant | HAAPSTQRAEDSSESREEEQAPEGRDLDMLFPGGAGRLP |
| | of β4GalNAcT4 | LNFTHQTPPWREEYKGQVNLHVFEDWCGGAVGHLRRN |
| | of Homo sapiens | LHFPLFPHTRTTVKKLAVSPKWKNYGLRIFGFIHPARDG |
| | L. tarentolae | DVQFSVASDDNSEFWLSLDESPAAAQLVAFVGKTGSEW |
| | signal peptide | TAPGEFTKFSSQVSKPRRLMASRRYYFELLHKQDDRGSD |
| | and Flag tag are | HVEVGWRAFLPGLKFEVISSAHISLYTDESALKMDHVAH |
| | fused to the | VPQSPASHVGGRPPQEETSADMLRPDPRDTFFLTPRMES |
| | putative | SSLENVLEPCAYAPTYVVKDFPIARYQGLQFVYLSFVYP |
| | catalytic domain | NDYTRLTHMETDNKCFYRESPLYLERFGFYKYMKMDK |
| | of human | EEGDEDEEDEVQRRAFLFLNPDDFLDDEDEGELLDSLEP |
| | β4GalNAc-T4 | TEAAPPRSGPQSPAPAAPAQPGATLAPPTPPRPRDGGTPR |
| | (amino acids 62- | HSRALSWAARAARPLPLFLGRAPPPRPAVEQPPPKVYVT |
| | 1039) | RVRPGQRASPRAPAPRAPWPPFPGVFLHPRPLPRVQLRA |
| | | PPRPPRPHGRRTGGPQATQPRPPARAQATQGGREGQART |
| | | LGPAAPTVDSNLSSEARPVTSFLSLSQVSGPQLPGEGEEE |
| | | EEGEDDGAPGDEAASEDSEEAAGPALGRWREDAIDWQR |
| | | TFSVGAVDFELLRSDWNDLRCNVSGNLQLPEAEAVDVT |
| | | AQYMERLNARHGGRFALLRIVNVEKRRDSARGSRFLLE |
| | | LELQERGGGRLRLSEYVFLRLPGARVGDADGESPEPAPA |
| | | ASVRPDGRPELCRPLRLAWRQDVMVHFIVPVKNQARW |
| | | VAQFLADMAALHARTGDSRFSVVLVDFESEDMDVERAL |
| | | RAARLPRYQYLRRTGNFERSAGLQAGVDAVEDASSIVFL |
| | | CDLHIHFPPNILDGIRKHCVEGRLAFAPVVMRLSCGSSPR |
| | | DPHGYWEVNGFGLFGIYKSDFDRVGGMNTEEFRDQWG |
| | | GEDWELLDRVLQAGLEVERLRLRNFYHHYHSKRGMWS |
| | | VRSRKGSRTGAS |
| 5 | Ceβ4GalNAcT | MAFRHLAVARLKSLLVLCAVLLLVHAMIYKIPSLYENLT |
| | (β4GalNAcT of | IGSSTLIADVDAMEAVLGNTASTSDDLLDTWNSTFSPISE |
| | Caenorhabditis | VNQTSFMEDIRPILFPDNQTLQFCNQTPPHLVGPIRVFLD |
| | elegans) | EPDFKTLEKIYPDTHAGGHGMPKDCVARHRVAIIVPYRD |
| | | REAHLRIMLHNLHSLLAKQQLDYAIFIVEQVANQTFNRG |
| | | KLMNVGYDVASRLYPWQCFIFHDVDLLPEDDRNLYTCP |
| | | IQPRHMSVAIDKFNYKLPYSAIFGGISALTKDHLKKINGF |
| | | SNDFWGWGGEDDDLATRTSMAGLKVSRYPTQIARYKM |
| | | IKHSTEATNPVNKCRYKIMGQTKRRWTRDGLSNLKYKL |
| | | VNLELKPLYTRAVVDLLEKDCRRELRRDFPTCF |
| 6 | N-terminally | MIASSVRHAVILLLVAVAMMAAAVIAMGSHHHHHHHH |
| | truncated variant | HHKIPSLYENLTIGSSTLIADVDAMEAVLGNTASTSDDLL |
| | of | DTWNSTFSPISEVNQTSFMEDIRPILFPDNQTLQFCNQTPP |
| | Ceβ4GalNAcT | HLVGPIRVFLDEPDFKTLEKIYPDTHAGGHGMPKDCVAR |
| | of | HRVAIIVPYRDREAHLRIMLHNLHSLLAKQQLDYAIFIVE |
| | Caenorhabditis | QVANQTFNRGKLMNVGYDVASRLYPWQCFIFHDVDLLP |
| | elegans | EDDRNLYTCPIQPRHMSVAIDKFNYKLPYSAIFGGISALT |
| | L. tarentolae | KDHLKKINGFSNDFWGWGGEDDDLATRTSMAGLKVSR |
| | signal peptide | YPTQIARYKMIKHSTEATNPVNKCRYKIMGQTKRRWTR |
| | and His tag are | DGLSNLKYKLVNLELKPLYTRAVVDLLEKDCRRELRRD |
| | fused to the | FPTCF |
| | putative | |
| | catalytic domain | |
| 7 | Ptβ4GalNAcT | MAQHSYSRITFSRTLICKAVILLVFTVMLLDYSYYLFSDR |
| | (β4GalNAcT of | KPINKIVAPGLAWISHLNQKLSDTKALSNNSFLPLKPHNI |
| | Parasteatoda | LLNRSLVSESSNSSKLYSSKVENKNISLNFINSSHLEPSTE |
| | tepidariorum) | CPLIPPKLVGRLKVLTESPPFVALEQMFTDLNPGGRFKPN |
| | | DCLSRSKVALIIPYRDREEHLRIFLHNVHPILQRQQLDYGI |
| | | YIIEETANMKFNRAMLMNIGYVEASKQYPYDCFIFHDVD |
| | | LIPEDDRNLYNCPEQPRHMSVAINTMNYRLPYKDIFGGV |
| | | SALSKKHMKIVNGFSNEFWGWGGEDDDMSNRIKHHGL |
| | | KISRYSSKIARYTMLKHKKDEPNPDRYNKLYKGKYRYT |
| | | KDGLSNLKYKRLEIVFKKLYTWVLVEIEPIK |
| 8 | N-terminally | MIASSVRHAVILLLVAVAMMAAAVIAMGSHHHHHHHH |
| | truncated variant | HHSDRKPINKIVAPGLAWISHLNQKLSDTKALSNNSFLPL |
| | of | KPHNILLNRSLVSESSNSSKLYSSKVENKNISLNFINSSHL |
| | Ptβ4GalNAcT | EPSTECPLIPPKLVGRLKVLTESPPFVALEQMFTDLNPGG |
| | of Parasteatoda | RFKPNDCLSRSKVALIIPYRDREEHLRIFLHNVHPILQRQQ |
| | tepidariorum | LDYGIYIIEETANMKFNRAMLMNIGYVEASKQYPYDCFI |
| | L. tarentolae | FHDVDLIPEDDRNLYNCPEQPRHMSVAINTMNYRLPYK |
| | signal peptide | DIFGGVSALSKKHMKIVNGFSNEFWGWGGEDDDMSNRI |
| | and His tag are | KHHGLKISRYSSKIARYTMLKHKKDEPNPDRYNKLYKG |
| | fused to the | KYRYTKDGLSNLKYKRLEIVFKKLYTWVL VEIEPIK |
| | putative | |
| | catalytic domain | |
| 9 | Stβ4GalNAcT | MGRVEGKGVLLWDQTVMCSPQYKGQANLHTFEDWCG |
| | (β4GalNAcT of | SSTAQLRMNLHYPLYPHSRTTVKKLAVSPRWMNYGLRI |
| | Salmo trutta) | FGYLHPYTDGEFVFAVSSDDNSEFWLSRDNSPLNLQLLA |
| | | WIGKTGMEWTAPGEFGKYASQTSRPVRLSVQRRYFFEIV |
| | | HKQNDRGTDHIEVAWQLNQEGLRFTVISSKYISLYSNES |
| | | ALRMSDIIHVPQTAASHTRSPSNQPDNQLSNQPAADMLR |
| | | VDPRDILYQIPALDSRFLQGVLPDCSYKPSYIIKGFPLLRY |
| | | QGLQFVHMSYIYPNDYTRLTHMETDNNCFYHDSPLYLD |
| | | RYGFSRYMRLDEPEGQEGESRGRGRGDTFFFGMDRLVE |
| | | GVDRGGGGERKGEEVGVSESLWGLEGDSEEVEGLSYDS |
| | | TPRPVFDPEVNWAQTFQVTPLDLQAMRSDWIDLRCNVS |
| | | GNLMLRSRDALPVVKAFMDKLNHRHHGRFTLVRVLNV |
| | | ERRVDGAQGSRYFLELELRDVNGQHLRLSHYIYALIRHR |
| | | LSRPRGLPPLRPAPEMLLCNPVGFHWNPTATVHFIVPVK |
| | | NQARWVQQLIVDMEGLYKTTGDPNFNLIITDYNSTDMDI |
| | | ETTLKASTLHRYQYLKLSGNFERSAGLQAGIDLITHSIVF |
| | | LCDLHIHFPPSIIDSVRKHCVEGHMAFAPIVMRLDCGATP |
| | | REPRGYWEVNGFGLLGIYKSDLDAAGGMNTHDFTDRW |
| | | GGEDWELLDRILQSGLEVERIYMRNFLHHYHSKRGMWN |
| | | RRTQRSGT |
| 10 | Hhβ4GalNAcT | MCVFQFGGDASEDSEAWRSSYTPQPWRPEYKGQANLHT |
| | (β4GalNAcT of | FEDWCGSSTAQLRMNLHYPLYPHSRTTVKKLAVSPRW |
| | Hucho hucho) | MNYGLRIFGYLHPYTDGEFVFAVSSDDNSEFWLSRDNSP |
| | | LNLQLLAWIGKTGLEWTAPGEFGKYASQTSRPVRLSVQ |
| | | RRYFFEIVHKQNDRGTDHIEVAWQLNQEGLRFTVISSKYI |
| | | SLYSNESALRMSDITHVPQTAASHTRSPSNQLDNQLSNQ |
| | | PAADMLRDDPRDTLYQIPALDSRFLQGVLPDCSYKPSYII |
| | | KGFPLLRYQGLQFVHMSYIYPNDYTRLTHMETDNTCFY |
| | | HDSPLYLDRYGFSRYMRLDESEGQEGESRVRGRVVGILR |
| | | RRKAVPEEGLDGEAPPNVGGASKKDSAPPDYGDDYDDY |
| | | AQRRRRKLFSLPQADKAPTHRNMSDTLTYKKKKRRKRR |
| | | ETNAAAVVVQQAVVQTRATEQDGLEIPPGPGSHTSLQTT |
| | | PAEQLRLPDRPAEQLRPVKSRAGEQKPEGKPADREDMG |
| | | PADRKQPRPRERPAEQNSAGLKILPAPQKLDHNLPVEGD |
| | | GPTVWLPQLQGRHQRVQQLGPRGWGNATPNPLKHLTP |
| | | RTVSNWSTRVGRGRRLVNRTLELWSEVTKQSTTQLPEV |
| | | KPPLIKLDHKATVTRKRVQVLETDIILRGQPQIKSRPQPD |
| | | YDNTLHGDLPPGVVRGMNRLVEGVDRGGGGEREGEEV |
| | | GVSESLWGLEGDSEEVEGLSYDSTPRPVFDPEVNWAQTF |
| | | QVTPLDLQAMRSDWIDLRCNVSGNLLLRPRDALPVVKA |
| | | FMDKLNHRHHGRFTLVRVLNVERRVDGAQGSRYFLELE |
| | | LRDVNGQHLRLSHYIYALIRHRRSRPQGLPPLRPAPEMLL |
| | | CNPVGFHWNPSATVHFIVPVKNQARWVQQLIVDMEGLY |
| | | KTTGDPNFNLIITDYNSTDMDIETTLKASTLHRYQYLKLS |
| | | GNFERSAGLQAGIDLITNEHSIVFLCDLHIYFPPSIIDSVRK |
| | | HCVEGHMAFAPIVMRLDCGATPREPRGYWEVNGFGLLG |
| | | IYKSDLDAAGGMNTHDFTDRWGGEDWELLDRILQSGLE |
| | | VERIYMRNFLHHYHSKRGMWNRRTQRRST″ |
| 11 | SPinv, a | MIASSVRHAVILLLVAVAMMAAVIA |
| | modified signal | |
| | peptide from | |
| | Leishmania | |
| | tarentolae | |
| | invertase | |
| 12 | SPinv, the | MIASSVRHAVILLLVAVAMMAAAVIA |
| | native signal | |
| | peptide from | |
| | Leishmania | |
| | tarentolae | |
| | invertase | |
| 13 | MGAT1 | MLKKQSAGLVLWGAILFVAWNALLLLFFWTRPAPGRPP |
| | (Alpha-1,3- | SVSALDGDPASLTREVIRLAQDAEVELERQRGLLQQIGD |
| | mannosyl- | ALSSQRGRVPTAAPPAQPRVPVTPAPAVIPILVIACDRST |
| | glycoprotein 2- | VRRCLDKLLHYRPSAELFPIIVSQDCGHEETAQAIASYGS |
| | beta-N- | AVTHIRQPDLSSIAVPPDHRKFQGYYKIARHYRWALGQV |
| | acetylglucosaminyl- | FRQFRFPAAVVVEDDLEVAPDFFEYFRATYPLLKADPSL |
| | transferase) | WCVSAWNDNGKEQMVDASRPELLYRTDFFPGLGWLLL |
| | of Homo sapiens | AELWAELEPKWPKAFWDDWMRRPEQRQGRACIRPEISR |
| | | TMTFGRKGVSHGQFFDQHLKFIKLNQQFVHFTQLDLSYL |
| | | QREAYDRDFLARVYGAPQLQVEKVRINDRKELGEVRV |
| | | QYTGRDSFKAFAKALGVMDDLKSGVPRAGYRGIVTFQF |
| | | RGRRVHLAPPLTWEGYDPSWN |
| 14 | MGAT2 | MRFRIYKRKVLILTLVVAACGFVLWSSNGRQRKNEALA |
| | (Alpha-1,6- | PPLLDAEPARGAGGRGGDHPSVAVGIRRVSNVSAASLVP |
| | mannosyl- | AVPQPEADNLTLRYRSLVYQLNFDQTLRNVDKAGTWAP |
| | glycoprotein 2- | RELVLVVQVHNRPEYLRLLLDSLRKAQGIDNVLVIFSHD |
| | beta-N- | FWSTEINQLIAGVNFCPVLQVFFPFSIQLYPNEFPGSDPRD |
| | acetylglucosaminyl- | CPRDLPKNAALKLGCINAEYPDSFGHYREAKFSQTKHH |
| | transferase) | WWWKLHFVWERVKILRDYAGLILFLEEDHYLAPDFYH |
| | of Homo sapiens | VFKKMWKLKQQECPECDVLSLGTYSASRSFYGMADKV |
| | | DVKTWKSTEHNMGLALTRNAYQKLIECTDTFCTYDDYN |
| | | WDWTLQYLTVSCLPKFWKVLVPQIPRIFHAGDCGMHHK |
| | | KTCRPSTQSAQIESLLNNNKQYMFPETLTISEKFTVV |
| | | AISPPRKNGGWGDIRDHELCKSYRRLQ |
| 15 | UAP1 (Isoform | MNINDLKLTLSKAGQEHLLRFWNELEEAQQVELYAELQ |
| | AGX1 of UDP- | AMNFEELNFFFQKAIEGFNQSSHQKNVDARMEPVPREVL |
| | N-acetyl | GSATRDQDQLQAWESEGLFQISQNKVAVLLLAGGQGTR |
| | hexosamine | LGVAYPKGMYDVGLPSRKTLFQIQAERILKLQQVAEKY |
| | pyrophosphorylase | YGNKCIIPWYIMTSGRTMESTKEFFTKHKYFGLKKENVIF |
| | of Homo sapiens) | FQQGMLPAMSFDGKIILEEKNKVSMAPDGNGGLYRALA |
| | | AQNIVEDMEQRGIWSIHVYCVDNILVKVADPRFIGFCIQK |
| | | GADCGAKVVEKTNPTEPVGVVCRVDGVYQVVEYSEISL |
| | | ATAQKRSSDGRLLFNAGNIANHFFTVPFLRDVVNVYEPQ |
| | | LQHHVAQKKIPYVDTQGQLIKPDKPNGIKMEKFVFDIFQ |
| | | FAKKFVVYEVLREDEFSPLKNADSQNGKDNPTTARHAL |
| | | MSLHHCWVLNAGGHFIDENGSRLPAIPRLKDANDVPIQC |
| | | EISPLISYAGEGLESYVADKEFHAPLIIDENGVHELVKNGI |
| 16 | GALK2 | MATESPATRRVQVAEHPRLLKLKEMFNSKFGSIPKFYVR |
| | (N-acetyl | APGRVNIIGEHIDYCGYSVLPMAVEQDVLIAVEPVKTYA |
| | galactosamine | LQLANTNPLYPDFSTSANNIQIDKTKPLWHNYFLCGLKGI |
| | kinase of Homo | QEHFGLSNLTGMNCLVDGNIPPSSGLSSSSALVCCAGLV |
| | sapiens) | TLTVLGRNLSKVELAEICAKSERYIGTEGGGMDQSISFLA |
| | | EEGTAKLIEFSPLRATDVKLPSGAVFVIANSCVEMNKAA |
| | | TSHFNIRVMECRLAAKLLAKYKSLQWDKVLRLEEVQAK |
| | | LGISLEEMLLVTEDALHPEPYNPEEICRCLGISLEELRTQI |
| | | LSPNTQDVLIFKLYQRAKHVYSEAARVLQFKKICEEAPE |
| | | NMVQLLGELMNQSHMSCRDMYECSCPELDQLVDICRKF |
| | | GAQGSRLTGAGWGGCTVSMVPADKLPSFLANVHKAYY |
| | | QRSDGSLAPEKQSLFATKPGGGALVLLEA |
| 17 | GalE | MAEKVLVTGGAGYIGSHTVLELLEAGYLPVVIDNFHNA |
| | (UDP-glucose | FRGGGSLPESLRRVQELTGRSVEFEEMDILDQGALQRLF |
| | 4-epimeraseof | KKYSFMAVIHFAGLKAVGESVQKPLDYYRVNLTGTIQL |
| | Homo sapiens) | LEIMKAHGVKNLVFSSSATVYGNPQYLPLDEAHPTGGCT |
| | | NPYGKSKFFIEEMIRDLCQADKTWNAVLLRYFNPTGAH |
| | | ASGCIGEDPQGIPNNLMPYVSQVAIGRREALNVFGNDYD |
| | | TEDGTGVRDYIHVVDLAKGHIAALRKLKEQCGCRIYNL |
| | | GTGTGYSVLQMVQAMEKASGKKIPYKVVARREGDVAA |
| | | CYANPSLAQEELGWTAALGLDRMCEDLWRWQKQNPSG |
| | | FGTQA |
| 18 | CjGne | MKILISGGAGYIGSHTLRQFLKTDHEICVLDNLSKGSKIAI |
| | (UDP- | EDLQKTRAFKFFEQDLSDFQGVKALFEREKFDAIVHFAA |
| | GlcNAc/Glc 4- | SIEVFESMQNPLKYYMNNTVNTTNLIETCLQTGVNKFIFS |
| | epimerase of | STAATYGEPQTPVVSETSPLAPINPYGRSKLMSEEVLRDA |
| | Campylobacter | SMANPEFKHCILRYFNVAGACMDYTLGQRYPKATLLIK |
| | jejuni) | VAAECAAGKRDKLFIFGDDYDTKDGTCIRDFIHVDDISS |
| | | AHLAALDYLKENESNVFNVGYGHGFSVKEVIEAMKKVS |
| | | GVDFKVELAPRRAGDPSVLISDASKIRNLTSWQPKYDDL |
| | | ELICKSAFDWEKQC |
| 19 | UDP-GalNAc | MNRANDTSSNLKLISLVVLIVQTTALVLTLRYSQTQKSE |
| | transporter of C. | GPRYLSSTAVVCAEIIKLITCFFVIYRNNGYRFSGMLNEL |
| | elegans | NREIFASPQTRADSLKVAVPAIMYVIQNNLLFFALKKLD |
| | (CeC03H5.2) | AATYQVTYQLKILTTAIFSVTMLGKSLHRYNWMALILLT |
| | | AGVALVQYPSGDSTTSKSTAAEHDASDNILGLGAVLAA |
| | | CFSSGFAGVYFEKILKTSKVSLWIRNIQLAFFSVFGALLV |
| | | CWLYDWQAISDDGFLRGYNGVIWIVVLLQAYGGLVIAL |
| | | VVKYADNILKGFAVSLSIILSSFTSWLVLGDLTITTTFAIG |
| | | ATVVIFATFLYGHEPKSTPAEAHNA |
| 20 | tracrRNA | AGU UAA AAU AAG GCU AGU CCG UUA UCA A |
| 21 | OGNT1_3prime | GAA AUC CGA UGG UUU UAG AGC UAU |
| 22 | OGNT2_3prime | GGA ACG GCC UGG UUU UAG AGC UAU |
| 23 | OGNT2_5prime | CAA CCG AAU CCG UUU UAG AGC UAU |
| 24 | OGNTL_5prime | CGA ACU CGC AGG UUU UAG AGC UAU |
| 25 | OGNT1_5prime | CGG GUG AGG UGG UUU UAG AGC UAU |
| 26 | OGNTL_3prime | GUG GCG CCA AGG UUU UAG AGC UAU |
| 27 | Primer 360 | CCATGGCCAGATGCCAAGCTTACTC |
| 28 | Primer 363 | CGGAACACAGACCTGCCATCTGTGG |
| 29 | Primer 362 | CCACAGATGGCAGGTCTGTGTTCCG |
| 30 | Primer 361 | CATCCACCTCTGCTCGTCGTCCTTC |
| 31 | Primer 290 | CGCACCTCGGCTTCAACTGCCAATG |
| 32 | Primer 291 | AACGTGCCTGAAGCCCGTAGCTTTG |
| 33 | Primer 292 | GGCGACGGATGATGCCCACCATTTG |
| 34 | Primer 293 | GGAGGCAACCAAGAAGGCGTCAGAG |
| 35 | Primer 127 | GCTCAGCTGCGAGAGCGTAAACCTCCGTAACAATG |
| 36 | Primer 128 | AGCTAAACTCTGCACTCATCTGCGCTTCCGCAAGG |
| 37 | Primer 161 | CGACCGCGATATCTGCTTTCCATTCTGCAGTTCTC |
| 38 | Primer 160 | CGCACAGGGTGACGAGGAAAGCAGTATCGAAACAG |

What is claimed:

1. A glycoengineered bifunctional degrader, wherein the bifunctional degrader (i) specifically binds to a target protein and (ii) comprises an N-glycan of the structure: 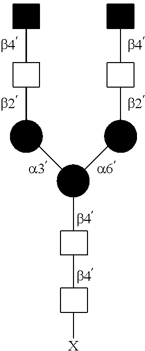
linked to the bifunctional degrader at one or more N-glycosylation sites, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader.

2. The glycoengineered bifunctional degrader of claim 1, wherein the N-glycan is linked to the bifunctional degrader at least one N-glycosylation site.

3. The glycoengineered bifunctional degrader of claim 2, wherein the N-glycan is linked to the bifunctional degrader at least two N-glycosylation sites.

4. The glycoengineered bifunctional degrader of any one of claims 1-3, wherein the N-glycan is linked to the bifunctional degrader at one, two, three, or four N-glycosylation sites.

5. The glycoengineered bifunctional degrader of claim 4, wherein the N-glycan is linked to the bifunctional degrader at one N-glycosylation site.

6. The glycoengineered bifunctional degrader of claim 4, wherein the N-glycan is linked to the bifunctional degrader at two N-glycosylation sites.

7. The glycoengineered bifunctional degrader of claim 4, wherein the N-glycan is linked to the bifunctional degrader at three N-glycosylation sites.

8. The glycoengineered bifunctional degrader of claim 4, wherein the N-glycan is linked to the bifunctional degrader at four N-glycosylation sites.

9. The glycoengineered bifunctional degrader of any one of claims 1-8, wherein the one or more N-glycosylation sites are distal to a target-specific binding location of the bifunctional degrader.

10. The glycoengineered bifunctional degrader of claim 9, wherein at least one or at least two N-glycosylation sites are distal to the target-specific binding location.

11. The glycoengineered bifunctional degrader of claim 9, wherein all of the N-glycosylation sites are distal to the target-specific binding location.

12. The glycoengineered bifunctional degrader of any one of claims 9-11, wherein the target-specific binding location is a variable region of an antibody or antigen-binding fragment (Fab), or an ectodomain of an Fc-fusion protein.

13. The glycoengineered bifunctional degrader of any one of claims 1-12, wherein the one or more N-glycosylation sites are not present in a wild-type, natural, synthetic, or commercial precursor of the bifunctional degrader.

14. The glycoengineered bifunctional degrader of any one of claims 1-13, wherein one or more N-glycosylation sites present in a wild-type, natural, synthetic, or commercial precursor of the bifunctional degrader (“natural N-glycosylation site”) have been deleted, mutated, or functionally inactivated.

15. The glycoengineered bifunctional degrader of claim 14, wherein at least one or at least two natural N-glycosylation sites have been deleted, mutated, or functionally inactivated.

16. The glycoengineered bifunctional degrader of claim 15, wherein all natural N-glycosylation sites have been deleted, mutated, or functionally inactivated.

17. The glycoengineered bifunctional degrader of any one of claims 14-16, wherein the one or more natural N-glycosylation sites are located at or proximal to the target-specific binding location of the wild-type, natural, synthetic, or commercial precursor of the bifunctional degrader.

18. The glycoengineered bifunctional degrader of any one of claims 1-17, wherein the amino acid residue is Asn.

19. The glycoengineered bifunctional degrader of any one of claims 4-18, wherein the N-glycosylation site comprises a consensus sequence of N-X-S/T or N-X-C, wherein X is any amino acid except proline.

20. The glycoengineered bifunctional degrader of any one of claims 1-19, wherein the bifunctional degrader is an antibody or fragment thereof.

21. The glycoengineered bifunctional degrader of any one of claims 1-20, wherein the bifunctional degrader is a Fab fragment of an antibody.

22. The glycoengineered bifunctional degrader of any one of claims 1-21, wherein the glycoengineered bifunctional degrader is an antibody.

23. The glycoengineered bifunctional degrader of claim 22, wherein the antibody is a monoclonal or polyclonal antibody.

24. The glycoengineered bifunctional degrader of claim 22, wherein the antibody is a recombinant antibody.

25. The glycoengineered bifunctional degrader of claim 22, wherein the antibody is humanized, chimeric or fully human.

26. The glycoengineered bifunctional degrader of claim 22, wherein the antibody has a glycan to protein ratio of 2 to 1, 4 to 1, 6 to 1, 8 to 1, or 10 to 1.

27. The glycoengineered bifunctional degrader of any one of claims 20-26, wherein the N-glycan is linked to an N-glycosylation site of the light chain of the antibody or fragment thereof.

28. The glycoengineered bifunctional degrader of any one of claims 20-27, wherein the N-glycan is linked to an N-glycosylation site of the heavy chain of the antibody or fragment thereof.

29. The glycoengineered bifunctional degrader of any one of claims 20-28, wherein one or more N-glycosylation sites are located in a constant domain of the antibody or fragment thereof.

30. The glycoengineered bifunctional degrader of any one of claims 20-29, wherein one or more N-glycosylation sites are located in a variable domain of the antibody or fragment thereof.

31. The glycoengineered bifunctional degrader of any one of claims 20-30, wherein one or more N-glycosylation sites are located on the Fab region of the antibody.

32. The glycoengineered bifunctional degrader of any one of claims 20-31, wherein one or more N-glycosylation sites are located on the Fc region of the antibody.

33. The glycoengineered bifunctional degrader of any one of claims 20-32, wherein one or more N-glycosylation sites are located in the hinge region of the antibody.

34. The glycoengineered bifunctional degrader of any one of claims 31-33, wherein at least one of the N-glycosylation sites is not present in a wild-type form of the antibody.

35. The glycoengineered bifunctional degrader of any one of claims 1-34, wherein at least two N-glycosylation sites of the Fab region of the antibody are glycosylated by the N-glycan.

36. The glycoengineered bifunctional degrader of any one of claims 1-35, wherein one N-glycosylation site of the Fab region is located on each of the two heavy chain polypeptides of the antibody, and wherein each of said N-glycosylation sites is glycosylated by the N-glycan.

37. The glycoengineered bifunctional degrader of any one of claims 1-36, wherein at least two N-glycosylation sites of the Fc region of the antibody are glycosylated by the N-glycan.

38. The glycoengineered bifunctional degrader of any one of claims 1-37, wherein the Fab region contains more of the N-glycans than the Fc region.

39. The glycoengineered bifunctional degrader of any one of claims 1-38, wherein, the Fab region contains two more of the N-glycans than the Fc region.

40. The glycoengineered bifunctional degrader of any one of claims 1-37, wherein the Fc region contains more of the N-glycans than the Fab region.

41. The glycoengineered bifunctional degrader of any one of claims claims 1-37 or 40, wherein the Fc region contains two or four more of the N-glycans than the Fab region.

42. The glycoengineered bifunctional degrader of any one of claims 1-37, wherein the Fc region and the Fab region contain the same number of the N-glycans.

43. The glycoengineered bifunctional degrader of any one of claims 1-42, wherein the glycoengineered bifunctional degrader binds to an autoantibody and comprises an autoantigen or immunogenic fragment thereof.

44. The glycoengineered bifunctional degrader of any one of claims 1-43, wherein the glycoengineered bifunctional degrader comprises a moiety that specifically binds to the target protein, and wherein the target protein is associated with a disease.

45. The glycoengineered bifunctional degrader of any one of claims 1-44, wherein the target protein is a cell surface molecule or a non-cell surface molecule.

46. The glycoengineered bifunctional degrader of claim 45, wherein the cell surface molecule is a receptor.

47. The glycoengineered bifunctional degrader of claim 45, wherein the non-cell surface molecule is an extracellular protein.

48. The glycoengineered bifunctional degrader of claim 47, wherein the extracellular protein is an autoantibody, a hormone, a cytokine, a chemokine, a blood protein, or a central nervous system (CNS) protein.

49. The glycoengineered bifunctional degrader of any one of claims 44-48, wherein the target protein associated with a disease is upregulated in the disease compared to a non-disease state.

50. The glycoengineered bifunctional degrader of any one of claims 44-49, wherein the target protein associated with a disease is expressed in the disease compared to a non-disease state.

51. The glycoengineered bifunctional degrader of any one of claims 44-50, wherein the target protein associated with a disease is involved in cancer progression.

52. The glycoengineered bifunctional degrader of any one of claims 44-51, wherein the target protein associated with disease comprises TNFα, HER2, EGFR, HER3, VEGFR, CD20, CD19, CD22, αvβ3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPα, CD38, CD73, or TGF-β, Bombesin R, CAIX, CD13, CD44, v6, Emmprin, Endoglin, EpCAM, EphA2, FAP-α, Folate R, GRP78, IGF-1R, Matriptase, Mesothelin, sMET/HGFR, MT1-MMP, MT6-MMP, PSCA, PSMA, Tn antigen, and uPAR, TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, LILRB1, LILRB2, VEGF-R, CXCR4, CXCL12, CSF-1, CD47, aggregated light chains or aggregated transthyretin.

53. The glycoengineered bifunctional degrader of any one of claims 44-52, wherein the target protein associated with disease is involved in an autoimmune disease, and wherein the target protein is an antibody binding to TSHRα, MOG, AChR-α1, noncollagen domain 1 of the α3 chain of type IV collagen (α3NC1), ADAMTS13, Desmoglein-1/3, or GPIb/IX, GPIIb/IIIa, GPIa/IIa, NMDA receptor, glutamic acid decarboxylase (GAD), amphiphysin, or gangliosides GM1, GD3 or GQ1B.

54. The glycoengineered bifunctional degrader of any one of claims 44-53, wherein the disease comprises a cancer.

55. The glycoengineered bifunctional degrader of any one of claims 44-53, wherein the disease comprises an autoimmune disease.

56. A composition comprising a population of bifunctional degraders according to any one of claims 1-56, wherein the population of bifunctional degraders has an N-glycan profile that is at least 30%, at least 40%, at least 50%, at least 60%, at least 70%, at least 80%, at least 90%, at least 95%, at least 98%, or about 100% homogeneous at one or more of the N-glycosylation site(s).

57. The composition of claim 56, wherein the N-glycan profile is at least 30%, at least 40%, at least 50%, at least 60%, at least 70%, at least 80%, at least 90%, at least 95%, or at least 98% homogenous at one of the N-glycosylation sites.

58. The composition of claim 56 or 57, wherein the N-glycan profile is at least 30%, at least 40%, at least 50%, at least 60%, at least 70%, at least 80%, at least 90%, at least 95%, or at least 98% homogeneous at two of the N-glycosylation sites.

59. The composition of any one of claims 56-58, wherein the N-glycan profile is at least 50% homogeneous at one or more of the N-glycosylation site(s).

60. The composition of any one of claims 56-58, wherein the N-glycan profile is at least 60% homogeneous at one or more of the N-glycosylation site(s).

61. The composition of any one of claims 56-58, wherein the N-glycan profile is at least 70% homogenous at one or more of the N-glycosylation site(s).

62. The composition of any one of claims 56-58, wherein the N-glycan profile is at least 80% homogeneous at one or more of the N-glycosylation site(s).

63. The composition of any one of claims 56-58, wherein the N-glycan profile is at least 90% homogeneous at one or more of the N-glycosylation site(s).

64. The composition of any one of claims 56-58, wherein the N-glycan profile is at least 95% homogenous at one or more of the N-glycosylation site(s).

65. The composition of any one of claims 56-58, wherein the N-glycan profile is at least 98% homogenous at one or more of the N-glycosylation site(s).

66. The composition of any one of claims 56-65, wherein the homogeneity of the N-glycan profile at one or more of the N-glycosylation sites is determined by N-glycan analysis, glycopeptide analysis or intact protein analysis.

67. The composition of any one of claims 56-66, wherein the N-glycan profile comprises about 30% to 40%, about 40% to about 50%, about 50% to about 60%, about 60% to about 70%, about 70% to about 80%, about 80% to about 90%, or about 90% to about 100% of an N-glycan of the structure: 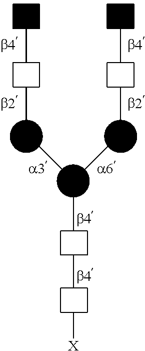
at one or more of the N-glycosylation site(s), wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader.

68. The composition of claim 67, wherein the N-glycan profile comprises about 90% to about 100% of the N-glycan at one N-glycosylation site.

69. The composition of claim 67 or 68, wherein the N-glycan profile comprises about 95% to about 100% of the N-glycan at one N-glycosylation site.

70. The composition of any one of claims 67-69, wherein the N-glycan profile comprises about 90% to about 100% of the N-glycan at two N-glycosylation sites, respectively.

71. The composition of any one of claims 67-69, wherein the N-glycan profile comprises about 80% to about 90% of the N-glycan at two N-glycosylation sites, collectively.

72. The composition of any one of claims 67-69, wherein the N-glycan profile comprises about 90% to about 100% of the N-glycan at two N-glycosylation sites, collectively.

73. The composition of any one of claims 67-69, wherein the N-glycan profile comprises about 90% to about 100% of the N-glycan at three or more of the N-glycosylation site(s), respectively.

74. The composition of any one of claims 67-69, wherein the N-glycan profile comprises about 70% to about 100% of the N-glycan at three or more of the N-glycosylation site(s), collectively.

75. The composition of any one of claims 67-74, wherein the relative amount of the N-glycan at one or more of the N-glycosylation sites in determined by N-glycan analysis or glycopeptide analysis.

76. The composition of any one of claims 56-75, wherein the population of bifunctional degraders has an N-glycan profile comprising at least 30%, at least 40%, at least 50%, at least 60%, at least 70%, at least 80%, at least 90%, at least 95%, or at least 98% of an N-glycan of the structure: 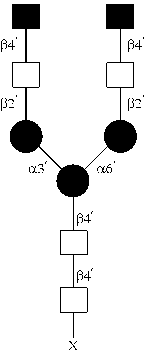
among all glycans in the N-glycan profile, wherein the black square represents an N-acetyl galactosamine (GalNAc), the white square represents an N-acetylglucosamine (GlcNAc) residue and the black circle represents a mannose (Man) residue, and wherein X represents an amino acid residue of the bifunctional degrader.

77. The composition of any one of claims 56-76, wherein the population of bifunctional degraders has an N-glycan profile comprising at least 30% of the N-glycan among all glycans in the N-glycan profile.

78. The composition of any one of claims 56-76, wherein the population of bifunctional degraders has an N-glycan profile comprising at least 40% of the N-glycan among all glycans in the N-glycan profile.

79. The composition of any one of claims 56-76, wherein the population of bifunctional degraders has an N-glycan profile comprising at least 50% of the N-glycan among all glycans in the N-glycan profile.

80. The composition of any one of claims 56-76, wherein the population of bifunctional degraders has an N-glycan profile comprising at least 60% of the N-glycan among all glycans in the N-glycan profile.

81. The composition of any one of claims 56-76, wherein the population of bifunctional degraders has an N-glycan profile comprising at least 70% of the N-glycan among all glycans in the N-glycan profile.

82. The composition of any one of claims 56-76, wherein the population of bifunctional degraders has an N-glycan profile comprising at least 80% of the N-glycan among all glycans in the N-glycan profile.

83. The composition of any one of claims 56-76, wherein the population of bifunctional degraders has an N-glycan profile comprising at least 90% of the N-glycan among all glycans in the N-glycan profile.

84. The composition of any one of claims 56-76, wherein the population of bifunctional degraders has an N-glycan profile comprising at least 95% of the N-glycan among all glycans in the N-glycan profile.

85. The composition of any one of claims 56-76, wherein the population of bifunctional degraders has an N-glycan profile comprising at least 98% of the N-glycan among all glycans in the N-glycan profile.

86. The composition of any one of claims 56-85, wherein the population of bifunctional degraders has an N-glycan profile comprising about 30% to about 40%, about 40% to about 50%, about 50% to about 60%, about 60% to about 70%, about 70% to about 80%, about 80% to about 90%, or about 90% to about 100% of the N-glycan among all glycans in the N-glycan profile.

87. The composition of any one of claims 56-86, wherein the population of bifunctional degraders has an N-glycan profile comprising about 30% to about 40% of the N-glycan among all glycans in the N-glycan profile.

88. The composition of any one of claims 56-86, wherein the population of bifunctional degraders has an N-glycan profile comprising about 40% to about 50% of the N-glycan among all glycans in the N-glycan profile.

89. The composition of any one of claims 56-86, wherein the population of bifunctional degraders has an N-glycan profile comprising about 50% to about 60% of the N-glycan among all glycans in the N-glycan profile.

90. The composition of any one of claims 56-86, wherein the population of bifunctional degraders has an N-glycan profile comprising about 60% to about 70% of the N-glycan among all glycans in the N-glycan profile.

91. The composition of any one of claims 56-86, wherein the population of bifunctional degraders has an N-glycan profile comprising about 70% to about 80% of the N-glycan among all glycans in the N-glycan profile.

92. The composition of any one of claims 56-86, wherein the population of bifunctional degraders has an N-glycan profile comprising about 80% to about 90% of the N-glycan among all glycans in the N-glycan profile.

93. The composition of any one of claims 56-86, wherein the population of bifunctional degraders has an N-glycan profile comprising about 90% to about 100% of the N-glycan among all glycans in the N-glycan profile.

94. The composition of any one of claims 76-93, wherein the relative amount of the N-glycan among all glycans in the N-glycan profile is determined by N-glycan analysis, glycopeptide analysis or intact protein analysis.

95. The composition of any one of claims 56-94, wherein the population has an N-glycan profile that is at least 60%, at least 70%, at least 80%, at least 90%, at least 95%, or at least 98% homogenous.

96. The composition of claim 95, wherein the population has an N-glycan profile that is at least 60% homogeneous.

97. The composition of claim 95, wherein the population has an N-glycan profile that is at least 70% homogeneous.

98. The composition of claim 95, wherein the population has an N-glycan profile that is at least 80% homogeneous.

99. The composition of claim 95, wherein the population has an N-glycan profile that is at least 90% homogeneous.

100. The composition of claim 95, wherein the population has an N-glycan profile that is at least 95% homogeneous.

101. The composition of claim 95, wherein the population has an N-glycan profile that is at least 98% homogeneous.

102. The composition of any one of claims 56-101, wherein the population has an N-glycan profile that is about 60% to about 70%, about 70% to about 80%, about 80% to about 90%, or about 90% to about 100% homogeneous.

103. The composition of claim 102, wherein the population has an N-glycan profile that is about 60% to about 70% homogeneous.

104. The composition of claim 102, wherein the population has an N-glycan profile that is about 70% to about 80% homogeneous.

105. The composition of claim 102, wherein the population has an N-glycan profile that is about 80% to about 90% homogeneous.

106. The composition of claim 102, wherein the population has an N-glycan profile that is about 90% to about 100% homogeneous.

107. The composition of claim 95-106, wherein the homogeneity of the N-glycan profile is determined by N-glycan analysis, glycopeptide analysis or intact protein analysis.

108. The composition of any one of claim 56-107, wherein the bifunctional degrader in said population is expressed from one or more nucleic acid sequences in a Leishmania host cell.
